(19) United States
(12) Patent Application Publication
(10) Pub. No.: US 2011/0028509 A1
(43) Pub. Date: Feb. 3, 2011

(54) Sulfonamides

(75) Inventors: Stefano Crosignani, St. Genis-Pouilly (FR); Christophe Cleva, La Tour (FR); Christos Tsaklakidis, Weinheim (DE); Lars Burgdorf, Frankfurt Am Main (DE)

Correspondence Address: SALIWANCHIK LLOYD & SALIWANCHIK;A PROFESSIONAL ASSOCIATION, PO Box 142950, GAINESVILLE, FL, 32614, US

(73) Assignee: MERCK SERONO SA, COINSINS, VAUD (CH)

(21) Appl. No.: 12/937,074

(22) PCT Filed: Apr. 8, 2009
(86) PCT No.: PCT/EP09/54204

§ 371 Date: Oct. 8, 2010

Related U.S. Application Data
(60) Provisional application No. 61/124,601, filed on Apr. 17, 2008.

(30) Foreign Application Priority Data
Apr. 11, 2008 (EP) 08154366.2

(51) Int. Cl. A61K 31/18; C07C 311/19; C07D 307/14; C07D 213/42; C07D 333/20; C07D 401/12; C07D 413/12; C07D 257/04; C07D 471/04; A61K 31/341; A61K 31/4402; A61K 31/381; A61K 31/4439; A61K 31/41; A61K 31/444; A61K 31/4725; A61K 31/4375; A61P 29/00; A61P 19/02; A61P 3/10; A61P 1/00; A61P 1/16; A61P 25/00; A61P 27/02; A61P 1/04; A61P 17/06; A61P 11/06

(52) U.S. Cl. 514/299; 564/92; 549/493; 546/337; 549/77; 546/268.4; 564/89; 546/269.1; 548/252; 546/261; 546/148; 546/122; 514/604; 514/471; 514/357; 514/438; 514/340; 514/381; 514/335; 514/307

(57) ABSTRACT

The invention relates to compounds of formula I

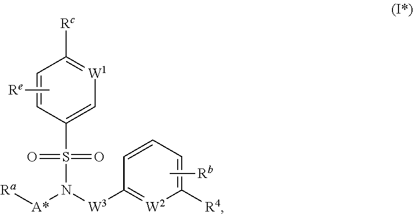

wherein R¹, R², R⁴, R^{a}, R^{b}, R^{c}, R^{e}, A*, W¹, W² and W³ are as defined in claim 16, for the treatment of CXCR3 related diseases.

The present invention is directed to compounds, which are modulators of chemokine receptor activity, preferably CXCR3 activity, and are useful in the prevention or treatment of certain inflammatory and immunoregulatory disorders and diseases, including asthma and allergic diseases, as well as autoimmune pathologies such as multiple sclerosis, rheumatoid arthritis and atherosclerosis. Compounds of the present invention are also useful for the treatment and prophylaxis of cancers. The present invention is also directed to compounds which are useful in the treatment and prophylaxis of other diseases such as angiogenesis, tumour formation, growth and propagation, ocular diseases, choroidal neovascularisation and diabetic retinopathy, neurodegeneration. The invention is also directed to pharmaceutical compositions comprising these compounds and the use of these compounds and compositions in the prevention or treatment of other diseases in which CXCR3 chemokine receptors are involved.

In a first embodiment, the present invention relates to compounds according to formula I*:

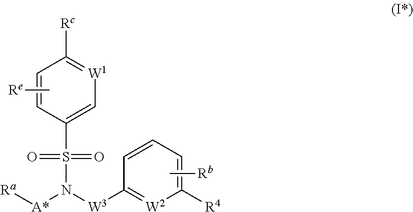

wherein
A* represents V, a C-1 to C-6 alkylen group that is unsubstituted or substituted by R^{f}, R^{g}, carbonyl (═O), or by a group —C(O)—OR^{f} or —C(O)NR^{f}R^{g}
V represents a —CO— group, a linear or branched (C1-C6)-alkylen group, or a bond.
W¹, W² are independently of one another N or CH,
W³ represents CR¹R² or a C-1 to C-6 alkylen group that is unsubstituted or substituted by R^{f}, R^{g}, carbonyl (═O), or by a group —C(O)—OR^{f} or —C(O)NR^{f}R^{g}
R^{a} denotes Ar or Het,
R^{b} denotes Hal, Ar, CN, Het, —NO₂, —N(R³)₂, —NH—C(O)A, —COOR³, —COOA, —C(O)—NHSO₂A, —C(O)—NHSO₂Het, —C(O)—NHSO₂Ar, Cyc, CONHZ, OR^{f} or a group —C(O)—NHQR^{d}, —NH—C(O)QR^{d}, —COOH or tetrazolyl or oxadiazolyl, hydroxyl-substituted oxadiazolyl, which may all be unsubstituted or substituted by alkyl having 1 to 8 carbon atoms
   or if R^{a} is substituted Ar or substituted Het, also H,
   or, if R^{a} is Het or substituted Ar, or if R^{c} is H, F, Br, I, CN, CF₃, OCF₃, NO₂, Het, tetrazol, alkyl having 1 to 6 carbon atoms, or alkoxy having 1 to 6 carbon atoms, or if W² is N, or if W¹ is N, or if R¹ and R² are alkyl having 1 to 3 carbon atoms, or R¹ and R² build together with the atom to which they are attached a carbocyclic or heterocyclic ring having 3 to 7 atoms, or if V represents a CO or a linear or branched (C2-C6)-alkylen group, or a bond, or if W3 represents a C-2 to C-5 alkylen group that is unsubstituted or substituted by R^{f}, R^{g}, carbonyl (═O), or by a group —C(O)—OR^{f} or —C(O)NR^{f}R^{g},
   or if A* represents C-2 to C-5 alkylen group that is unsubstituted or substituted by R^{f}, R^{g}, carbonyl (═O), or by a group —C(O)—OR^{f}, —C(O)NR^{f}R^{g},
   then R^{b} also denotes a group —C(O)—NHA, —C(O)—NHHet, —C(O)—NHQR^{d} or —C(O)—NHAr
Z denotes one of the following groups:

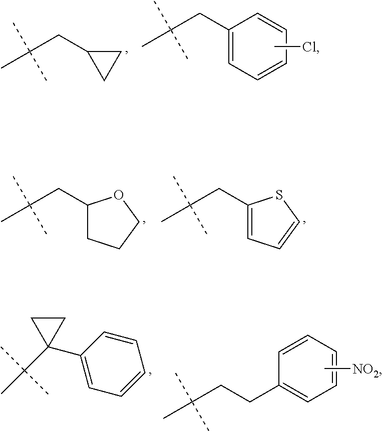

A denotes a branched or linear alkylen having 1 to 12 carbon atoms wherein one or more, preferably 1 to 7H atoms may be replaced by Hal, OR³, N(R³)₂, Het, Ar, NHCOOR³, COOR³, —CON(R³)₂, and wherein one or more CH₂-groups may be replaced by O, NR³, OCO, NHCO, SO₂, and/or by —CH═CH—, —C≡C—, or denotes cycloalkyl, cycloalkylen or cycloalkylalkylen having 3 to 7 ring C-atoms.
R³ denotes H, alkyl having 1 to 6 carbon atoms wherein 1 or more H atom may be replaced by Ar.
R^{c} denotes H, Hal, CN, CF₃, OCF₃, Het, NO₂, tetrazol, alkyl having 1 to 6 carbon atoms or alkoxy having 1 to 6 carbon atoms,

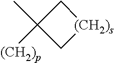

Q is (CR¹R²)ₚ, (CH₂)ₚ, (CH₂)ₚSO₂(CH₂)_{p′}, or
R^{d} denotes H, Ar, Het or cycloalkyl having 3 to 7 carbon atoms
R^{e} denotes H, Hal, NH₂, NO₂, Ar, O—Ar, preferably O-phenyl, Het or cycloalkyl having 3 to 7 carbon atoms, or R^{f}
R^{f}, R^{g} are independently of one another H, Ar, Het, or low alkyl or R^{f} and R^{g} build together with the atom or atoms at which they are attached a carbocyclic or heterocyclic ring having 3 to 7 atoms
R¹, R² are independently of one another H, alkyl, alkyloxy, hydroxy, hydroxyalkyl, amino, aminoalkyl, alkylamino, alkylaminoalkyl, carboxy, alkyloxycarbonyl, aminocarbonyl or alkylaminocarbonyl, or R¹ and R² build together with the atom or atoms at which they are attached a carbocyclic or heterocyclic ring having 3 to 7 atoms
   or R¹, R² are independently of one another H, alkyl having 1 to 3 carbon atoms, or R¹ and R² build together with the atom to which they are attached a carbocyclic or heterocyclic ring having 3 to 7 atoms, or R¹ is a (C1-C5)-alkylen linked to R^{a};
R⁴ denotes H or OR³
Hal denotes F, Cl, Br, or I.
Ar denotes a monocyclic or bicyclic, saturated, unsaturated or aromatic carbocyclic ring having 6 to 14 carbon atoms, which is unsubstituted or monosubstituted, disubstituted or trisubstituted by alkyl having 1 to 8 carbon atoms, alkoxy having 1 to 8 carbon atoms, Hal, CF₃, OCF₃, NO₂, N(R³)₂, COOR³, COR³, SO₂N(R³)₂, COHet, Het, OHet, OR³, CONH(CH₂)ₚN(R³)₂, Cyc, SO₂N(R³)₂, CN, and/or acyl.
Het denotes a monocyclic or bicyclic, saturated, unsaturated or aromatic heterocyclic ring having 1 to 4 N, O and/or S atoms or one CO function, which is unsubstituted or monosubstituted, disubstituted or trisubstituted by alkyl having 1 to 8 carbon atoms, alkoxy having 1 to 8 carbon atoms, Hal, CF₃, OCF₃, NO₂CN, N(R³)₂, COOR³, COR³, SO₂N(R³)₂, COAr, OR³, Ar, CONH(CH₂)ₚN(R³)₂, Cyc, SO₂N(R³)₂, Ar, OAr, and/or acyl,
Cyc denotes a cycloalkyl having 3 to 12 carbon atoms, which is unsubstituted or monosubstituted, disubstituted, trisubstituted by OR³, Hal, CN,
p, p′ are each independently of one another 0, 1, 2, 3, 4, 5 or 6,
s is 0, 1, 2, 3 or 4
   and pharmaceutically acceptable derivatives, solvates, tautomers, salts and stereoisomers thereof, including mixtures thereof in all ratios.

C-1 to C-5 alkylen group denotes methylen, ethylen propylene, butylen or pentylen that is unsubstituted or mono-, di- or trisubstituted by low alkyl, preferably methylen or propylen

Low alkyl denotes methyl, ethyl, propyl or butyl preferably methyl, ethyl or tert-butyl

The carbocyclic or heterocyclic ring having 3 to 7 atoms denotes the cyclopropyl, cyclobutyl, cyclopentyl, cyclohexyl, oxetanyl, tetrahydrofuranyl, tetrahydropyranyl, pyrrolidinyl or piperidinyl ring preferably the cyclopropyl or piperidinyl ring

Acyl denotes a group —C(O)—OR^{f} or —C(O)NR^{f}R^{g}

Hal denotes preferably F, Cl, Br or I, preferably F, Cl or Br.

In a second embodiment, the present invention relates to compounds according to formula (I):

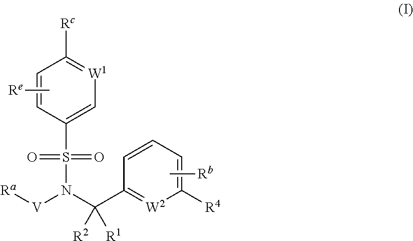

wherein
V represents a —CO— group, a linear or branched (C1-C6)-alkylen group, or a bond.
W¹, W² are independently of one another N or CH,
R^{a} denotes Ar or Het,
R^{b} denotes CN, Ar, Het, —NO₂, —N(R³)₂, —NH—C(O)A, —COOR³, —COOA, —C(O)—NHSO₂A, —C(O)—NHSO₂Het, —C(O)—NHSO₂Ar, Cyc, —CONHZ, or if R^{a} is substituted Ar or substituted Het, also H,
   or, if R^{a} is Het or substituted Ar, or if R^{c} is H, F, Br, I, CN, CF₃, OCF₃, NO₂, Het, tetrazol, alkyl having 1 to 6 carbon atoms, or alkoxy having 1 to 6 carbon atoms, or if W² is N, or if W¹ is N, or if R¹ and R² are alkyl having 1 to 3 carbon atoms, or R¹ and R² build together with the atom to which they are attached a carbocyclic or heterocyclic ring having 3 to 7 atoms, or if V represents a CO or a linear or branched (C2-C6)-alkylen group, or a bond, R^{b} also denotes a group —C(O)—NHA, —C(O)—NHHet, or —C(O)—NHAr
Z denotes one of the following groups:

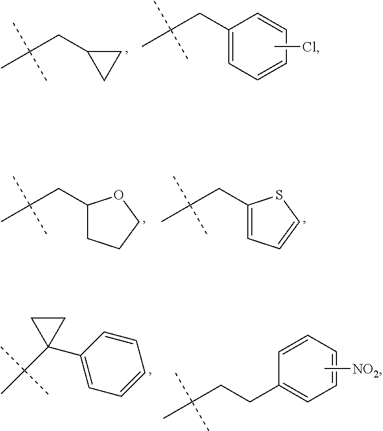

A denotes a branched or linear alkylen having 1 to 12 carbon atoms wherein one or more, preferably 1 to 7H atoms may be replaced by Hal, OR³, N(R³)₂, Het, Ar, NHCOOR³, COOR³, —CON(R³)₂, and wherein one or more CH₂-groups may be replaced by O, NR³, OCO, NHCO, SO₂, and/or by —CH═CH—, —C≡C—, or denotes cycloalkyl, cycloalkylen or cycloalkylalkylen having 3 to 7 ring C-atoms.
R³ denotes H, alkyl having 1 to 6 carbon atoms wherein 1 or more H atom may be replaced by Ar.
R^{c} denotes H, Hal, CN, CF₃, OCF₃, NO₂, Het, tetrazol, alkyl having 1 to 6 carbon atoms, or alkoxy having 1 to 6 carbon atoms,
O is (CH²)ₚ, (CH₂)ₚSO₂(CH₂)_{p′}, or

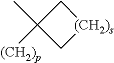

R^{e} denotes H, Hal, NH₂, NO₂, Ar, O—Ar, preferably O-phenyl, Het, alkyl having 1 to 6 carbon atoms, cycloalkyl having 3 to 7 carbon atoms
R¹, R² are independently of one another H, alkyl having 1 to 3 carbon atoms, or R¹ and R² build together with the atom to which they are attached a carbocyclic or heterocyclic ring having 3 to 7 atoms, or R¹ is a (C1-C6)-alkylen linked to R^{a};
R⁴ denotes H or OR³,
Hal denotes F, Cl, Br, or I.
Ar denotes a monocyclic or bicyclic, saturated, unsaturated or aromatic carbocyclic ring having 6 to 14 carbon atoms, which is unsubstituted or monosubstituted, disubstituted or trisubstituted by alkyl having 1 to 8 carbon atoms, alkoxy having 1 to 8 carbon atoms, Hal, CF₃, OCF₃, NO₂, N(R³)₂, COOR³, COR³, SO₂N(R³)₂, COHet, Het, OHet, OR³, CONH(CH₂)ₚN(R³)₂, Cyc, SO₂N(R³)₂, and/or CN,
Het denotes a monocyclic or bicyclic, saturated, unsaturated or aromatic heterocyclic ring having 1 to 4 N, O and/or S atoms or one CO function, which is unsubstituted or monosubstituted, disubstituted or trisubstituted by alkyl having 1 to 8 carbon atoms, alkoxy having 1 to 8 carbon atoms, Hal, CF₃, OCF₃, NO₂, N(R³)₂, COOR³, COR³, SO₂N(R³)₂, COAr, OR³, Ar, CONH(CH₂)ₚN(R³)₂, Cyc, SO₂N(R³)₂, Ar, OAr, and/or CN,
Cyc denotes a cycloalkyl having 3 to 12 carbon atoms, which is unsubstituted or monosubstituted, disubstituted, trisubstituted by OR³, Hal, CN,
p, p′ are each independently of one another 0, 1, 2, 3, 4, 5 or 6,
s is 0, 1, 2, 3 or 4
   and pharmaceutically acceptable derivatives, solvates, tautomers, salts and stereoisomers thereof, including mixtures thereof in all ratios.

Chemokines are chemotactic cytokines that are released by a wide variety of cells to attract macrophages, T cells, eosinophils, basophils and neutrophils to sites of inflammation (reviewed in Schall, Cytokine, 3:165-183 (1991), Schall, et al., Curr. Opin. Immunol., 6:865-873 (1994) and Murphy, Rev. Immun., 12:593-633 (1994)). In addition to stimulating chemotaxis, other changes can be selectively induced by chemokines in responsive cells, including changes in cell shape, transient rises in the concentration of intracellular free calcium ions, granule exocytosis, integrin upregulation, formation of bioactive lipids (e.g., leukotrienes) and respiratory burst, associated with leukocyte activation. Thus, the chemokines are early triggers of the inflammatory response, causing inflammatory mediator release, chemotaxis and extravasation to sites of infection or inflammation.

There are four classes of chemokines, CXC (α), CC (β), C(γ), and CX3C (δ), depending on whether the first two cysteines are separated by a single amino acid (C—X—C), are adjacent (C—C), have a missing cysteine pair (C), or are separated by three amino acids (CXC3). The α-chemokines, such as interleukin-8 (IL-8), melanoma growth stimulatory activity protein (MGSA), and stromal cell derived factor 1 (SDF-1) are chemotactic primarily for neutrophils and lymphocytes, whereas β-chemokines, such as RANTES, MIP-1α, MIP-1β, monocyte chemotactic protein-1 (MCP-1), MCP-2, MCP-3 and eotaxin are chemotactic for macrophages, T-cells, eosinophils and basophils (Deng, et al., Nature, 381:661-666 (1996)). The C chemokine lymphotactin shows specificity for lymphocytes (Kelner, et al., Science, 266:1395-1399 (1994)) while the CX3C chemokine fractalkine shows specificity for lymphocytes and monocytes (Bazan, et al., Nature, 385:640-644 (1997).

Chemokine receptors, such as CCR1, CCR2, CCR2A, CCR2B, CCR3, CCR4, CCR5, CCR6, CCR7, CCR8, CCR9, CXCR1, CXCR2, CXCR3, CXCR4, CXCR5, CX3CR1, and XCR1 have been implicated as being important mediators of inflammatory and immunoregulatory disorders and diseases, including asthma and allergic diseases, as well as autoimmune pathologies such as rheumatoid arthritis and atherosclerosis.

The CXCR3 chemokine receptor is expressed primarily in T lymphocytes, and its functional activity can be measured by cytosolic calcium elevation or chemotaxis. The receptor was previously referred to as GPR9 or CKR-L2. Its chromosomal location is unusual among the chemokine receptors in being localized to Xq13. Ligands that have been identified that are selective and of high affinity are the CXC chemokines, IP10, MIG and ITAC.

The highly selective expression of CXCR3 makes it an ideal target for intervention to interrupt inappropriate T cell trafficking. The clinical indications for such intervention are in T-cell mediated autoimmune diseases such as multiple sclerosis, rheumatoid arthritis, and type I diabetes. Inappropriate T-cell infiltration also occurs in psoriasis and other pathogenic skin inflammation conditions, although the diseases may not be true autoimmune disorders. In this regard, up-regulation of IP-10 expression in keratinocytes is a common feature in cutaneous immunopathologies. Inhibition of CXCR3 can be beneficial in reducing rejection in organ transplantation (Hancock, J. exp. Med. Vol 192, 2000). Ectopic expression of CXCR3 in certain tumors, especially subsets of B cell malignancies indicate that selective inhibitors of CXCR3 will have value in tumor immunotherapy, particularly attenuation of metastasis. In view of the clinical importance of CXCR3, the identification of compounds that modulate CXCR3 function represents an attractive avenue into the development of new therapeutic agents. It has been found that the compounds of formula I are preferably binding selectively to CXCR3.

Therefore, the compounds of formula I are useful in treating disorders or conditions influenced by CXCR3, such as an inflammatory or immune condition or disease in a subject. Preferably, the compounds of formula I are useful in the treatment of an inflammatory or immune condition or disease is selected from the group consisting of neurodegenerative diseases, multiple sclerosis, systemic lupus erythematosus, rheumatoid arthritis, atherosclerosis, encephalitis, meningitis, hepatitis, nephritis, sepsis, sarcoidosis, psoriasis, eczema, uticaria, type I diabetes, asthma, conjunctivitis, otitis, allergic rhinitis, chronic obstructive pulmonary disease, sinusitis, dermatitis, inflammatory bowel disease, Behcet's syndrome, gout, viral infections, bacterial infections, organ transplant conditions and skin transplant conditions.

The invention further relates to the manufacture of a medicament for the improvement of vascular function, either alone or in combination with other active compounds or therapies. In one embodiment, the present invention relates to compounds according to formula (I′):

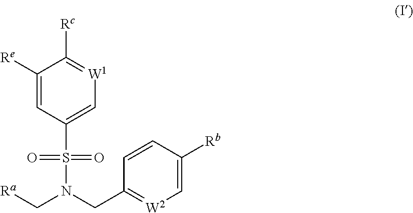

wherein
W¹, W² are independently of one another N or CH,
R^{a} denotes phenyl or pyridyl,
R^{b} denotes a group —C(O)—NHQR^{d} or tetrazolyl or oxadiazolyl, hydroxyl-substituted oxadiazolyl which may all be unsubstituted or substituted by alkyl having 1 to 8 carbon atoms
R^{c} denotes Hal, CN, CF₃, OCF₃, NO₂ or alkoxy having 1 to 6 carbon atoms,

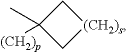

Q is (CH₂)ₚ, (CH₂)ₚSO₂(CH₂)_{p′}, or
   R^{d} denotes H, Ar, Het or cycloalkyl having 3 to 7 carbon atoms
   R^{e} denotes H or Hal
   Hal denotes F, Cl, Br, or I.
   Ar denotes a monocyclic or bicyclic, saturated, unsaturated or aromatic carbocyclic ring having 6 to 14 carbon atoms, which is unsubstituted or monosubstituted, disubstituted or trisubstituted by alkyl having 1 to 8 carbon atoms, alkoxy having 1 to 8 carbon atoms, Hal, CF₃, OCF₃, NO₂ and/or CN

Het denotes a monocyclic or bicyclic, saturated, unsaturated or aromatic heterocyclic ring having 1 to 4 N, O and/or S atoms which is unsubstituted or monosubstituted, disubstituted or trisubstituted by alkyl having 1 to 8 carbon atoms, alkoxy having 1 to 8 carbon atoms, Hal, CF₃, OCF₃, NO₂ and/or CN,

p, p′ are each independently of one another 0, 1, 2, 3, 4, 5 or 6,
s is 0, 1, 2, 3 or 4
and pharmaceutically acceptable derivatives, solvates, tautomers, salts and stereoisomers thereof, including mixtures thereof in all ratios.

The compounds according to Formula (I) and related formulae may be prepared from readily available starting materials using the following general methods and procedures. It will be appreciated that where typical or preferred experimental conditions (i.e. reaction temperatures, time, moles of reagents, solvents etc.) are given, other experimental conditions can also be used unless otherwise stated. Optimum reaction conditions may vary with the particular reactants or solvents used, but such conditions can be determined by the person skilled in the art, using routine optimisation procedures.

The following abbreviations refer respectively to the definitions below: aq (aqueous), h (hour), g (gram), L (liter), mg (milligram), MHz (Megahertz), min. (minute), mm (millimeter), mmol (millimole), mM (millimolar), m.p. (melting point), eq (equivalent), mL (milliliter), L (microliter), ACN (acetonitrile), AcOH (acetic acid), CDCl₃ (deuterated chloroform), CD₃OD (deuterated methanol), CH₃CN (acetonitrile), c-hex (cyclohexane), DCC (dicyclohexyl carbodiimide), DCM (dichloromethane), DIC (diisopropyl carbodiimide), DIEA (diisopropylethyl-amine), DMF (dimethylformamide), DMSO (dimethylsulfoxide), DMSO-d₆ (deuterated dimethylsulfoxide), EDC (1-(3-dimethyl-amino-propyl)-3-ethylcarbodiimide), ESI (Electro-spray ionization), EtOAc (ethyl acetate), Et₂O (diethyl ether), EtOH (ethanol), HATU (dimethylamino-([1,2,3]triazolo[4,5-b]pyridin-3-yloxy)-methylene]-dimethyl-ammonium hexafluorophosphate), HPLC (High Performance Liquid Chromatography), i-PrOH (2-propanol), K₂CO₃ (potassium carbonate), LC (Liquid Chromatography), MeOH (methanol), MgSO₄ (magnesium sulfate), MS (mass spectrometry), MTBE (Methyl tert-butyl ether), NaHCO₃ (sodium bicarbonate), NaBH₄ (sodium borohydride), NMM (N-methyl morpholine), NMR (Nuclear Magnetic Resonance), PyBOP (benzotriazole-1-yl-oxy-tris-pyrrolidino-phosphonium hexafluorophosphate), RT (room temperature), Rt (retention time), SPE (solid phase extraction), TBTU (2-(1-H-benzotriazole-1-yl)-1,1,3,3-tetramethyluronium tetrafluoro borate), TEA (triethylamine), TFA (trifluoroacetic acid), THF (tetrahydrofuran), TLC (Thin Layer Chromatography), UV (Ultraviolet).

Depending on the nature of W¹, W², V, R^{a}, R^{b}, R^{c}, R^{e}, R¹, R² and R⁴, in Formula (I) and related formulae, different synthetic strategies may be selected for the synthesis of compounds of Formula (I). In the process illustrated in the following schemes R^{a}, R^{c}, R^{d}, R^{e}, V, W¹, W², R¹, R² and R⁴, are as above defined in the description. Y and L denote a leaving group.

Throughout the specification, the term leaving group preferably denotes Cl, Br, I or a reactively modified OH group, such as, for example, an activated ester, an imidazolide or alkylsulfonyloxy having 1 to 6 carbon atoms (preferably methylsulfonyloxy or trifluoromethylsulfonyloxy) or arylsulfonyloxy having 6 to 10 carbon atoms (preferably phenyl- or p tolylsulfonyloxy).

Leaving groups of this type for activation of the carboxyl group in typical acylation reactions are described in the literature (for example in the standard works, such as Houben-Weyl, Methoden der organischen Chemie [Methods of Organic Chemistry], Georg-Thieme-Verlag, Stuttgart).

Activated esters are advantageously formed in situ, for example through addition of HOBt (1-Hydroxybenzotriazol) or N-hydroxysuccinimide. Preferably, L is a Cl or Br.

In general, the compounds according to Formula (I) and related formulae may be prepared from readily available starting materials. If such starting materials are not commercially available they may be prepared by standard synthetic techniques. The following general methods and procedures described hereinafter in the examples may be employed to prepare compounds of Formula I.

Generally, compounds of Formula (II) or (IIa) can by prepared by coupling a carboxylic acid of Formula V wherein R^{a}, R^{c}, R^{e}, V, W¹, W², R¹, R² and R⁴, are defined as above with an amine of Formula VI, wherein Q and R^{d} are as above defined, as outlined in Scheme 1. General protocols for such coupling are given below in the Examples, using conditions and methods well known to those skilled in the art to prepare an amide bond from an amine and a carboxylic acid, with standard coupling agents, such as but not limited to 1-alkyl-2-chloropyridinium salt or preferably polymer-supported 1-alkyl-2-chloropyridinium salt (polymer-supported Mukaiyama's reagent), 1-methyl-2-chloropyridinium iodide (Mukaiyama's reagent), DCC, DIC, preferably EDC, in the presence or absence of bases such as TEA, DIEA, NMM in a suitable solvent such as DCM, THF or DMF, at a temperature between 20° C. to 50° C., preferably at room temperature, for a few hours, e.g. one hour to 24 h. Alternatively, a carboxylic acid derivative (e.g. acyl chloride (Vb)) may be coupled with the amine, using conditions and methods well known to those skilled in the art, in the presence of bases such as TEA, DIEA, NMM in a suitable solvent such as DCM, THF or DMF, at a temperature from about 20° C. to about 50° C., preferably at room temperature, for a few hours, e.g. one hour to 24 h.

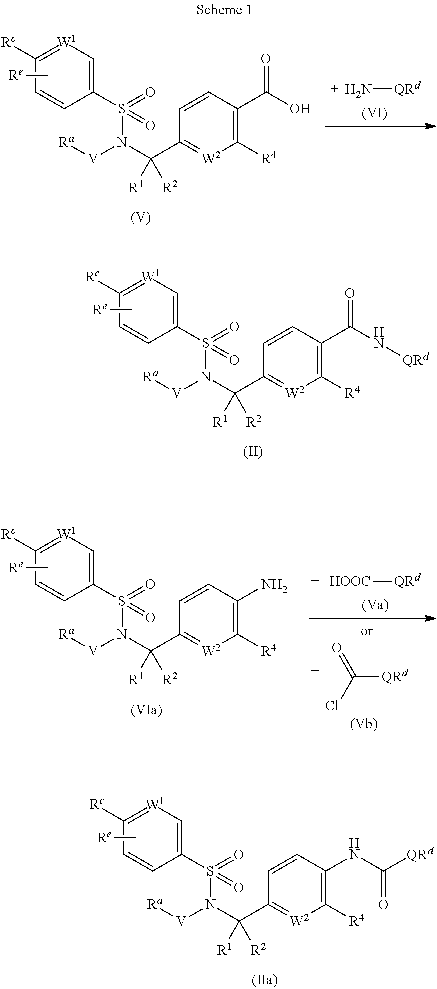

Alternatively, Compounds of formula (II*) and (IIa*) can by prepared by coupling a carboxylic acid of Formula (V) with an amine of Formula (VI*), (Va*) or (Vb*), (scheme 1b), following the above protocol, wherein G′ denotes Het or a linear or branched (C1-C6)alkylene, wherein 1, 2 or 3H atoms may be replaced by OR³, CON(R³)₂, CO₂R³, an aryl group, preferably a phenyl, and/or 2 geminal H atom may form a Cyc group, and wherein 1 or 2 CH₂ group may be replaced by SO₂, wherein R³ is as defined above.

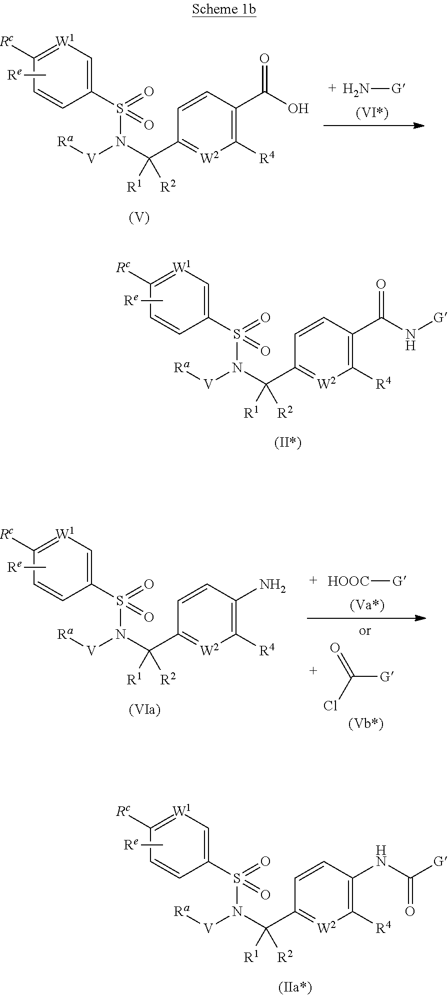

The compounds of Formula V, wherein V, R^{a}, R^{c}, R^{e}, W¹, W², R¹, R² and R⁴ are defined as above, are commercially available or can be obtained in a 3-step protocol as outlined in Scheme 2.

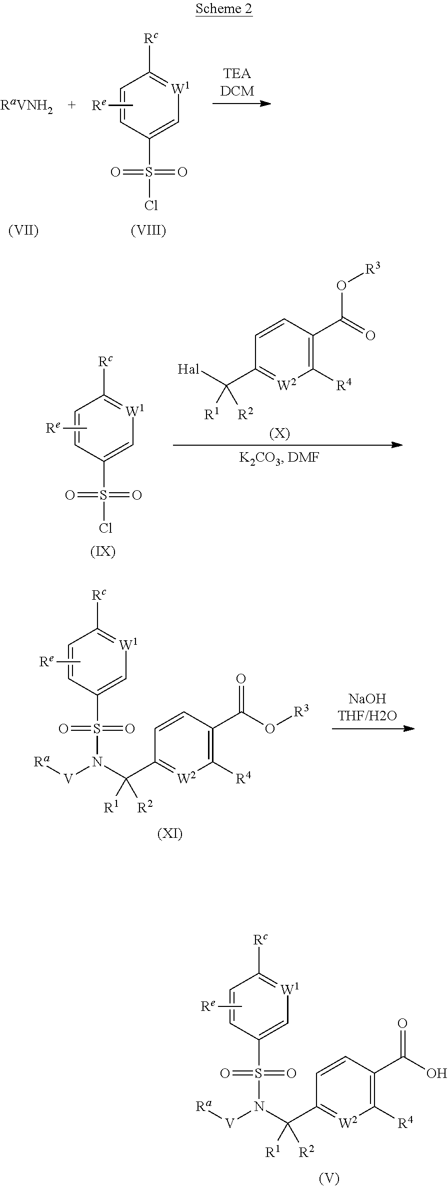

The first step, preferably consists in the reaction of an amine of Formula (VII), wherein v and R^{a} is defined as above, with a sulfonyl chloride of Formula (VIII), wherein W₁, R^{c} and R^{e} are defined as above, or another analogous activated sulfonyl derivative bearing a different leaving group instead of Cl at the sulfonyl group, in the presence or absence of bases such as TEA, DIEA, NMM in a suitable solvent such as DCM, THF or DMF, at a temperature from about 20° C. to about 50° C., preferably at room temperature, for a few hours, e.g. one hour to 24 h.

The second step consists in the reaction of a sulfonamide of Formula (IX) wherein V, R^{a}, W₁, R^{a} and R^{e} are defined as above, with an halide of Formula (X), wherein R¹, R², R³, R⁴, W² are defined as above, in presence of a suitable base, such as NaH, KOtBu, K₂CO₃, Na₂CO₃, NaHCO₃ or KHCO₃, preferably K₂CO₃ or Na₂CO₃, eventually in the presence of an iodine (−1) salt, such as but not limited to NaI or KI, in a suitable solvent such as DMF, at a temperature from about −20° C. to about 100° C., for a few hours, e.g. one hour to 24 h. The hydrolysis of the ester (XI) to give the compounds of Formula (V) can be accomplished using conditions and methods well known to those skilled in the art, such as but not limited to the use of a metal hydroxide, e.g. lithium hydroxide, sodium hydroxide or potassium hydroxide, in a suitable solvent such as THF, methanol or water or mixtures thereof, at a temperature rising from 20° C. to 50° C., preferably at room temperature, for a few hours, e.g. one hour to 24 h. The compounds of Formula (Via), wherein A, R^{a}, R^{c}, R^{e}, W¹, W² and W³ are defined as above, can be obtained from sulfonamide IX in a 2-step protocol as outlined in Scheme 2a

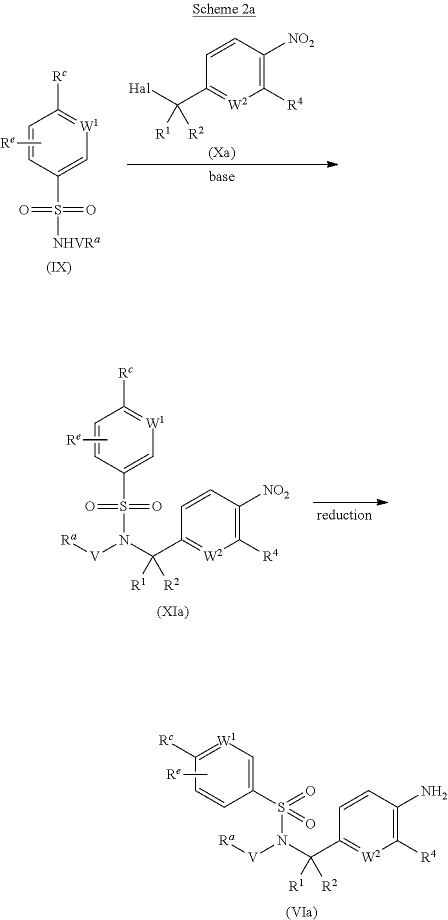

The first step consists in the reaction of a sulfonamide of Formula (IX) with a halide of Formula (Xa), wherein Hal, R^{c}, R^{e}, R¹, R², W² and W² are defined as above, in presence of a suitable base, such as NaH, KOtBu, K₂CO₃, Na₂CO₃, NaHCO₃ or KHCO₃, preferably K₂CO₃ or Na₂CO₃, eventually in the presence of an iodine (−1) salt, such as but not limited to NaI or KI, in a suitable solvent such as DMF, at a temperature from about −20° C. to about 100° C., for a few hours, e.g. one hour to 24 h. The second step consists in the reduction of the nitro group in (XIa) to give an amine of Formula (VIa). The reduction can be accomplished using conditions and methods well known to those skilled in the art, such as but not limited to the use of a metal salt, e.g. zinc(II)chloride or stannus(II)chloride, or a metal, e.g. iron dust/acetic acid or hydrogenolytically e.g. palladium-carbon/Hydrogen or raney-nickel/Hydrogen, in a suitable solvent such as THF, methanol, ethanol, dimethylformamide or water or mixtures thereof, at a temperature rising from 20° C. to 100° C., preferably at room temperature, for a few hours, e.g. one hour to 24 h.

Alternatively, compounds of Formula V can be prepared according to Scheme 3.

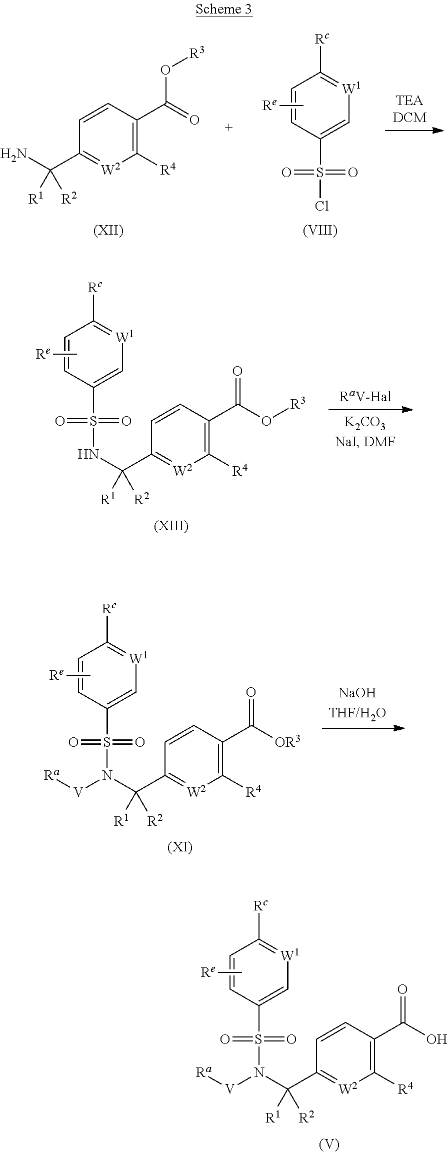

The first step consists in the reaction of an amine of Formula (XII), wherein W², R¹, R², R³ and R⁴ are defined as above, with a sulfonyl chloride of Formula (VIII), wherein W¹, R^{e} and R^{c} are as above defined, in the presence or absence of bases such as TEA, DIEA, NMM in a suitable solvent such as DCM, THF or DMF, at a temperature from about 20° C. to about 50° C., preferably at room temperature, for a few hours, e.g. one hour to 24 h. The sulfonamide of Formula (XIII) thus obtained can be alkylated with an Halide of Formula R^{a}VBr, wherein R^{a} and V are as defined above and Hal is Cl, Br, or I, preferably Br, in the presence of a suitable base such as NaH, KOtBu, K₂CO₃, Na₂CO₃, NaHCO₃ or KHCO₃, preferably K₂CO₃, eventually in the presence of an iodine (−1) salt, such as but not limited to NaI or KI, in a suitable solvent such as DMF, at a temperature from about −20° C. to about 100° C., preferably 100° C., for a few hours, e.g. one hour to 24 h. The hydrolysis of the ester XI to give the compounds of Formula V can be accomplished using conditions and methods well known to those skilled in the art, such as but not limited to the use of a metal hydroxide, e.g. lithium hydroxide, sodium hydroxide or potassium hydroxide, in a suitable solvent such as THF, methanol or water or mixtures thereof, at a temperature from about 20° C. to about 50° C., preferably at room temperature, for a few hours, e.g. one hour to 24 h.

Alternatively, compounds of Formula (XI) can be prepared according to Scheme 3b.

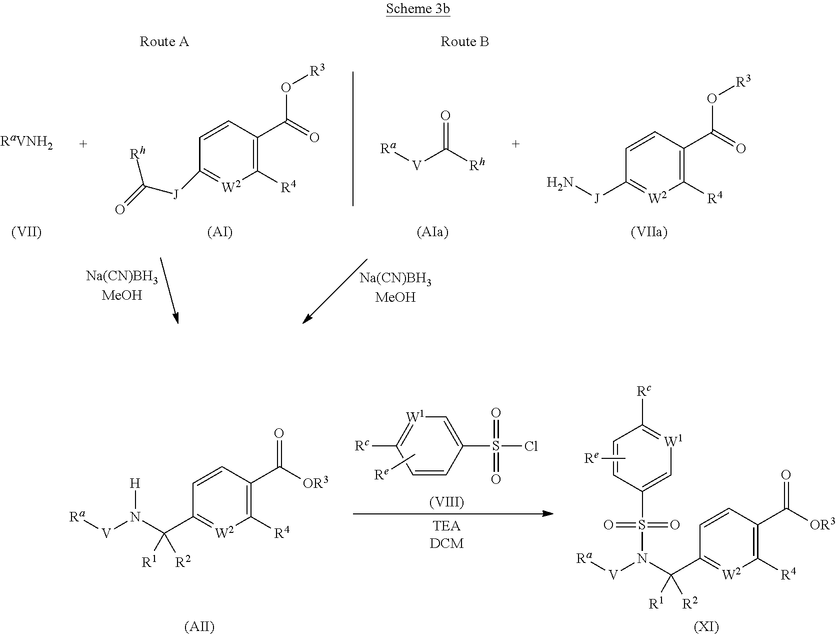

The first step that leads to amine (AII) consists in the reaction of an amine of Formula (VII), with an carbonyl compound of Formula (AI) (route A) or an amine of formula (VIIa) with a carbonyl compound of formula (AIa) (route B), wherein R^{a}, R^{e}, R¹, R², R³, R⁴, V, W¹, W² are defined as above, R^{h} denotes hydrogen or (C1-C6)alkyl, J denotes a valence bond or a linear or branched (C1-C6)alkylen group, under reductive amination conditions, using conditions and methods well known to those skilled in the art, in the presence of a reducing agent such as but not limited to Na(CN)BH₃ or NaB(OAc)₃H, in a suitable solvent such as MeOH, DCM or THF, at a temperature from about 20° C. to about 50° C., preferably at room temperature, for a few hours, e.g. one hour to 24 h. The amines of Formula All thus obtained can be reacted with a sulfonamide of Formula VIII, in the presence or absence of bases such as TEA, DIEA, NMM in a suitable solvent such as DCM, THF or DMF, at a temperature from about 20° C. to about 50° C., preferably at room temperature, for a few hours, e.g. one hour to 24 h.

Alternatively, compounds of Formula XIa can be prepared according to Scheme 3c.

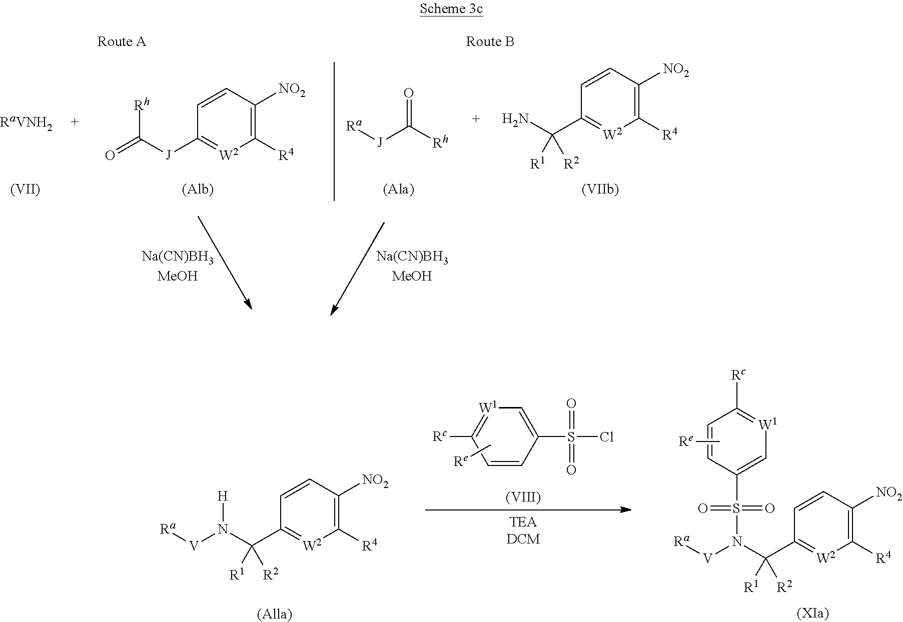

The first step that leads to amine (AIIa) consists in the reaction of an amine of Formula (VII) with a carbonyl compound of Formula (AIb) (route A) or of an amine of Formula (VIIb) with a carbonyl compound of Formula (AIa) (route B), wherein V, R^{a}, R^{e}, R^{c}, R¹, R², R⁴, R^{h}, W², and J are defined as above, under reductive amination conditions, using conditions and methods well known to those skilled in the art, in the presence of a reducing agent such as but not limited to Na(CN)BH₃ or NaB(OAc)₃H, in a suitable solvent such as MeOH, DCM or THF, at a temperature from about 20° C. to about 50° C., preferably at room temperature, for a few hours, e.g. one hour to 24 h. The amines of Formula AIIa thus obtained can be reacted with a sulfonamide of Formula (VIII), in the presence or absence of bases such as TEA, DIEA, NMM in a suitable solvent such as DCM, THF or DMF, at a temperature from about 20° C. to about 50° C., preferably at room temperature, for a few hours, e.g. one hour to 24 h.

An alternative route for the preparation of the compounds of Formula (II) or (IIa) may be the reaction of a sulfonamide of Formula (IX), either commercially available or prepared as described above, with an halide of Formula (XIV) or (XIVa), wherein V, R^{a}, R^{c}, R^{e}, R^{d}, Q, R¹, R², R⁴, W¹, W² and Y are defined as above (Scheme 4).

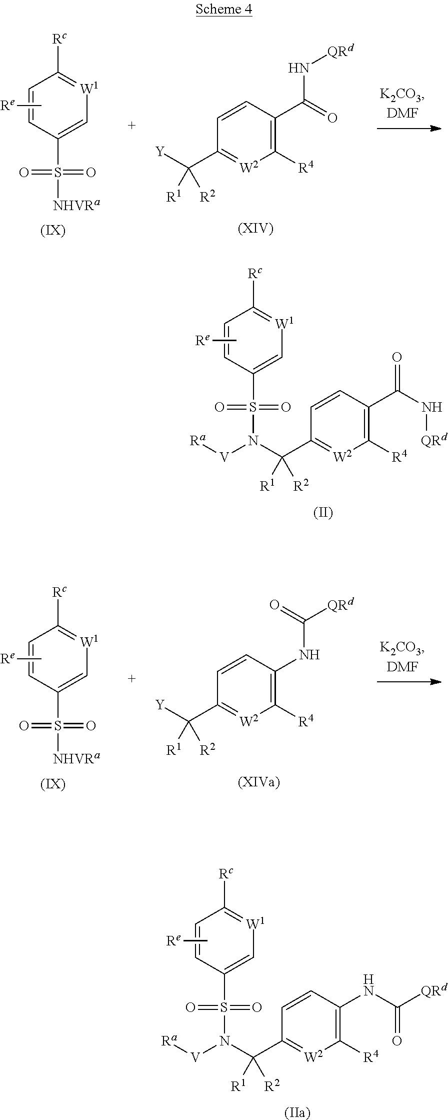

Following the same protocol as before, compounds of formula (II*) and (IIa*) can be obtained by reacting compounds of formula (IX) with compounds of Formula (XIV*) or (XIVa*) wherein G′ is as defined above (scheme 4b).

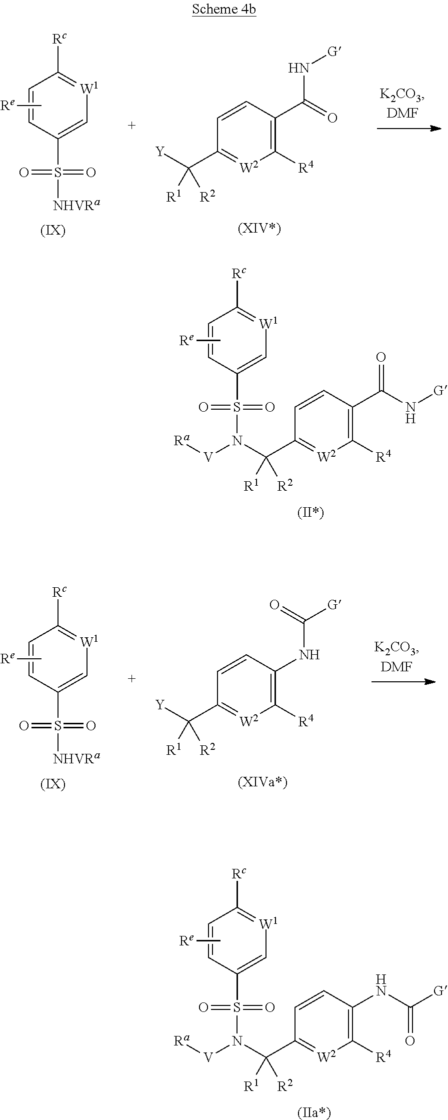

The reaction can be performed in the presence of a suitable base such as NaH, KOtBu, K₂CO₃, Na₂CO₃, NaHCO₃ or KHCO₃, preferably K₂CO₃, eventually in the presence of an iodine (−1) salt, such as but not limited to NaI or KI, in a suitable solvent such as DMF, at a temperature between −20° C. to 100° C., preferably 100° C., for a few hours, e.g. one hour to 24 h.

Generally, halides of Formula (XIV) or (XIVa) can by prepared as outlined in scheme 5, by coupling a carboxylic acid of Formula (XV) or (VIa) with an amine of Formula (VI) or (VIa), wherein R^{d}, Q, W² and Y are defined as above, using conditions and methods well known to those skilled in the art to prepare an amide bond from an amine and a carboxylic acid, with standard coupling agents, such as but not limited to polymer-supported 1-alkyl-2-chloropyridinium salt (polymer-supported Mukaiyama's reagent), 1-methyl-2-chloropyridinium iodide (Mukaiyama's reagent), DCC, DIC, preferably EDC, in the presence or absence of bases such as TEA, DIEA, NMM in a suitable solvent such as DCM, THF or DMF, at a temperature from about 20° C. to about 50° C., preferably at room temperature, for a few hours, e.g. one hour to 24 h. Alternatively, a carboxylic acid derivative (e.g. acyl chloride) of formula (XVI) or (XVIa), wherein Y and L are as defined above, may be coupled with the amine (VI) or (VIa), using conditions and methods well known to those skilled in the art, in the presence of bases such as TEA, DIEA, NMM in a suitable solvent such as DCM, THF or DMF, at a temperature from about 20° C. to about 50° C., preferably at room temperature, for a few hours, e.g. one hour to 24 h.

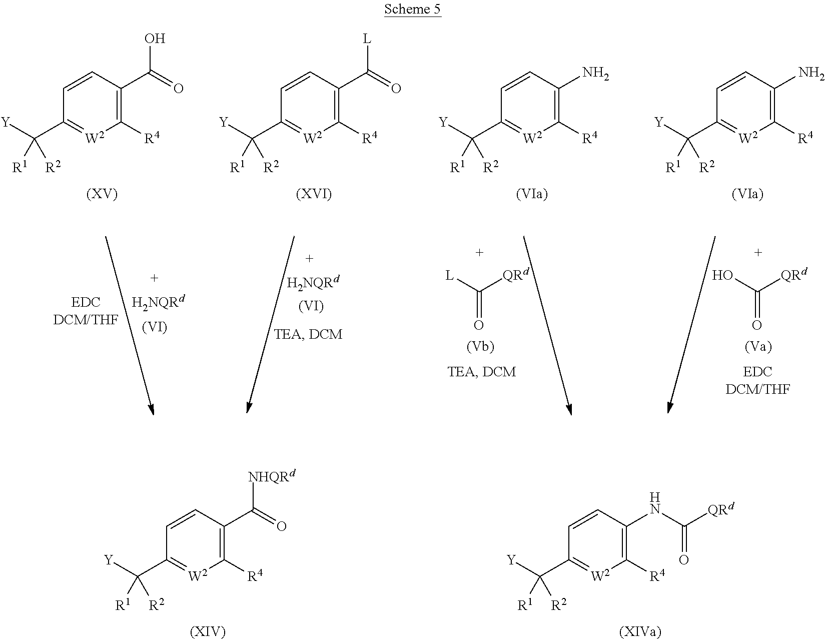

The compounds of Formula (III), wherein R^{a}, R^{c}, R^{d}, R^{e}, W¹ and W² are defined as above, can be prepared by coupling a carboxylic acid of Formula V, commercially available or prepared as described above and wherein R^{a}, R^{c}, R^{e}, W¹ and W² are defined as above, with a sulfonamide of Formula XVII as outlined in Scheme 6, using conditions and methods well known to those skilled in the art, with an appropriate coupling agents, such as but not limited to DCC, DIC or preferably EDC, in the presence dimethylaminopyridine in a suitable solvent such as DCM, THF or DMF, at a temperature from about 20° C. to about 50° C., preferably at room temperature, for a few hours, e.g. one hour to 24 h.

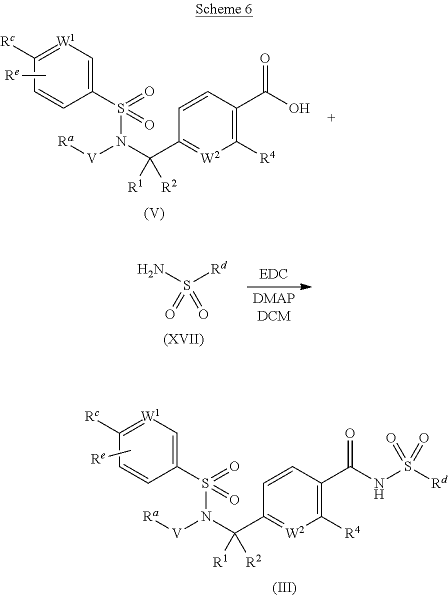

The sulfonamides of Formula XVII, wherein R^{d} is defined as above, are either commercially available or may be prepared by standard synthetic techniques, as hereinafter described in the examples, for example by reaction of ammonia with a sulfonyl chloride in the presence of a suitable solvent.

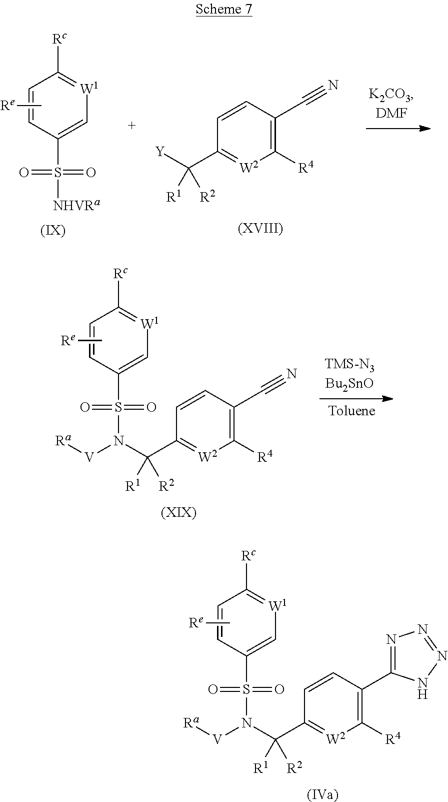

Compounds of Formula IVa, wherein V, R¹, R²R⁴, R^{a}, R^{c}, R^{e}, W¹ and W² are defined as above, can be prepared according to Scheme 7, by reaction of sulfonamide of Formula IX, wherein R^{a}, R^{c}, R^{e} and W¹ are as defined above, commercially available or prepared as described above, with a compound of Formula XVIII, wherein Y is as defined above, in the presence of a suitable base such as NaH, KOtBu, K₂CO₃, Na₂CO₃, NaHCO₃ or KHCO₃, preferably K₂CO₃, preferably in the presence of an iodine (−1) salt, such as but not limited to NaI or KI, at a temperature between −20° C. to 100° C., preferably 100° C., for a few hours, e.g. one hour to 24 h. The conversion of the compounds of Formula XIX to the corresponding compounds of Formula IVa can be accomplished by any of the methods known to those skilled in the art for the conversion of a nitrile to a tetrazole group, such as but not limited to the use of trimethylsilyl azide in the presence of dibutyltin oxide, at a temperature from about 20° C. to about 100° C., preferably 90° C., for a few hours, e.g. one hour to 24 h. Alternatively, the compounds of Formula XIX can be prepared according to Scheme 8, by reaction of an amine of Formula XX with a sulfonyl chloride of Formula VIII, in the presence or absence of bases such as TEA, DIEA, NMM in a suitable solvent such as DCM, THF or DMF, at a temperature from about 20° C. to about 50° C., preferably at room temperature, for a few hours, e.g. one hour to 24 h. The sulfonamide of Formula XXI thus obtained can be alkylated with an alkyl bromide in the presence of a suitable base such as NaH, KOtBu, K₂CO₃, Na₂CO₃, NaHCO₃ or KHCO₃, preferably K₂CO₃, eventually in the presence of an iodine (−1) salt, such as but not limited to NaI or KI, in a suitable solvent such as DMF, at a temperature between −20° C. to 100° C., preferably 100° C., for a few hours, e.g. one hour to 24 h.

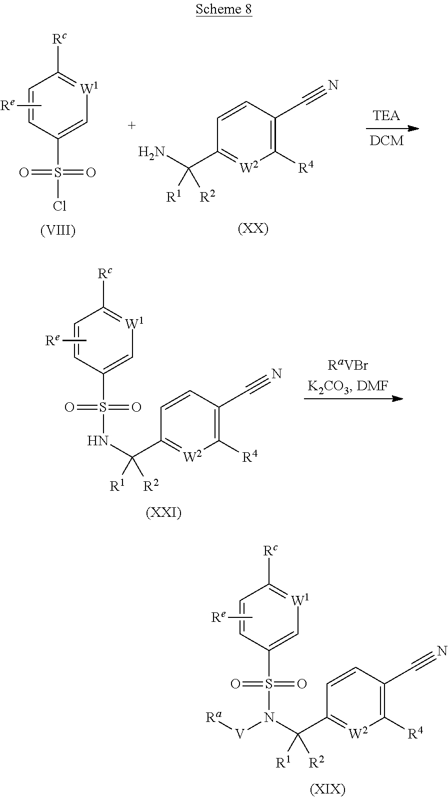

Compounds of Formula IVb, wherein R^{a}, R^{c}, R^{e}, W¹ and W² are defined as above, can be prepared according to Scheme 9, by reaction of an ester of Formula XI with hydrazine in a suitable solvent such as THF, MeOH or DMF, at a temperature from about 20° C. to about 50° C., preferably at room temperature, for a few hours, e.g. one hour to 24 h to give an intermediate of Formula XXII. This intermediates can be cyclized to the desired product of Formula IVb using any protocol known in the art for the conversion of an acylhydrazine into a 5-hydroxy-1,3,4-oxadiazole, such as but not limited to treatment with carbonyldiimidazole in the presence of a suitable base, such as TEA, in a suitable solvent such as DMF, at a temperature from about 20° C. to about 50° C., preferably at room temperature, for a few hours, e.g. one hour to 24 h.

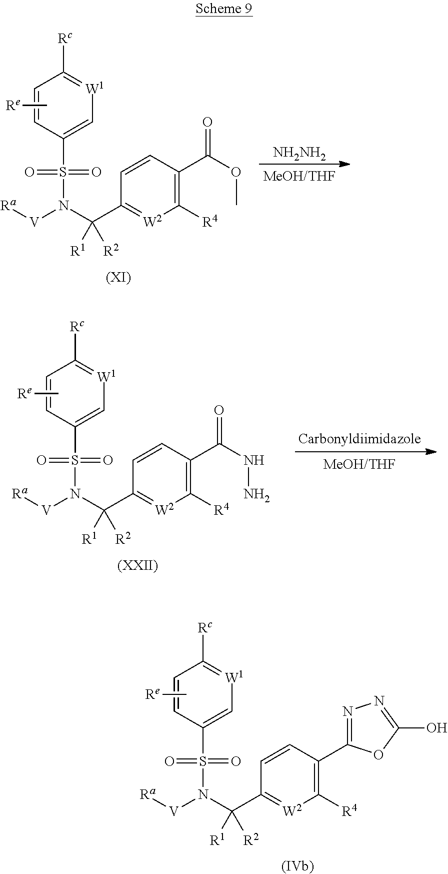

Compounds of Formula (XXIII), wherein R^{a}, R^{c}, R^{e}, R^{d}, V, Q, W¹ and W² are defined as above, can be prepared according to Scheme 10, by reaction of a sulfonamide of Formula (IX) with a carboxylic acid, with standard coupling agents, such as but not limited to 1-alkyl-2-chloropyridinium salt or preferably polymer-supported 1-alkyl-2-chloropyridinium salt (polymer-supported Mukaiyama's reagent), 1-methyl-2-chloropyridinium iodide (Mukaiyama's reagent), DCC, DIC, preferably EDC, in the presence or absence of bases such as TEA, DIEA, NMM in a suitable solvent such as DCM, THF or DMF, at a temperature between 20° C. to 50° C., preferably at room temperature, for a few hours, e.g. one hour to 24 h. Alternatively, a carboxylic acid derivative (e.g. acyl chloride; Vb) may be coupled with the amine, using conditions and methods well known to those skilled in the art, in the presence of bases such as TEA, DIEA, NMM in a suitable solvent such as DCM, THF or DMF, at a temperature from about 20° C. to about 50° C., preferably at room temperature, for a few hours, e.g. one hour to 24 h

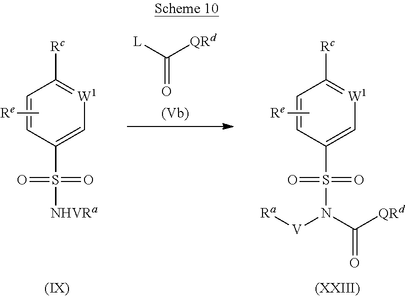

Compounds of Formula (XXIV), wherein R^{a}, R^{b}, R^{c}, R^{e}, R¹, R², R⁴, V, W¹ and W² are defined as above, can be prepared according to Scheme 11, by reaction of a sulfonamide of Formula (IX) with an alcohol (XXV) under mitsonobu conditions, like diethyldiazadicarboxylate and triphenylphosphine, in the presence or absence of bases such as TEA, DIEA, NMM in a suitable solvent such as Toluene, DCM, THF or DMF, at a temperature between −10° C. to 50° C., preferably at 0° C., for a few hours, e.g. one hour to 24 h.

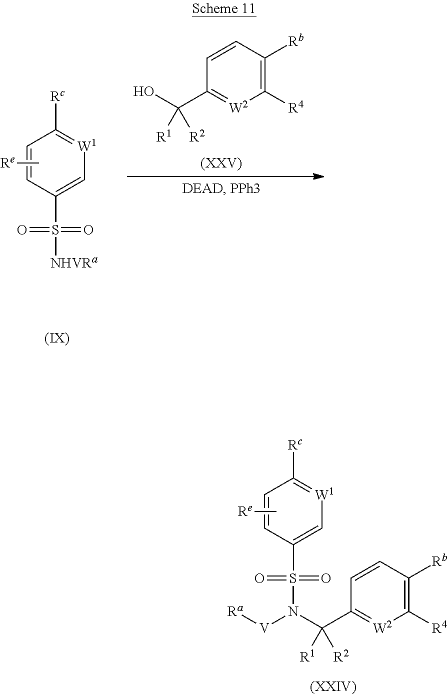

Compounds of Formula (XXVI), wherein R^{c}, R^{d}, R^{e}, R¹, R², R⁴, V, W¹ and W² are defined as above, can be prepared according to Scheme 12, by reaction of a sulfonamide of Formula II with an oxidation agent like 3-Chloroperbenzoic acid in a suitable solvent such as Toluene, DCM, THF or DMF, at a temperature between −10° C. to 50° C., preferably at room temperature, for a few hours, e.g. one hour to 24 h.

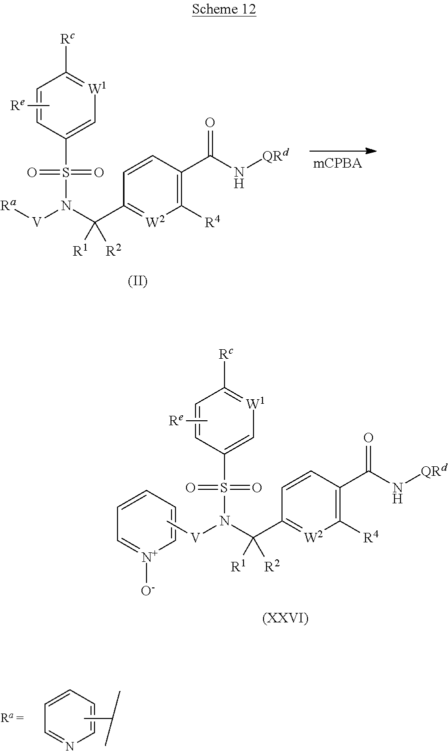

Compounds of Formula (XXVII), wherein V, G′, R^{a}, R^{c}, R^{e}, R¹, R², R³, R⁴, W¹ and W² are defined as above, can be prepared according to Scheme 13, by reaction of a sulfonamide of Formula (II) with a bases such as TEA, DIEA, NMM, NaH or an acid like HCl, TFA in a suitable solvent such as DCM, THF, Dioxan or DMF, at a temperature between 0° C. to 50° C., preferably at room temperature, for a few hours, e.g. one hour to 24 h.

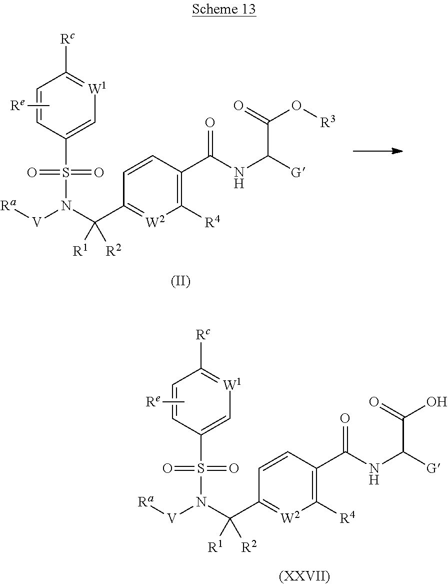

The above set out general synthetic methods may be modified for the obtention of compounds of Formula (I), since various suitable methods of preparation known by a person skilled in the art are available.

According to a further general process, compounds of Formula I, II and IVa can be converted to alternative compounds of Formula I, II and III, employing suitable interconversion techniques well known by a person skilled in the art.

Suitable methods of preparation for the compounds and intermediates of the invention as known by a person skilled in the art should be used. In general, the synthesis pathways for any individual compound of Formula I will depend on the specific substitutents of each molecule and upon the ready availability of intermediates necessary; again such factors being appreciated by those of ordinary skill in the art.

Compounds of this invention can be isolated in association with solvent molecules by crystallization through evaporation of an appropriate solvent. The pharmaceutically acceptable acid addition salts of the compounds of Formula I, which contain a basic center, may be prepared in a conventional manner. For example, a solution of the free base may be treated with a suitable acid, either neat or in a suitable solution, and the resulting salt isolated either by filtration or by evaporation under vacuum of the reaction solvent. Pharmaceutically acceptable base addition salts may be obtained in an analogous manner by treating a solution of compound of Formula I, which contain an acid center, with a suitable base. Both types of salts may be formed or interconverted preferably using ion-exchange resin techniques.

Depending on the conditions used, the reaction times are generally between a few minutes and 14 days, and the reaction temperature is between about −30° C. and 140° C., normally between −10° C. and 120° C., in particular between about 0° C. and about 90° C.

Compounds of the formula I can furthermore be obtained by treating functional derivatives of formula I with a solvolysing or hydrogenolysing agent.

Preferred functional derivatives of formula I for the solvolysis or hydrogenolysis are those which contain corresponding protected amino and/or hydroxyl groups instead of one or more free amino and/or hydroxyl groups. Preferred embodiments are functional derivatives those which carry an amino-protecting group instead of an H atom bonded to an N atom, in particular those which carry an R′—N group, in which R′ denotes an amino-protecting group, instead of an HN group. Preferred alternative embodiments are functional derivatives which carry a hydroxyl-protecting group instead of the H atom of a hydroxyl group, for example those which conform to the formula I, but carry a —COOR″ group, in which R″ denotes a hydroxylprotecting group, instead of a —COOH group.

It is also possible for a plurality of—identical or different—protected amino and/or hydroxyl groups to be present in the molecule of the starting material. If the protecting groups present are different from one another, they can in many cases be cleaved off selectively.

The term “amino-protecting group” is known in general terms and relates to groups which are suitable for protecting (blocking) an amino group against chemical reactions, but which are easy to remove after the desired chemical reaction has been carried out elsewhere in the molecule. Typical of such groups are, in particular, unsubstituted or substituted acyl, aryl, aralkoxymethyl or aralkyl groups. Since the amino-protecting groups are removed after the desired reaction (or reaction sequence), their type and size are furthermore not crucial; however, preference is given to those having 1-20, in particular 1-8, carbon atoms. The term “acyl group” is to be understood in the broadest sense in connection with the present process. It includes acyl groups derived from aliphatic, araliphatic, aromatic or hetero-cyclic carboxylic acids or sulfonic acids, and, in particular, alkoxy-carbonyl, aryloxycarbonyl and especially aralkoxycarbonyl groups. Examples of such acyl groups are alkanoyl, such as acetyl, propionyl and butyryl; aralkanoyl, such as phenylacetyl; aroyl, such as benzoyl and tolyl; aryloxyalkanoyl, such as POA (phenoxyacetyl), alkoxycarbonyl, such as methoxy-carbonyl, ethoxycarbonyl, 2,2,2-trichloroethoxycarbonyl, BOC (tert-butoxy-carbonyl) and 2-iodoethoxycarbonyl; aralkoxycarbonyl, such as CBZ (“carbo-benz-oxy”), 4-methoxybenzyloxycarbonyl and FMOC (9H-fluoren-9-ylmethoxycarbonyl); and aryl-sulfonyl, such as Mtr (4-Methoxy-2,3,6-trimethylbenzenesulphonyl). Preferred amino-protecting groups are BOC and Mtr, further-more CBZ, Fmoc, benzyl and acetyl.

The term “hydroxyl-protecting group” is likewise known in general terms and relates to groups which are suitable for protecting a hydroxyl group against chemical reactions, but are easy to remove after the desired chemical reaction has been carried out elsewhere in the molecule. Typical of such groups are the above-mentioned unsubstituted or substituted aryl, aralkyl or acyl groups, furthermore also alkyl groups. The nature and size of the hydroxyl-protecting groups are not crucial since they are removed again after the desired chemical reaction or reaction sequence; preference is given to groups having 1-20, in particular 1-10, carbon atoms. Examples of hydroxyl-protecting groups are, inter alia, benzyl, 4-methoxybenzyl, p-nitro-benzoyl, p-toluenesulfonyl, tert-butyl and acetyl, where benzyl and tert-butyl are particularly preferred.

The compounds of the formula I are liberated from their functional derivatives—depending on the protecting group used—for example using strong acids, advantageously using TFA or perchloric acid, but also using other strong inorganic acids, such as hydrochloric acid or sulfuric acid, strong organic carboxylic acids, such as trichloroacetic acid, or sulfonic acids, such as benzene- or p-toluenesulfonic acid. The presence of an additional inert solvent is possible, but is not always necessary. Suitable inert solvents are preferably organic, for example carboxylic acids, such as acetic acid, ethers, such as tetrahydrofuran or dioxane, amides, such as DMF, halogenated hydrocarbons, such as dichloromethane, furthermore also alcohols, such as methanol, ethanol or isopropanol, and water. Mixtures of the above-mentioned solvents are furthermore suitable. TFA is preferably used in excess without addition of a further solvent, and perchloric acid is preferably used in the form of a mixture of acetic acid and 70% perchloric acid in the ratio 9:1. The reaction temperatures for the cleavage are advantageously between about 0 and about 50° C., preferably between 15 and 30° C. (room temperature).

The BOC, OBut and Mtr groups can, for example, preferably be cleaved off using TFA in dichloromethane or using approximately 3 to 5N HCl in dioxane at 15-30° C., and the FMOC group can be cleaved off using an approximately 5 to 50% solution of dimethylamine, diethylamine or piperidine in DMF at 15-30° C.

Protecting groups which can be removed hydrogenolytically (for example CBZ, benzyl or the liberation of the amidino group from the oxadiazole derivative thereof) can be cleaved off, for example, by treatment with hydrogen in the presence of a catalyst (for example a noble-metal catalyst, such as palladium, advantageously on a support, such as carbon). Suitable solvents here are those indicated above, in particular, for example, alcohols, such as methanol or ethanol, or amides, such as DMF. The hydrogenolysis is generally carried out at temperatures between about 0 and 100° C. and pressures between about 1 and 200 bar, preferably at 20-30° C. and 1-10 bar. Hydrogenolysis of the CBZ group succeeds well, for example, on 5 to 10% Pd/C in methanol or using ammonium formate (instead of hydrogen) on Pd/C in methanol/DMF at 20-30° C.

Examples of suitable inert solvents are hydrocarbons, such as hexane, petroleum ether, benzene, toluene or xylene; chlorinated hydrocarbons, such as trichloroethylene, 1,2-dichloroethane, tetrachloromethane, tri-fluoro-methylbenzene, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide, N-methylpyrrolidone (NMP) or dimethyl-formamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

Esters can be saponified, for example, using acetic acid or using LiOH, NaOH or KOH in water, water/THF, water/THF/ethanol or water/dioxane, at temperatures between 0 and 100° C.

Free amino groups can furthermore be acylated in a conventional manner using an acid chloride or anhydride or alkylated using an unsubstituted or substituted alkyl halide or reacted with CH₃—C(═NH)—OEt, advantageously in an inert solvent, such as dichloromethane or THF and/or in the presence of a base, such as triethylamine or pyridine, at temperatures between −60° C. and +30° C.

Therefore, the invention also relates to a process for the preparation of the compounds of formula I and related formulae, wherein R^{b} denotes CONHQR^{d}, and salts thereof, characterized in that

a compound of formula V*

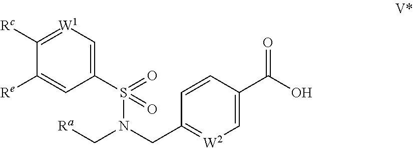

wherein R^{c}, R^{a}, R^{e}, W¹ and W², are as defined above, is reacted with a compound of formula

H₂NQR^{d}

wherein R^{d}, and Q are as defined above,
preferably in the presence of a coupling reagent such as 1-alkyl-2-chloropyridinium salt or polymer-supported 1-alkyl-2-chloropyridinium salt (polymer-supported Mukaiyama's reagent), 1-methyl-2-chloropyridinium iodide (Mukaiyama's reagent), DCC, DIC, EDC, in the presence or absence of bases such as TEA, DIEA, NMM in a suitable solvent such as DCM, THF or DMF, preferably at a temperature between about 20° C. to about 50° C., more preferably at room temperature, for a few hours, e.g. one hour to 24 h,
or
b) a compound of formula Va*

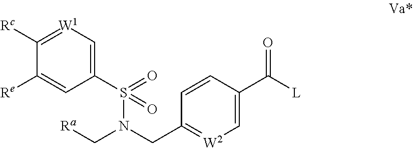

wherein R^{c}, R^{a}, R^{e}, W¹, W² and L are as defined above,
is reacted with a compound of formula

H₂NQR^{d}

wherein R^{d}, and Q are as defined above, preferably in the presence of a base such as TEA, DIEA, NMM in a suitable solvent such as DCM, THF or DMF, at a temperature from about 20° C. to about 50° C., preferably at room temperature, for a few hours, e.g. one hour to 24 h,
or
c) a compound of formula IX*

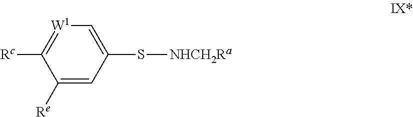

wherein R^{a}, R^{c} R^{e} and W¹ are as defined above,
is reacted with a compound of formula XIV*

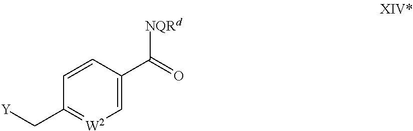

wherein Y, Q, R^{d} and W² are as defined above, preferably in presence of a suitable base, such as NaH, KOtBu, K₂CO₃, Na₂CO₃, NaHCO₃ or KHCO₃, preferably K₂CO₃ or Na₂CO₃, preferably in the presence of an iodine (−1) salt, such as but not limited to NaI or KI, in a suitable solvent such as DMF, at a temperature between −20° C. to 100° C., for a few hours, e.g. one hour to 24 h.

The invention also relates to a process for the preparation of the compounds of formula (I) and related formulae, wherein R^{b} denotes oxadiazolyl or hydroxyl-substituted oxadiazolyl, and salts thereof, characterized in that a compound of formula XIa*

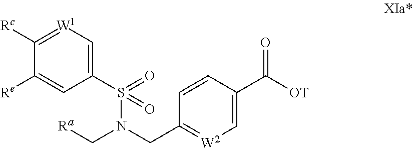

wherein R^{a}, R^{c}, R^{e}, W¹ and W² are as defined above and T denotes alkyl having 1 to 12 carbon atoms, preferably methyl, or ethyl, is firstly reacted with hydrazine and subsequently with carbonyldiimidazole, preferably in the presence of a suitable base, such as TEA, in a suitable solvent such as DMF, at a temperature from about 20° C. to about 50° C., preferably at room temperature, for a few hours, e.g. one hour to 24 h.

The invention also relates to a process for the preparation of the compounds of formula (I) and related formulae, wherein R^{b} denotes tetrazolyl, and salts thereof, characterized in that a compound of formula XIX*

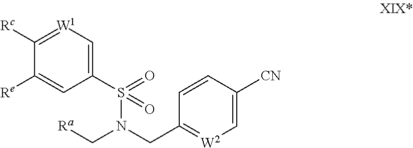

wherein R^{a}, R^{c}, R^{e}, W¹ and W² are as defined above,
is reacted with an azide, preferably trimethylsilyl azide, preferably in the presence of dibutyltin oxide, at a temperature from about 20° C. to about 100° C., preferably 90° C., for a few hours, e.g. one hour to 24 h.

The formula (I) also encompasses the optically active forms (stereoisomers), the enantiomers, the racemates, the diastereomers and the hydrates and solvates of these compounds. The term “solvates of the compounds” is taken to mean adductions of inert solvent molecules onto the compounds, which form owing to their mutual attractive force. Solvates are, for example, mono- or dihydrates or alcoholates.

The term “pharmaceutically usable derivatives” is taken to mean, for example, the salts of the compounds of the formula I and so-called prodrug compounds.

The term “prodrug derivatives” is taken to mean compounds of the formula I which have been modified with, for example, alkyl or acyl groups, sugars or oligopeptides and which are rapidly cleaved in the organism to form the active compounds.

These also include biodegradable polymer derivatives of the compounds according to the invention, as described, for example, in Int. J. Pharm. 115, 61-67 (1995).

The formula (I) also encompasses mixtures of the compounds of the formula I, for example mixtures of two diastereomers, for example in the ratio 1:1, 1:2, 1:3, 1:4, 1:5, 1:10, 1:100 or 1:1000.

These are particularly preferably mixtures of stereoisomeric compounds.

For all radicals, which occur more than once in a single chemical formula, their meanings are independent of one another.

Above and below, the groups or parameters R^{a}, R^{b}, R^{c}, R^{d}, R^{e}, Q, W¹, W², T, Ar, Het, p, p′ and s have the meaning indicated under the formulae (I) and related formulae, unless expressly stated otherwise.

T denotes alkyl, is unbranched (linear) or branched, and has 1, 2, 3, 4, 5, 6, 7, 8, 9, 10, 11 or 12 carbon atoms. A preferably denotes methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, furthermore preferably, for example, trifluoromethyl. In a preferred embodiment, A is a perfluoroalkyl or a partially fluorinated alkyl. For example, A is trifluoromethyl, pentafluoromethyl, 1,1,1-trifluoroethyl.

T furthermore denotes (CH₂)ₙO(CH₂)ₙOR⁵, especially (CH₂)₂O(CH₂)₂OR⁵, (CH₂)ₙNR⁵(CH₂)₂N(R⁵)₂, especially (CH₂)₂NH(CH)₂N(R⁵)₂.

R⁵ denotes H, Alkyl or Ar.

Cyc preferably denotes a cycloalkyl having 3 to 8 carbon atoms, which is unsubstituted or monosubstituted, disubstituted, trisubstituted by OH, Hal, CN,

Cycloalkyl preferably denotes cyclopropyl, cyclobutyl, cyclopentyl, cyclohexyl or cycloheptyl.

A preferably denotes a branched or linear alkylen having 1 to 6 carbon atoms wherein one or more, preferably 1 to 7H atoms may be replaced by Hal, OR³, N(R³)₂, Het, Ar, NHCOOR³, COOR³, —CON(R³)₂, and wherein one or more CH₂-groups may be replaced by O, NR³, OCO, NHCO, SO₂, and/or by —CH═CH—, —C≡C—, or denotes cycloalkyl, cycloalkylen or cycloalkylalkylen having 3 to 7 ring C-atoms.

A more preferably denotes a branched or linear alkylen having 1 to 6 carbon atoms wherein 1 or 2 H atoms may be replaced by Hal, OR³, N(R³)₂, Het, Ar, NHCOOR³, COOR³, —CON(R³)₂, and wherein 1 or 2 CH₂-groups may be replaced by O, NR³, OCO, NHCO, SO₂, or denotes cycloalkyl, cycloalkylen or cycloalkylalkylen having 3 to 7 ring C-atoms.

R^{a} is preferably phenyl, which is unsubstituted or preferably substituted by one or more of the groups Hal, CN, NO₂, CF₃, OCF₃, SCN or alkoxy having 1 to 8 carbon atoms, especially F, Cl or methoxy. Furthermore, R^{a} is preferably unsubstituted 2-, 3- or 4-pyridyl, especially 2-pyridyl.

R^{a} is most preferably one of the following groups:

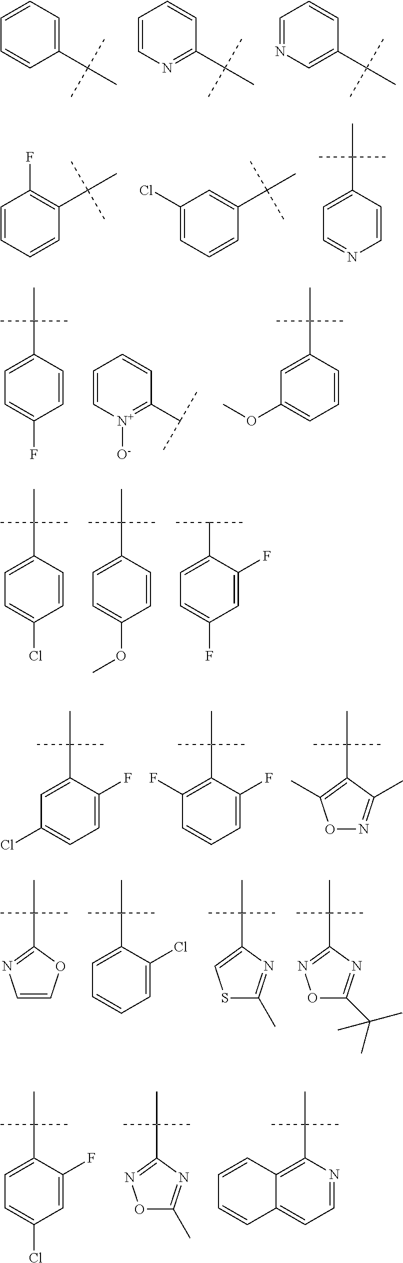

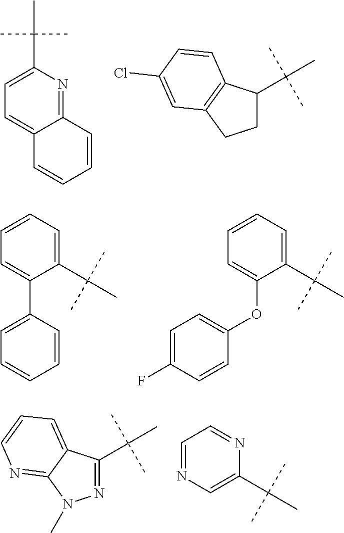

R^{b} preferably denotes CN, Het, Hal, NO₂, or a group —CONHA or —NHCOA, —CO—NHSO₂A, wherein A is as defined above.

R^{b} is preferably a group —C(O)—NHQR^{d}, wherein Q is preferably (CH₂)ₚ or (CH₂)ₚSO₂(CH₂)_{p′}, especially CH₂, CH₂CH₂ or SO₂ and R^{d} is preferably Ar or cycloalkyl having 3 to 7 carbon atoms, or a saturated heterocyclic ring having 3, 4 or 5 carbon atoms and 1 or 2 N or O atoms, especially phenyl. Phenyl is preferably unsubstituted or substituted by one or more of the groups Hal, CN, NO₂, CF₃, OCF₃, SCN or alkoxy having 1 to 8 carbon atoms, or cyclopropyl, cyclopentyl or cyclohexyl or tetrahydrofuranyl, dioxanyl, pyrrolidinyl or morpholinyl. Furthermore, R^{b} is preferably 1- or 5-tetrazolyl, 1,2,3-oxadiazol-4- or -5-yl, 1,2,4-oxadiazol-3- or -5-yl or 5-hydroxy-1,3,4-oxadiazol-2yl.

R^{b} more preferably denotes F, Cl, OMe, NH₂, OEt, or one of the following groups:

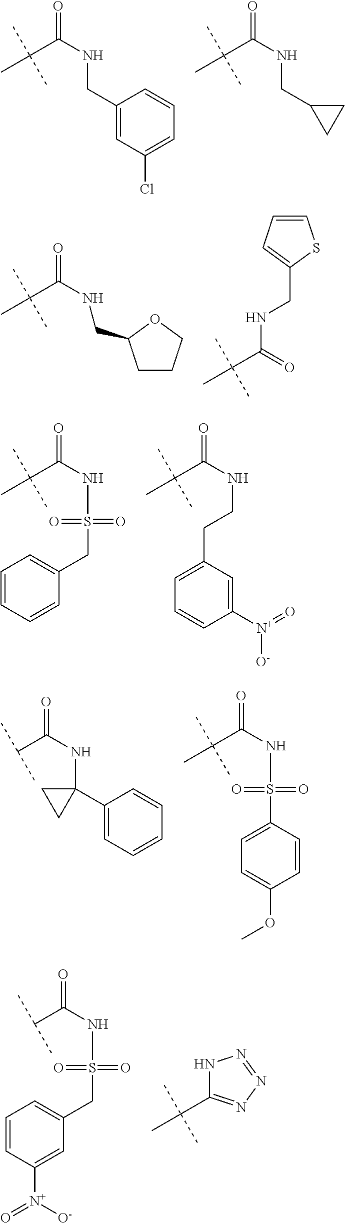

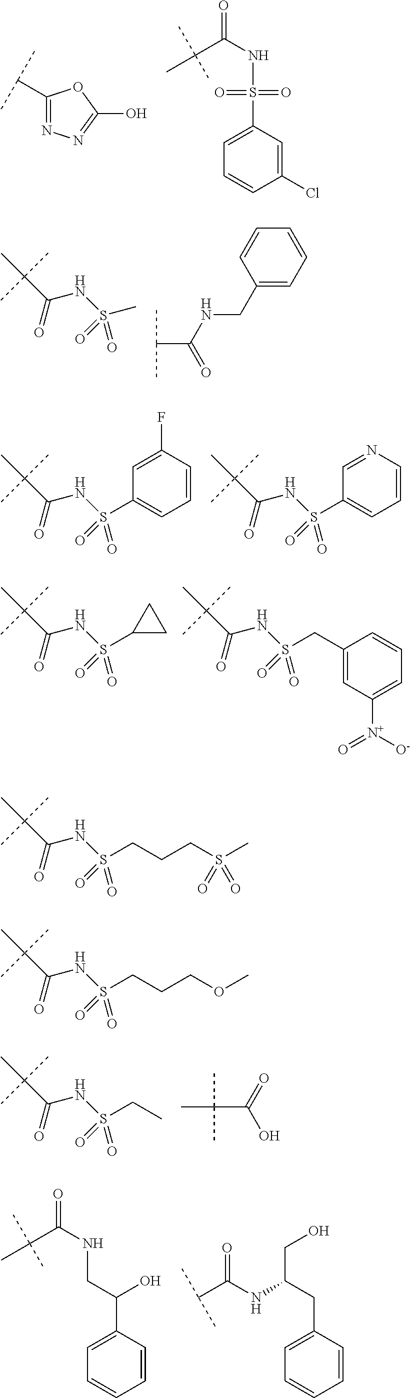

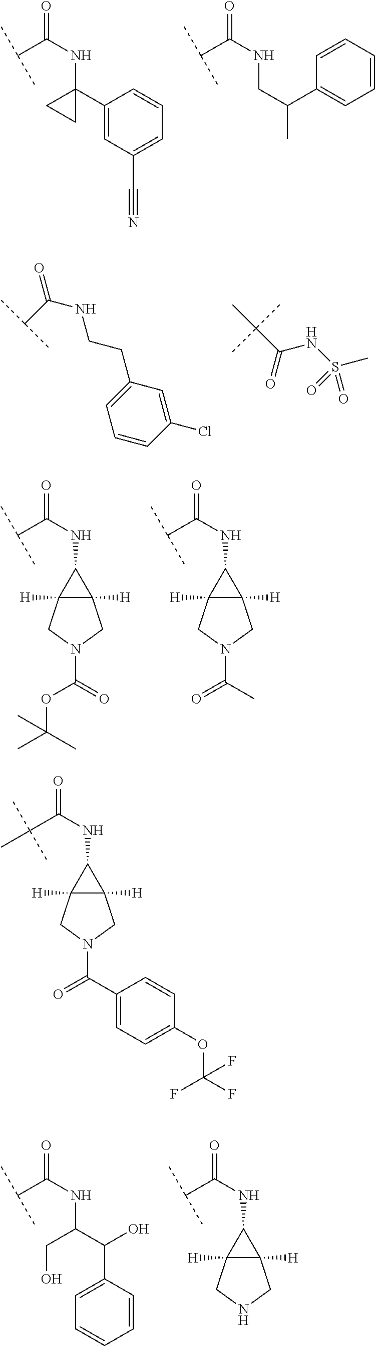

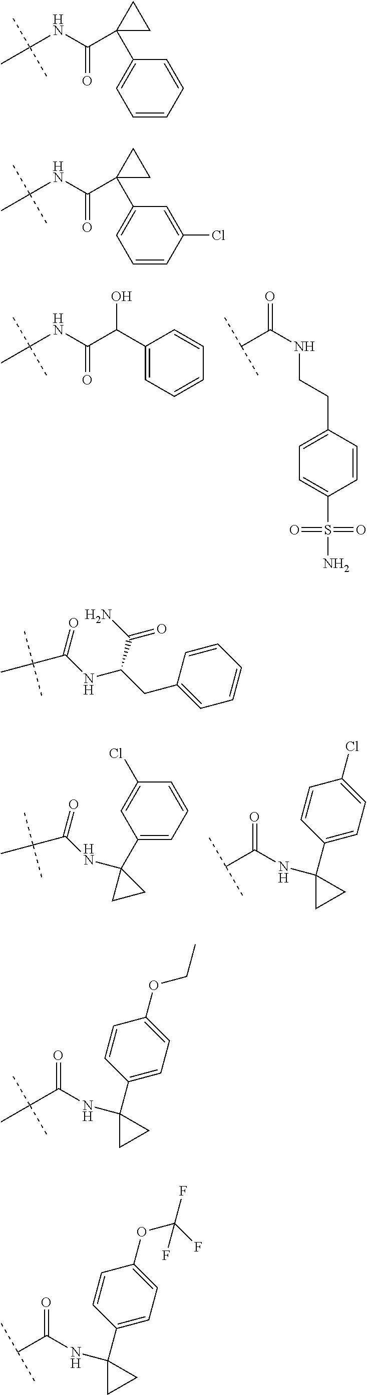

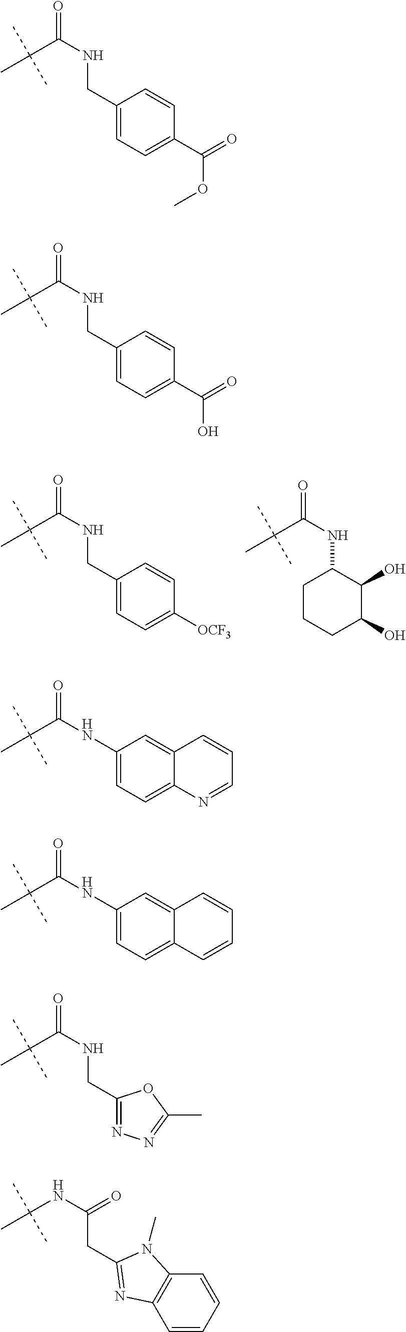

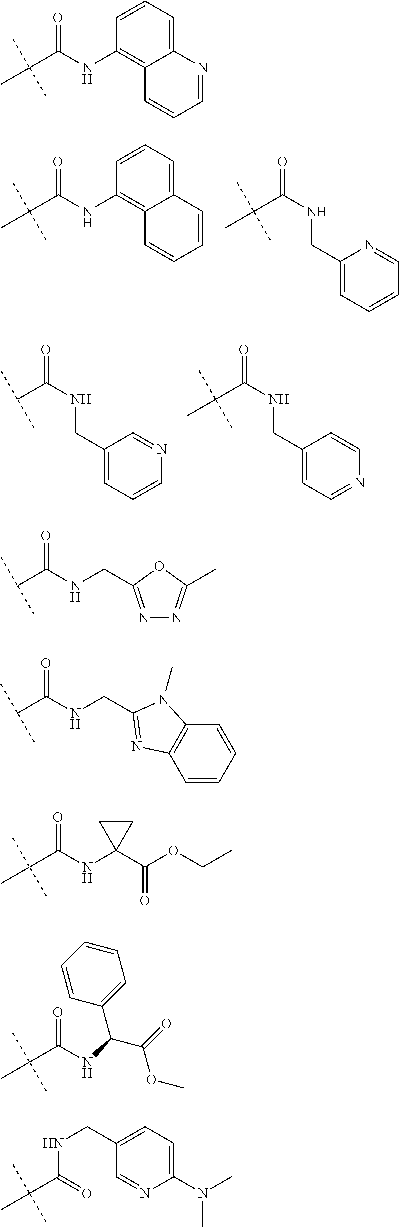

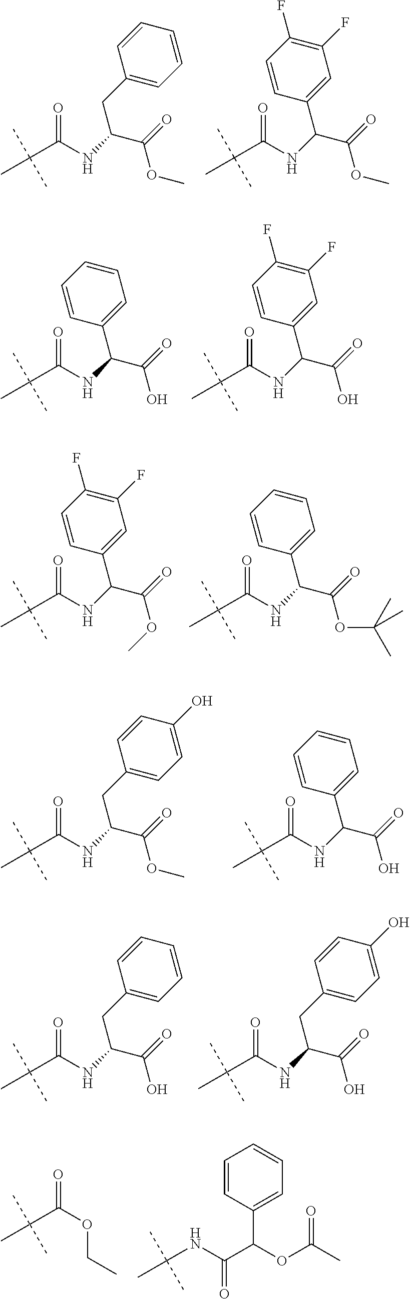

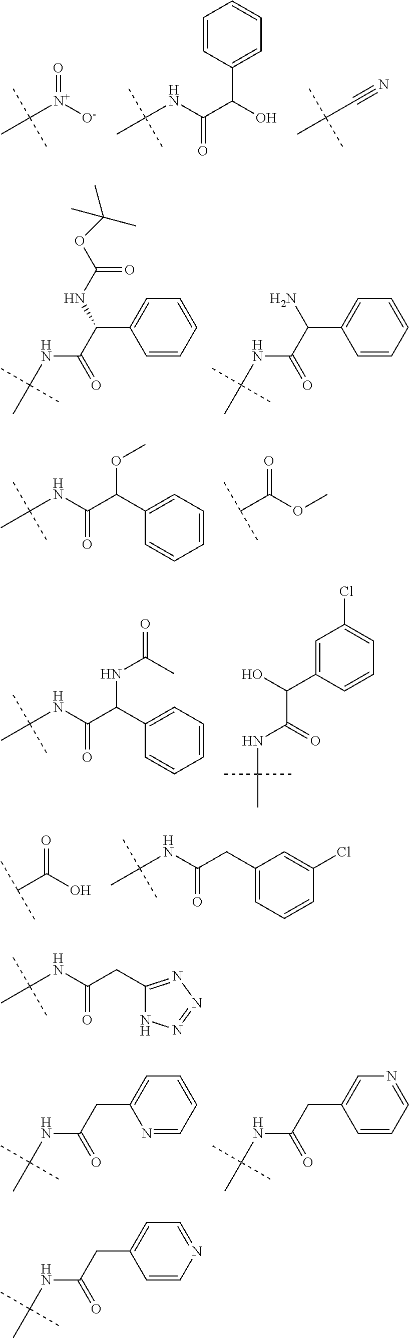

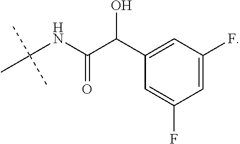

R^{c} preferably denotes H, Hal, Het, CN, NO₂, OCF₃, an alkyl having 1 to 6 carbon atoms, or alkoxy having 1 to 6 carbon atoms
R^{c} more preferably denotes Hal, CN or alkoxy having 1 to 6 carbon atoms, especially Cl, F methoxy, trifluoromethoxy or ethoxy.
R^{e} is preferably H, Hal, NO₂, phenyl or phenoxy, more preferably H or Hal and most preferably denotes H or Cl.

Compounds of formula (I) and related formulae, wherein R^{b} denotes COOH, COOT, wherein T is as defined above and preferably is alkyl having 1 to 8 carbon atoms, or CN are preferred as intermediates for the synthesis of other compounds of formula (I) and related formulae.

Hal is preferably F, Cl or Br and especially F or Cl.

Preferably, at least one of W¹ and W² is CH, more preferably W¹ and W² simultaneously denote CH, also preferably W¹ is CH.

p and p′ is preferably 0, 1 or 2, especially 0 or 1. Most preferably, one of p and p′ is 0.

s is preferably 0 or 2, especially 0.

Ar preferably denotes a monocyclic or bicyclic, unsaturated or aromatic carbocyclic ring having 6 to 14 carbon atoms, which is unsubstituted or monosubstituted, disubstituted by alkyl having 1 to 8 carbon atoms, alkoxy having 1 to 8 carbon atoms, Hal, CF₃, OCF₃, NO₂, N(R³)₂, COOR³, COR³, SO₂N(R³)₂, COHet, tetrazole, O-pyridine, morpholine, OR³, CONH(CH₂)ₚN(R³)₂, and/or CN,

An aromatic carbocyclic ring preferably denotes phenyl, naphthyl or biphenyl.

Ar denotes, for example, phenyl. Throughout the specification, phenyl can be preferably unsubstituted or monosubstituted, disubstituted or trisubstituted by Hal, CF₃, OCF₃, NO₂, OH, alkyl, O-alkyl and/or CN. Also phenyl preferably can be o-, m- or p-tolyl, o-, m- or p-ethyl-phenyl, o-, m- or p-propylphenyl, o-, m- or p-isopropylphenyl, o-, m- or p-tert-butylphenyl, o-, m- or p-hydroxyphenyl, o-, m- or p-nitrophenyl, o-, m- or p-aminophenyl, o-, m- or p-(N-methylamino)phenyl, o-, m- or p-(N-methylaminocarbonyl)phenyl, o-, m- or p-acetamido-phenyl, o-, m- or p-methoxyphenyl, o-, m- or p-ethoxyphenyl, o-, m- or p-ethoxycarbonyl-phenyl, o-, m- or p-(N,N-dimethylamino)phenyl, o-, m- or p-(N,N-dimethylaminocarbonyl)-phenyl, o-, m- or p-(N-ethylamino)phenyl, o-, m- or p-(N,N-diethylamino)phenyl, o-, m- or p-fluorophenyl, o-, m- or p-bromophenyl, o-, m- or p-chlorophenyl, o-, m- or p-(methylsulfonamido)phenyl, o-, m- or p-(methylsulfonyl)phenyl, o, m or p amino-sulfanyl-phenyl, o-, m- or p-phenoxyphenyl, further preferably 2,3-, 2,4-, 2,5-, 2,6-, 3,4- or 3,5-dimethylphenyl, 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2,4- or 2,5-dinitrophenyl, 2,5- or 3,4-dim ethoxyphenyl, 3-nitro-4-chlorophenyl, 3-amino-4-chloro-, 2-amino-3-chloro-, 2-amino-4-chloro-, 2-amino-5-chloro- or 2-amino-6-chlorophenyl, 2-nitro-4-N,N-dimethylamino- or 3-nitro-4-N,N-dimethylaminophenyl, 2,3-diaminophenyl, 2,3,4-, 2,3,5-, 2,3,6-, 2,4,6- or 3,4,5-trichlorophenyl, 2,4,6-trimethoxyphenyl, 2-hydroxy-3,5-dichlorophenyl, p-iodophenyl, 3,6-dichloro-4-aminophenyl, 4-fluoro-3-chlorophenyl, 2-fluoro-4-bromophenyl, 2,5-difluoro-4-bromophenyl, 3-bromo-6-methoxyphenyl, 3-chloro-6-methoxyphenyl, 3-chloro-4-acetamidophenyl, 3-fluoro-4-methoxyphenyl, 3-amino-6-methylphenyl, 3-chloro-4-acetamidophenyl or 2,5-dimethyl-4-chlorophenyl.

Ar preferably denotes phenyl.

Ar particularly preferably denotes, for example, phenyl which is unsubstituted or monosubstituted by F, Cl, methoxy or NO₂.

Het preferably denotes a monocyclic or bicyclic, saturated, unsaturated or aromatic heterocyclic ring having 1 to 3 N, O atoms or one CO function, which is unsubstituted or monosubstituted, disubstituted or trisubstituted by alkyl having 1 to 8 carbon atoms, alkoxy having 1 to 8 carbon atoms, Hal, CF₃, OCF₃, NO₂, N(R³)₂, COOR³, COR^{S}, SO₂N(R³)₂, COAr, OR³, Ar, CONH(CH₂)ₚN(R³)₂, Cyc, SO₂N(R³)₂, and/or CN.

In a preferred embodiment Het denote unsubstituted tetrazole

Het is preferably a 6 to 14 membered ring system and denotes, not withstanding further substitutions, for example, 2- or 3-furyl, 2- or 3-thienyl, 1-, 2- or 3-pyrrolyl, 1-, 2-, 4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -3- or -5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2-, 3-, 4-, 5-, 6- or 7-indolyl, indazolyl, 4- or 5-isoindolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-isoquinolyl, 3-, 4-, 5-, 6-, 7- or 8-cinnolinyl, 2-, 4-, 5-, 6-, 7- or 8-quinazolinyl, 5- or 6-quinoxalinyl, 2-, 3-, 5-, 6-, 7- or 8-2H-benzo-1,4-oxazinyl, furthermore preferably 1,3-benzodioxol-5-yl, 1,4-benzodioxane-6-yl, 2,1,3-benzothiadiazol-4- or -5-yl or 2,1,3-benzoxadiazol-5-yl.

The heterocyclic groups may also be partially or fully hydrogenated.

Het can thus also denote, for example, 2,3-dihydro-2-, -3-, -4- or -5-furyl, 2,5-dihydro-2-, -3-, -4- or -5-furyl, tetrahydro-2- or -3-furyl, 1,3-dioxolan-4-yl, tetrahydro-2- or -3-thienyl, 2,3-dihydro-1-, -2-, -3-, -4- or -5-pyrrolyl, 2,5-dihydro-1-, -2-, -3-, -4- or -5-pyrrolyl, 1-, 2- or 3-pyrrolidinyl, tetrahydro-1-, -2- or -4-imidazolyl, 2,3-dihydro-1-, -2-, -3-, -4- or -5-pyrazolyl, tetrahydro-1-, -3- or -4-pyrazolyl, 1,4-dihydro-1-, -2-, -3- or -4-pyridyl, 1,2,3,4-tetrahydro-1-, -2-, -3-, -4-, -5- or -6-pyridyl, 1-, 2-, 3- or 4-piperidinyl, 2-, 3- or 4-morpholinyl, tetrahydro-2-, -3- or -4-pyranyl, 1,4-dioxaneyl, 1,3-dioxane-2-, -4- or -5-yl, hexahydro-1-, -3- or -4-pyridazinyl, hexahydro-1-, -2-, -4- or -5-pyrimidinyl, 1-, 2- or 3-piperazinyl, 1,2,3,4-tetrahydro-1-, -2-, -3-, -4-, -5-, -6-, -7- or -8-quinolyl, 1,2,3,4-tetrahydro-1-, -2-, -3-, -4-, -5-, -6-, -7- or -8-isoquinolyl, 2-, 3-, 5-, 6-, 7- or 8-3,4-dihydro-2H-benzo-1,4-oxazinyl, furthermore preferably 2,3-methylenedioxyphenyl, 3,4-methylenedioxyphenyl, 2,3-ethylenedioxyphenyl, 3,4-ethylenedioxyphenyl, 3,4-(difluoromethylenedioxy)phenyl, 2,3-dihydrobenzofuran-5- or -6-yl, 2,3-(2-oxomethylenedioxy)phenyl or also 3,4-dihydro-2H-1,5-benzodioxepin-6- or -7-yl, furthermore preferably 2,3-dihydrobenzofuranyl or 2,3-dihydro-2-oxofuranyl. Throughout the specification, pyridyl is 2-, 3- or 4-pyridyl, which can be preferably unsubstituted or mono-substituted, disubstituted or trisubstituted by Hal, CF₃, OCF₃, NO₂, OH, alkyl, O-alkyl and/or CN.

If Het denotes a N-Atom bearing saturated heterocycle, Het is preferably linked to the rest of the molecule via an N-Atom.

The compounds of the formula (I) and related formulae can have one or more centres of chirality and can therefore occur in various stereoisomeric forms. The formula I covers all these forms.

Accordingly, the invention relates, in particular, to the use of those compounds of the formula I, wherein at least one of the said groups has one of the preferred meanings indicated above. Some preferred groups of compounds can be expressed by the following sub-formulae I-b to I-e, which conform to the formula I and in which the radicals not designated in greater detail have the meaning indicated under the formula I, but in which

### in I-b

R^{a} is phenyl
in I-c R^{a} is phenyl

### R^{b} is CONHQR^{d},

Q is CH₂, CH₂CH₂ or SO₂,
R^{d} is Ar or cycloalkyl having 3 to 7 carbon atoms, or a saturated heterocyclic ring having 3, 4 or 5 carbon atoms and 1 or 2 N or O atoms,
   in I-d R^{a} is 2-pyridyl

### R^{b} is CONHQR^{d},

Q is CH₂, CH₂CH₂ or SO₂,
R^{d} is Ar or cycloalkyl having 3 to 7 carbon atoms, or a saturated heterocyclic ring having 3, 4 or 5 carbon atoms and 1 or 2 N or O atoms,
   in I-e R^{a} is phenyl
R^{b} is tetrazolyl or oxadiazolyl,
R^{c} is Cl
   and pharmaceutically usable derivatives, solvates, salts and stereoisomers thereof, including mixtures thereof in all ratios.

A further preferred embodiment of the compounds of formula (I) is that of sub-formula Ia:

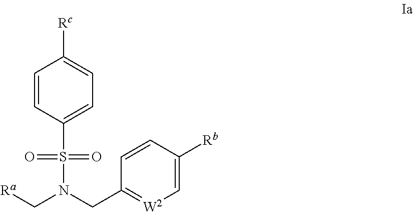

wherein the R^{a}, R^{b}, R^{c} and W² are as defined above.

In another preferred embodiment, the invention provides compounds of Formula (Ib):

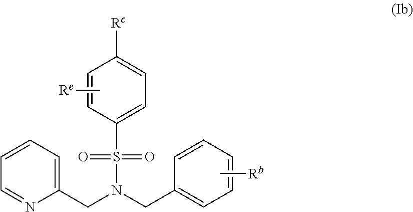

Wherein R^{b}, R^{c} and R^{e} are as above defined,
and pharmaceutically acceptable derivatives, solvates, tautomers, salts and stereoisomers thereof, including mixtures thereof in all ratios.

In another embodiment, the invention provides compounds of Formula (Ic)

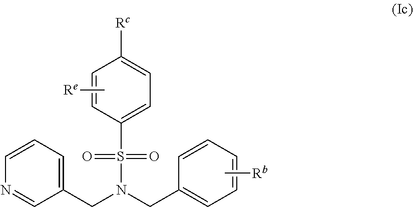

Wherein R^{b}, R^{c} and R^{e} are as above defined
and pharmaceutically acceptable derivatives, solvates, tautomers, salts and stereoisomers thereof, including mixtures thereof in all ratios.

In another preferred embodiment, the invention provides compounds of Formula (Id)

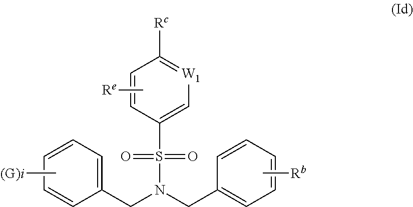

Wherein G is H, Hal, OR³, tetrazole, phenyl, pyrazol, CONH(CH₂)ₚN(R³)₂,
i is 1 or 2
R^{b}, W₁, R^{c}, R^{e} and p are as defined above
and pharmaceutically acceptable derivatives, solvates, tautomers, salts and stereoisomers thereof, including mixtures thereof in all ratios.

In another embodiment, the invention provides compounds of formula (Ie):

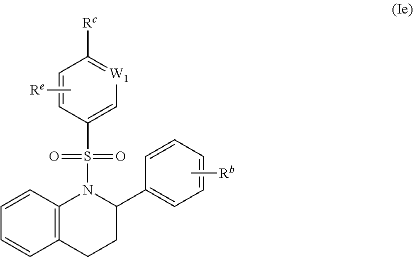

Wherein R^{b}, W₁, R^{c}, R^{e} are as defined above
and pharmaceutically acceptable derivatives, solvates, tautomers, salts and stereoisomers thereof, including mixtures thereof in all ratios

In another embodiment, the invention provides compounds of formula (If):

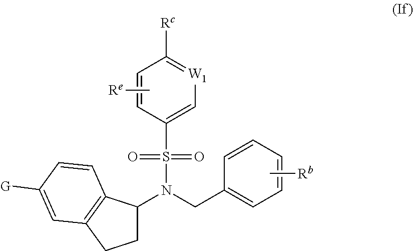

Wherein R^{b}, W₁, R^{c}, R^{e} and G are as defined above
and pharmaceutically acceptable derivatives, solvates, tautomers, salts and stereoisomers thereof, including mixtures thereof in all ratios.

In another embodiment, the invention provides compounds of formula (Ig):

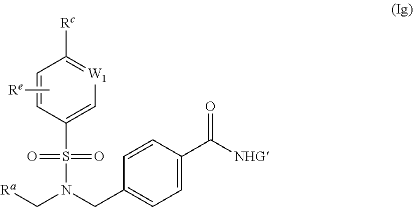

Wherein R^{a}, W₁, R^{c}, R^{e} are as defined above and G′ denotes Het or a linear or branched (C1-C6)alkylene, wherein 1, 2 or 3 H atoms may be replaced by OR³, CON(R³)₂, CO₂R³, an aryl group, preferably a phenyl, and/or 2 geminal H atom may form a Cyc group, and wherein 1 or 2 CH₂ group may be replaced by SO₂.
and pharmaceutically acceptable derivatives, solvates, tautomers, salts and stereoisomers thereof, including mixtures thereof in all ratios.

In another embodiment, the invention provides compounds of formula (Ih):

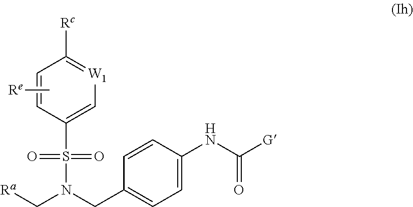

Wherein R^{a}, W₁, R^{c}, R^{e}, G′ are as above defined.
and pharmaceutically acceptable derivatives, solvates, tautomers, salts and stereoisomers thereof, including mixtures thereof in all ratios.

In embodiment 1), the invention provides compounds of Formula (I′) wherein R^{a} is phenyl, which is unsubstituted or substituted by one or more of the groups Hal, CN, NO₂, CF₃, OCF₃, SCN or alkoxy having 1 to 8 carbon atoms

In embodiment 2), the invention provides compounds of Formula (I′), wherein R^{b} denotes a group C(O)NHQR^{d}, wherein Q and R^{d} are as defined above, or denotes 1 or 5 tetrazolyl, 1,2,3-oxadiazol-4- or 5-yl, 1,2,4-oxadiazol-3- or 5-yl or 5-hydroxy-1,3,4-oxadiazol-2yl or 5-hydroxy-1,3,4-oxadiazol-2-yl.

In embodiment 3), the invention provides compounds of Formula (I′) wherein R^{c} preferably denotes Hal, CN or alkoxy having 1 to 6 carbon atoms.

In embodiment 4), the invention provides compounds of Formula (I′), wherein R^{d} is preferably Ar or cycloalkyl having 3 to 7 carbon atoms or a saturated heterocyclic ring having 3, 4 or 5 carbon atoms and 1 or 2 N or O atoms.

In embodiment 5) the invention provides compounds of Formula (I′), wherein W¹ preferably denotes CH.

In embodiment 6), the invention provides compounds of Formula (I′), wherein one of p and p′ is O.

Particular preference is given to the compounds of the present invention selected from the following group 1 to 371:

| structure | Ex |
|---|---|
| 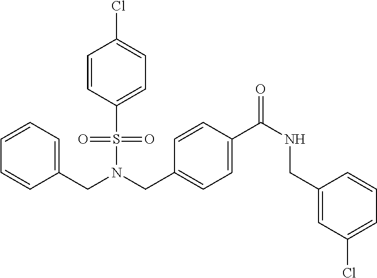 | 1 |
| 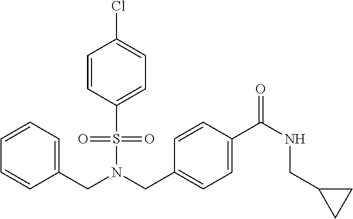 | 2 |
| 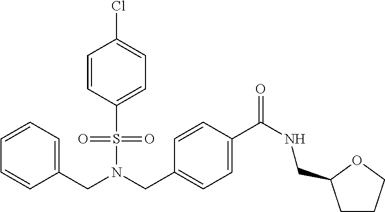 | 3 |
| 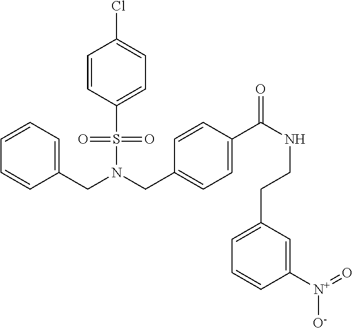 | 4 |
| 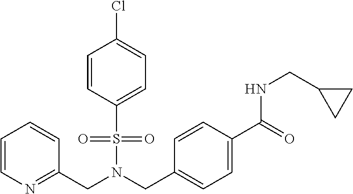 | 5 |
| 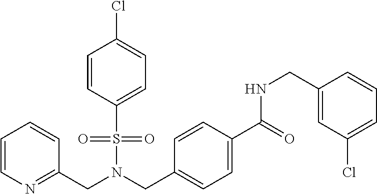 | 6 |
| 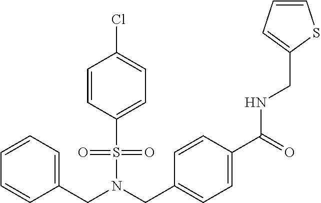 | 7 |
| 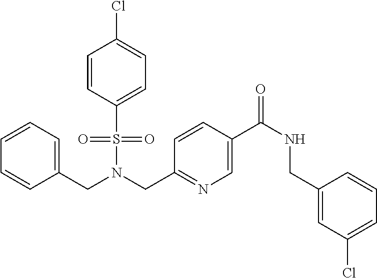 | 8 |
| 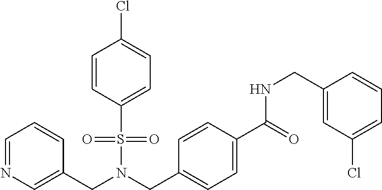 | 9 |
| 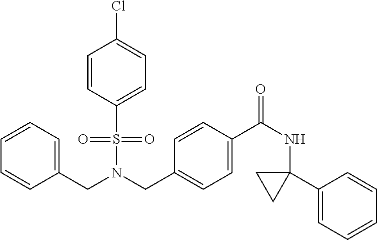 | 10 |
| 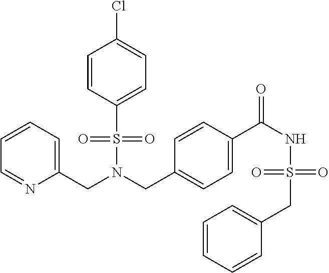 | 11 |
| 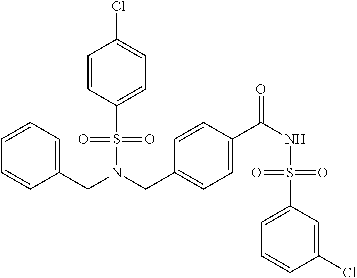 | 12 |
| 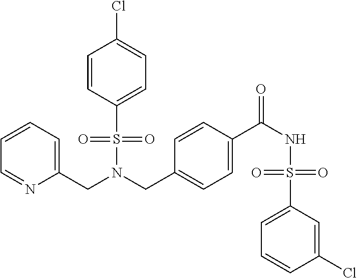 | 13 |
| 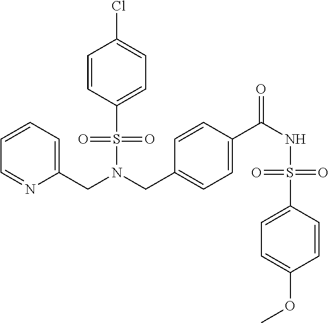 | 14 |
| 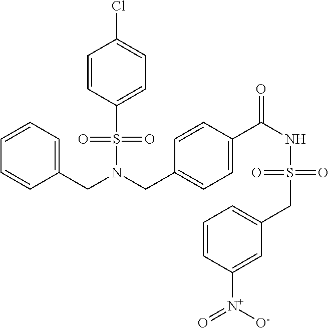 | 15 |
| 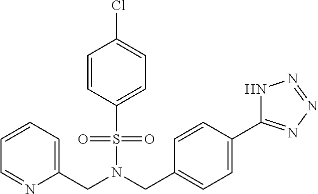 | 16 |
| 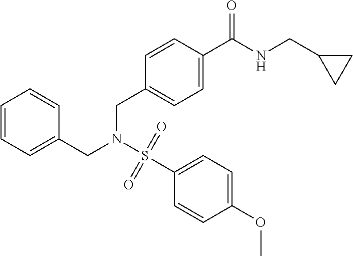 | 17 |
| 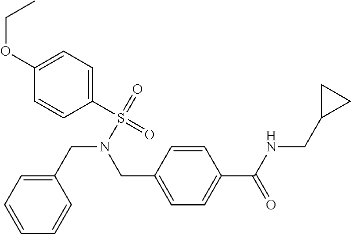 | 18 |
| 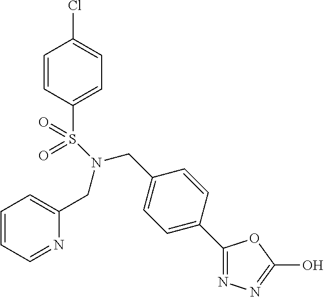 | 19 |
| 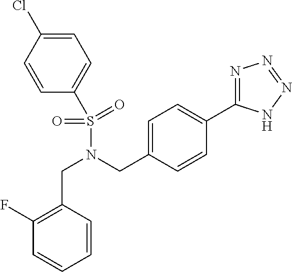 | 20 |
| 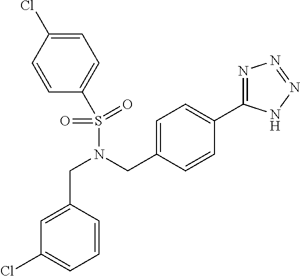 | 21 |
| 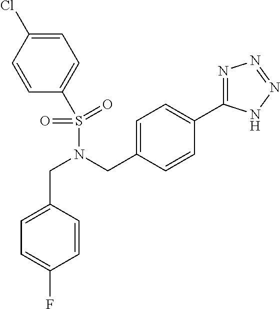 | 22 |
| 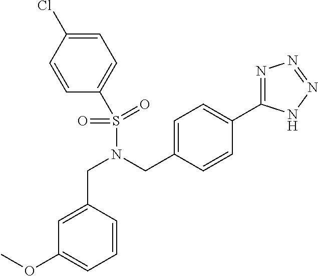 | 23 |
| 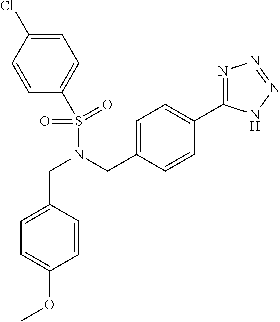 | 24 |
| 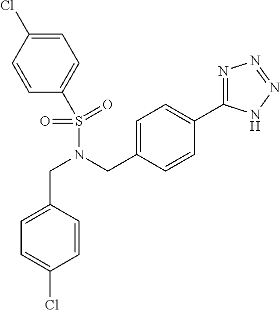 | 25 |
| 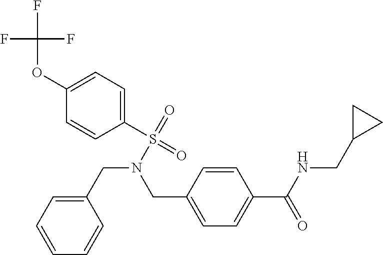 | 26 |
| 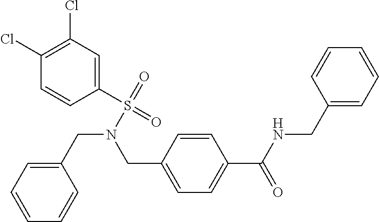 | 27 |
| 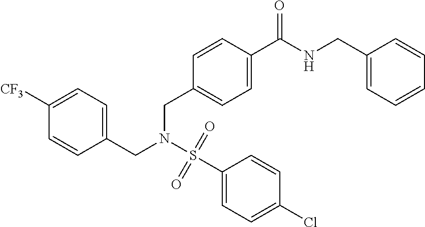 | 28 |
| 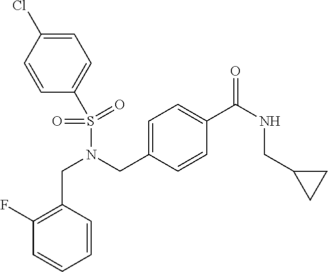 | 29 |
| 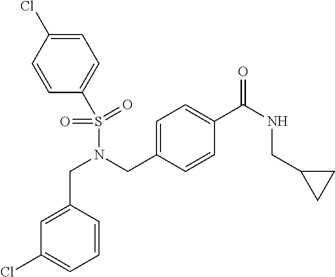 | 30 |
| 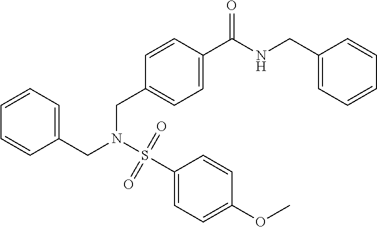 | 31 |
| 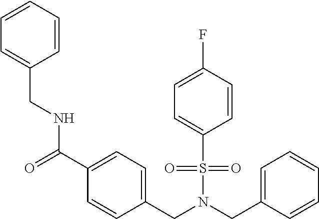 | 32 |
| 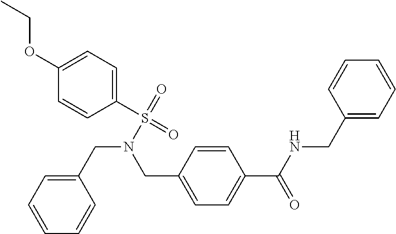 | 33 |
| 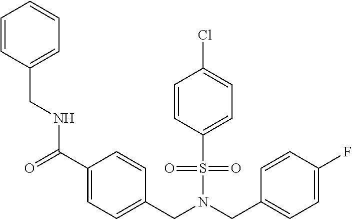 | 34 |
| 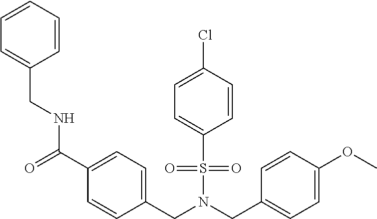 | 35 |
| 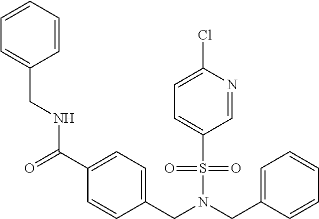 | 36 |
| 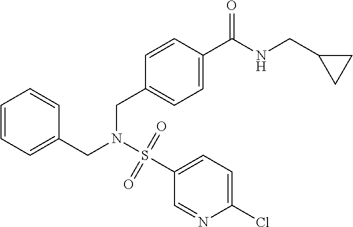 | 37 |
| 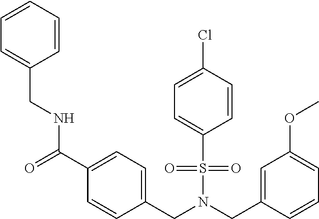 | 38 |
| 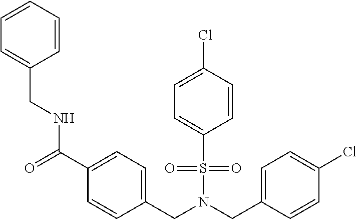 | 39 |
| 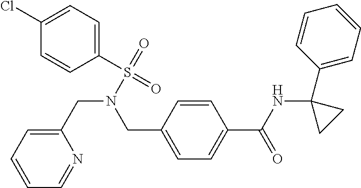 | 40 |
| 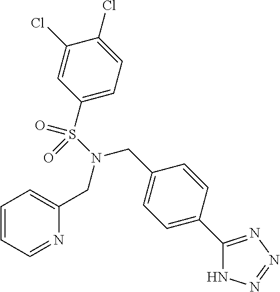 | 41 |
| 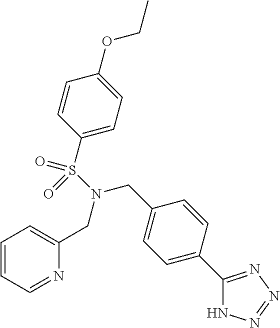 | 42 |
| 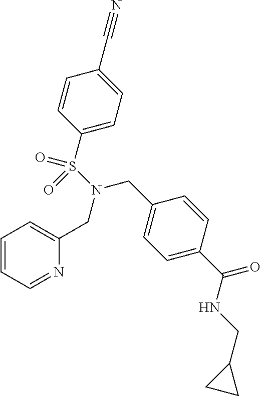 | 43 |
| 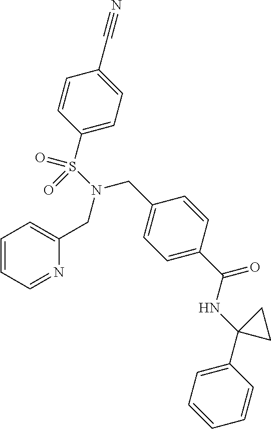 | 44 |
| 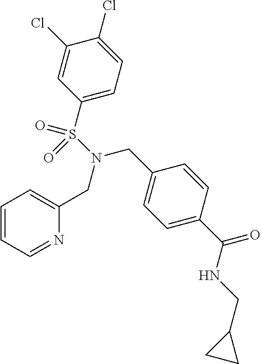 | 45 |
| 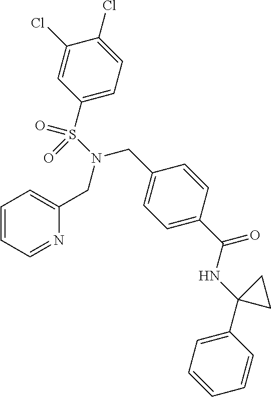 | 46 |
| 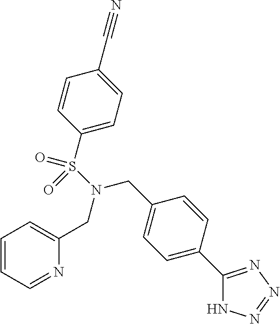 | 47 |
| 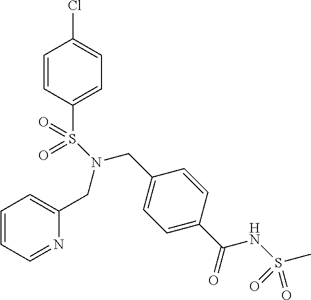 | 48 |
| 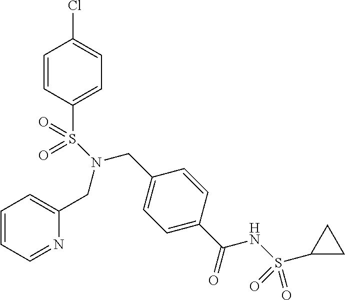 | 49 |
| 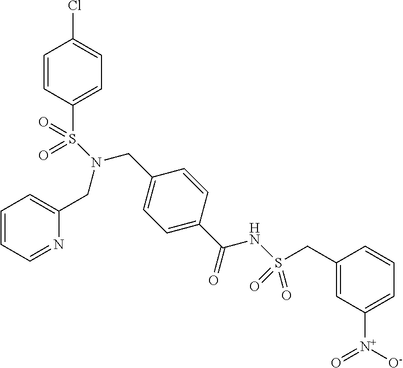 | 50 |
| 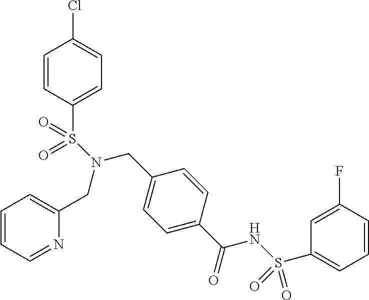 | 51 |
| 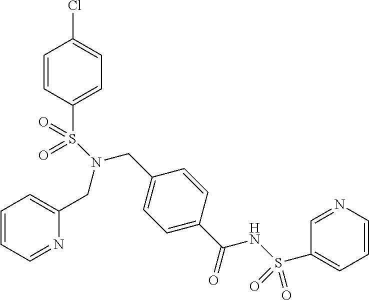 | 52 |
| 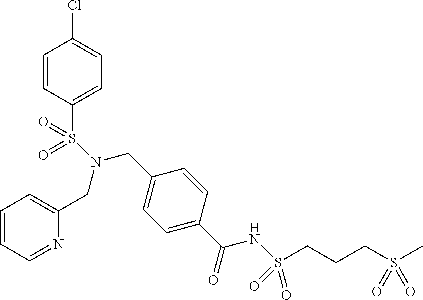 | 53 |
| 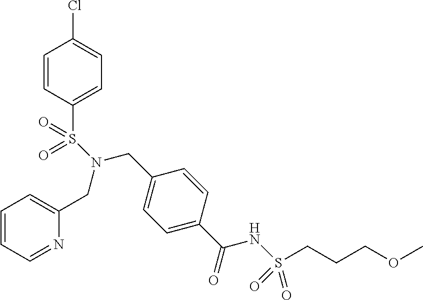 | 54 |
| 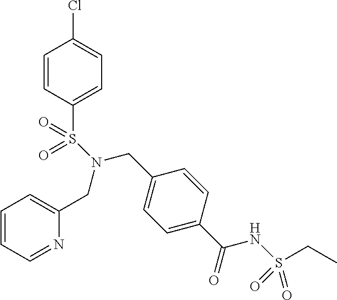 | 55 |
| 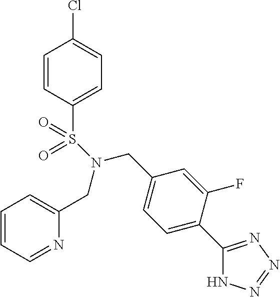 | 56 |
| 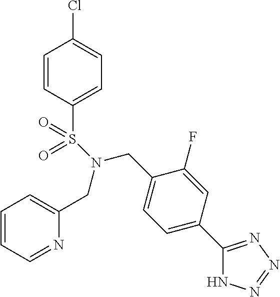 | 57 |
| 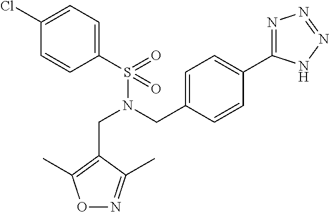 | 58 |
| 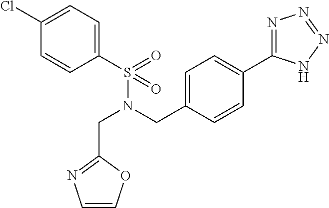 | 49 |
| 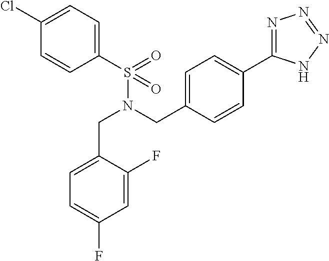 | 60 |
| 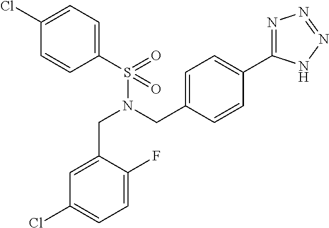 | 61 |
| 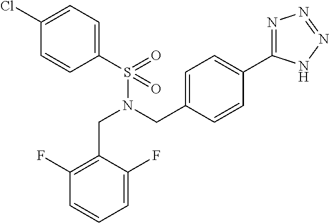 | 62 |
| 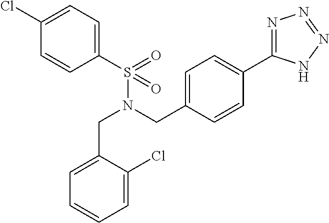 | 63 |
| 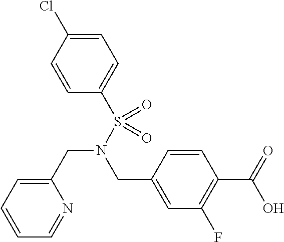 | 64 |
| 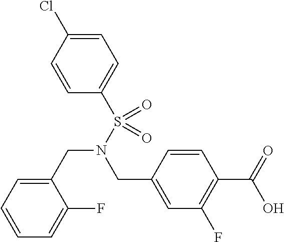 | 65 |
| 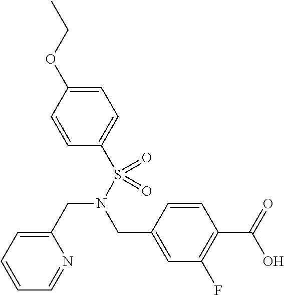 | 66 |
| 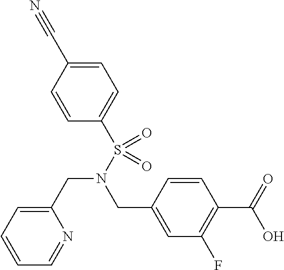 | 67 |
| 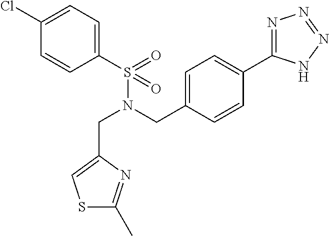 | 68 |
| 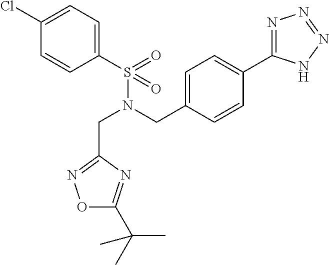 | 69 |
| 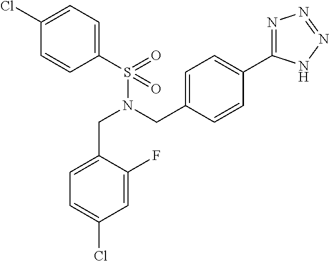 | 70 |
| 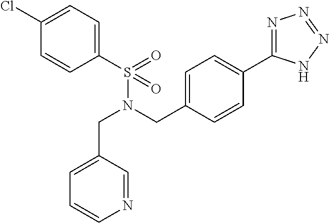 | 71 |
| 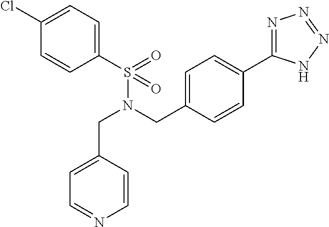 | 72 |
| 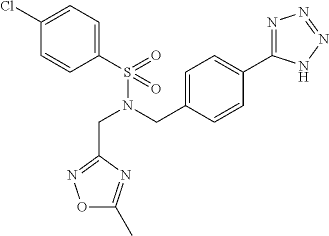 | 73 |
| 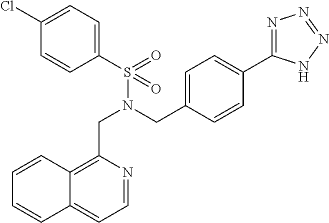 | 74 |
| 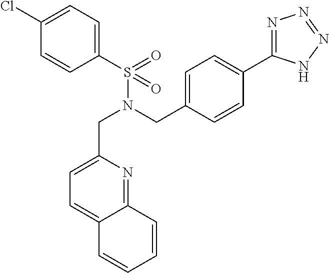 | 75 |
| 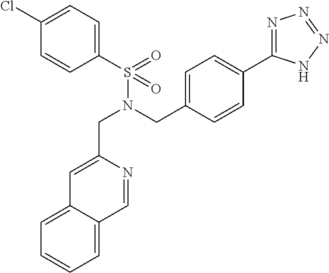 | 76 |
| 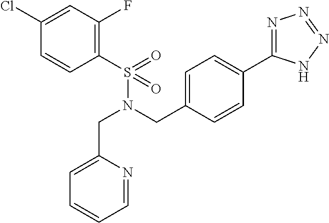 | 77 |
| 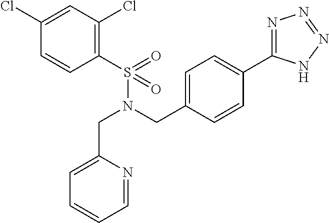 | 78 |
| 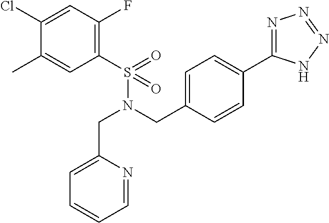 | 79 |
| 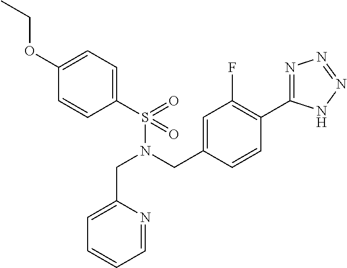 | 80 |
| 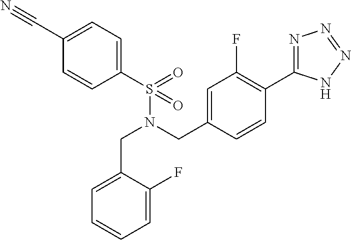 | 81 |
| 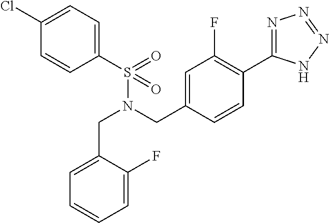 | 82 |
| 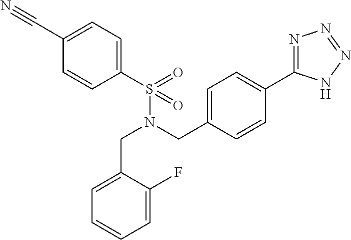 | 83 |
| 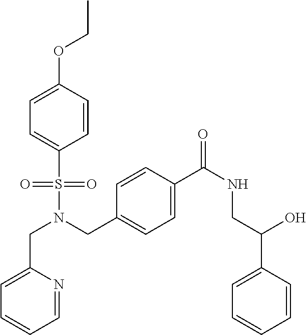 | 84 |
| 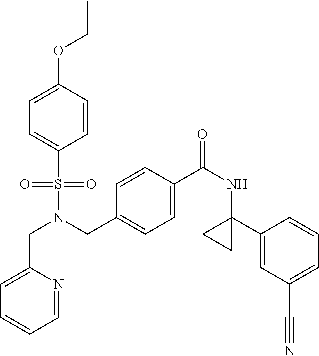 | 85 |
| 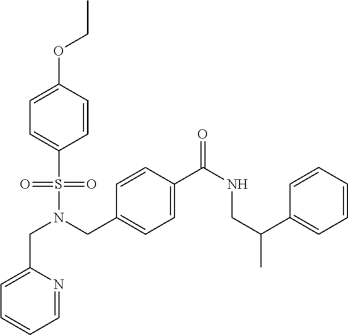 | 86 |
| 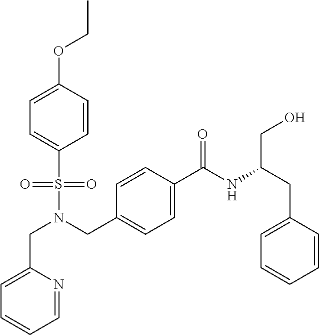 | 87 |
| 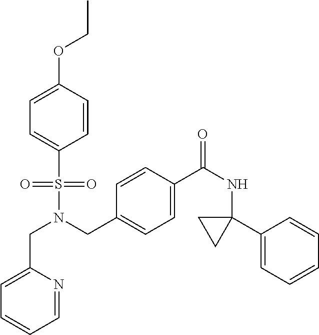 | 88 |
| 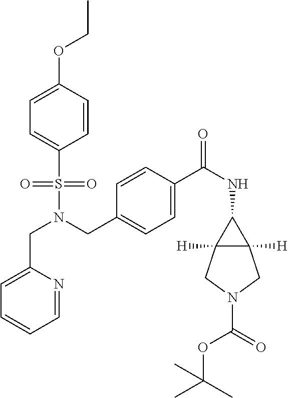 | 89 |
| 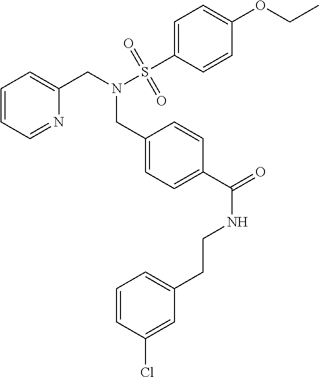 | 90 |
| 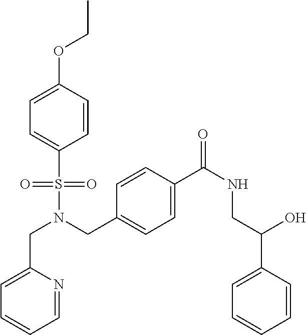 | 91 |
| 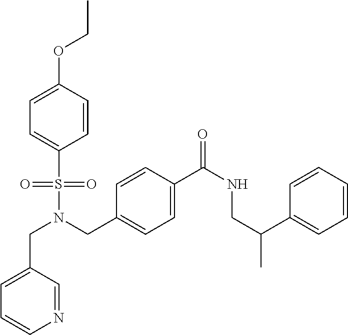 | 92 |
| 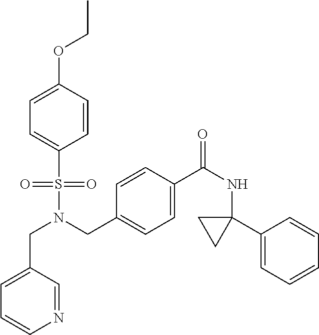 | 93 |
| 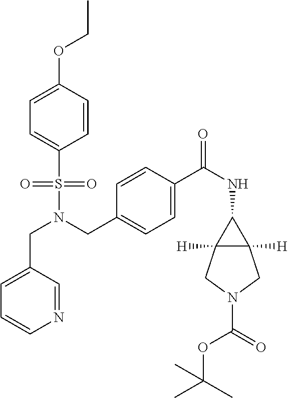 | 94 |
| 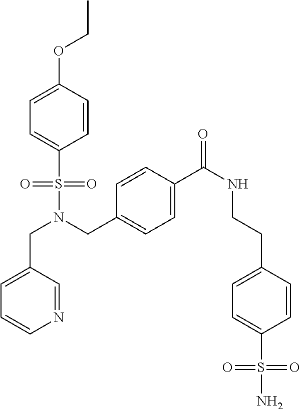 | 95 |
| 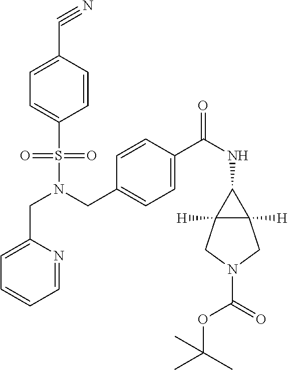 | 96 |
| 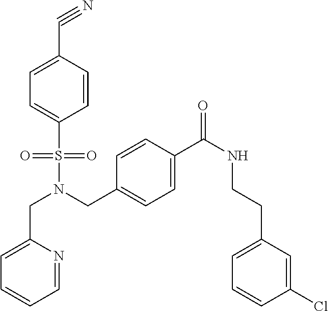 | 97 |
| 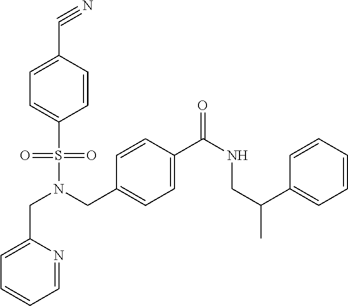 | 98 |
| 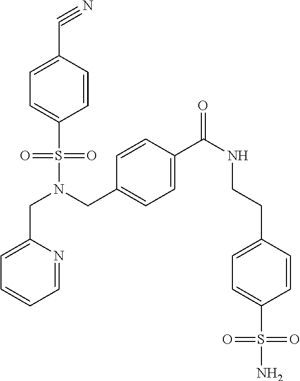 | 99 |
| 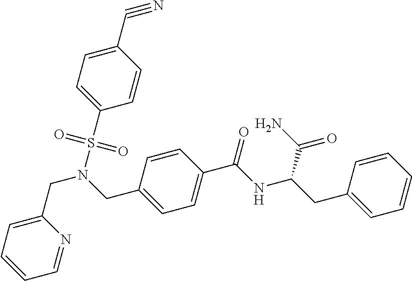 | 100 |
| 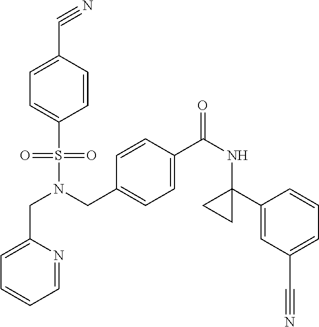 | 101 |
| 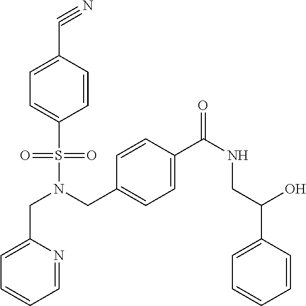 | 102 |
| 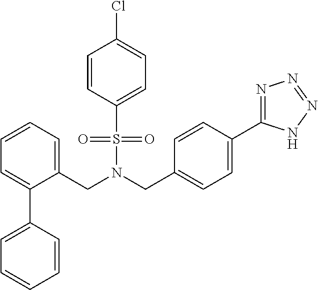 | 103 |
| 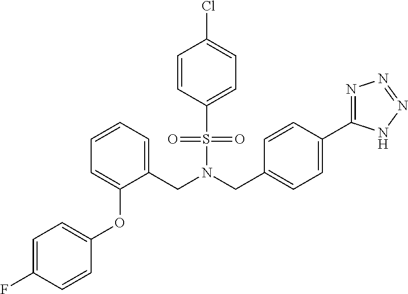 | 104 |
| 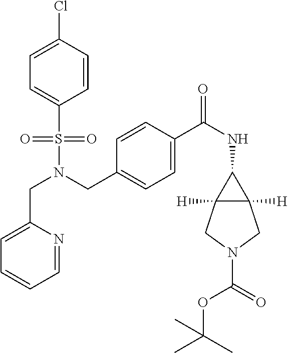 | 105 |
| 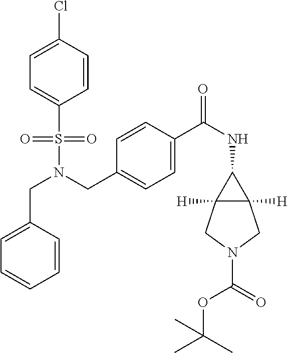 | 106 |
| 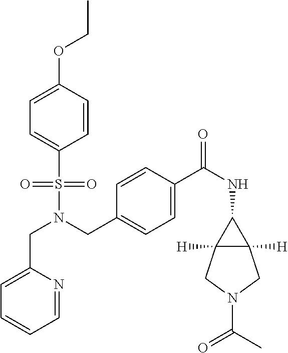 | 107 |
| 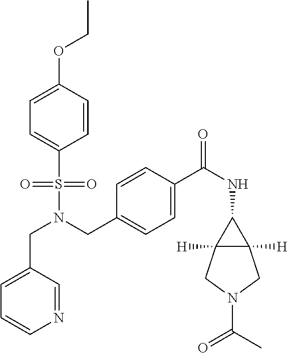 | 108 |
| 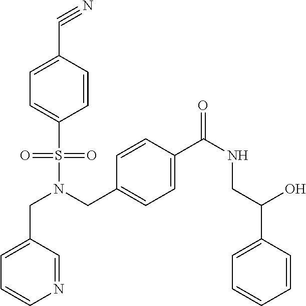 | 109 |
| 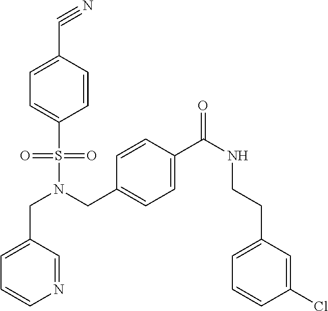 | 110 |
| 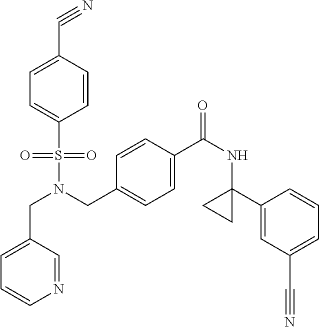 | 111 |
| 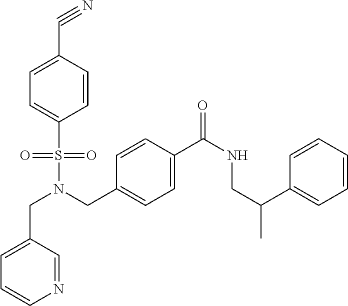 | 112 |
| 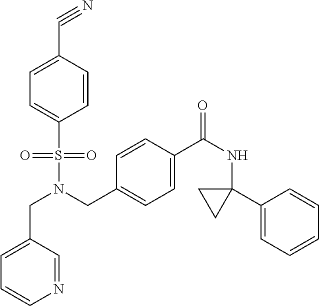 | 113 |
| 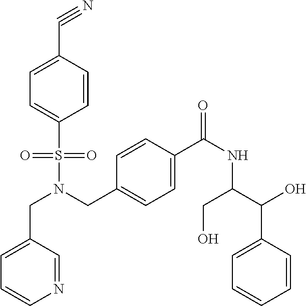 | 114 |
| 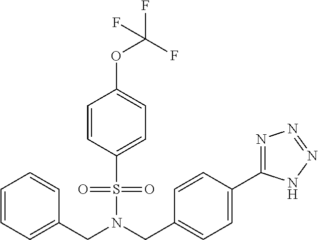 | 115 |
| 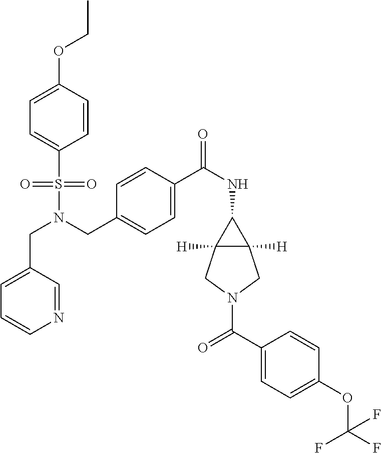 | 116 |
| 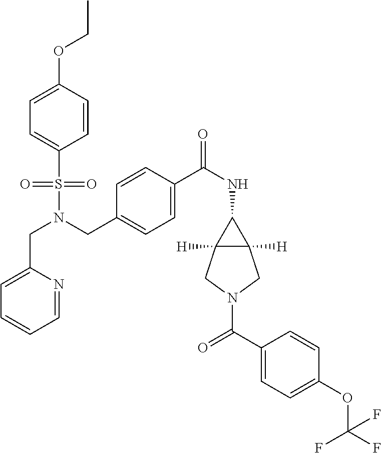 | 117 |
| 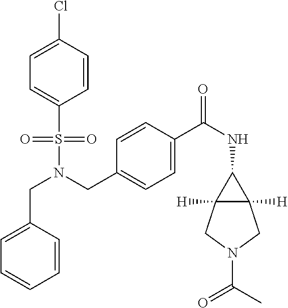 | 118 |
| 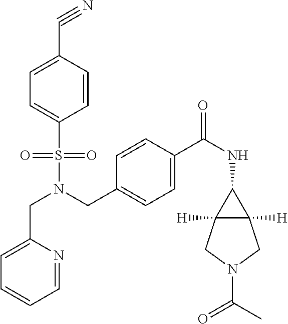 | 119 |
| 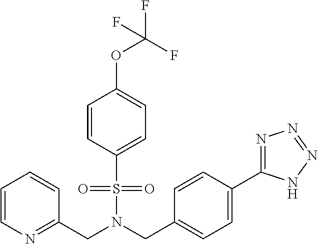 | 120 |
| 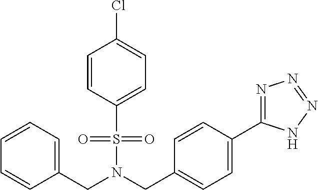 | 121 |
| 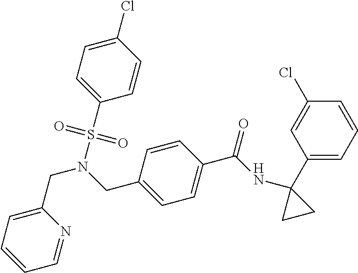 | 122 |
| 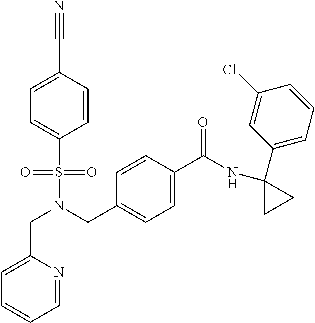 | 123 |
| 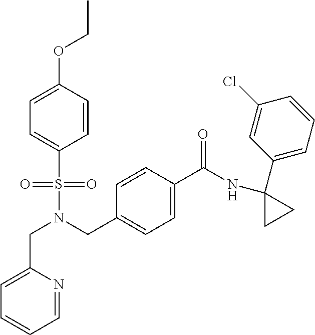 | 124 |
| 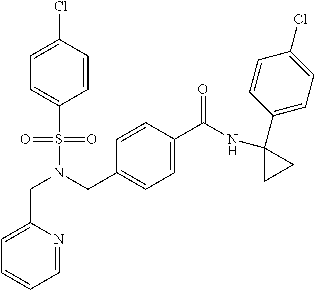 | 125 |
| 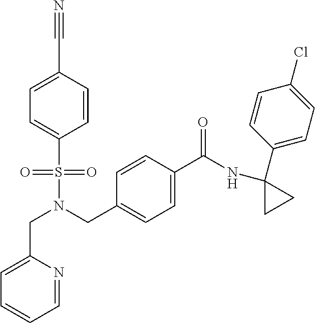 | 126 |
| 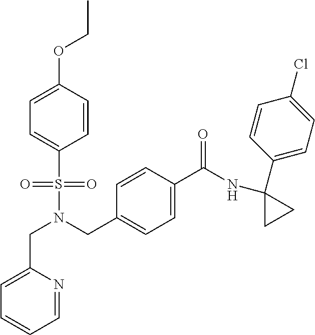 | 127 |
| 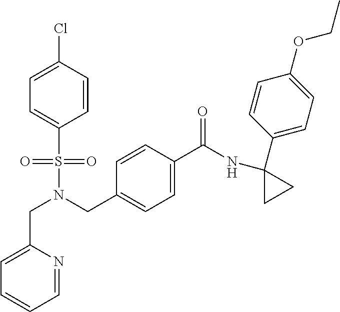 | 128 |
| 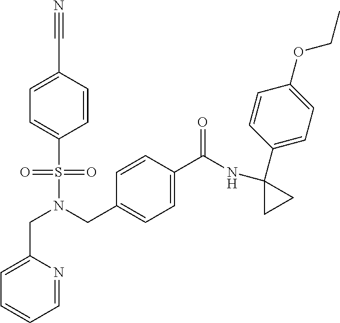 | 129 |
| 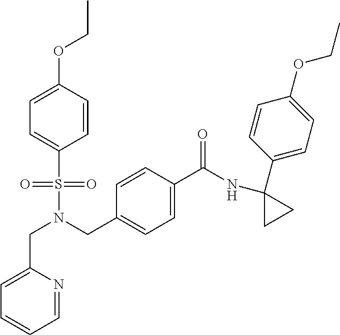 | 130 |
| 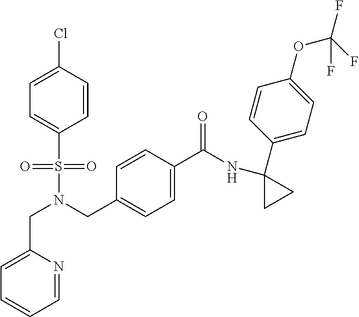 | 131 |
| 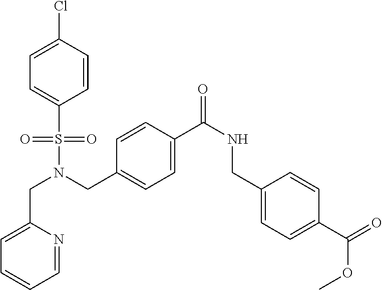 | 132 |
| 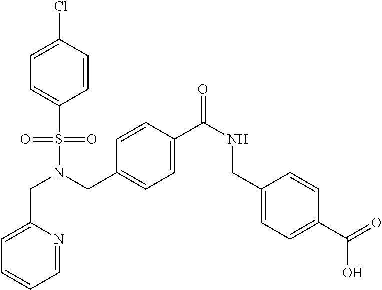 | 133 |
| 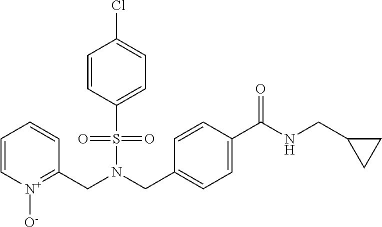 | 134 |
| 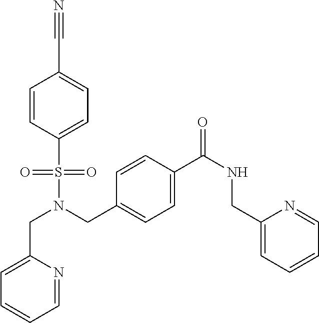 | 135 |
| 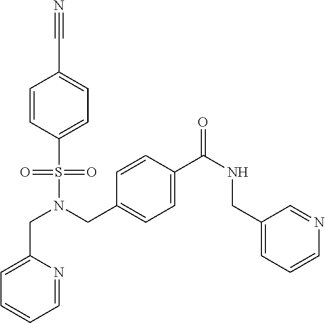 | 136 |
| 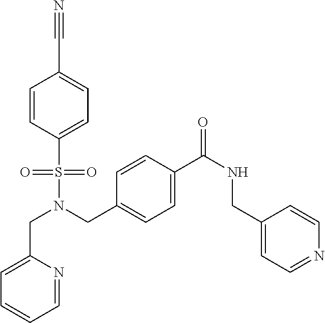 | 137 |
| 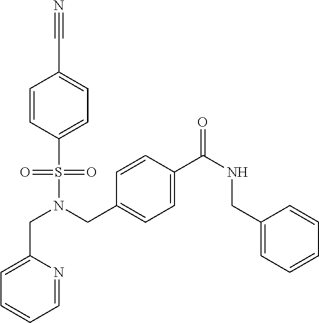 | 138 |
| 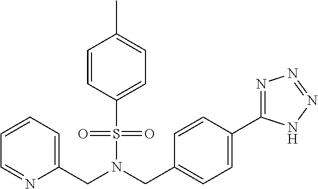 | 139 |
| 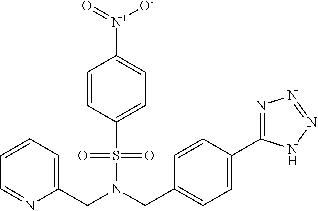 | 140 |
| 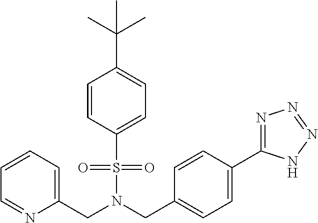 | 141 |
| 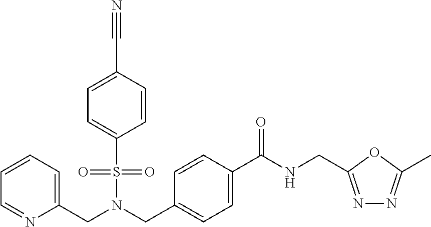 | 142 |
| 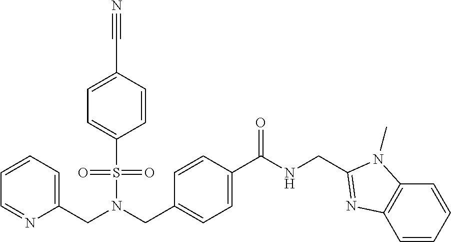 | 143 |
| 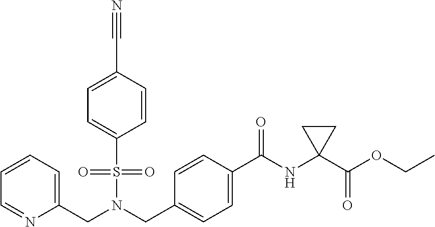 | 144 |
| 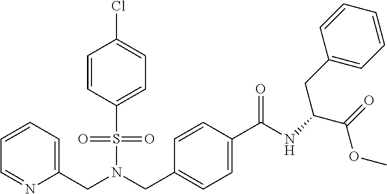 | 145 |
| 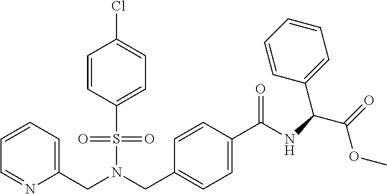 | 146 |
| 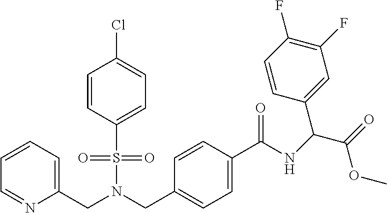 | 147 |
| 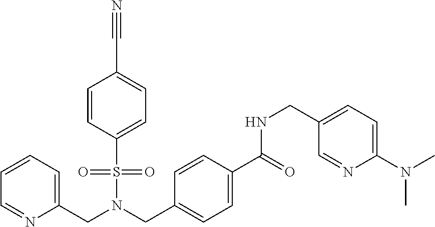 | 148 |
| 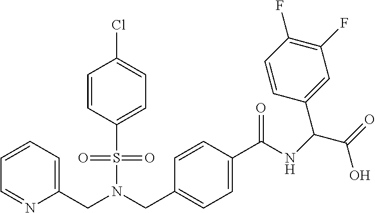 | 149 |
| 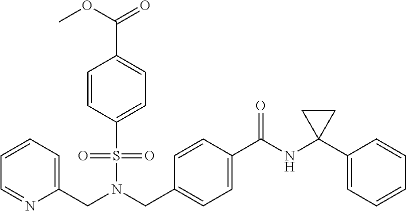 | 150 |
| 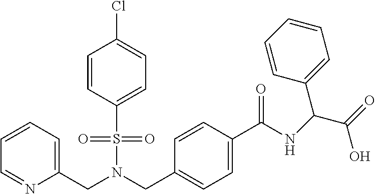 | 151 |
| 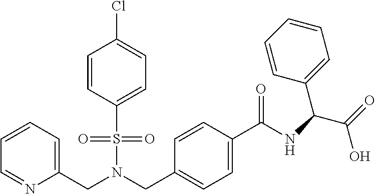 | 152 |
| 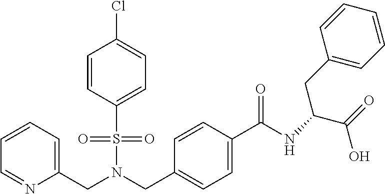 | 153 |
| 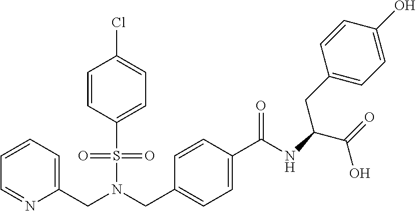 | 154 |
| 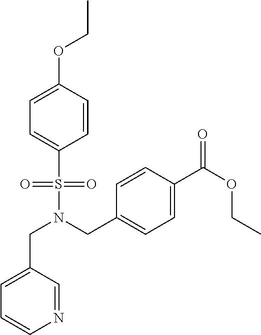 | 155 |
| 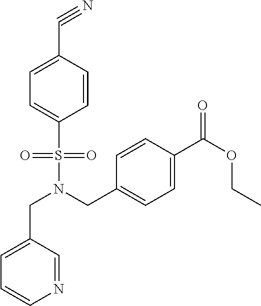 | 156 |
| 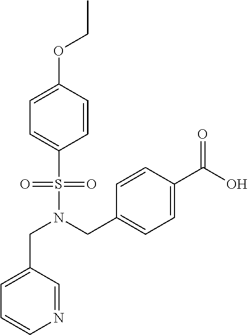 | 157 |
| 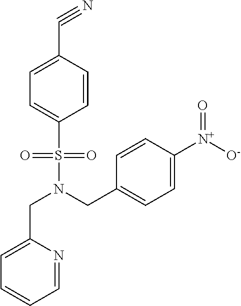 | 158 |
| 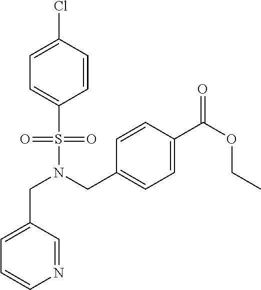 | 159 |
| 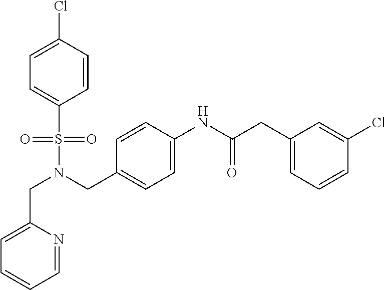 | 160 |
| 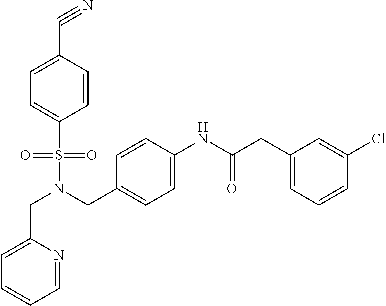 | 161 |
| 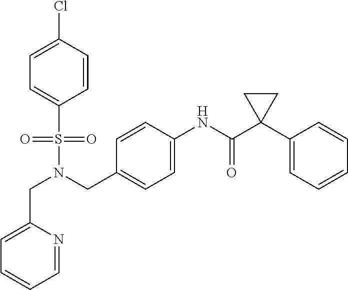 | 162 |
| 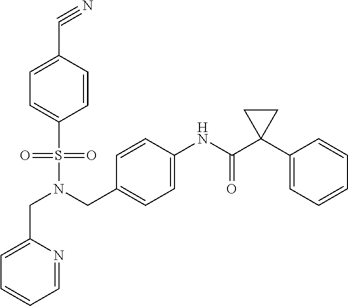 | 163 |
| 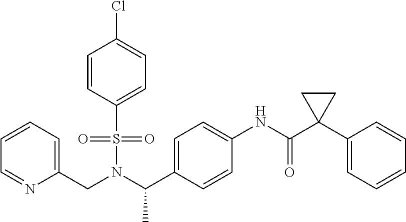 | 164 |
| 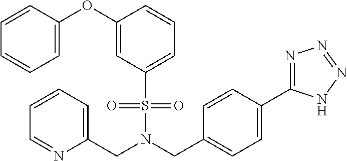 | 165 |
| 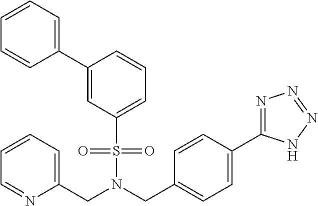 | 166 |
| 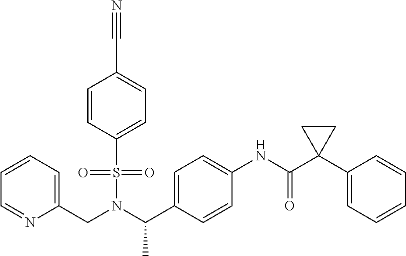 | 167 |
| 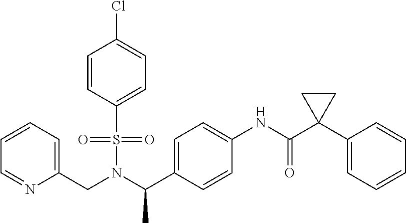 | 168 |
| 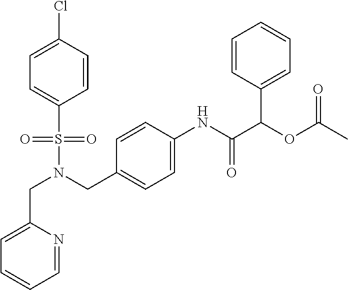 | 169 |
| 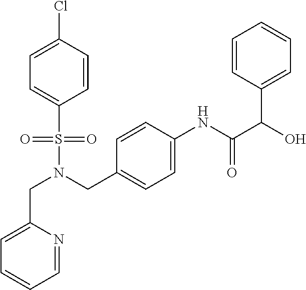 | 170 |
| 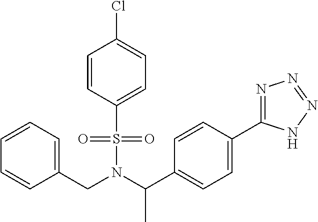 | 171 |
| 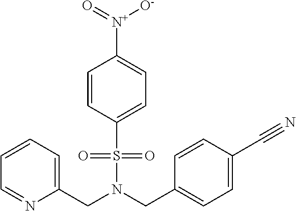 | 172 |
| 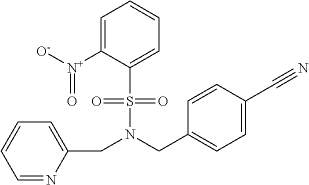 | 173 |
| 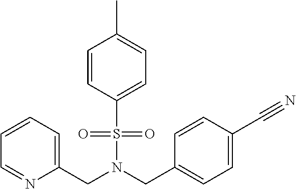 | 174 |
| 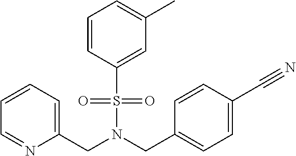 | 175 |
| 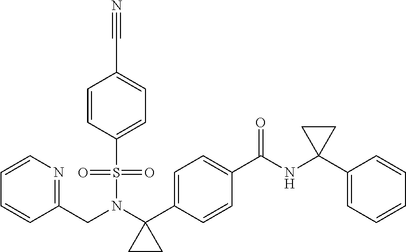 | 176 |
| 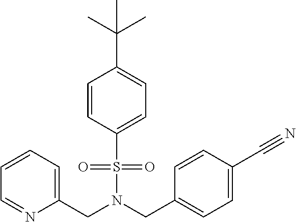 | 177 |
| 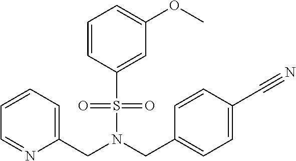 | 178 |
| 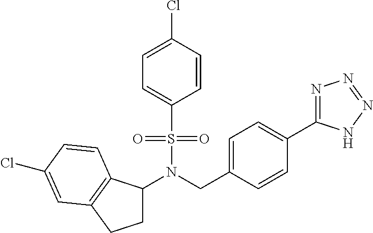 | 179 |
| 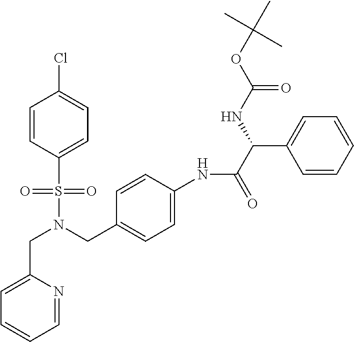 | 180 |
| 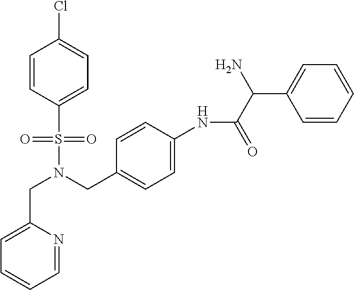 | 181 |
| 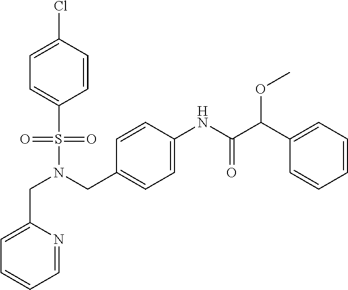 | 182 |
| 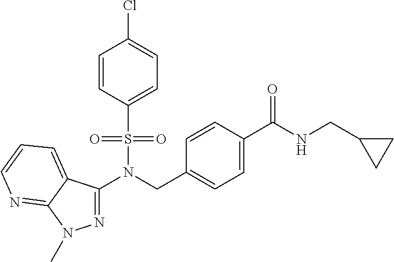 | 183 |
| 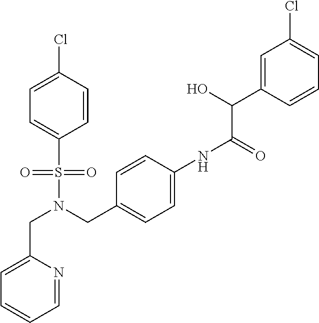 | 184 |
| 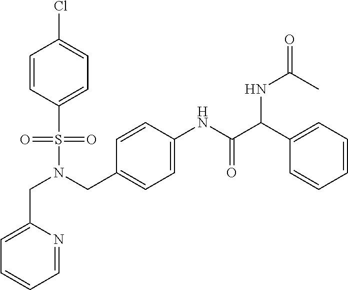 | 185 |
| 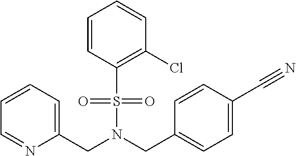 | 186 |
| 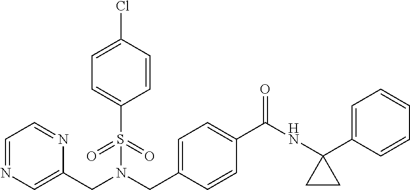 | 187 |
| 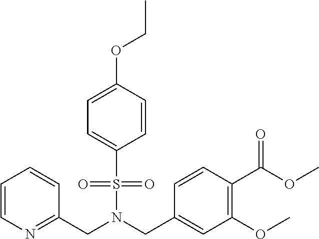 | 188 |
| 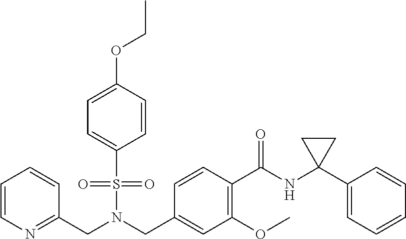 | 189 |
| 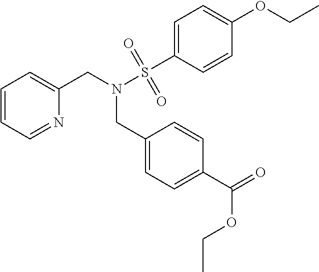 | 190 |
| 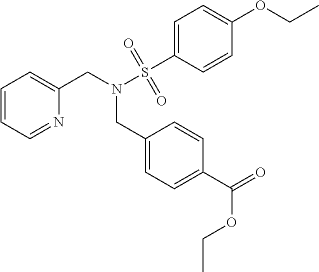 | 191 |
| 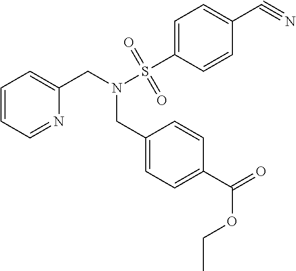 | 192 |
| 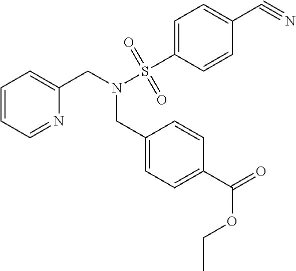 | 193 |
| 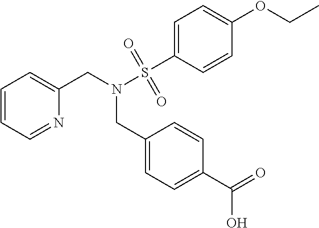 | 194 |
| 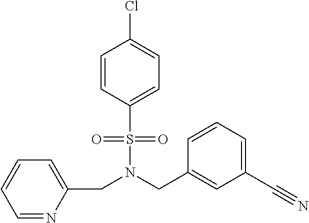 | 195 |
| 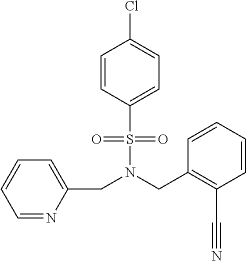 | 196 |
| 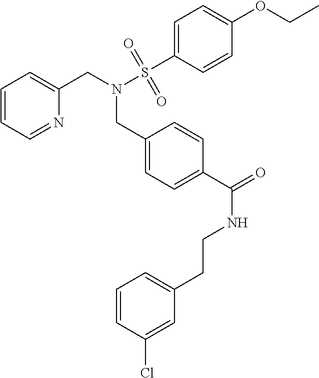 | 197 |
| 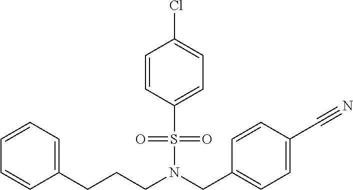 | 198 |
| 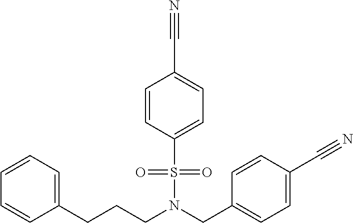 | 199 |
| 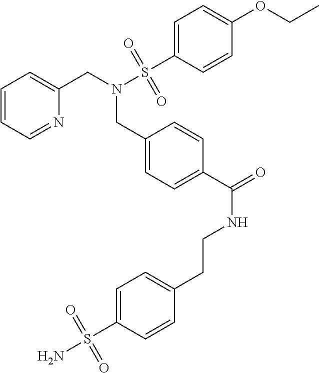 | 200 |
| 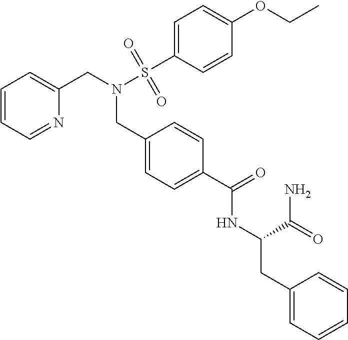 | 201 |
| 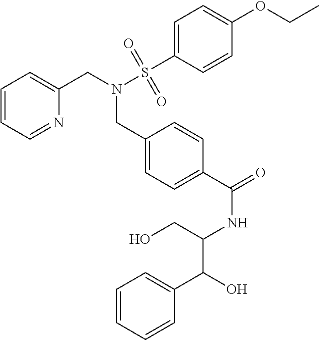 | 202 |
| 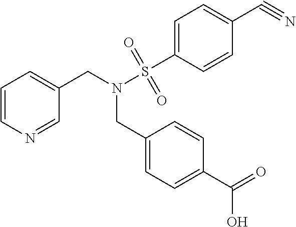 | 203 |
| 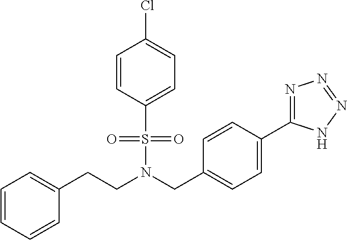 | 204 |
| 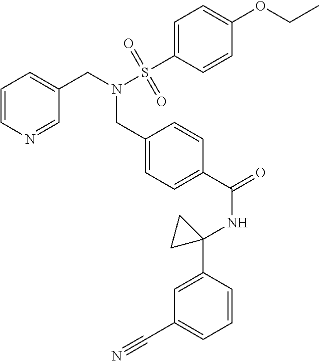 | 205 |
| 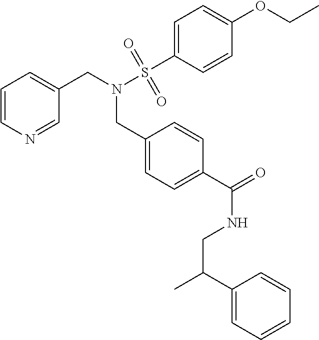 | 206 |
| 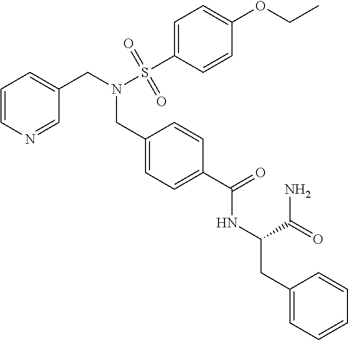 | 207 |
| 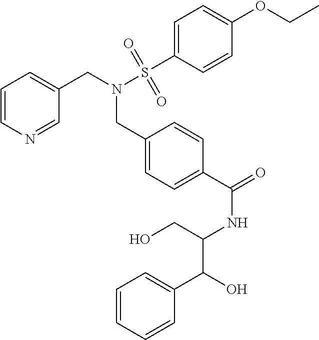 | 208 |
| 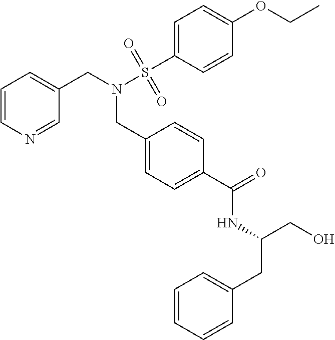 | 209 |
| 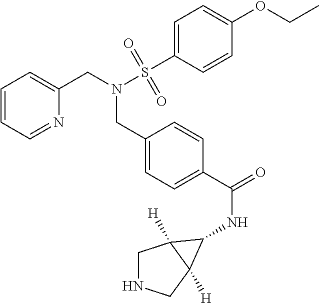 | 210 |
| 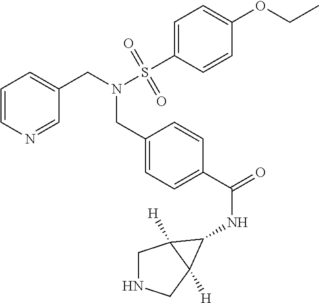 | 211 |
| 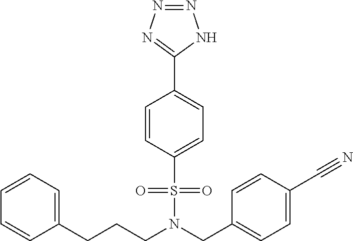 | 212 |
| 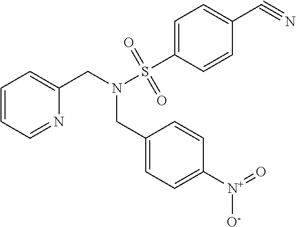 | 213 |
| 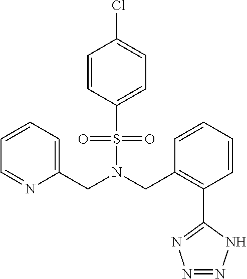 | 214 |
| 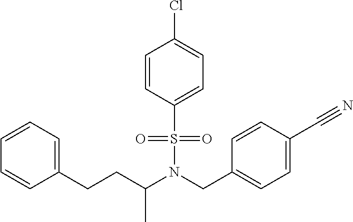 | 215 |
| 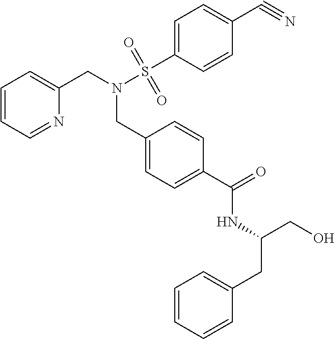 | 216 |
| 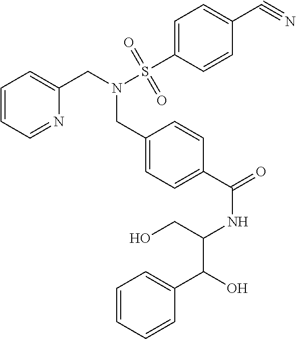 | 217 |
| 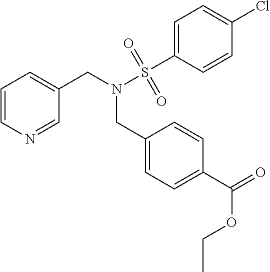 | 218 |
| 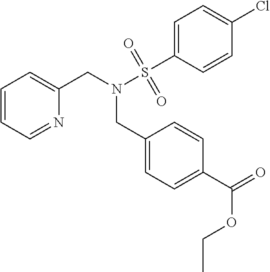 | 219 |
| 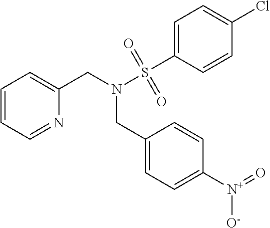 | 220 |
| 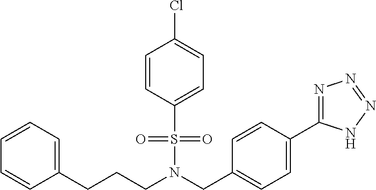 | 221 |
| 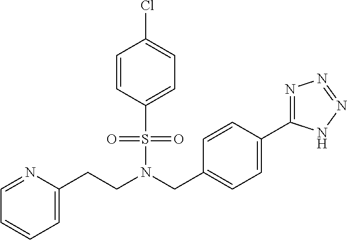 | 222 |
| 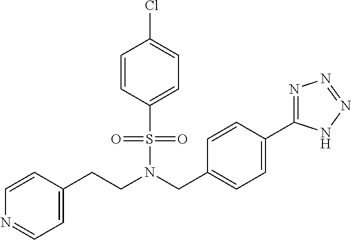 | 223 |
| 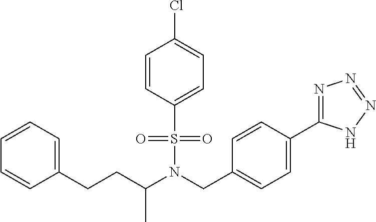 | 224 |
| 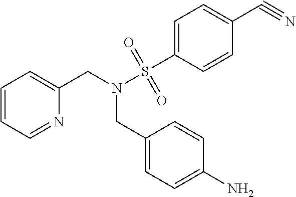 | 225 |
| 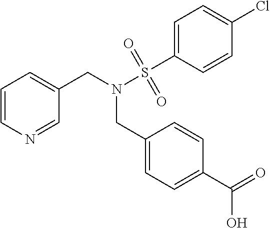 | 226 |
| 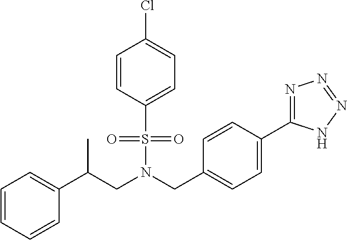 | 227 |
| 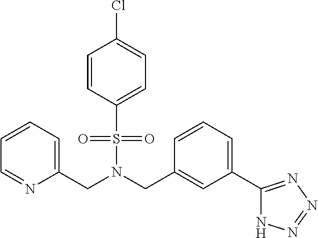 | 228 |
| 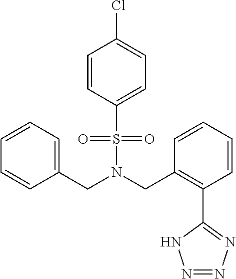 | 229 |
| 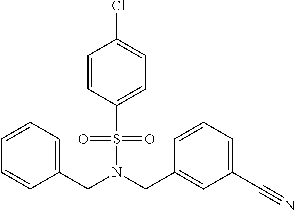 | 230 |
| 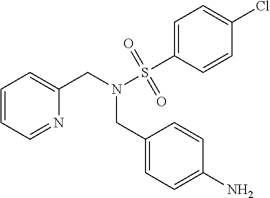 | 231 |
| 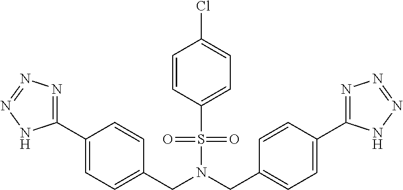 | 233 |
| 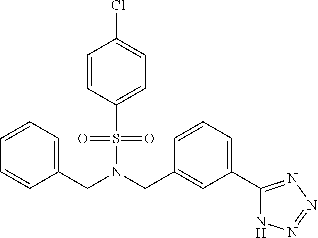 | 234 |
| 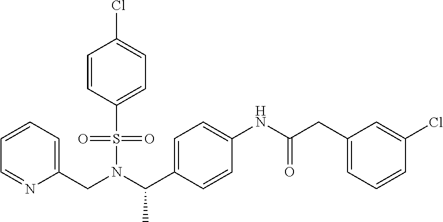 | 235 |
| 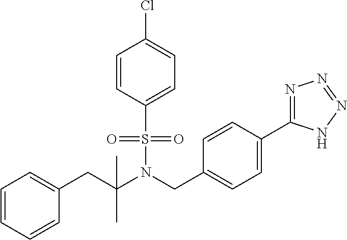 | 236 |
| 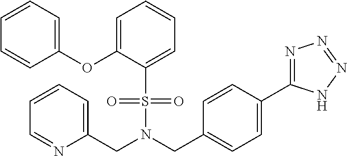 | 237 |
| 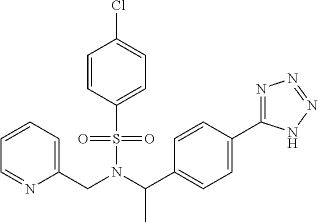 | 238 |
| 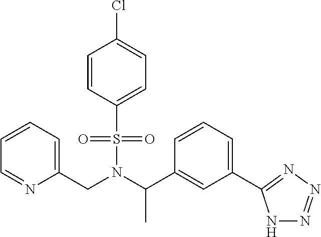 | 239 |
| 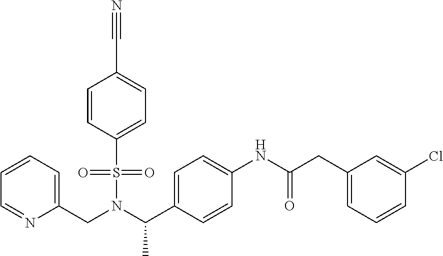 | 240 |
| 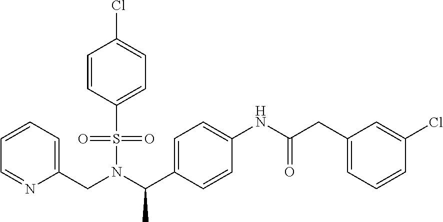 | 241 |
| 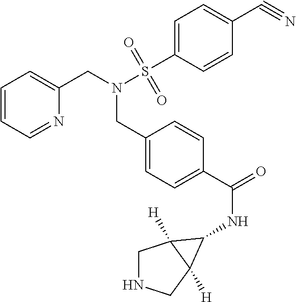 | 242 |
| 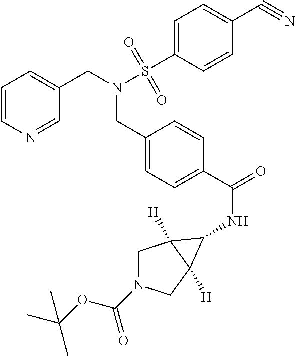 | 243 |
| 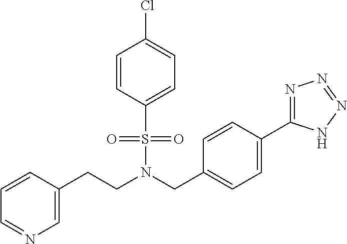 | 244 |
| 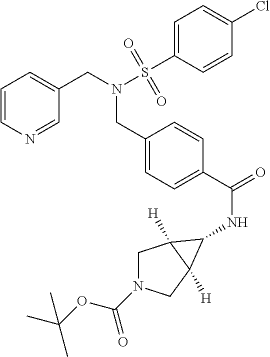 | 245 |
| 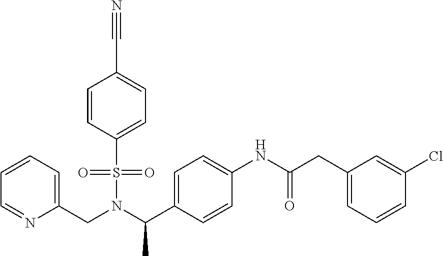 | 246 |
| 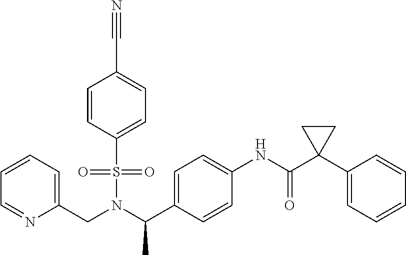 | 247 |
| 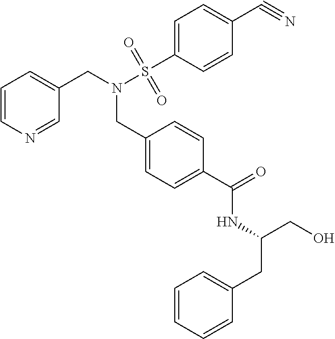 | 248 |
| 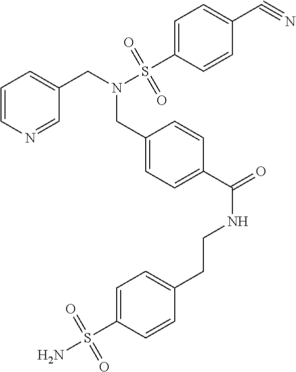 | 250 |
| 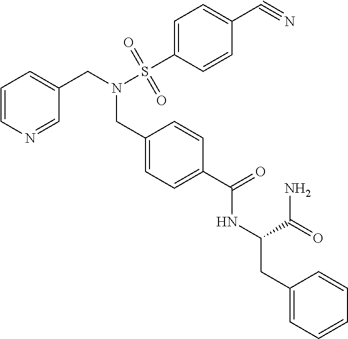 | 251 |
| 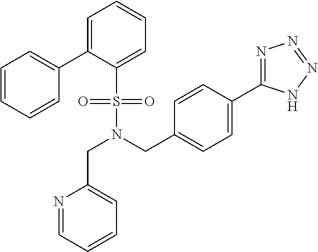 | 253 |
| 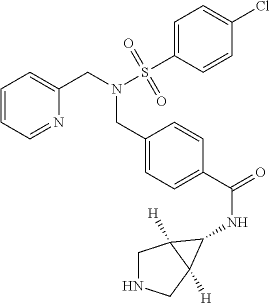 | 254 |
| 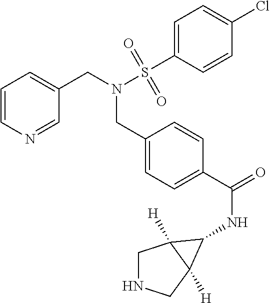 | 255 |
| 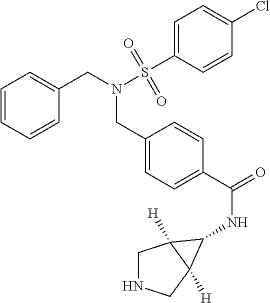 | 256 |
| 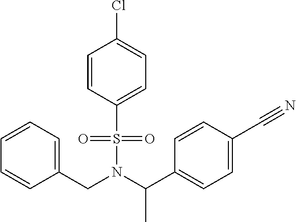 | 257 |
| 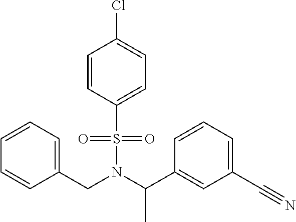 | 258 |
| 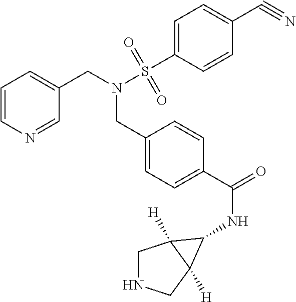 | 259 |
| 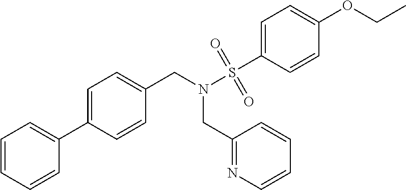 | 260 |
| 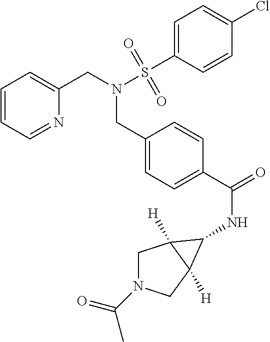 | 261 |
| 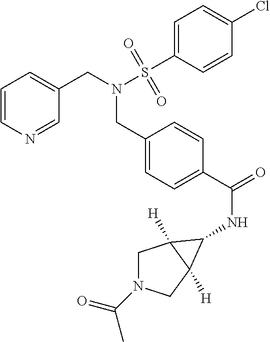 | 262 |
| 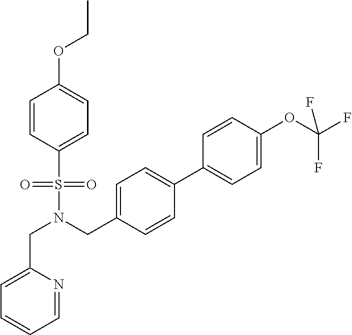 | 263 |
| 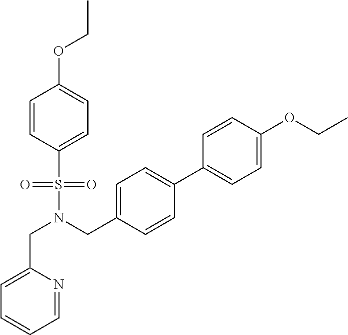 | 264 |
| 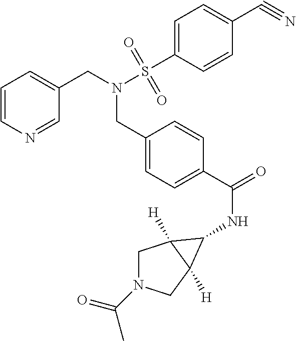 | 265 |
| 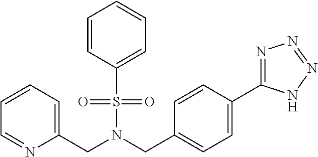 | 266 |
| 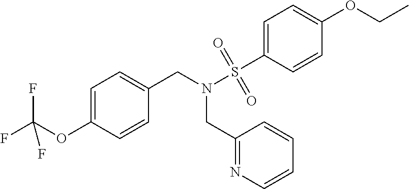 | 268 |
| 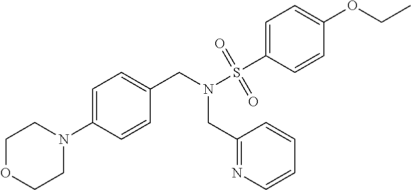 | 269 |
| 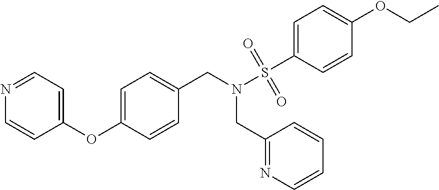 | 270 |
| 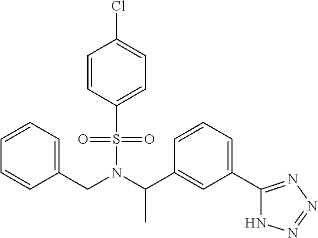 | 271 |
| 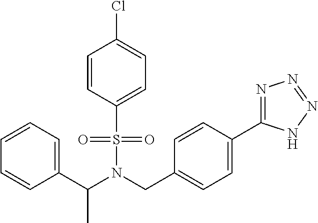 | 272 |
| 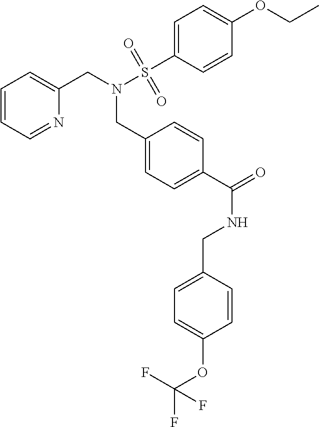 | 273 |
| 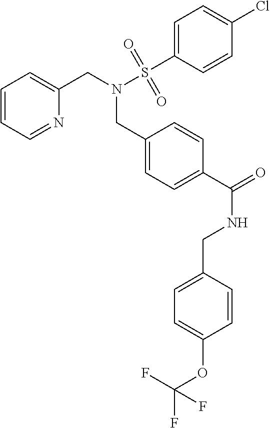 | 274 |
| 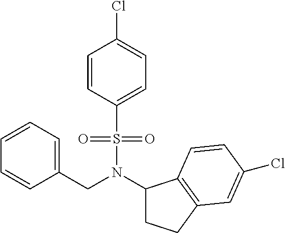 | 275 |
| 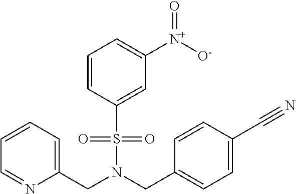 | 276 |
| 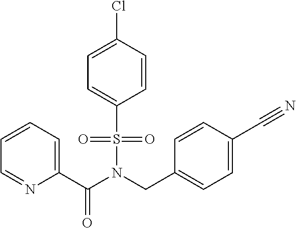 | 279 |
| 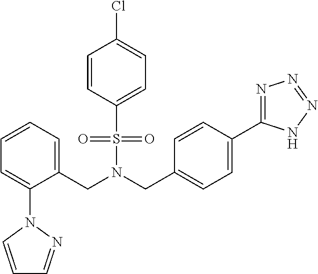 | 280 |
| 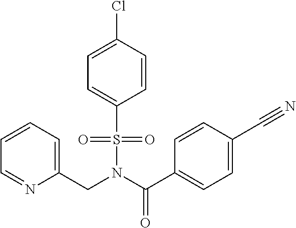 | 281 |
| 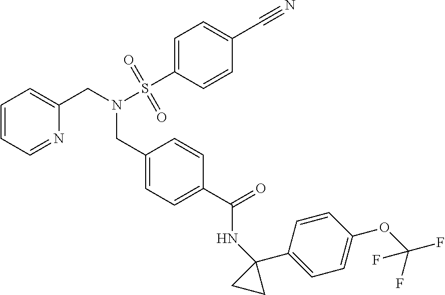 | 282 |
| 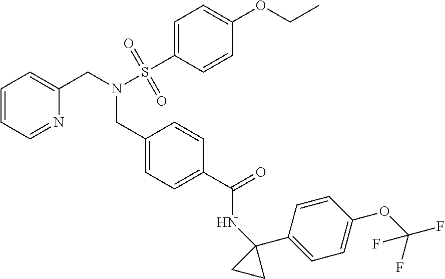 | 283 |
| 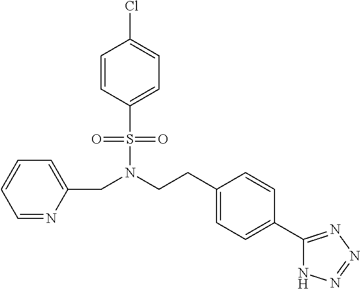 | 285 |
| 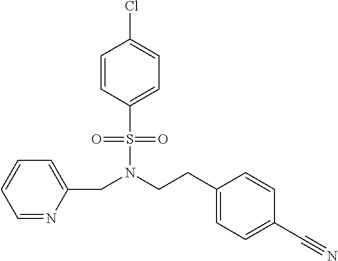 | 286 |
| 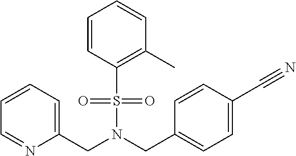 | 287 |
| 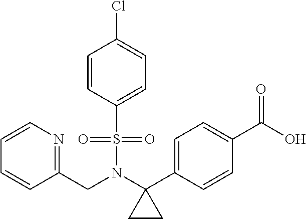 | 288 |
| 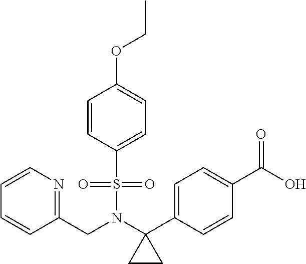 | 289 |
| 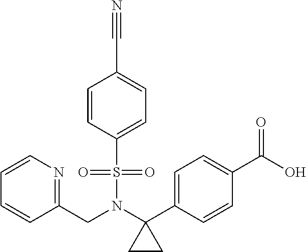 | 290 |
| 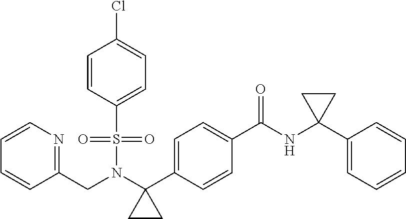 | 291 |
| 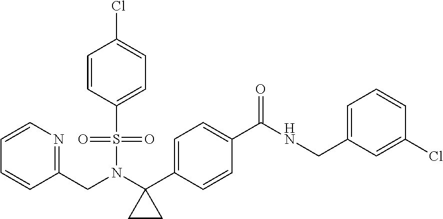 | 292 |
| 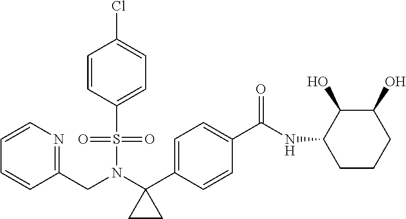 | 293 |
| 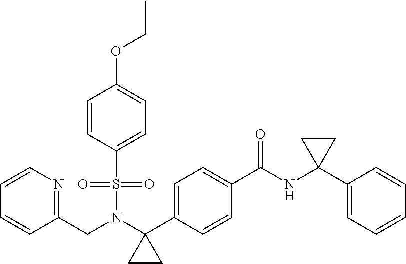 | 294 |
| 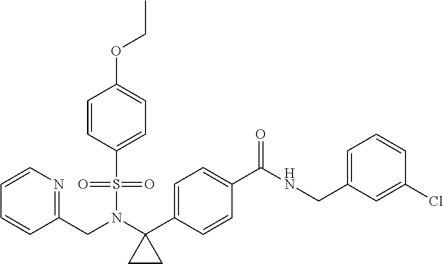 | 295 |
| 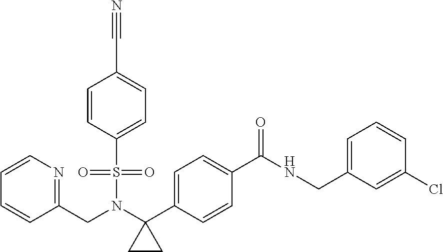 | 297 |
| 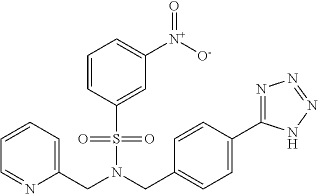 | 298 |
| 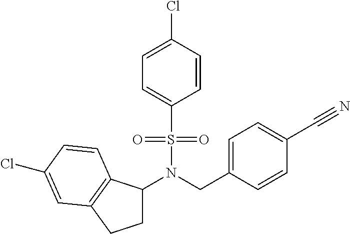 | 299 |
| 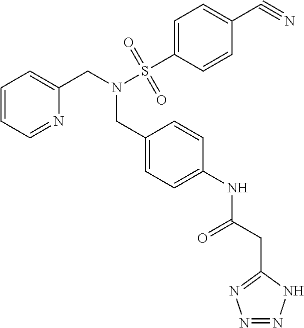 | 300 |
| 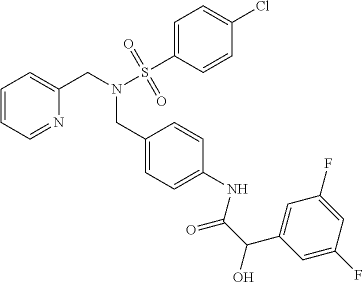 | 309 |
| 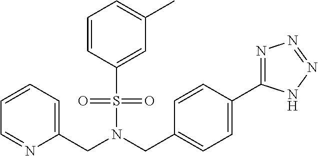 | 311 |
| 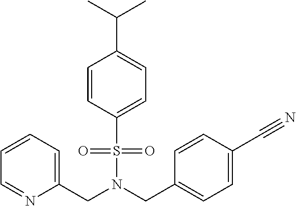 | 312 |
| 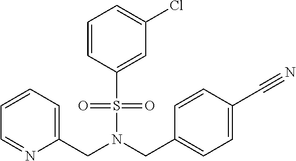 | 313 |
| 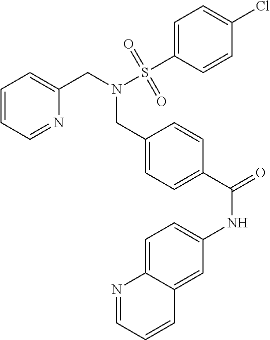 | 314 |
| 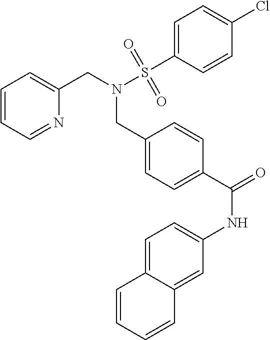 | 315 |
| 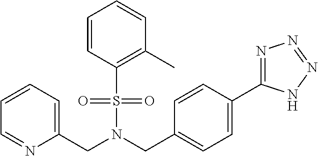 | 316 |
| 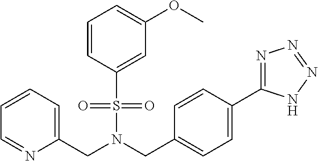 | 318 |
| 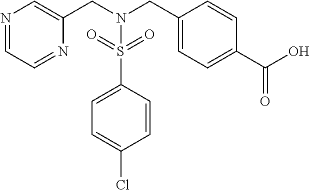 | 319 |
| 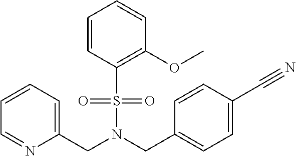 | 320 |
| 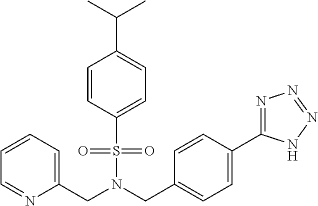 | 321 |
| 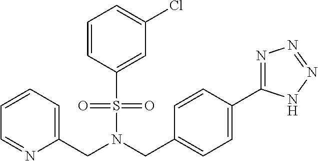 | 322 |
| 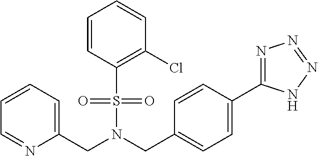 | 323 |
| 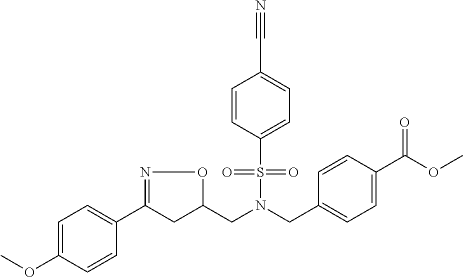 | 324 |
| 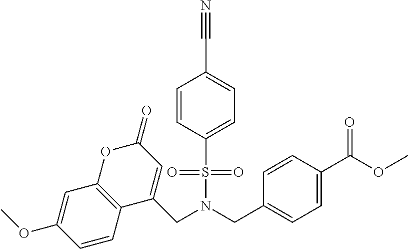 | 325 |
| 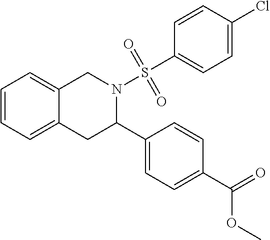 | 326 |
| 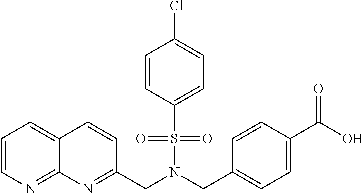 | 327 |
| 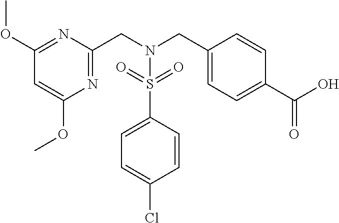 | 328 |
| 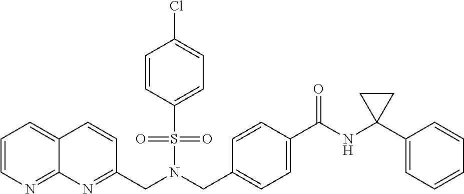 | 330 |
| 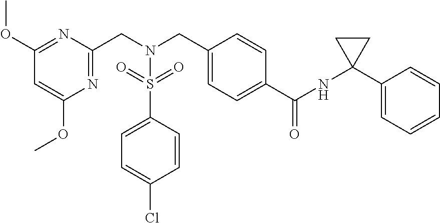 | 331 |
| 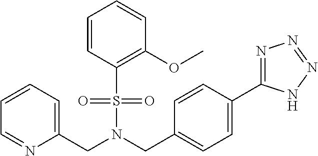 | 333 |
| 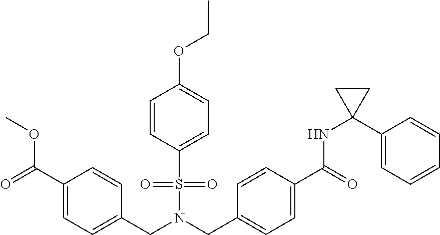 | 335 |
| 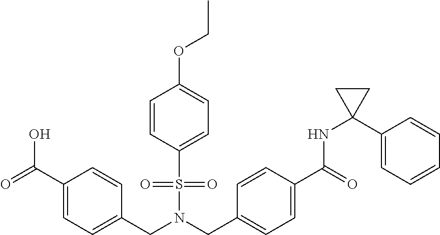 | 336 |
| 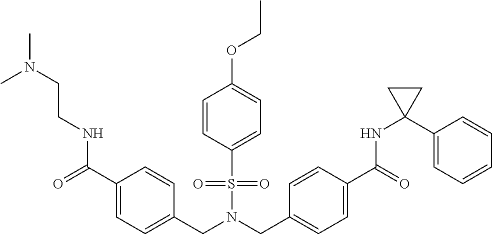 | 337 |
| 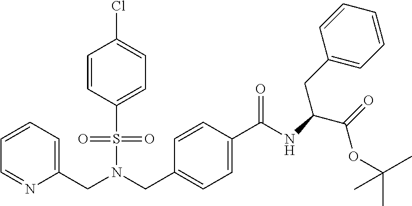 | 339 |
| 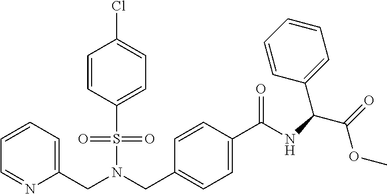 | 342 |
| 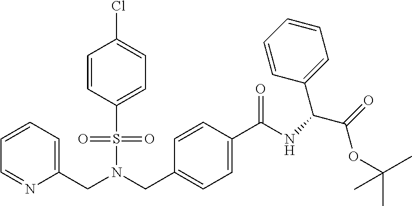 | 343 |
| 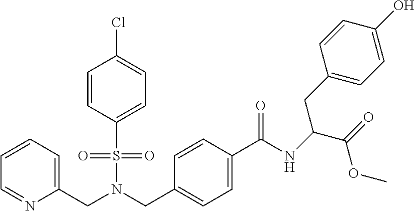 | 345 |
| 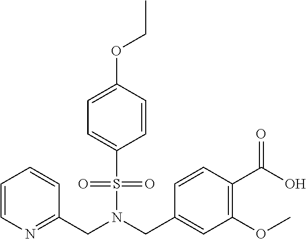 | 346 |
| 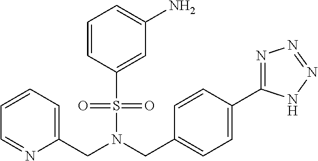 | 347 |
| 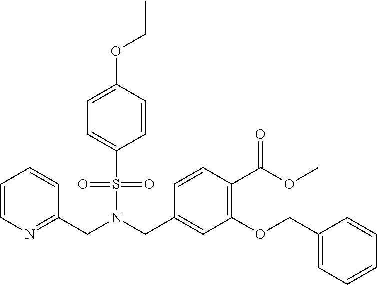 | 349 |
| 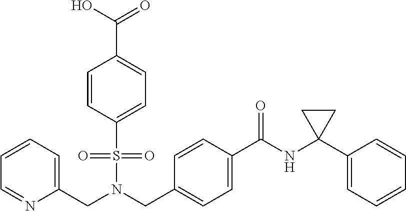 | 351 |
| 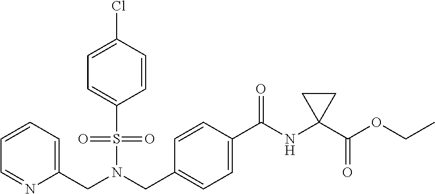 | 352 |
| 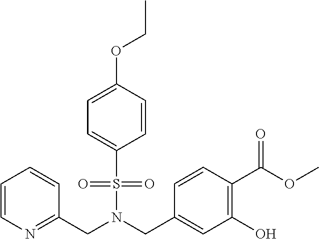 | 353 |
| 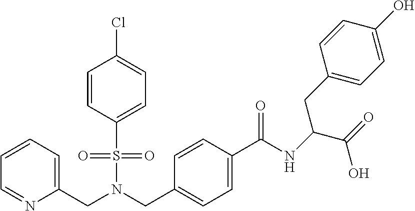 | 354 |
| 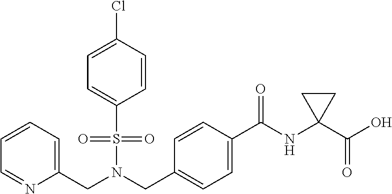 | 355 |
| 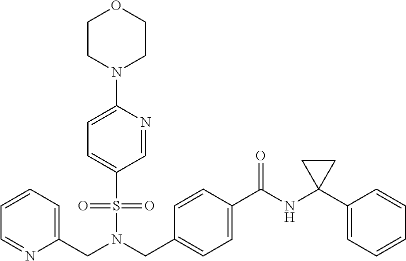 | 356 |
| 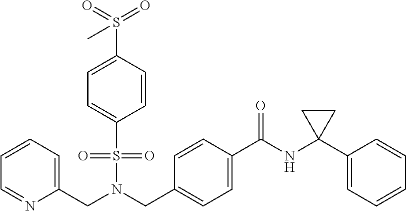 | 357 |
| 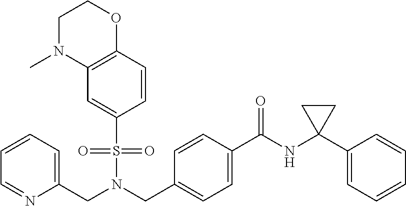 | 358 |
| 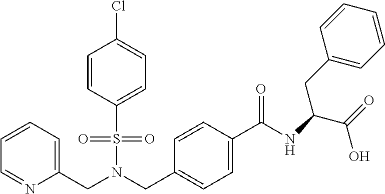 | 359 |
| 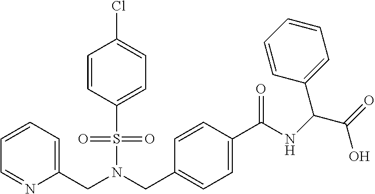 | 360 |
| 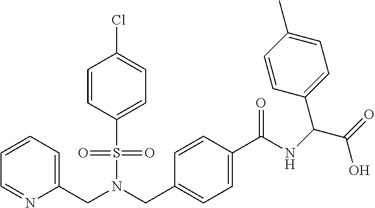 | 361 |
| 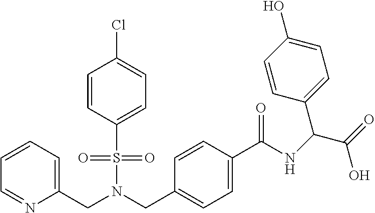 | 362 |
| 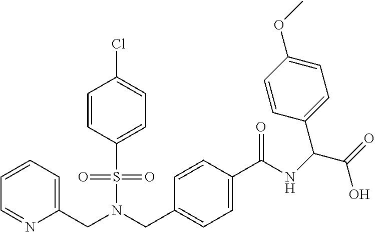 | 363 |
| 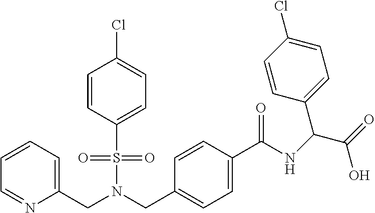 | 364 |
| 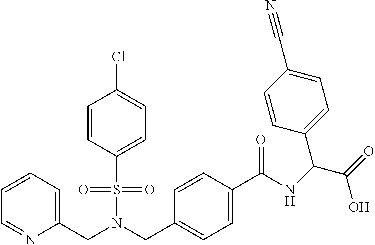 | 365 |
| 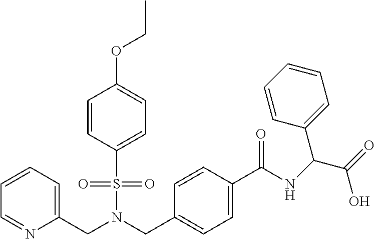 | 366 |
| 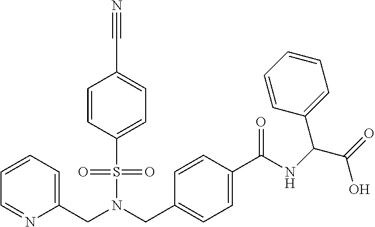 | 367 |
| 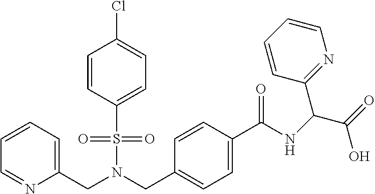 | 368 |
| 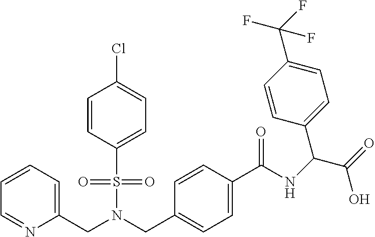 | 369 |
| 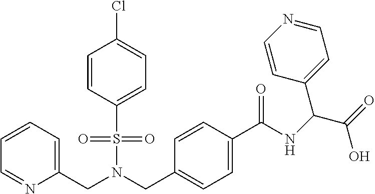 | 370 |
| 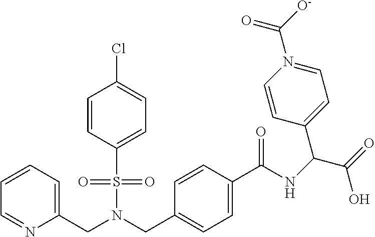 | 371 |

and pharmaceutically usable derivatives, solvates, salts and stereoisomers thereof, including mixtures thereof in all ratios.

The compounds of the formula I and also the starting materials for the preparation thereof are, in addition, prepared by methods known per se, as described in the literature (for example in the standard works, such as Houben-Weyl, Methoden der organischen Chemie [Methods of Organic Chemistry], Georg-Thieme-Verlag, Stuttgart), under reaction conditions which are known and suitable for the said reactions. For all the protection and deprotection methods, see Philip J. Kocienski, in “Protecting Groups”, Georg Thieme Verlag Stuttgart, New York, 1994 and, Theodora W. Greene and Peter G. M. Wuts in “Protective Groups in Organic Synthesis”, Wiley Interscience, 3^{rd} Edition 1999.

Use can also be made here of variants which are known per se, but are not mentioned here in greater detail.

If desired, the starting materials can also be formed in situ so that they are not isolated from the reaction mixture, but instead are immediately converted further into the compounds of the formula I.

The starting compounds for the preparation of compounds of formula I are generally known. If they are novel, they can, however, be prepared by methods known per se.

The reactions are preferably carried out in an inert solvent.

Examples of suitable inert solvents are hydrocarbons, such as hexane, petroleum ether, benzene, toluene or xylene; chlorinated hydrocarbons, such as trichloroethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide or dimethyl-formamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

### Pharmaceutical Salts and Other Forms

The said compounds of the formula I can be used in their final non-salt form. On the other hand, the present invention also relates to the use of these compounds in the form of their pharmaceutically acceptable salts, which can be derived from various organic and inorganic acids and bases by procedures known in the art. Pharmaceutically acceptable salt forms of the compounds of the formula I are for the most part prepared by conventional methods. If the compound of the formula I contains an acidic center, such as a carboxyl group, one of its suitable salts can be formed by reacting the compound with a suitable base to give the corresponding base-addition salt. Such bases are, for example, alkali metal hydroxides, including potassium hydroxide, sodium hydroxide and lithium hydroxide; alkaline earth metal hydroxides, such as barium hydroxide and calcium hydroxide; alkali metal alkoxides, for example sodium- or potassium ethoxide and sodium or potassium propoxide, alkalihydrides, such as sodium- or potassium hydride; and various organic bases, such as piperidine, diethanolamine and N-methyl-glutamine, benzathine, choline, diethanolamine, ethylenediamine, meglumine, benethamine, diethylamine, piperazine and tromethamine. The aluminium salts of the compounds of the formula I are likewise included. In the case of certain compounds of the formula I, which contain a basic center, acid-addition salts can be formed by treating these compounds with pharmaceutically acceptable organic and inorganic acids, for example hydrogen halides, such as hydrogen chloride, hydrogen bromide or hydrogen iodide, other mineral acids and corresponding salts thereof, such as sulfate, nitrate or phosphate and the like, and alkyl- and monoaryl-sulfonates, such as ethanesulfonate, toluenesulfonate and benzene-sulfonate, and other organic acids and corresponding salts thereof, such as acetate, trifluoro-acetate, tartrate, maleate, succinate, citrate, benzoate, salicylate, ascorbate and the like. Accordingly, pharmaceutically acceptable acid-addition salts of the compounds of the formula I include the following: acetate, adipate, alginate, arginate, aspartate, benzoate, benzene-sulfonate (besylate), bisulfate, bisulfite, bromide, butyrate, camphorate, camphor-sulfonate, caprylate, chloride, chlorobenzoate, citrate, cyclo-pentane-propionate, digluconate, dihydrogen-phosphate, dinitrobenzoate, dodecyl-sulfate, ethanesulfonate, fumarate, galacterate (from mucic acid), galacturonate, glucoheptanoate, gluconate, glutamate, glycerophosphate, hemi-succinate, hemisulfate, heptanoate, hexanoate, hippurate, hydro-chloride, hydrobromide, hydroiodide, 2-hydroxy-ethane-sulfonate, iodide, isethionate, isobutyrate, lactate, lactobionate, malate, maleate, malonate, mandelate, metaphosphate, methanesulfonate, methylbenzoate, mono-hydrogen-phosphate, 2-naphthalenesulfonate, nicotinate, nitrate, oxalate, oleate, palmoate, pectinate, persulfate, phenylacetate, 3-phenylpropionate, phosphate, phosphonate, phthalate, but this does not represent a restriction. Both types of salts may be formed or interconverted preferably using ion-exchange resin techniques.

Furthermore, the base salts of the compounds of the formula I include aluminium, ammonium, calcium, copper, iron(III), iron(II), lithium, magnesium, manganese(III), manganese(II), potassium, sodium and zinc salts, but this is not intended to represent a restriction. Of the above-mentioned salts, preference is given to ammonium; the alkali metal salts sodium and potassium, and the alkaline earth metal salts calcium and magnesium. Salts of the compounds of the formula I which are derived from pharmaceutically acceptable organic non-toxic bases include salts of primary, secondary and tertiary amines, substituted amines, also including naturally occurring substituted amines, cyclic amines, and basic ion exchanger resins, for example arginine, betaine, caffeine, chloroprocaine, choline, N,N′-dibenzyl-ethylenediamine (benzathine), dicyclohexylamine, diethanol-amine, diethyl-amine, 2-diethyl-amino-ethanol, 2-dimethyl-amino-ethanol, ethanolamine, ethylenediamine, N-ethylmorpholine, N-ethyl-piperidine, glucamine, glucosamine, histidine, hydrabamine, isopropyl-amine, lidocaine, lysine, meglumine (N-methyl-D-glucamine), morpholine, piperazine, piperidine, polyamine resins, procaine, purines, theobromine, triethanol-amine, triethylamine, trimethylamine, tripropyl-amine and tris(hydroxy-methyl)-methylamine (tromethamine), but this is not intended to represent a restriction.

Compounds of the formula I of the present invention which contain basic nitrogen-containing groups can be quaternised using agents such as (C1-C4)-alkyl halides, for example methyl, ethyl, isopropyl and tert-butyl chloride, bromide and iodide; di(C1-C4)alkyl sulfates, for example dimethyl, diethyl and diamyl sulfate; (C10-C18)alkyl halides, for example decyl, do-decyl, lauryl, myristyl and stearyl chloride, bromide and iodide; and aryl-(C1-C4)alkyl halides, for example benzyl chloride and phenethyl bromide. Both water- and oil-soluble compounds of the formula I can be prepared using such salts.

The above-mentioned pharmaceutical salts which are preferred include acetate, trifluoroacetate, besylate, citrate, fumarate, gluconate, hemisuccinate, hippurate, hydrochloride, hydrobromide, isethionate, mandelate, meglumine, nitrate, oleate, phosphonate, pivalate, sodium phosphate, stearate, sulfate, sulfosalicylate, tartrate, thiomalate, tosylate and tromethamine, but this is not intended to represent a restriction.

The acid-addition salts of basic compounds of the formula I are preferably prepared by bringing the free base form into contact with a sufficient amount of the desired acid, causing the formation of the salt in a conventional manner. The free base can be regenerated by bringing the salt form into contact with a base and isolating the free base in a conventional manner. The free base forms differ in a certain respect from the corresponding salt forms thereof with respect to certain physical properties, such as solubility in polar solvents; for the purposes of the invention, however, the salts other-wise correspond to the respective free base forms thereof.

As mentioned, the pharmaceutically acceptable base-addition salts of the compounds of the formula I are formed with metals or amines, such as alkali metals and alkaline earth metals or organic amines. Preferred metals are sodium, potassium, magnesium and calcium. Preferred organic amines are N,N′-dibenzylethylenediamine, chloroprocaine, choline, diethanol-amine, ethylenediamine, N-methyl-D-glucamine and procaine.

The base-addition salts of acidic compounds of the formula I are preferably prepared by bringing the free acid form into contact with a sufficient amount of the desired base, causing the formation of the salt in a conventional manner. The free acid can be regenerated by bringing the salt form into contact with an acid and isolating the free acid in a conventional manner. The free acid forms differ in a certain respect from the corresponding salt forms thereof with respect to certain physical properties, such as solubility in polar solvents; for the purposes of the invention, however, the salts other-wise correspond to the respective free acid forms thereof.

If a compound of the formula (I) contains more than one group which is capable of forming pharmaceutically acceptable salts of this type, the formula I also encompasses multiple salts. Typical multiple salt forms include, for example, bitartrate, diacetate, difumarate, dimeglumine, di-phosphate, disodium and trihydrochloride, but this is not intended to represent a restriction.

With regard to that stated above, it can be seen that the term “pharmaceutically acceptable salt” in the present connection is taken to mean an active ingredient which comprises a compound of the formula I in the form of one of its salts, in particular if this salt form imparts improved pharmacokinetic properties on the active ingredient compared with the free form of the active ingredient or any other salt form of the active ingredient used earlier. The pharmaceutically acceptable salt form of the active ingredient can also provide this active ingredient for the first time with a desired pharmacokinetic property which it did not have earlier and can even have a positive influence on the pharmacodynamics of this active ingredient with respect to its therapeutic efficacy in the body.

Owing to their molecular structure, the compounds of the formula I can be chiral and can accordingly occur in various enantiomeric forms. They can therefore exist in racemic or in optically active form.

Since the pharmaceutical activity of the racemates or stereoisomers of the compounds according to the invention may differ, it may be desirable to use the enantiomers. In these cases, the end product or even the intermediates can be separated into enantiomeric compounds by chemical or physical methods known to the person skilled in the art or even employed as such in the synthesis.

In the case of racemic amines, diastereomers are formed from the mixture by reaction with an optically active resolving agent. Examples of suitable resolving agents are optically active acids, such as the R and S forms of tartaric acid, diacetyltartaric acid, dibenzoyltartaric acid, mandelic acid, malic acid, lactic acid, suitable N-protected amino acids (for example N-benzoylproline or N-benzenesulfonylproline), or the various optically active camphorsulfonic acids. Also advantageous is chromatographic enantiomer resolution with the aid of an optically active resolving agent (for example dinitrobenzoylphenylglycine, cellulose triacetate or other derivatives of carbohydrates or chirally derivatised methacrylate polymers immobilised on silica gel). Suitable eluents for this purpose are aqueous or alcoholic solvent mixtures, such as, for example, hexane/isopropanol/acetonitrile, for example in the ratio 82:15:3.

The invention furthermore relates to the use of compounds of formula (I), in combination with at least one further medicament active ingredient, preferably medicaments used in the treatment of multiple sclerosis such as cladribine or another co-agent, such as interferon, e.g. pegylated or non-pegylated interferons, preferably interferon beta and/or with compounds improving vascular function. These further medicaments, such as interferon beta, may be administered concomitantly or sequentially, e.g. by subcutaneous, intramuscular or oral routes.

These compositions can be used as medicaments in human and veterinary medicine.

The invention furthermore relates to the use of compounds of formula (I), in combination with at least one further medicament active ingredient used in the treatment of cancer. Known anti-cancer which can be used in combination with compounds of Formula (I) include the following: oestrogen receptor modulators, androgen receptor modulators, retinoid receptor modulators, cytotoxic agents, antiproliferative agents, prenyl-protein transferase inhibitors, HMG-CoA reductase inhibitors, HIV protease inhibitors, reverse transcriptase inhibitors and other angiogenesis inhibitors.

“Oestrogen receptor modulators” refers to compounds which interfere with or inhibit the binding of oestrogen to the receptor, regardless of mechanism. Examples of oestrogen receptor modulators include, but are not limited to, tamoxifen, raloxifene, idoxifene, LY353381, LY 117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]phenyl 2,2-dimethylpropanoate, 4,4′-dihydroxybenzophenone-2,4-dinitrophenylhydrazone and SH646.

“Androgen receptor modulators” refers to compounds which interfere with or inhibit the binding of androgens to the receptor, regardless of mechanism. Examples of androgen receptor modulators include finasteride and other 5[alpha]-reductase inhibitors, nilutamide, flutamide, bicalutamide, liarozole and abiraterone acetate.

“Retinoid receptor modulators” refers to compounds which interfere with or inhibit the binding of retinoids to the receptor, regardless of mechanism. Examples of such retinoid receptor modulators include bexarotene, tretinoin, 13-cis-retinoic acid, 9-cis-retinoic acid, [alpha]-difluoromethylornithine, ILX23-7553, trans-N-(4′-hydroxyphenyl)retinamide and N-4-carboxyphenyl-retinamide.

“Cytotoxic agents” refers to compounds which result in cell death primarily through direct action on the cellular function or inhibit or interfere with cell myosis, including alkylating agents, tumour necrosis factors, intercalators, microtubulin inhibitors and topoisomerase inhibitors. Examples of cytotoxic agents include, but are not limited to, tirapazamine, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosylate, trofosfamide, nimustine, dibrospidium chloride, pumitepa, lobaplatin, satraplatin, profiromycin, cisplatin, irofulven, dexifosfamide, cis-am inedichloro(2-methylpyridine)platinum, benzylguanine, glufosfamide, GPX100, (trans,trans,trans)bis-mu-(hexane-1,6-diamine)mu-[diamine-platinum(II)]bis[diamine(chloro)platinum(II)]tetrachloride, diarizidinyl-spermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplastone, 3′-deamino-3′-morpholino-13-deoxo-10-hydroxycaminomycin, annamycin, galarubicin, elinafide, MEN10755 and 4-demethoxy-3-deamino-3-aziridinyl-4-methylsulfonyldaunorubicin (see WO 00/50032).

“Antiproliferative agents” include antisense RNA and DNA oligonucleotides such as G3139, ODN698, RVASKRAS, GEM231 and INX3001 and anti-metabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydrobenzofuryl)sulfonyl]-N′-(3,4-dichlorophenyl)urea, N6-[4-deoxy-4-[N2-[2(E),4(E)-tetradecadienoyl]glycylamino]-L-glycero-B-L-mannoheptopyranosyl]adenine, aplidine, ecteinascidin, troxacitabine, 4-[2-amino-4-oxo-4,6,7,8-tetrahydro-3H-pyrimidino[5,4-b]-1,4-thiazin-6-yl-(S)-ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,11-diazatetracyclo-(7.4.1.0.0)tetradeca-2,4,6-trien-9-ylacetic acid ester, swainsonine, lometrexol, dexrazoxane, methioninase, 2′-cyano-2′-deoxy-N4-palmitoyl-1-B-D-arabinofuranosyl cytosine and 3-aminopyridine-2-carboxaldehyde thiosemicarbazone.

“Antiproliferative agents” also include monoclonal anti-bodies to growth factors other than those listed under “angiogenesis inhibitors”, such as trastuzumab, and tumour suppressor genes, such as p53, which can be delivered via recombinant virus-mediated gene transfer

In a further aspect, compounds of the present invention can be used in the treatment and prophylaxis of tumor. The tumour is preferably selected from the group of tumours of the squamous epithelium, of the bladder, of the stomach, of the kidneys, of head and neck, of the oesophagus, of the cervix, of the thyroid, of the intestine, of the liver, of the brain, of the prostate, of the urogenital tract, of the lymphatic system, of the stomach, of the larynx and/or of the lung. The tumour is furthermore preferably selected from the group of lung adenocarcinoma, small-cell lung carcinomas, pancreatic cancer, glioblastomas, colon carcinoma and breast carcinoma. Preference is furthermore given to the use for the treatment of a tumour of the blood and immune system, preferably for the treatment of a tumour selected from the group of acute myelotic leukaemia, chronic myelotic leukaemia, acute lymphatic leukaemia and/or chronic lymphatic leukaemia.

In one aspect, the present invention provides a pharmaceutical composition comprising at least one compound according to formula (I) and related formulae and/or pharmaceutically usable derivatives, tautomers, salts, solvates and stereoisomers thereof, including mixtures thereof in all ratios, and optionally excipients and/or adjuvants.

In a second aspect, the present invention provides a pharmaceutical composition comprising at least one compound according to Formula (I) and related formulae and/or pharmaceutically usable derivatives, tautomers, salts, solvates and stereoisomers thereof, including mixtures thereof in all ratios, and at least one further active ingredient.

In a third aspect, the present invention provides the use of compounds of formula (I) and related formulae, as a medicament.

In a fourth aspect, the present invention provides compounds according to formula (I) and related formulae, and pharmaceutically usable derivatives, salts, tautomers, solvates and stereoisomers thereof, including mixtures thereof in all ratios, for the treatment and/or prophylaxis of diseases in which the inhibition, activation, regulation, and/or modulation of CXCR3 receptor signal transduction plays a role.

In a fifth aspect, the present invention provides compounds according to formula (I) and related formulae, and pharmaceutically usable derivatives, salts, tautomers, solvates and stereoisomers thereof, including mixtures thereof in all ratios, for the treatment and/or prophylaxis of a CXCR3 associated disorder.

In a sixth aspect, the invention provides the use of compounds of formula (I) and related formula according to the fifth aspect, wherein the CXCR3 associated disorder is an autoimmune disorder or condition associated with an overactive immune response.

In a seventh aspect, the present invention provides the use of compounds according to formula (I) and related formulae, and pharmaceutically usable derivatives, salts, tautomers, solvates and stereoisomers thereof, including mixtures thereof in all ratios, for the preparation of a medicament for the treatment and/or prophylaxis of an immunoregulatory abnormality.

In a height aspect, the present invention provides the use according to the seventh aspect, wherein the immunoregulatory abnormality is an autoimmune or chronic inflammatory disease selected from the group consisting of: systemic lupus erythematosis, chronic rheumatoid arthritis, type I diabetes mellitus, inflammatory bowel disease, biliary cirrhosis, uveitis, multiple sclerosis, amyotrophic lateral sclerosis (ALS), Crohn's disease, ulcerative colitis, bullous pemphigoid, sarcoidosis, psoriasis, autoimmune myositis, Wegener's granulomatosis, ichthyosis, Graves ophthalmopathy and asthma.

In a ninth aspect, the present invention provides the use according to the height aspect, wherein the immunoregulatory abnormality is bone marrow or organ transplant rejection or graft-versus-host disease.

In a tenth aspect, the invention further relates to a kit or a set comprising at least one compound of Formula (I), preferably in combination with immunomodulating agents.

Alternatively, the kit consists of separate packs of:

(a) an effective amount of a compound of the formula (I) and/or pharmaceutically usable derivatives, solvates and stereoisomers thereof, including mixtures thereof in all ratios, and
(b) an effective amount of a further medicament active ingredient.

Pharmaceutical formulations can be administered in the form of dosage units, which comprise a predetermined amount of active ingredient per dosage unit. Such a unit can comprise, for example, 0.5 mg to 1 g, preferably 1 mg to 700 mg, particularly preferably 5 mg to 100 mg, of a compound according to the invention, depending on the disease condition treated, the method of administration and the age, weight and condition of the patient, or pharmaceutical formulations can be administered in the form of dosage units which comprise a predetermined amount of active ingredient per dosage unit. Preferred dosage unit formulations are those which comprise a daily dose or part-dose, as indicated above, or a corresponding fraction thereof of an active ingredient. Furthermore, pharmaceutical formulations of this type can be prepared using a process, which is generally known in the pharmaceutical art.

Pharmaceutical formulations can be adapted for administration via any desired suitable method, for example by oral (including buccal or sublingual), rectal, nasal, topical (including buccal, sublingual or transdermal), vaginal or parenteral (including subcutaneous, intramuscular, intravenous or intradermal) methods. Such formulations can be prepared using all processes known in the pharmaceutical art by, for example, combining the active ingredient with the excipient(s) or adjuvant(s).

Pharmaceutical formulations adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions.

Thus, for example, in the case of oral administration in the form of a tablet or capsule, the active-ingredient component can be combined with an oral, non-toxic and pharmaceutically acceptable inert excipient, such as, for example, ethanol, glycerol, water and the like. Powders are prepared by comminuting the compound to a suitable fine size and mixing it with a pharmaceutical excipient comminuted in a similar manner, such as, for example, an edible carbohydrate, such as, for example, starch or mannitol. A flavour, preservative, dispersant and dye may likewise be present.

Capsules are produced by preparing a powder mixture as described above and filling shaped gelatine shells therewith. Glidants and lubricants, such as, for example, highly disperse silicic acid, talc, magnesium stearate, calcium stearate or polyethylene glycol in solid form, can be added to the powder mixture before the filling operation. A disintegrant or solubiliser, such as, for example, agar-agar, calcium carbonate or sodium carbonate, may likewise be added in order to improve the availability of the medicament after the capsule has been taken.

In addition, if desired or necessary, suitable binders, lubricants and disintegrants as well as dyes can likewise be incorporated into the mixture. Suitable binders include starch, gelatine, natural sugars, such as, for example, glucose or beta-lactose, sweeteners made from maize, natural and synthetic rubber, such as, for example, acacia, tragacanth or sodium alginate, carboxymethylcellulose, polyethylene glycol, waxes, and the like. The lubricants used in these dosage forms include sodium oleate, sodium stearate, magnesium stearate, sodium benzoate, sodium acetate, sodium chloride and the like. The disintegrants include, without being restricted thereto, starch, methylcellulose, agar, bentonite, xanthan gum and the like. The tablets are formulated by, for example, preparing a powder mixture, granulating or dry-pressing the mixture, adding a lubricant and a disintegrant and pressing the entire mixture to give tablets. A powder mixture is prepared by mixing the compound comminuted in a suitable manner with a diluent or a base, as described above, and optionally with a binder, such as, for example, carboxymethylcellulose, an alginate, gelatine or polyvinyl-pyrrolidone, a dissolution retardant, such as, for example, paraffin, an absorption accelerator, such as, for example, a quaternary salt, and/or an absorbant, such as, for example, bentonite, kaolin or dicalcium phosphate. The powder mixture can be granulated by wetting it with a binder, such as, for example, syrup, starch paste, acadia mucilage or solutions of cellulose or polymer materials and pressing it through a sieve. As an alternative to granulation, the powder mixture can be run through a tableting machine, giving lumps of non-uniform shape which are broken up to form granules. The granules can be lubricated by addition of stearic acid, a stearate salt, talc or mineral oil in order to prevent sticking to the tablet casting moulds. The lubricated mixture is then pressed to give tablets. The active ingredients can also be combined with a free-flowing inert excipient and then pressed directly to give tablets without carrying out the granulation or dry-pressing steps. A transparent or opaque protective layer consisting of a shellac sealing layer, a layer of sugar or polymer material and a gloss layer of wax may be present. Dyes can be added to these coatings in order to be able to differentiate between different dosage units.

Oral liquids, such as, for example, solution, syrups and elixirs, can be prepared in the form of dosage units so that a given quantity comprises a pre-specified amount of the compounds. Syrups can be prepared by dissolving the compounds in an aqueous solution with a suitable flavour, while elixirs are prepared using a non-toxic alcoholic vehicle. Suspensions can be formulated by dispersion of the compounds in a non-toxic vehicle. Solubilisers and emulsifiers, such as, for example, ethoxylated isostearyl alcohols and polyoxyethylene sorbitol ethers, preservatives, flavour additives, such as, for example, peppermint oil or natural sweeteners or saccharin, or other artificial sweeteners and the like, can likewise be added.

The dosage unit formulations for oral administration can, if desired, be encapsulated in microcapsules. The formulation can also be prepared in such a way that the release is extended or retarded, such as, for example, by coating or embedding of particulate material in polymers, wax and the like.

The compounds of the formula (I) and salts, solvates and physiologically functional derivatives thereof and the other active ingredients can also be administered in the form of liposome delivery systems, such as, for example, small unilamellar vesicles, large unilamellar vesicles and multilamellar vesicles. Liposomes can be formed from various phospholipids, such as, for example, cholesterol, stearylamine or phosphatidylcholines.

Pharmaceutical formulations adapted for transdermal administration can be administered as independent plasters for extended, close contact with the epidermis of the recipient. Thus, for example, the active ingredient can be delivered from the plaster by iontophoresis, as described in general terms in Pharmaceutical Research, 3(6), 318 (1986).

Pharmaceutical compounds adapted for topical administration can be formulated as ointments, creams, suspensions, lotions, powders, solutions, pastes, gels, sprays, aerosols or oils.

For the treatment of the eye or other external tissue, for example mouth and skin, the formulations are preferably applied as topical ointment or cream. In the case of formulation to give an ointment, the active ingredient can be employed either with a paraffinic or a water-miscible cream base. Alternatively, the active ingredient can be formulated to give a cream with an oil-in-water cream base or a water-in-oil base.

Pharmaceutical formulations adapted for topical application to the eye include eye drops, in which the active ingredient is dissolved or suspended in a suitable carrier, in particular an aqueous solvent.

Pharmaceutical formulations adapted for topical application in the mouth encompass lozenges, pastilles and mouthwashes.

Pharmaceutical formulations adapted for rectal administration can be administered in the form of suppositories or enemas.

Pharmaceutical formulations adapted for nasal administration in which the carrier substance is a solid comprise a coarse powder having a particle size, for example, in the range 20-500 microns, which is administered in the manner in which snuff is taken, i.e. by rapid inhalation via the nasal passages from a container containing the powder held close to the nose. Suitable formulations for administration as nasal spray or nose drops with a liquid as carrier substance encompass active-ingredient solutions in water or oil.

Pharmaceutical formulations adapted for administration by inhalation encompass finely particulate dusts or mists, which can be generated by various types of pressurised dispensers with aerosols, nebulisers or insufflators.

Pharmaceutical formulations adapted for vaginal administration can be administered as pessaries, tampons, creams, gels, pastes, foams or spray formulations.

Pharmaceutical formulations adapted for parenteral administration include aqueous and non-aqueous sterile injection solutions comprising antioxidants, buffers, bacteriostatics and solutes, by means of which the formulation is rendered isotonic with the blood of the recipient to be treated; and aqueous and non-aqueous sterile suspensions, which may comprise suspension media and thickeners. The formulations can be administered in single-dose or multidose containers, for example sealed ampoules and vials, and stored in freeze-dried (lyophilised) state, so that only the addition of the sterile carrier liquid, for example water for injection purposes, immediately before use is necessary.

Injection solutions and suspensions prepared in accordance with the recipe can be prepared from sterile powders, granules and tablets.

It goes without saying that, in addition to the above particularly mentioned constituents, the formulations may also comprise other agents usual in the art with respect to the particular type of formulation; thus, for example, formulations which are suitable for oral administration may comprise flavours.

A therapeutically effective amount of a compound of the formula I and of the other active ingredient depends on a number of factors, including, for example, the age and weight of the animal, the precise disease condition which requires treatment, and its severity, the nature of the formulation and the method of administration, and is ultimately determined by the treating doctor or vet. However, an effective amount of a compound is generally in the range from 0.1 to 100 mg/kg of body weight of the recipient (mammal) per day and particularly typically in the range from 1 to 10 mg/kg of body weight per day. Thus, the actual amount per day for an adult mammal weighing 70 kg is usually between 70 and 700 mg, where this amount can be administered as an individual dose per day or usually in a series of part-doses (such as, for example, two, three, four, five or six) per day, so that the total daily dose is the same. An effective amount of a salt or solvate or of a physiologically functional derivative thereof can be determined as the fraction of the effective amount of the compound per se.

The present invention furthermore relates to a method for treating a subject suffering from a CXCR3 associated disorder, comprising administering to said subject an effective amount of a compound of formula I. The present invention preferably relates to a method, wherein the CXCR3 associated disorder is an autoimmune disorder or condition associated with an overactive immune response.

The present invention furthermore relates to a method of treating a subject suffering from an immunoregulatory abnormality, comprising administering to said subject a compound of formula I in an amount that is effective for treating said immunoregulatory abnormality. The present invention preferably relates to a method wherein the immunoregulatory abnormality is an autoimmune or chronic inflammatory disease selected from the group consisting of: amyotrophic lateral sclerosis (ALS), systemic lupus erythematosus, chronic rheumatoid arthritis, type I diabetes mellitus, inflammatory bowel disease, biliary cirrhosis, uveitis, multiple sclerosis, Crohn's disease, ulcerative colitis, bullous pemphigoid, sarcoidosis, psoriasis, autoimmune myositis, Wegener's granulomatosis, ichthyosis, Graves ophthalmopathy and asthma. The present invention furthermore relates to a method wherein the immunoregulatory abnormality is bone marrow or organ transplant rejection or graft-versus-host disease. The present invention furthermore relates to a method wherein the immunoregulatory abnormality is selected from the group consisting of: transplantation of organs or tissue, graft-versus-host diseases brought about by transplantation, autoimmune syndromes including rheumatoid arthritis, systemic lupus erythematosus, Hashimoto's thyroiditis, multiple sclerosis, myasthenia gravis, type I diabetes, uveitis, posterior uveitis, allergic encephalomyelitis, glomerulonephritis, post-infectious autoimmune diseases including rheumatic fever and post-infectious glomerulonephritis, inflammatory and hyperproliferative skin diseases, psoriasis, atopic dermatitis, contact dermatitis, eczematous dermatitis, seborrhoeic dermatitis, lichen planus, pemphigus, bullous pemphigoid, epidermolysis bullosa, urticaria, angioedemas, vasculitis, erythema, cutaneous eosinophilia, lupus erythematosus, acne, alopecia greata, keratoconjunctivitis, vernal conjunctivitis, uveitis associated with Behcet's disease, keratitis, herpetic keratitis, conical cornea, dystrophia epithelialis corneae, corneal leukoma, ocular pemphigus, Mooren's ulcer, scleritis, Graves' opthalmopathy, Vogt-Koyanagi-Harada syndrome, sarcoidosis, pollen allergies, reversible obstructive airway disease, bronchial asthma, allergic asthma, intrinsic asthma, extrinsic asthma, dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, necrotizing enterocolitis, intestinal lesions associated with thermal burns, coeliac diseases, proctitis, eosinophilic gastroenteritis, mastocytosis, Crohn's disease, ulcerative colitis, migraine, rhinitis, eczema, interstitial nephritis, Goodpasture's syndrome, hemolytic-uremic syndrome, diabetic nephropathy, multiple myositis, Guillain-Barre syndrome, Meniere's disease, polyneuritis, multiple neuritis, mononeuritis, radiculopathy, hyperthyroidism, Basedow's disease, pure red cell aplasia, aplastic anemia, hypoplastic anemia, idiopathic thrombocytopenic purpura, autoimmune hemolytic anemia, agranulocytosis, pernicious anemia, megaloblastic anemia, anerythroplasia, osteoporosis, sarcoidosis, fibroid lung, idiopathic interstitial pneumonia, dermatomyositis, leukoderma vulgaris, ichthyosis vulgaris, photoallergic sensitivity, cutaneous T cell lymphoma, chronic lymphocytic leukemia, arteriosclerosis, atherosclerosis, aortitis syndrome, polyarteritis nodosa, myocardosis, scleroderma, Wegener's granuloma, Sjogren's syndrome, adiposis, eosinophilic fascitis, lesions of gingiva, periodontium, alveolar bone, substantia ossea dentis, glomerulonephritis, male pattern alopecia or alopecia senilis by preventing epilation or providing hair germination and/or promoting hair generation and hair growth, muscular dystrophy, pyoderma and Sezary's syndrome, Addison's disease, ischemia-reperfusion injury of organs which occurs upon preservation, transplantation or ischemic disease, endotoxin-shock, pseudomembranous colitis, colitis caused by drug or radiation, ischemic acute renal insufficiency, chronic renal insufficiency, toxinosis caused by lung-oxygen or drugs, lung cancer, pulmonary emphysema, cataracta, siderosis, retinitis pigmentosa, senile macular degeneration, vitreal scarring, corneal alkali burn, dermatitis erythema multiforme, linear IgA ballous dermatitis and cement dermatitis, gingivitis, periodontitis, sepsis, pancreatitis, diseases caused by environmental pollution, aging, carcinogenesis, metastasis of carcinoma and hypobaropathy, disease caused by histamine or leukotriene-C₄ release, Behcet's disease, autoimmune hepatitis, primary biliary cirrhosis, sclerosing cholangitis, partial liver resection, acute liver necrosis, necrosis caused by toxin, viral hepatitis, shock, or anoxia, B-virus hepatitis, non-A/non-B hepatitis, cirrhosis, alcoholic cirrhosis, hepatic failure, fulminant hepatic failure, late-onset hepatic failure, “acute-on-chronic” liver failure, augmentation of chemotherapeutic effect, cytomegalovirus infection, HCMV infection, AIDS, cancer, senile dementia, trauma, and chronic bacterial infection.

Preferred compounds of formula I exhibit a binding constant Ki for the binding to CXCR3 of less than about 5 μM, preferably less than about 1 μM and even more preferred less than about 0.1 μM.

Nomenclature of the compounds of this invention has been determined using ACD/Name Version 7.10 software.

In the following the present invention shall be illustrated by means of some examples, which are not construed to be viewed as limiting the scope of the invention.

### EXAMPLES

### General

The HPLC data provided in the examples described below were obtained as followed.

Condition A: 8 min gradient from 0.1% TFA in H₂O to 0.07% TFA in CH₃CN. HPLC column: Xbridge™ C₈ column 50 mm×4.6 mm at a flow of 2 mL/min. UV detection (maxplot)
Condition B: 8 min gradient from 0.1% TFA in H₂O to 0.07% TFA in CH₃CN. HPLC column: Atlantis C18 75 mm×4.6 mm at a flow of 0.8 mL/min. UV detection (maxplot)
Condition C: Solvent A: H2O (0.01% TFA); Solvent B: ACN (0.01% TFA); In 2 min from 90% A to 100% B. Followed by 3 min 100% B and 1 min 90% A.; Column: Chromolith SpeedROD RP-18e 50-4.6; DAD 220 nm; Flow: 3 ml/min; Solvent: LiChrosolv-quality from the company Merck KGaA;
Condition D: Solvent A: H2O (0.01% TFA); Solvent B: ACN (0.01% TFA); 1 min 100% A. In 2.5 min from 100% A to 100% B. Followed by 1.5 min 100% B and 1 min 100% A. Column: Chromolith SpeedROD RP-18e 50-4.6; DAD 220 nm; Flow: 3 ml/Min; Solvent: LiChrosolv-quality from the company Merck KGaA;

The MS data provided in the examples described below were obtained as followed: Mass spectrum: LC/MS Waters ZMD (ESI) or Hewlett Packard System of the HP 1100 series (Ion source: Electrospray (positive mode); Scan: 100-1000 m/z; Fragmentation-voltage: 60 V; Gas-temperature: 300° C., DAD: 220 nm. Flow rate: 2.4 ml/Min. The used splitter reduced the flow rate after the DAD for the MS to 0.75 ml/Min; Column: Chromolith Speed ROD RP-18e 50-4.6; Solvent: LiChrosolv-quality from the company Merck KGaA; Solvent A: H2O (0.01% TFA); Solvent B: ACN (0.01% TFA); Gradient a) In 2.8 min from 80% A to 100% B. Followed by 0.2 min 100% B and 1 min 80% A or b) in 3 min from 95% A to 100% B. Followed by 0.8 min 95% A

The NMR data provided in the examples described below were obtained as followed: ¹H-NMR: Bruker DPX-300 or DRX-500 or DRX-400 or AVII-400

The microwave chemistry is performed on a single mode microwave reactor Emrys™ Optimiser from Personal Chemistry.

Preparative HPLC was performed on a mass directed autopurification Fractionlynx system from Waters. Column: Sunfire prep C18 OBD 19×100 mm; 5 microns. Mobile phase: 0.1% formic acid in water/0.1% formic acid in acetonitrile.

### Intermediate 1

### methyl 4-[(benzyl amino)methyl]benzoate hydrochloride

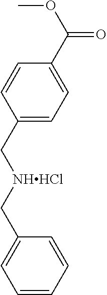

A solution of benzyl amine (5.00 g, 51.5 mmol) and methyl-4-formyl benzoate (9.20 g, 57 mmol) in toluene (100 ml) was refluxed for 2 h with azeotropic removal of water. The toluene was evaporated off under reduced pressure and the residue was taken in methanol (100 ml) and cooled to 0° C. Then Na(CN)BH₃ (6.40 g, 103 mmol) was added portion wise and the reaction mixture was stirred at 0° C. for 2 h. The reaction mixture was poured into water and extracted with ethyl acetate; the organic layer was washed with brine and dried over sodium sulfate. The organic layer was concentrated and the residue was diluted with dioxane (100 ml). A cold HCl solution (1N HCl in dioxane, 50 ml) was added to the crude mixture slowly, a white solid precipitated out which was filtered, washed with chloroform and dried under vacuum to get the title compound (11.0 g, 73%) as a white solid.

¹H NMR (DMSO-d6, 400 MHz): δ 9.91 (2H, br s), 7.98 (2H, d, J=8.0 Hz), 7.71 (2H, d, J=8.0 Hz), 7.57-7.55 (2H, m), 7.43-7.40 (3H, m) 4.22-4.20 (2H, m), 4.15-4.13 (2H, m), 3.85 (3H, s). MS (ESI+): 255.1. HPLC (Condition B): Rt 1.80 min (HPLC purity 95.2%).

### Intermediate 1a

### 4-{1-[(Pyridin-2-ylmethyl)-amino]-cyclopropyl}-benzoic acid methyl ester

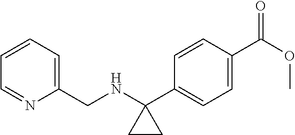

a) Ethylmagnesium bromide (22.75 ml; 68.26 mmol, 3 M in ether) was added at −70° C. to a solution of a nitrile (5 g; 31.03 mmol) and Ti(Oi-Pr)₄ (10.1 mL, 34.13 mmol) in Et₂O (160 mL). The yellow solution was stirred for 30 min. After the solution was warmed to rt (1 h), BF₃—OEt₂ (7.8 mL, 62.05 mmol) was added. After the mixture was stirred for 2 h, 1 N HCl (110 mL) and ether (ca. 15 mL) were added. NaOH (10% aq, ca. 10 mL) was added to the resulting two clear phases and the mixture was extracted with ether. The combined ether layers were dried (Na2SO4), filtered, and concentrated under reduced pressure. The residue was purified by flash chromatography on silica gel (n-heptan/EtOAc) to give 4-(1-Amino-cyclopropyl)-benzoic acid methyl ester as a yellow oil (2.45 g; 41.3% yield. (MS: m/z: 192)

b) A solution of 4-(1-Amino-cyclopropyl)-benzoic acid methyl ester (2.45 g, 12.8 mmol) and Pyridine-2-carbaldehyde (1.22 ml, 12.8 mmol) in methanol (50 ml) was stirred for 12 h at RT.) and then cooled to 0° C. Then NaBH₄ (291 mg; 7.7 mmol) was added and the reaction mixture was stirred at 0° C. for 30 min and 1 h at RT. The reaction mixture was poured into water (30 ml), concentrated and aqueous layer was extracted with ethyl acetate; the organic layer was washed with brine and dried over sodium sulfate. The organic layer was concentrated and the residue was purified by flash chromatography on silica gel (n-heptan/EtOAc) to give the title as a yellow oil (2.8 g; 77.1% yield. (MS: m/z: 283).

### Intermediate 2

### 4-{[benzyl-(4-chloro-benzenesulfonyl)-amino]-methyl}-benzoic acid methyl ester

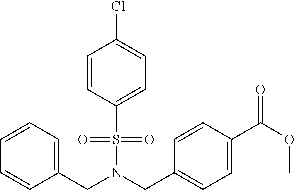

A cooled (0° C.) solution of methyl 4-[(benzylamino)methyl]benzoate hydrochloride (Intermediate 1; 1.5 g, 5.2 mmol) in dichloromethane (75 ml) was treated with triethylamine (1.58 g, 15 mmol) and 4-chlorobenzenesulfonyl chloride (1.21 g, 5.7 mmol) and stirred overnight. The reaction mixture was quenched with ice, diluted with DCM and successively washed with 10% aqueous sodium bicarbonate and brine. The organic layer was dried over sodium sulfate, concentrated and recrystallised from DCM/hexane to afford the title compound as an off white solid (1.8 g; 81%).

¹H NMR (DMSO-d6, 400 MHz): δ 7.91 (2H, d, J=8.5 Hz), 7.76 (2H, d, J=8.0 Hz), 7.68 (2H, d, J=8.5 Hz), 7.22-7.10 (5H, m), 7.09-7.07 (2H, m), 4.38 (2H, s), 4.33 (2H, s), 3.81 (3H, s). MS (ESI+): 430.0. HPLC (Condition B): Rt 3.63 min (HPLC purity 92.3%).

### Intermediate 2b

### 4-([benzyl-(4-chloro-benzenesulfonyl)-amino]-methyl)-benzoic acid

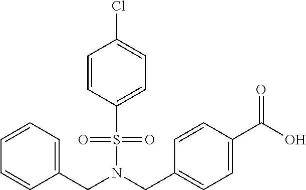

A solution 4-{[benzyl-(4-chloro-benzenesulfonyl)-amino]methyl}benzoic acid methyl ester (Intermediate 2; 1.80 g; 4.19 mmol) in THF:MeOH:H2O (8:1:1, 30 ml) was treated with lithium hydroxide monohydrate (350 mg, 8.4 mmol). After stirring for 16 h the solvents were concentrated under vacuum. The mixture was diluted with water and neutralised with 10% citric acid solution. At neutral pH a precipitate was obtained which was filtered. The precipitate was washed with water dried under vacuum to afford the title compound as a yellow solid (1.21 g; 74%)

¹H NMR (DMSO-d6, 400 MHz): δ 7.87 (2H, d, J=8.5 Hz), 7.70-7.63 (4H, m), 7.23-7.18 (3H, m), 7.10-7.05 (2H, m), 6.93 (2H, d, J=8.0 Hz), 4.29 (2H, s), 4.28 (2H, s). MS (ESI−): 413.8. HPLC (Condition B): Rt 1.80 min (HPLC purity 96.6%).

### Intermediate 3

### 4-chloro-N-(midin-2-ylmethyl)benzenesulfonamide

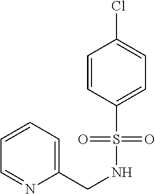

A cooled (0° C.) solution of 2-(aminomethyl)pyridine (1.00 g; 9.25 mmol) in DCM (20 ml) and triethylamine (1.28 ml; 9.25 mmol) was treated with a solution of 4-Chlorobenzene sulfonyl chloride (1.95 g; 9.25 mmol) in DCM (10 mL). After stirring at room temperature for 16 hours, the mixture was diluted with 30 ml of DCM and washed with water and with a NaHCO₃ solution. The organic phase was separated, dried over magnesium sulfate, filtered and evaporated in vacuo and concentrated to yellow residue. The residue was suspended into 50 ml of n-pentane and filtered, to give the title compound (2.46 g, 94%) as a pale yellow solid.

¹H NMR (DMSO-d6, 400 MHz): δ 8.42-8.38 (2H, m), 7.76 (2H, d, J=8.5 Hz), 7.70 (1H, td, J=7.5 Hz, J=2.0 Hz), 7.60 (2H, d, J=8.5 Hz), 7.31 (1H, d, J=8.0 Hz), 7.22 (1H, d J=7.0 Hz, J=5.0 Hz), 4.10 (2H, d, J=6.0 Hz). MS (ESI+): 282.8. HPLC (Condition A): Rt 2.02 min (HPLC purity 98.9%).

### Intermediate 3a

### 4-Ethoxy-N-pyridin-2-ylmethyl-benzenesulfonamide

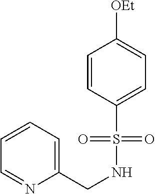

Following the general method as outlined for Intermediate 3, starting from 4-ethoxy-benzene-sulfonyl chloride the title compound was obtained as a white solid in 95% yield. (MS: m/z: 293).

### Intermediate 4

### Methyl 4-{[[(4-chlorophenyl)sulfonyl](pyridin-2-ylmethyl)amino]methyl}benzoate

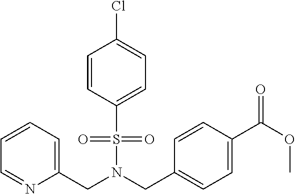

A cooled (−20° C.) solution of 4-chloro-N-(pyridin-2-ylmethyl)benzenesulfonamide (Intermediate 3; 500 mg; 1.77 mmol) dissolved in anhydrous DMF (2 ml) was treated with sodium hydride (60% suspension in mineral oil, 42.4 mg; 1.77 mmol). After stirring for 10 min, methyl 4-(bromomethyl)benzoate (425.3 mg; 1.86 mmol) was added. The cold bath was removed and the reaction was allowed to reach RT. After stirring for 24 h, the mixture was diluted with DCM and extracted with sat. NaHCO₃ solution and brine. The organic phase was concentrated to an oily red residue, which was purified by slurrying in ether and ethanol to give the title compound as a yellow powder (222 mg, 29%).

¹H NMR (DMSO-d6, 400 MHz): δ 8.30 (1H, ddd, J=5.0 Hz, J=2.0 Hz, J=1.0 Hz), 7.86-7.80 (4H, m), 7.65-7.58 (3H, m), 7.32 (2H, d, J=8.5 Hz), 7.18-7.14 (2H, m), 4.53 (2H, s), 4.42 (2H, s), 3.82 (3H, s). MS (ESI+): 294.1. HPLC (Condition A): Rt 3.54 min (HPLC purity 99.3%).

### Intermediate 4a

### 4-{[(4-Ethoxy-benzenesulfonyl)-pyridin-2-ylmethyl-amino]-methyl}-benzoic acid ethyl ester

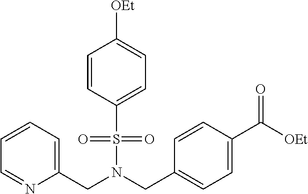

Following the general method as outlined for Intermediate 4, starting from 4-Ethoxy-N-pyridin-2-ylmethyl-benzenesulfonamide (intermediate 3a) and ethyl 4-(bromomethyl)benzoate the title compound was obtained as a brown solid in 98% yield. (MS: m/z: 455).

### Intermediate 4b

### 4-Chloro-N-(4-nitro-benzyl)-N-pyridin-2-ylmethyl-benzenesulfonamide

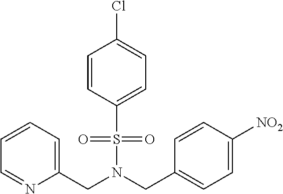

Following the general method as outlined for Intermediate 4, starting from 4-chloro-N-(pyridin-2-ylmethyl)benzenesulfonamide (intermediate 3; 1.5 g, 5.3 mmol) and 1-Bromomethyl-4-nitro-benzene (1.15 g; 5.3 mmol) the title compound was obtained as a brown solid (2.1 g: 93.3% yield). (MS: m/z: 418).

### Intermediate 4c

### N-(4-Amino-benzyl)-4-chloro-N-pyridin-2-ylmethyl-benzenesulfonamide

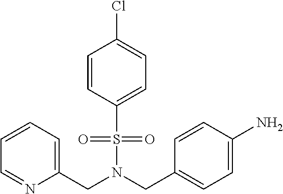

A solution of 4-Chloro-N-(4-nitro-benzyl)-N-pyridin-2-ylmethyl-benzenesulfonamide (intermediate 4b; 2.0 g, 4.78 mmol) in 60 ml THF, 80 ml ethanol and 30 ml water was treated with 435.2 mg (8.12 mmol) ammoniumchloride and 1.2 g iron dust and refluxed for 2 h. After cooling to RT the mixture was filtered over celite and concentrated in vacuo and the remained aqueous mixture was extracted with EtOAc. The organic phase was dried over sodium sulfate, filtered and concentrated to give a brown crystalline solid (1.67 g; 89.9% yield). (MS: m/z: 388)

### Intermediate 5

### 4-{[[(4-chlorophenyl)sulfonyl](pyridin-2-ylmethyl)amino]methyl}benzoic acid

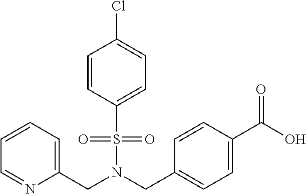

A solution of methyl 4-{[[(4-chlorophenyl)sulfonyl](pyridin-2-ylmethyl)amino]methyl}benzoate (Intermediate 4; 222 mg; 0.52 mmol) in THF (2 ml) was treated with a solution of sodium hydroxide (5 M) in water (0.52 ml; 2.58 mmol). After stirring at 40° C. for 20 h, the solution was diluted with ACN (50 ml), stirred 2 hours and filtered. The solid, which was purified by slurrying in EtOH and Et₂O to give the title compound as an ivory solid (201 mg, 94%).

¹H NMR (DMSO-d6, 400 MHz): δ 12.9 (1H, bs), 8.30 (1H, d, J=4.5 Hz), 7.84 (2H, d, J=8.5 Hz), 7.79 (2H, d, J=8.0 Hz), 7.63-7.59 (3H, m), 7.29 (2H, d, J=8.0 Hz), 7.16 (2H, m), 4.52 (2H, s), 4.42 (2H, s). MS (ESI+): 417.2. HPLC (Condition A): Rt 3.12 min (HPLC purity 99.73%).

### Intermediate 5a

### 4-{[(4-Ethoxy-benzenesulfonyl)-pyridin-2-ylmethyl-amino]-methyl}-benzoic acid

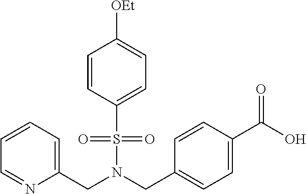

Following the general method as outlined for Intermediate 5, starting from 4-{[(4-Ethoxy-benzenesulfonyl)-pyridin-2-ylmethyl-amino]-methyl}-benzoic acid ethyl ester (intermediate 4a) the title compound was obtained as a brown solid in 98% yield. (MS: m/z: 427).

### Intermediate 6

### 4-(bromomethyl)-N-(cyclopropylmethyl)benzamide

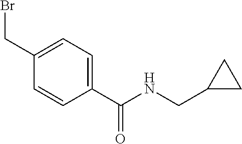

A solution of 4-(bromomethyl)benzoic acid (2.00 g; 9.30 mmol) and aminomethylcyclopropane (661.46 mg; 9.30 mmol) in a mixture of DCM (40 ml) and THF (10 ml) was treated with 1-(3-dimethylaminopropyl)-3-ethylcarbodiimide hydrochloride (2.14 g; 11.16 mmol). After stirring for 5 h, the solvents were evaporated in vacuo to give an orange oily residue, which was dissolved in DCM and extracted with water. The organic phase was concentrated in vacuo to afford a solid which was purified by column chromatography (silica) eluting with cyclohexane containing increasing amounts of EtOAc, to afford the title compound as a white solid (631 mg, 25%).

¹H NMR (DMSO-d6, 300 MHz): δ 8.56 (1H, t, J=5.5 Hz), 7.84 (2H, d, J=8.5 Hz), 7.50 (4H, d, J=8.5 Hz), 4.80 (2H, s), 3.13 (2H, t, J=6.0 Hz), 1.01 (1H, m), 0.44-0.39 (2H, m), 0.24-0.19 (2H, m). HPLC (Condition A): Rt 3.24 min (HPLC purity 86.2%).

### Intermediate 7

### N-(3-chlorobenzyl)-4-(chloromethyl)benzamide

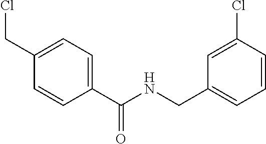

A cooled (0° C.) solution of 3-chlorobenzylamine (1.12 g; 7.93 mmol) and triethylamine (1.10 ml; 7.93 mmol) in DCM (30 mL) was treated with a solution of 4-chloromethylbenzoyl chloride (1.50 g; 7.93 mmol) in DCM (10 ml). After stirring at 0° C. for 2 h, the mixture was diluted with DCM and extracted with brine. The organic phase was dried over magnesium sulfate, filtered and concentrated to give a pale yellow solid, which was crystallised from DCM/Cyclohexane to afford the title compound as a white solid (1.97 g, 84%).

¹H NMR (DMSO-d6, 300 MHz): δ 9.12 (1H, t, J=6.0 Hz), 7.90 (2H, d, J=8.5 Hz), 7.54 (2H, d, J=8.5 Hz), 7.39-7.27 (4H, m), 4.82 (2H, s), 4.48 (2H, d, J=6.0 Hz). MS (ESI+): 294.1. HPLC (Condition A): Rt 4.44 min (HPLC purity 98.6%).

### Intermediate 8

### methyl 6-({[(4-chlorophenyl)sulfonyl]amino}methyl)nicotinate

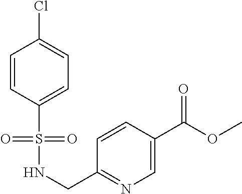

A cooled (0° C.) solution of methyl-6-aminomethylpyridine-3-carboxylate.HCl (700 mg; 3.45 mmol) and triethylamine (0.96 ml; 6.91 mmol) in DCM (14 ml) was treated with a solution of 4-chlorobenzenesulfonyl chloride (729 mg; 3.45 mmol) in DCM (10 mL). After stirring for 20 h, the mixture was diluted with DCM and washed with water and sat. NaHCO₃ solution. The organic phase was separated, dried over magnesium sulfate, filtered and concentrated to give solid, which was crystallised from DCM/Cyclohexane to afford the title compound as a grey solid (524 mg, 45%).

¹H NMR (DMSO-d6, 300 MHz): δ 8.91 (1H, d, J=1.5 Hz), 8.54 (1H, t, J=6.5 Hz), 8.23 (1H, dd, J=8.0 Hz, J=2.0 Hz), 7.76 (2H, d, J=8.5 Hz), 7.62 (2H, d, J=8.5 Hz), 7.50 (1H, d, J=8.0 Hz) 4.21 (2H, d, J=6.5 Hz), 3.88 (3H, s). MS (ESI+): 341.1. HPLC (Condition A): Rt 3.37 min (HPLC purity 97.7%).

### Intermediate 9

### methyl 6-({benzyl[(4-chlorophenyl)sulfonyl]amino}methyl)nicotinate

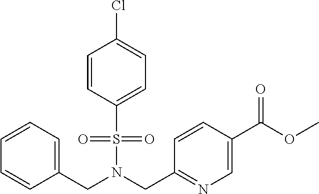

A mixture of methyl 6-({[(4-chlorophenyl)sulfonyl]amino}methyl)nicotinate (Intermediate 8, 300 mg; 0.88 mmol), benzyl bromide (151 mg; 0.88 mmol), potassium carbonate (128 mg; 0.92 mmol) and sodium iodide (2.6 mg; 0.02 mmol) in DMF (3 ml) was heated to 100° C. for 1.5 hours. The mixture was diluted with DCM and extracted with brine. The organic phase was separated, dried over magnesium sulfate, filtered and concentrated to give solid, which was crystallised from isopropyl alcohol to afford the title compound as a pale yellow solid (106 mg, 28%).

¹H NMR (DMSO-d6, 300 MHz): δ 8.80 (1H, d, J=2.0 Hz), 8.12 (1H, dd, J=8.0 Hz, J=2.0 Hz), 7.87 (2H, d, J=8.5 Hz), 7.65 (2H, d, J=8.5 Hz), 7.32 (1H, d, J=7.5 Hz) 7.26-7.16 (5H, m), 4.50 (2H, d), 4.47 (4H, s), 3.86 (3H, s). MS (ESI+): 431.2 (M+H₂O). HPLC (Condition A): Rt 4.88 min (HPLC purity 99.1%).

### Intermediate 10

### 6-({benzyl[(4-chlorophenyl)sulfonyl]amino}methyl)nicotinic acid

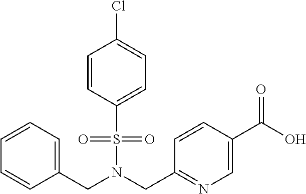

A solution of methyl 6-({benzyl[(4-chlorophenyl)sulfonyl]amino}methyl)nicotinate (Intermediate 9, 100 mg, 0.23 mmol) in THF (0.5 ml) was treated with a solution (5M) of sodium hydroxide in water (0.23 ml; 1.16 mmol). After stirring for 24 h, the mixture was acidified to pH 7 with HCl (1N). EtOAc was added and the organic phase was washed with a citric acid solution (10%). The organic phase was separated, dried over magnesium sulfate, filtered and concentrated to give solid, which was slurried in Et₂O to afford the title compound as an ivory solid (98 mg, quant.).

¹H NMR (DMSO-d6, 300 MHz): δ 8.78 (1H, bs), 8.08 (1H, d, J=8.0 Hz), 7.86 (2H, d, J=8.5 Hz), 7.65 (2H, d, J=8.5 Hz), 7.29 (1H, d, J=8.0 Hz), 7.22-7.18 (5H, m), 4.48 (2H, s), 4.47 (2H, s). MS (ESI+): 417.2. HPLC (Condition A): Rt 4.27 min (HPLC purity 99.0%).

### Intermediate 11

### 4-{[[(4-methoxyphenyl)sulfonyl](pyridin-3-ylmethyl)amino]methyl}benzoic acid

### Step 1—4-methoxy-N-pyridin-3-ylmethyl-benzenesulfonamide

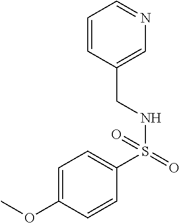

A solution of 1-pyridin-3-ylmethanamine (11.89 g, 110 mmol) and triethylamine (14.0 mL, 110 mmol) in anhydrous acetonitrile (150 ml) was treated with 4-methoxybenzenesulfonyl chloride (20.66 g, 100 mmol). After stirring for 15 min, the mixture was filtered, the filtrate was concentrated to ca. 50 mL and diluted with hot water (150 mL). Upon cooling, the precipitate was filtered to give the title compound (19.29 g, 63%).

### Step 2—4-{[[(4-methoxyphenyl)sulfonyl](pyridin-3-ylmethyl)amino]methyl}benzoate

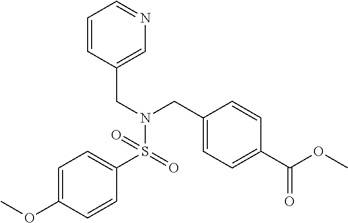

A cooled (−20° C.) solution of 4-methoxy-N-pyridin-3-ylmethyl-benzenesulfonamide (10.63 g, 38.2 mmol) in anhydrous DMF (20 mL) was treated portionwise with NaH (1.53 g, 60% in mineral oil; 38.2 mmol), followed by methyl 4-chloromethylbenzoate (7.38 g, 40 mmol). The resulting mixture was allowed to attain room temperature and stirred for 1 h. Then the reaction mixture was heated to 40° C., diluted with hot water (10 ml) and extracted with hexane to remove the mineral oil. The aqueous solution was diluted with water 1:1. The precipitated product was washed with 50% aqueous methanol to give the title compound, which was used in the next step without additional purification.

### Step 3—4-{[[(4-methoxyphenyl)sulfonyl](pyridin-3-ylmethyl)amino]methyl}benzoic acid

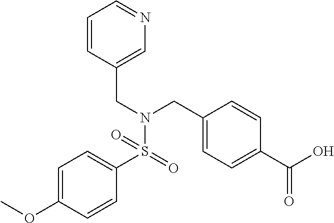

A solution of methyl 4-{[[(4-methoxyphenyl)sulfonyl](pyridin-3-ylmethyl)amino]methyl}benzoate (1.00 g; 2.34 mmol) in THF (10 ml) was treated with a sodium hydroxide solution (5 N) in water (2.3 ml; 12 mmol). After stirring for 18 h, the mixture was diluted with ether. The precipitate was filtered and purified by crystallisation from ethanol to give the title compound as a white powder (554.6 mg, 57%).

¹H NMR (DMSO-d6, 300 MHz): δ 12.89 (1H, bs), 8.34 (1H, d, J=4.0 Hz), 8.25 (1H, d, J=1.5 Hz), 7.86 (2H, d, J=8.5 Hz), 7.75 (2H, d, J=8.0 Hz), 7.51 (1H, d, J=8.0 Hz), 7.24-7.15 (5H, m), 4.37 (2H, s), 4.33 (2H, s), 3.88 (3H, s). MS (ESI+): 413.2. HPLC (Condition A): Rt 2.68 min (HPLC purity 98.1%).

### Intermediate 12

### 1-(3-nitrophenyl)methanesulfonamide

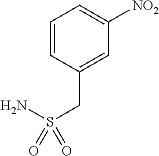

A cooled (0° C.) solution of 3-nitrophenylmethanesulfonyl chloride (1.00 g; 4.24 mmol) in dioxane (20 mL) was carefully treated with a solution of ammonia in dioxane (42 ml; 0.50 M; 21 mmol). After stirring for 0.5 hours, the white precipitate was filtered off, the solvent was removed in vacuo and the residue dissolved in DCM and extracted with brine. The organic phase was separated, dried over magnesium sulfate, filtered and concentrated to give the title compound as a white solid (606 mg, 66%).

¹H NMR (DMSO-d6, 300 MHz): δ 8.27-8.21 (2H, m), 7.83 (1H, dt, J=8.0 Hz, J=1.0 Hz), 7.69 (1H, t, J=8.0 Hz), 6.94 (2H, bs), 4.48 (2H, s). MS (ESI−): 215.1. HPLC (Condition A): Rt 2.83 min (HPLC purity 95.8%).

### Intermediate 13

### 4-chloro-N-(4-cyanobenzyl)-N-(pyridin-2-ylmethyl)benzenesulfonamide

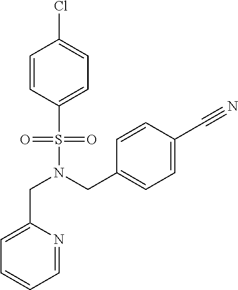

A mixture of 4-chloro-N-(pyridin-2-ylmethyl)benzenesulfonamide (Intermediate 3, 100 mg; 0.35 mmol), alpha-bromo-p-tolunitrile (69 mg; 0.35 mmol) potassium carbonate (49.9 mg; 0.36 mmol), sodium iodide (1 mg; 0.01 mmol) in anhydrous DMF (1 ml) was heated to 80° C. for 2 h. The mixture was diluted with DCM and extracted with brine. The organic phase was separated, dried over magnesium sulfate, filtered and concentrated to give solid, which was slurried in Et₂O to afford the title compound as a brown powder (83 mg, 59%).

¹H NMR (DMSO-d6, 400 MHz): δ 8.29 (1H, d, J=4.0 Hz), 7.84 (2H, d, J=8.5 Hz), 7.69 (2H, d, J=8.0 Hz), 7.64-7.59 (3H, m), 7.36 (2H, d, J=8.0 Hz), 7.16 (2H, t, J=6.5 Hz), 4.53 (2H, s), 4.43 (2H, s). MS (ESI+): 398.2. HPLC (Condition A): Rt 3.74 min (HPLC purity 93.9%).

### Intermediate 14

### methyl 4-{[benzyl(4-methoxysulfonyl)amino]methyl}benzoate

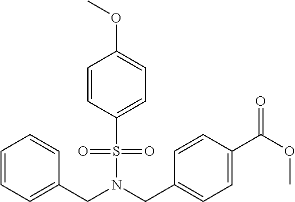

A cooled (0° C.) suspension of methyl 4-[(benzylamino)methyl]benzoate hydrochloride (Intermediate 2; 500 mg, 1.74 mmol) in anhydrous DCM (20 ml) was treated with triethylamine (0.75 ml, 5.2 mmol) and 4-methoxybenzenesulfonylchloride (intermediate 1: 430 mg, 2.08 mmol) under nitrogen atmosphere. After stirring at RT for 16 h, the reaction mixture was diluted with DCM and successively washed with 10% sodium bicarbonate, water and saturated brine. The organic layers were dried over sodium sulfate, concentrated and recrystallised with DCM/hexane to get the title compound as an off white solid (700 mg, 89%).

¹H NMR (DMSO-d6, 400 MHz): δ 7.83 (2H, d, J=8.5 Hz), 7.75 (2H, d, J=8.0 Hz), 7.22-7.08 (6H, m), 7.09-7.07 (2H, m), 4.31 (2H, s), 4.27 (2H, s), 3.85 (3H, s), 3.80 (3H, s). MS (ESI+): 426.0. HPLC (Condition B): Rt 4.22 min (HPLC purity 99.5%).

### Intermediate 15

### methyl 4-{[benzyl(4-methoxybenzenesulfonyl)amino]methyl}benzoic acid

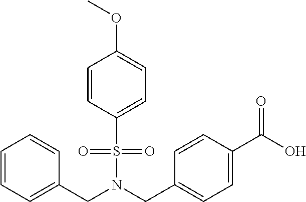

Following the general method as outlined for Intermediate 5, starting methyl-4-{[benzyl(4-methoxybenzenesulfonyl)amino]}benzoate (Intermediate 14; 350 mg; 0.82 mmol), the title compound was obtained as a yellow solid in 79% yield.

¹H NMR (DMSO-d6, 400 MHz): δ 12.8 (1H, br s), 7.82 (2H, d, J=9.0 Hz), 7.73 (2H, d, J=8.5 Hz), 7.20-7.14 (7H, m), 7.09-7.06 (2H, m), 4.31 (2H, s), 4.27 (2H, s), 3.85 (3H, s). MS (ESI+): 412.0. HPLC (Condition B): Rt 3.60 min (HPLC purity 98.7%).

### Intermediate 16

### 4-{[Benzyl-(4-ethoxy-benzenesulfonyl)-amino]-methyl}-benzoic acid methyl ester

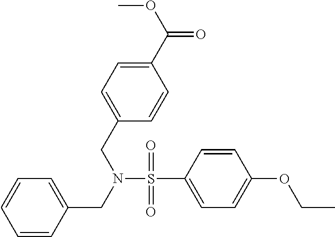

Following the general method as outlined for Intermediate 2, starting methyl 4-[(benzyl amino)methyl]benzoate hydrochloride (Intermediate 1; 500 mg, 1.74 mol) and 4-ethoxy benzene sulfonyl chloride (440 mg, 2.05 mol) the title compound was obtained as an off-white solid in 67% yield.

¹H NMR (DMSO-d6, 400 MHz): δ7.80 (2H, d, J=9.0 Hz), 7.75 (2H, d, J=8.5 Hz) 7.20-7.15 (5H, m), 7.12-7.06 (4H, m), 4.31 (2H, s), 4.27 (2H, s), 4.12 (2H, q, J=7.0 Hz), 3.80 (3H, s), 1.35 (3H, t, J=7.0 Hz). MS (ESI+): 440.3. HPLC (Condition B): Rt 4.34 min (HPLC purity 97.1%).

### Intermediate 16b

### 4-{[Benzyl-(4-ethoxy-benzenesulfonyl)-amino]-methyl}benzoic acid

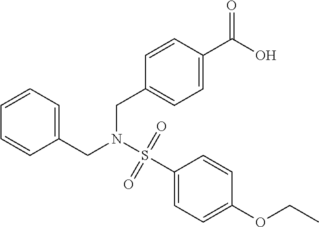

Following the general method as outlined in Example 1, starting from 4-{[Benzyl-(4-ethoxy-benzenesulfonyl)-amino]-methyl}-benzoic acid methyl ester (Intermediate 16; 500 mg; 1.1 mol), the title compound was obtained as a white solid in 86% yield.

¹H NMR (DMSO-d6, 400 MHz): δ 7.80 (2H, d, J=8.5 Hz), 7.70 (2H, d, J=8.0 Hz), 7.20-7.18 (3H, m), 7.11-7.04 (6H, m), 4.27 (2H, s), 4.25 (2H, s), 4.14 (2H, q, J=7.0 Hz), 1.35 (3H, t, J=7.0 Hz). MS (ESI−): 423.9. HPLC (Condition B): Rt 4.34 min (HPLC purity 95.3%).

### Intermediate 17

### 4-chloro-N-[4-(hydrazinomethyl)benzyl]-N-(pyridin-2-ylmethyl)benzene sulfonamide

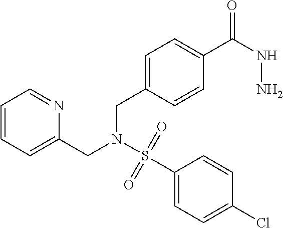

A solution of methyl 4-({benzyl[(4-chlorophenyl)sulfonylurea]amino}methyl)benzoate (Intermediate 4, 200 mg, 0.46 mmol) in MeOH:THF (3:1) was treated with hydrazine hydrate (37 mg, 7.42 mmol). The mixture was refluxed for 16 h under nitrogen. The reaction mixture was concentrated and washed with methanol to get the title compound (150 mg, 75%) as an off white solid.

¹H NMR (DMSO-d6, 400 MHz): δ 9.70 (1H, s), 8.30 (1H, m), 7.83 (2H, d, J=8.5 Hz), 7.68 (2H, d, J=8.5 Hz), 7.64-7.59 (3H, m), 7.16 (2H, d, J=8.5 Hz), 7.18-7.15 (2H, m), 4.49 (2H, s), 4.41 (2H, s). MS (ESI+): 430.9. HPLC (Condition B): Rt 4.29 min (HPLC purity 99.4%).

### Intermediate 18

### 4-chloro-N-(4-cyanobenzyl)benzenesulfonamide

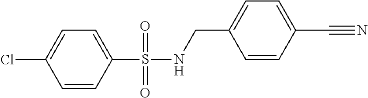

A cold (0° C.) solution of 4-(aminomethyl)benzonitrile hydrochloride (2.00 g; 11.8 mmol) in anhydrous DCM (40 ml) was treated with triethylamine (6.6 ml; 47.5 mol) followed by a solution of 4-chlorobenzenesulfonylchloride (2.80 g; 13.0 mmol). The reaction mixture was allowed to warm to room temperature and stirred overnight. The reaction mixture was quenched with ice, diluted with DCM (100 ml) and washed with 10% aqueous sodium bicarbonate followed by brine solution. The organic layer was dried over sodium sulfate, concentrated and purified by column chromatography (silica) eluting with chloroform containing increasing amounts of EtOAc to give the Title compound (2.80 g, 78%).

¹H NMR (DMSO-d6, 400 MHz): δ 8.45-8.42 (1H, m), 7.77-7.70 (4H, m), 7.62 (2H, d, J=8.5 Hz), 7.42 (2H, d, J=8.5 Hz), 4.11-4.09 (2H, m). MS (ESI−): 304.9. HPLC (Condition B): Rt 3.53 min (HPLC purity 99.9%).

### Intermediate 19

### 4-chloro-N-(4-cyanobenzyl)-N-(2-fluorobenzyl)benzenesulfonamide

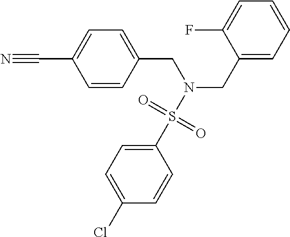

Following the general method as outlined for Intermediate 13, starting from 4-chloro-N-(4-cyanobenzyl)benzene sulfonamide (Intermediate 18; 500 mg; 1.63 mmol) and 2-fluorobenzylbromide (338 mg; 1.79 mmol), the title compound was obtained as a yellow solid in 88.7% yield after recrystallisation from DCM/Hexane.

¹H NMR (DMSO-d6, 400 MHz) δ 7.90-7.88 (2H, dt, J=8.5 Hz, J=2.0 Hz), 7.68 (2H, dt, J=8.5 Hz, J=2.0 Hz), 7.64 (2H, dt, J=8.5 Hz, J=2.0 Hz), 7.28 (2H, d, J=8.5 Hz), 7.22-7.17 (2H, m), 7.01-6.91 (2H, m), 4.42 (2H, s), 4.39 (2H, s). MS (ESI+): 415.1. HPLC (Condition B): Rt 4.21 min (HPLC purity 99.7%).

### Intermediate 20

### 4-chloro-N-(3-chloro-benzyl)-N-(4-cyano-benzyl)-benzenesulfonamide

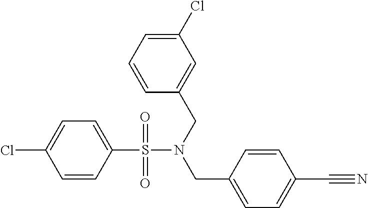

Following the general method as outlined for Intermediate 13, starting from 4-chloro-N-(4-cyanobenzyl)benzene sulfonamide (Intermediate 18; 500 mg; 1.63 mmol) and 3-chlorobenzyl bromide (367 mg; 1.79 mmol), the title compound was obtained as a white solid in 80% yield after recrystallisation from DCM/Hexane.

¹H NMR (DMSO-d6, 400 MHz): δ 7.91 (2H, d, J=8.5 Hz), 7.70 (2H, d, J=8.5 Hz), 7.65 (2H, d, J=8.5 Hz), 7.29 (2H, d, J=8.5 Hz), 7.22-7.18 (2H, m), 7.07-7.05 (1H, m), 6.97 (1H, s), 4.40 (2H, s), 4.30 (2H, s). MS (ESI+): 430.9. HPLC (Condition B): Rt 4.32 min (HPLC purity 99.8%).

### Intermediate 21

### 4-chloro-N-(4-cyanobenzyl)-N-[4-(Fluoro benzyl]benzenesulfonamide

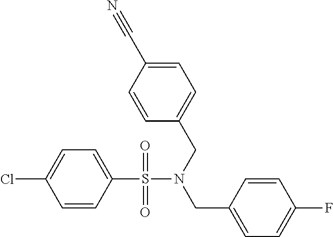

Following the general method as outlined for Intermediate 13, starting from 4-chloro-N-(4-cyanobenzyl)benzene sulfonamide (Intermediate 19; 500 mg; 1.63 mmol) and 4-fluorobenzyl bromide (340 mg, 1.79 mmol), the title compound was obtained as an off-white solid in 90% yield after recrystallisation from DCM/Hexane.

¹H NMR (DMSO-d6, 400 MHz): δ 7.91 (2H, d, J=8.5 Hz), 7.70 (2H, d, J=8.5 Hz), 7.64 (2H, d, J=8.0 Hz), 7.25 (2H, d, J=8.0 Hz), 7.15-7.11 (2H, m), 6.98 (2H, t, J=9.0 Hz), 4.40 (2H, s), 4.32 (2H, s). MS (ESI+): 414.9. HPLC (Condition B): Rt 4.20 min (HPLC purity 99.8%).

### Intermediate 22

### 4-chloro-N-(4-cyanobenzyl)-N-(3-methoxybenzyl)benzene sulfonamide

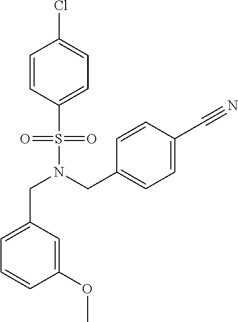

Following the general method as outlined for Intermediate 13, starting from 4-chloro-N-(4-cyanobenzyl)benzene sulfonamide (Intermediate 18; 500 mg; 1.63 mmol) and 3-methoxybenzylbromide (393 mg; 1.9 mmol), the title compound was obtained as a yellow solid in 79% yield after recrystallisation from DCM/Hexane.

¹H NMR (DMSO-d6, 400 MHz) δ 7.90 (2H, d, J=8.5 Hz), 7.70-7.64 (4H, m), 7.28 (2H, d, J=8.0 Hz), 7.08 (1H, t, J=8.0 Hz), 6.73-6.64 (2H, m), 6.55 (1H, s), 4.39 (2H, s), 4.30 (2H, s), 3.59 (3H, s). MS (ESI+): 426.9. HPLC (Condition B): Rt 4.18 min (HPLC purity 97.7%).

### Intermediate 23

### 4-chloro-N-(4-cyanobenzyl)-N-(4-methoxybenzyl)benzene sulfonamide

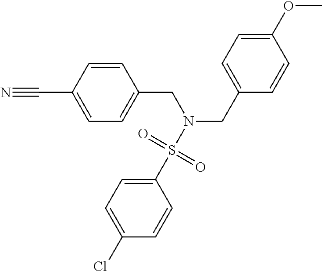

Following the general method as outlined for Intermediate 13, starting from 4-chloro-N-(4-cyanobenzyl)benzene sulfonamide (Intermediate 18; 500 mg; 1.63 mmol) and 4-methoxy benzyl bromide (343 mg, 1.7 mmol), the title compound was obtained as a white solid in 88% yield after recrystallisation from DCM/Hexane.

¹H NMR (DMSO-d6, 400 MHz): δ7.89 (2H, d, J=8.5 Hz), 7.66 (4H, m), 7.24 (2H, d, J=8.0 Hz) 6.99 (2H, d, J=8.5 Hz) 6.72 (2H, d, J=8.5 Hz), 4.36 (2H, s), 4.25 (2H, s), 3.67 (3H, s). MS (ESI+): 448.9 (M+Na). HPLC (Condition B): Rt 4.18 min (HPLC purity 99.2%).

### Intermediate 24

### 4-chloro-N-(4-chlorobenzyl)-N-(4-cyanobenzyl)benzenesulfonamide

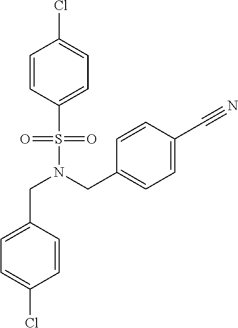

Following the general method as outlined for Intermediate 13, starting from 4-chloro-N-(4-cyanobenzyl)benzene sulfonamide (Intermediate 18; 500 mg; 1.63 mmol) and 4-chlorobenzyl bromide (367 mg; 1.79 mmol), the title compound was obtained as a yellow solid in 80% yield after recrystallisation from DCM/Hexane.

¹H NMR (DMSO-d6, 400 MHz): δ 7.91 (2H, d, J=8.5 Hz), 7.67 (2H, d, J=8.5 Hz), 7.64 (2H, d, J=8.0 Hz), 7.26 (2H, d, J=8.0 Hz), 7.22 (2H, d, J=8.5 Hz), 7.11 (2H, d, J=8.5 Hz), 4.40 (2H, s), 4.33 (2H, s). MS (ESI+): 430.9. HPLC (Condition B): Rt 4.32 min (HPLC purity 99.4%).

### Intermediate 25

### 4-[(benzyl{[4-(trifluoromethoxy)phenyl]sulfonyl}amino)methyl]benzoate

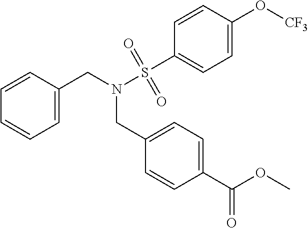

Following the general method as outlined for Intermediate 2, starting from methyl 4-[(benzyl amino)methyl]benzoate hydrochloride (Intermediate 1; 500 mg, 1.74 mmol) and 4-trifluoromethoxy benzene sulfonyl chloride (496 mg, 1.91 mmol) the title compound was obtained as an off-white solid (600 mg, 73%).

¹H NMR (DMSO-d6, 400 MHz): δ 8.01 (2H, d), 7.75 (2H, d), 7.57 (2H, d), 7.21-7.16 (5H, m), 7.07 (2H, m), 4.42 (2H, s), 4.37 (2H, s), 3.81 (3H, s). MS (ESI+): 479.9. HPLC (Condition B): Rt 4.42 min (HPLC purity 94.4%).

### Intermediate 26

### 4-[(benzyl{[4-(trifluoromethoxy)phenyl]sulfonyl}amino)methyl]benzoic acid

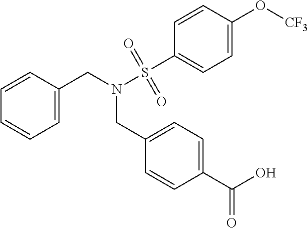

A mixture of 4-[(benzyl{[4-(trifluoromethoxy)phenyl]sulfonyl}amino)methyl]benzoate (Intermediate 25; 600 mg; 0.82 mmol) in THF:MeOH:Water (4.5:4.5:1) was treated with lithium hydroxide monohydrate (71 mg; 1.62 mmol) and stirred for 16 h. The reaction mixture was concentrated under vacuum, diluted with water and neutralized to pH 7 with a 10% citric acid solution. The resulting precipitate was filtered, washed with water and dried under vacuum to afford the title compound as a white solid.

¹H NMR (DMSO-d6, 400 MHz): δ 8.02-8.00 (2H, d), 7.75-7.73 (2H, d), 7.58-7.56 (2H, d), 7.21-7.11 (5H, m), 7.09-7.07 (2H, m), 4.40 (2H, s), 4.37 (2H, s). MS (ESI−): 463.8. HPLC (Condition B): Rt 3.99 min (HPLC purity 95.6%).

### Intermediate 27

### Methyl 4-({benzyl[(3,4-dichlorophenyl)sulfonylurea]amino}methyl)benzoate

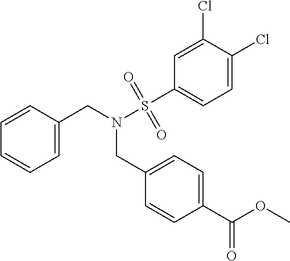

Following the general method as outlined for Intermediate 2, starting from methyl 4-[(benzyl amino)methyl]benzoate hydrochloride (Intermediate 1; 500 mg, 1.74 mmol) and 3,4-dichloro benzene sulfonyl chloride (524 mg, 2.05 mmol) the title compound was obtained as an off-white solid.

¹H NMR (DMSO-d6, 400 MHz): δ7.96-7.97 (1H, d), 7.84-7.85 (2H, t), 7.76-7.78 (2H, d), 7.23-7.25 (2H, d), 7.18-7.20 (3H, m), 7.12-7.13 (2H, t), 4.45 (2H, s), 4.40 (2H, s), 3.81 (3H, s). HPLC (Condition B): Rt 4.80 min (HPLC purity 97.0%).

### Intermediate 28

### 4-({benzyl[(3,4-dichlorophenyl)sulfonylurea]amino}methyl)benzoic acid

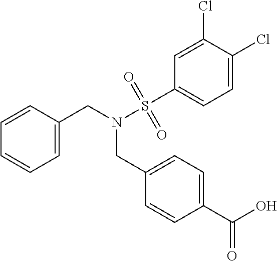

Following the general method as outlined for Intermediate 26, starting from methyl 4-({benzyl [(3,4-dichlorophenyl) sulfonylurea]amino}methyl)benzoate (Intermediate 27; 300 mg; 0.64 mmol), the title compound was obtained as a white solid in 86% yield.

¹H NMR (DMSO-d6, 400 MHz): δ7.962-7.966 (1H, d), 7.842-7.848 (2H, d), 7.72-7.74 (2H, d), 7.19-7.20 (7H, m), 4.42 (2H, s), 4.38 (2H, s). MS (ESI−): 447.7. HPLC (Condition B): Rt 4.11 min (HPLC purity 97.2%).

### Intermediate 29

### Methyl 4-({[(4-chlorophenyl)sulfonyl]amino}methyl)benzoate

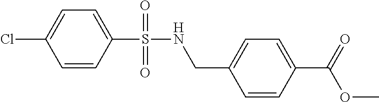

A cooled (0° C.) solution of methyl 4-(amino methyl)benzoate hydrochloride (5.00 g, 24.7 mmol) in dichloromethane (75 ml) was treated with triethylamine (7.4 g, 74.1 mmol) and stirred for 10 minutes, then treated with 4-chlorobenzene sulfonyl chloride (5.73 g, 27.1 mmol) and stirred overnight. The reaction mixture was quenched with 10% aqueous sodium bicarbonate and stirred for 15 min. The precipitated product was filtered, washed with water and dried to yield methyl 4-({[(4-chlorophenyl)sulfonyl]amino}methyl)benzoate (7 g, 84%) as a white solid.

¹H NMR (DMSO-d6, 400 MHz): δ8.37-8.40 (1H, t), 7.84-7.86 (2H, d), 7.75-7.77 (2H, m), 7.62-7.64 (2H, d), 7.35-7.38 (2H, d), 4.07-4.08 (2H, d), 3.82 (3H, s). MS (ESI+): 337.8. HPLC (Condition B): Rt 3.63 min (HPLC purity 99.2%).

### Intermediate 30

### Methyl 4-({[(4-chlorophenyl)sulfonyl][4-(trifluoromethyl)benzyl]amino}methyl)benzoate

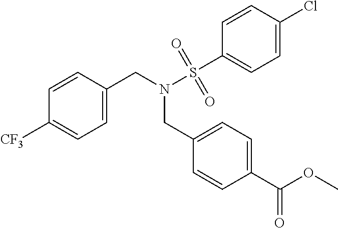

A cooled (−30° C.) solution of methyl 4-({[(4-chlorophenyl)sulfonyl]amino}methyl)benzoate (Intermediate 29, 0.50 g, 1.47 mmol) in dry DMF (12 ml) was treated with sodium hydride (77 mg, 1.61 mmol). After stirring for 15 min, 4-trifluoromethylbenzyl bromide (3.86 g, 1.67 mol) was added and the reaction mixture was stirred at RT for 12 h. The reaction mixture was quenched into water and extracted with ethyl acetate. The organic layer was washed with water, brine solution and dried over Na₂SO₄ and evaporated under vacuum. The crude was purified by column chromatography in silica gel to afford of the titled compound as a yellow solid.

¹H NMR (DMSO-d6, 400 MHz) δ 7.90-7.93 (2H, d), 7.68-7.73 (4H, m), 7.47-7.49 (2H, d), 7.28-7.30 (2H, d), 7.20-7.22 (2H, d), 4.43 (4H, s), 4 3.80 (3H, s). MS (ESI+): 497.9. HPLC (Condition B): Rt 4.48 min (HPLC purity 80%).

### Intermediate 31

### 4-({[(4-chlorophenyl)sulfonyl][4-(trifluoromethyl)benzyl]amino}methyl)benzoic acid

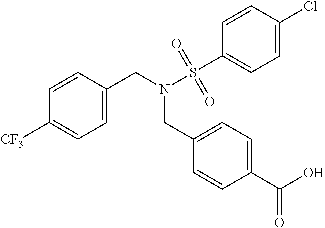

A cooled (0° C.) solution of methyl 4-({[(4-chlorophenyl)sulfonyl][4-trifluoromethyl)benzyl]amino}methyl)benzoate (Intermediate 30, 100 mg, 0.2 mmol) in THF (8 ml) and water (2 ml) was treated with lithium hydroxide (33 mg, 0.40 mmol) and the reaction mixture was stirred for 12 h. The reaction mixture was quenched with citric acid (10%) solution and filtered. The residue was washed with water and dried under vacuum to afford the title compound (80 mg, 91%) as white solid.

¹H NMR (DMSO-d6, 400 MHz) δ 7.90-7.92 (2H, d), 7.68-7.71 (4H, m), 7.48-7.50 (2H, d), 7.29-7.31 (2H, d), 7.17-7.19 (2H, d), 4.42 (4H, s). MS (ESI−): 481.6. HPLC (Condition B): Rt 4.07 min (HPLC purity 96.1%).

### Intermediate 32

### Methyl 4-{[[(4-chlorophenyl)sulfonyl](2-fluorobenzyl)amino]methyl}benzoate

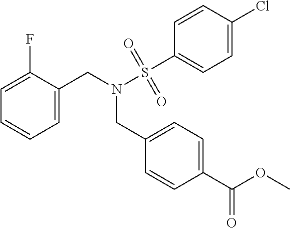

A stirred solution of methyl 4-({[(4-chlorophenyl)sulfonyl]amino}methyl)benzoate (Intermediate 29, 500 mg, 1.47 mmol) in dry DMF (20 ml) was treated with K₂CO₃ (207 mg, 1.50 mmol) and KI (5 mg, 0.03 mmol). After stirring for 15 min, 2-fluorobenzyl bromide (0.180 ml, 1.5 mmol) was added and the reaction mixture stirred at RT for 12 h. The reaction mixture was quenched into water and extracted with ethyl acetate. The organic layer was washed with water, brine solution and dried over Na₂SO₄ and evaporated under vacuum to afford the title compound (600 mg, 91%) as a yellow solid.

¹H NMR (DMSO-d6, 400 MHz) δ 7.89-7.90 (2H, d), 7.87-7.88 (2H, d), 7.74-7.77 (2H, d), 7.23-7.25 (2H, d), 7.17-7.20 (2H, m), 6.92-7.01 (2H, m), 4.41 (2H, s), 4.39 (2H, s), 3.81 (3H, s). MS (ESI+): 448.0. HPLC (Condition B): Rt 4.37 min (HPLC purity 98.6%).

### Intermediate 33

### 4-{[[(4-chlorophenyl)sulfonyl](2-fluorobenzyl)amino]methyl}benzoic acid

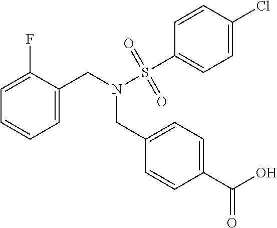

Following the general method as outlined for Intermediate 31, starting from methyl 4-{[[(4-chlorophenyl)sulfonyl](2-fluorobenzyl)amino]methyl}benzoate (Intermediate 32, 600 mg, 1.33 mmol), the title compound was obtained as a white solid in 86% yield.

¹H NMR (DMSO-d6, 400 MHz) δ 7.88-7.90 (2H, d), 7.71-7.36 (2H, d), 7.66-7.68 (2H, d), 7.16-7.21 (4H, m), 7.94-7.03 (2H, m), 4.39 (2H, s), 4.38 (2H, s). MS (ESI−): 432.0. HPLC (Condition B): Rt 3.91 min (HPLC purity 99.4%).

### Intermediate 34

### Methyl 4-({(3-chlorobenzyl)[(4-chlorophenyl)sulfonyl]amino}methyl)benzoate

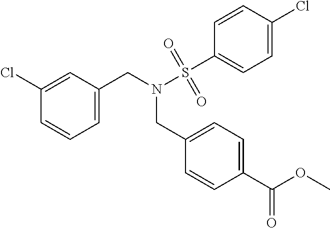

A solution of methyl 4-({[(4-chlorophenyl)sulfonyl]amino}methyl)benzoate (Intermediate 29, 500 mg, 1.47 mmol) in acetonitrile (20 mL) was treated with potassium carbonate (400 mg, 2.9 mmol) and 3-chlorobenzyl bromide (230 mg 2.9 mmol) and the mixture was refluxed to 70° C. for 3 h. Acetonitrile was removed under vacuum and the residue was dissolved in water and extracted with ethyl acetate (3×20 ml). The combined organic layer was washed with brine and then dried over anhydrous sodium sulphate and concentrated under vacuum to yield the title compound as white solid.

¹H NMR (DMSO-d6, 400 MHz): δ 7.89-7.92 (2H, m), 7.76-7.78 (2H, m), 7.68-7.71 (2H, m), 7.24-7.26 (2H, d), 7.18-7.19 (2H, m), 7.04-7.06 (1H, m), 6.98 (1H, s), 4.42 (2H, s), 4.3 (2H, s), 3.8 (3H, s). MS (ESI+): 464.1. HPLC (Condition B): Rt 4.80 min (HPLC purity 71.6%).

### Intermediate 35

### 4-({(3-chlorobenzyl)[(4-chlorophenyl)sulfonyl]amino}methyl)benzoic acid

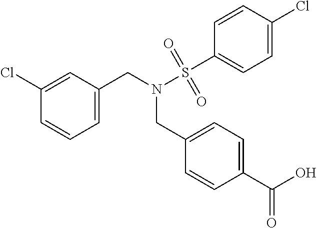

Following the general method as outlined for Intermediate 31, starting from methyl 4-({(3-chlorobenzyl)[(4-chlorophenyl)sulfonyl]amino}methyl)benzoate (Intermediate 34, 420 mg, 0.9 mmol), the title compound was obtained as a white solid in 81% yield.

¹H NMR (DMSO-d6, 400 MHz): δ 7.87-7.89 (2H, d), 7.66-7.69 (4H, d), 7.21-7.23 (2H, s), 7.01-7.02 (2H, s), 6.96-6.98 (2H, d), 4.32 (2H, s) 4.29 (2H, s). MS (ESI−): 447.7. HPLC (Condition B): Rt 4.03 min (HPLC purity 97.5%).

### Intermediate 36

### Methyl 4-{[benzyl(4-methoxysulfonyl)amino]methyl}benzoate

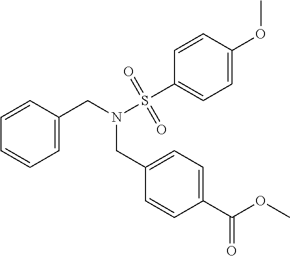

Following the general method as outlined for Intermediate 2, starting from methyl 4-[(benzyl amino)methyl]benzoate hydrochloride (Intermediate 1; 500 mg, 1.74 mmol) and 4-methoxybenzenesulfonylchloride (370 mg, 2.06 mmol) the title compound was obtained as an off-white solid, after recrystallization with dichloromethane/hexane.

¹H NMR (DMSO-d6, 400 MHz): δ 7.81-7.84 (2H, d), 7.73-7.76 (2H, d), 7.12-7.20 (6H, m), 7.07-7.09 (2H, t), 4.31 (1H, s), 4.27 (2H, s), 3.85 (3H, s), 3.80 (3H, s). MS (ESI+): 426.0. HPLC (Condition B): Rt 4.22 min (HPLC purity 99.5%).

### Intermediate 37

### Methyl 4-{[benzyl(4-methoxysulfonyl)amino]methyl}benzoic acid

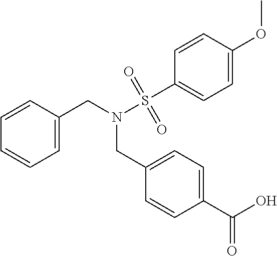

Following the general method as outlined for Intermediate 26, starting from 4-{[benzyl (4-methoxysulfonyl)amino]}benzoate (Intermediate 36, 350 mg; 0.82 mmol), the title compound was obtained as a white solid in 71% yield.

¹H NMR (DMSO-d6, 400 MHz): δ 7.81-7.83 (2H, d), 7.72-7.74 (2H, d), 7.14-7.2 (7H, m), 7.06-7.09 (2H, m), 4.3 (2H, s), 4.27 (2H, s), 3.85 (3H, s). MS (ESI+): 412.0. HPLC (Condition B): Rt 3.60 min (HPLC purity 98.7%).

### Intermediate 38

### Methyl 4-({benzyl[(4-fluorophenyl)sulfonyl]amino}methyl)benzoate

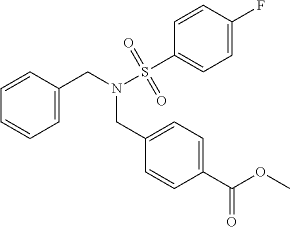

Following the general method as outlined for Intermediate 2, starting from methyl 4-[(benzyl amino)methyl]benzoate hydrochloride (Intermediate 1; 500 mg, 1.74 mmol) and 4-fluoro benzenesulfonylchloride (396 mg, 2.05 mmol) the title compound was obtained as an off-white solid in 78% yield.

¹H NMR (DMSO-d6, 400 MHz): δ7.94-7.98 (2H, m), 7.75-7.77 (2H, t) 7.43-7.47 (2H, m), 7.16-7.21 (5H, m), 7.07-7.09 (2H, m), 4.38 (2H, s), 4.32 (2H, s), 3.81 (3H, s). MS (ESI+): 413.9. HPLC (Condition B): Rt 4.21 min (HPLC purity 96.4%).

### Intermediate 39

### 4-({benzyl[(4-fluorophenyl)sulfonyl]amino}methyl)benzoic acid

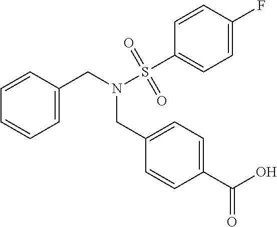

Following the general method as outlined for Intermediate 26, starting from methyl 4-({benzyl [(4-fluoro phenyl)sulfonyl]amino}methyl)benzoate (Intermediate 37, 500 mg; 1.2 mmol), the title compound was obtained as a white solid in 83% yield.

¹H NMR (DMSO-d6, 400 MHz): δ7.93-7.97 (2H, m), 7.71-7.73 (2H, d), 7.42-7.46 (2H, m), 7.17-7.21 (3H, m), 7.11-7.13 (2H, m), 7.06-7.09 (2H, m), 4.35 (2H, s), 4.31 (2H, s). MS (ESI−) δ 398.0. HPLC (Condition B): Rt 3.71 min (HPLC purity 97.6%).

### Intermediate 40

### Methyl 4-{[benzyl-(4-ethoxy-benzenesulfonyl)-amino]-methyl}-benzoate

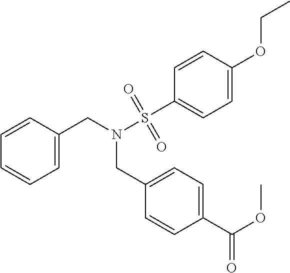

Following the general method as outlined for Intermediate 2, starting from methyl 4-[(benzyl amino)methyl]benzoate hydrochloride (Intermediate 1) and 4-ethoxy benzene sulfonyl chloride, the title compound was obtained as an off-white solid.

¹H NMR (DMSO-d6, 400 MHz): δ7.79-7.81 (2H, d), 7.74-7.76 (2H, d) 7.15-7.20 (5H, m), 7.06-7.12 (4H, m), 4.31 (2H, s), 4.27 (2H, s), 4.10-4.14 (2H, t), 3.80 (3H, s), 1.33-1.37 (3H, t). MS (ESI+): 440.3. HPLC (Condition B): Rt 4.34 min (HPLC purity 97.1%).

### Intermediate 41

### 4-{[Benzyl-(4-ethoxy-benzenesulfonyl)-amino]-methyl}-benzoic acid

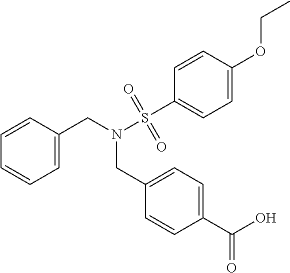

Following the general method as outlined for Intermediate 26, methyl 4-{[benzyl-(4-ethoxy-benzenesulfonyl)-amino]-methyl}-benzoate (Intermediate 40), the title compound was obtained as a white solid in 86% yield.

¹H NMR (DMSO-d6, 400 MHz): δ7.78-7.80 (2H, d), 7.68-7.70 (2H, d), 7.18-7.20 (3H, d), 7.04-7.11 (6H, m), 4.27 (2H, s), 4.25 (2H, s), 4.10-4.16 (2H, q), 1.33-1.37 (3H, t). MS (ESI−): 423.9. HPLC (Condition B): Rt 4.34 min (HPLC purity 95.3%).

### Intermediate 42

### Methyl 4-({(4-fluorobenzyl)[(4-chlorophenyl)sulfonyl]amino}methyl)benzoate

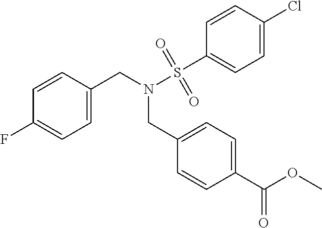

Following the general method as outlined for Intermediate 13, starting from methyl 4-({[(4-chlorophenyl)sulfonyl]amino}methyl)benzoate (Intermediate 29, 500 mg, 1.47 mmol) and 4-fluoro benzyl bromide (0.22 ml, 1.6 mmol), the title compound was obtained as white solid in 96% yield, after recrystallization from dichloromethane/hexane.

¹H NMR (DMSO-d6, 400 MHz): δ7.911-7.916 (2H, d), 7.76-7.89 (2H, d), 7.75-7.76 (2H, d) 7.19-7.21 (2H, d) 7.12-7.14 (2H, m), 6.96-6.98 (2H, t) 4.38 (2H, s), 4.31 (2H, s), 3.81 (3H, s). MS (ESI+): 448.0. HPLC (Condition B): Rt 4.36 min (HPLC purity 94.3%).

### Intermediate 43

### 4-{[[(4-chlorophenyl)sulfonyl](4-fluoro benzyl)amino]methyl}benzoic acid

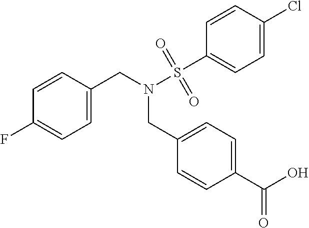

Following the general method as outlined for Intermediate 31, starting from methyl 4-({(4-fluorobenzyl)[(4-chlorophenyl)sulfonyl]amino}methyl)benzoate (Intermediate 42, 600 mg, 1.34 mmol), the title compound was obtained as a white solid in 86% yield.

¹H NMR (DMSO-d6, 400 MHz): δ 7.88-7.90 (2H, d), 7.67-7.72 (4H, m), 6.99-7.13 (6H, m) 4.38 (2H, s), 4.33 (2H, s). MS (ESI−): 432.0. HPLC (Condition B): Rt 3.88 min (HPLC purity 98.5%).

### Intermediate 44

### Methyl 4-({(4-methoxybenzyl)[(4-chlorophenyl)sulfonyl]amino}methyl)benzoate

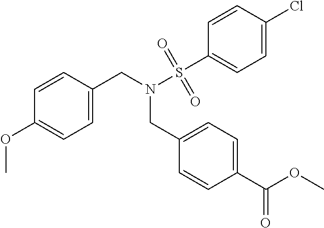

Following the general method as outlined for Intermediate 13, starting from methyl 4-({[(4-chlorophenyl)sulfonyl]amino}methyl)benzoate (Intermediate 29, 500 mg, 1.47 mmol) and 4-methoxy benzyl bromide (0.22 ml, 1.6 mmol), the title compound was obtained as an off-white solid in 92% yield.

¹H NMR (DMSO-d6, 400 MHz): δ 7.86-7.89 (2H, m), 7.76-7.78 (2H, d), 7.65-7.69 (2H, m), 7.19-7.21 (2H, d), 6.98-7.00 (2H, d), 6.71-6.74 (2H, m), 4.35 (2H, s), 4.25 (2H, s), 3.81 (3H, s), 3.66 (3H, s). MS (ESI+): 482.1. HPLC (Condition B): Rt 4.35 min (HPLC purity 99.6%).

### Intermediate 45

### 4-{[[(4-chlorophenyl)sulfonyl](4-methoxybenzyl)amino]methyl}benzoic acid

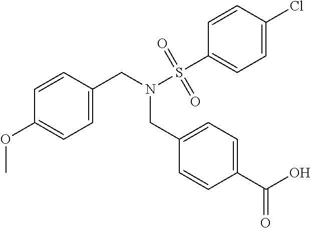

Following the general method as outlined for Intermediate 31, starting from methyl 4-({(4-methoxybenzyl)[(4-chlorophenyl)sulfonyl]amino}methyl)benzoate (Intermediate 44, 610 mg, 1.34 mmol), the title compound was obtained as a white solid in 72% yield.

¹H NMR (DMSO-d6, 400 MHz): δ 7.84-7.87 (2H, m), 7.71-7.73 (2H, d), 7.64-7.66 (2H, m), 6.97-7.00 (4H, m), 6.75-6.78 (2H, m), 4.27 (2H, s), 4.21 (2H, s), 3.68 (3H, s). MS (ESI−): 443.9. HPLC (Condition B): Rt 3.85 min (HPLC purity 99.5%).

### Intermediate 46

### Methyl 4-({benzyl[(4-chloropyridin-3-yl)sulfonyl]amino}methyl)benzoate

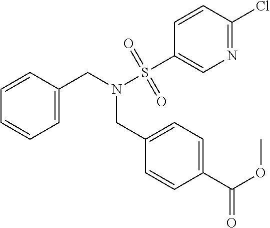

Following the general method as outlined for Intermediate 2, starting from methyl 4-[(benzyl amino)methyl]benzoate hydrochloride (Intermediate 1; 500 mg, 1.74 mmol) and 6-chloropyridin-3-sulfonylchloride (440 mg, 2.05 mol) the title compound was obtained as an off-white solid.

¹H NMR (DMSO-d6, 400 MHz): δ 8.84-8.85 (1H, d), 8.27-8.30 (1H, d), 7.73-7.78 (3H, m), 7.23-7.25 (2H, d), 7.16-7.19 (3H, m), 7.11-7.13 (2H, m) 4.46 (2H, s), 4.40 (2H, s), 3.81 (3H, s). MS (ESI+): 431.0. HPLC (Condition B): Rt 4.13 min (HPLC purity 98.0%).

### Intermediate 47

### 4-({benzyl[(4-chloropyridin-3-yl)sulfonyl]amino}methyl)benzoic acid

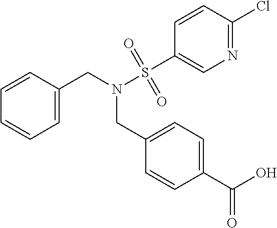

Following the general method as outlined for Intermediate 26, starting from methyl 4-({benzyl [(4-chloropyridin-3-yl)sulfonyl]amino}methyl)benzoate (Intermediate 46, 450 mg; 1.04 mmol), the title compound was obtained as a white solid.

¹H NMR (DMSO-d6, 400 MHz): δ12.91 (1H, bs), 8.84-8.85 (1H, d), 8.27-8.29 (1H, d), 7.73-7.76 (3H, m), 7.20-7.23 (5H, m), 7.12-7.19 (2H, m), 4.46 (2H, s), 4.40 (2H, s). MS (ESI−): 414.8. HPLC (Condition B): Rt 3.61 min (HPLC purity 99.7%).

### Intermediate 48

### Methyl 4-({(3-methoxybenzyl)[(4-chlorophenyl)sulfonyl]amino}methyl)benzoate

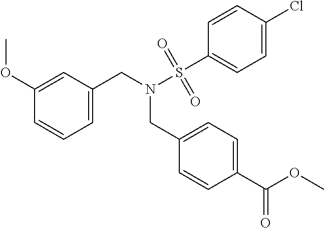

A solution of methyl 4-({[(4-chlorophenyl)sulfonyl]amino}methyl)benzoate (Intermediate 29, 500 mg, 1.47 mmol) in acetonitrile (20 ml) was treated with potassium carbonate (460 g, 2.95 mmol) and 3-methoxybenzyl bromide (0.22 ml, 1.6 mmol) and refluxed for 3 h under nitrogen atmosphere. The reaction mixture was evaporated under vacuum; the residue was dissolved in water and extracted with ethyl acetate. The combined organic layers were washed with brine, dried over anhydrous sodium sulphate and concentrated under vacuum to get a crude, which was recrystallized with MDC/hexane to get the title compound as an off white solid (0.62 g, 92% yield).

¹H NMR (DMSO-d6, 400 MHz): δ 7.89-7.91 (2H, t), 7.77-7.79 (2H, d), 7.67-7.69 (2H, t), 7.22-7.24 (2H, d), 7.07-7.11 (1H, t), 6.70-6.73 (1H, m), 6.65-6.67 (1H, m), 6.53 (1H, s), 4.38 (2H, s), 4.29 (2H, s), 3.81 (3H, s), 3.74 (3H, s). MS (ESI+): 482.1. HPLC (Condition B): Rt 4.36 min (HPLC purity 97.5%).

### Intermediate 49

### 4-{[[(4-chlorophenyl)sulfonyl](3-methoxybenzyl)amino]methyl}benzoic acid

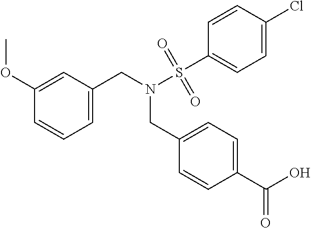

Following the general method as outlined for Intermediate 31, starting from methyl 4-({(3-methoxybenzyl)[(4-chlorophenyl)sulfonyl]amino}methyl)benzoate (Intermediate 48, 0.62 g, 1.3 mmol), the title compound was obtained as a white solid in 71% yield.

¹H NMR (DMSO-d6, 400 MHz): δ 7.88-7.90 (2H, d), 7.75-7.77 (2H, d), 7.67-7.69 (2H, d), 7.19-7.21 (2H, d), 7.08-7.12 (1H, t), 6.71-6.74 (1H, m), 6.65-6.67 (1H, d), 6.53 (1H, s), 4.37 (2H, s), 4.29 (2H, s), 3.58 (3H, s). MS (ESI−): 443.9. HPLC (Condition B): Rt 3.80 min (HPLC purity 99.8%).

### Intermediate 50

### Methyl 4{[[(4-chlorophenyl)sulfonyl](4-chlorobenzyl)amino]methyl}benzoate

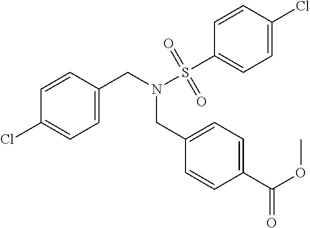

Following the general method as outlined for Intermediate 48, starting from methyl 4-({[(4-chlorophenyl)sulfonyl]amino}methyl)benzoate (Intermediate 29, 500 mg; 1.7 mmol) and 4-chloro benzyl bromide (370 mg, 1.76 mmol), the title compound was obtained as white solid.

¹H NMR (DMSO-d6, 400 MHz): δ7.89-7.916 (2H, d), 7.75-7.89 (2H, d), 7.75-7.77 (2H, d) 7.67-7.70 (2H, d) 7.21-7.23 (4H, m), 7.09-7.11 (2H, d) 4.39 (2H, s), 4.32 (2H, s), 3.81 (3H, s). MS (ESI+): 464.1. HPLC (Condition B): Rt 4.52 min (HPLC purity 94.2%).

### Intermediate 51

### 4-{[[(4-chlorophenyl)sulfonyl](4-chlorobenzyl)amino]methyl}benzoic acid

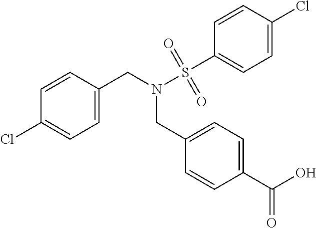

Following the general method as outlined for Intermediate 31, starting from methyl 4-({(4-chlorobenzyl)[(4-chlorophenyl)sulfonyl]amino}methyl)benzoate (Intermediate 50, 500 mg, 1.08 mmol), the title compound was obtained as a white solid in 88% yield.

¹H NMR (DMSO-d6, 400 MHz): δ 7.87-7.89 (2H, d), 7.66-7.68 (4H, m), 7.25-7.27 (2H, d), 7.09-7.11 (2H, d), 6.97-6.99 (2H, d), 4.30 (2H, s), 4.27 (2H, s). MS (ESI−): 449.8. HPLC (Condition B): Rt 3.88 min (HPLC purity 99.1%).

### Intermediate 52

### 4-{[(pyridin-2-ylmethyl)amino]methyl}benzonitrile

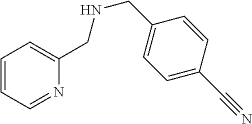

A cooled (0° C.) solution of 2-picolylamine (4.00 g, 36.9 mmol) in acetonitrile (50 ml) was treated with potassium carbonate (9.34 g, 67.2 mmol) and 4-(bromomethyl)benzonitrile (6.586 g, 33.6 mmol) and stirred for 1 hr. The reaction mixture was filtered and the filtrate was concentrated. The residue was purified by column chromatography to afford the title compound as a brownish liquid.

¹H NMR (DMSO-d6, 400 MHz): δ8.46-8.47 (1H, d), 7.72-7.77 (3H, m), 7.53-7.55 (2H, d), 7.43-7.45 (1H, d), 7.21-7.24 (1H, t), 3.79 (2H, d), 3.75 (2H, s). MS (ESI+): 224.1. HPLC (Condition B): Rt 3.77 min (HPLC purity 96.8%).

### Intermediate 53

### N-benzyl-3,4-dichloro-N-(4-cyanobenzyl)benzenesulfonamide

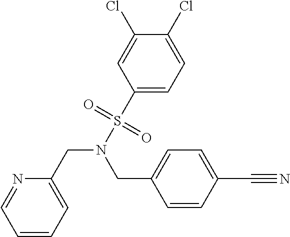

Following the general method as outlined for Intermediate 14, starting from 4-{[(pyridin-2-ylmethyl)amino]methyl}benzonitrile (Intermediate 52, 500 mg, 2.2 mmol) and 3,4-dichloro benzene sulfonyl chloride (630 mg, 2.4 mmol), the title compound was obtained as off white solid in 77% yield.

¹H NMR (DMSO-d6, 400 MHz): δ8.28-8.29 (1H, d), 7.962-7.7.967 (1H, d), 7.80-7.81 (2H, t), 7.71-7.73 (2H, d), 7.58-7.68 (1H, m), 7.39-7.41 (2H, d), 7.17-7.22 (2H, m), 4.61 (2H, s), 4.49 (2H, s). MS (ESI+): 432.0. HPLC (Condition B): Rt 3.49 min (HPLC purity 92.8%).

### Intermediate 54

### N-(4-cyanobenzyl)-4-ethoxy-N-(pyridin-2-ylmethyl)benzenesulfonamide

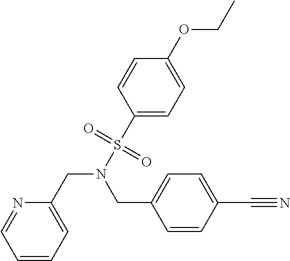

Following the general method as outlined for Intermediate 14, starting from 4-{[(pyridin-2-ylmethyl)amino]methyl}benzonitrile (Intermediate 52, 500 mg, 2.2 mmol) and 4-ethoxybenzenesulfonylchloride, the title compound was obtained as off white solid in 77% yield after recrystallization with ethyl acetate and pet ether.

¹H NMR (DMSO-d6, 400 MHz): δ8.31 (1H, S), 7.75-7.77 (2H, d), 7.66-7.68 (2H, d), 7.60 (1H, s), 7.33-7.35 (2H, d), 6.16-6.18 (2H, t), 7.06-7.08 (2H, d), 4.46 (2H, s), 4.38 (2H, s), 4.12-4.13 (2H, d), 1.33-1.37 (3H, t). HPLC (Condition B): Rt 2.97 min (HPLC purity 98.4%).

### Intermediate 55

### methyl 4-{[(pyridin-2-ylmethyl)amino]methyl}benzoate

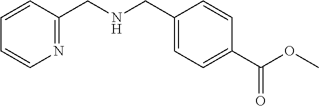

A cooled (0° C.) solution of 2-picolyl amine (1.00 g, 9.2 mmol) in dry DMF (20 ml) was treated with sodium hydride (488 mg, 10.1 mmol). After stirring for 15 minutes, methyl 4-(bromomethyl)benzoate (2.32 g, 10.1 mmol) was added and the mixture was allowed to warm to room temperature for 2 h. The reaction mixture was quenched with water and extracted with ethyl acetate. The organic layer was dried over sodium sulphate and concentrated. The residue was purified by chromatography eluting with chloroform/methanol (9.5/0.5) to afford the titled compound (1.2 g, 88%) as a brown liquid.

¹H NMR (CDCl3, 400 MHz) δ 8.57-8.58 (1H, m), 7.99-8.01 (2H, d), 7.63-7.67 (1H, t), 7.44-7.46 (2H, d), 7.27-7.31 (1H, m), 7.16-7.19 (1H, m), 3.91-3.93 (7H, m). MS (ESI+): 257.0. HPLC (Condition B): Rt 3.97 min (HPLC purity 95.9%).

### Intermediate 56

### Methyl 4{[[(4-cyanophenyl)sulfonyl](pyridin-2-ylmethyl)amino]methyl}benzoate

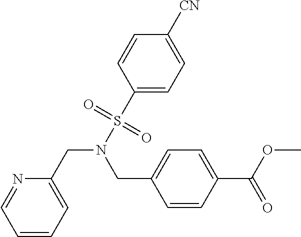

Following the general method as outlined for Intermediate 2, starting from methyl 4-{[(pyridin-2-ylmethyl)amino]methyl}benzoate (Intermediate 55, 500 mg, 1.95 mmol) and 4-cyanobenzenesulphonyl chloride (433 mg, 2.15 mmol) the title compound was obtained as an off-white solid in 86% yield.

¹H NMR (DMSO-d6, 400 MHz): δ8.25-8.27 (1H, d), 7.97-8.04 (4H, m), 7.81-7.84 (2H, d), 7.61 (1H, t), 7.31-7.33 (2H, d), 7.16-7.18 (2H, d), 4.58 (2H, s), 4.46 (2H, s), 3.82 (3H, s). MS (ESI+): 422.1. HPLC (Condition B): Rt 2.92 min (HPLC purity 96.2%).

### Intermediate 57

### 4-{[[(4-cyanophenyl)sulfonyl](pyridin-2-ylmethyl)amino]methyl}benzoic acid

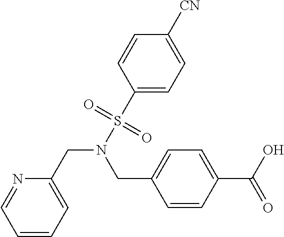

Following the general method as outlined for Intermediate 26, starting from methyl 4-{[[(4-cyanophenyl)sulfonyl](pyridin-2-ylmethyl)amino]methyl}benzoate (Intermediate 56, 450 mg, 1.06 mmol), the title compound was obtained as a white solid.

¹H NMR (DMSO-d6, 400 MHz): δ 12.92 (1H, bs), 8.26-8.27 (1H, d), 8.01-8.03 (4H, m), 7.79-7.99 (2H, d), 7.62-7.64 (1H, t), 7.28-7.30 (2H, d), 7.15-7.18 (2H, d), 4.57 (2H, s), 4.46 (2H, s). MS (ESI−): 406.0. HPLC (Condition B): Rt 2.36 min (HPLC purity 97.4%).

### Intermediate 58

### Methyl 4-{[[(3,4-dichlorophenyl)sulfonyl](pyridin-3-ylmethyl)amino]methyl}benzoate

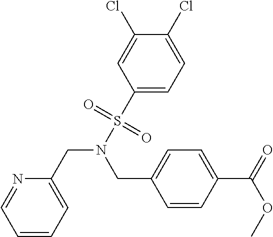

Following the general method as outlined for Intermediate 2, starting from methyl 4-{[(pyridin-2-ylmethyl)amino]methyl}benzoate (Intermediate 55, 500 mg, 1.95 mmol) and 3,4-dichlorobenzenesulphonyl chloride (330 mg, 2.14 mmol) the title compound was obtained as yellow solid in 95% yield after recrystallization from dichloromethane/hexane.

¹H NMR (DMSO-d6, 400 MHz): δ 8.28-8.30 (1H, m), 7.94 (1H, s), 7.79-7.84 (4H, m), 7.58-7.69 (2H, m), 7.33-7.36 (2H, d), 7.16-7.21 (2H, m), 4.59 (2H, s), 4.48 (2H, s), 3.82 (3H, s). MS (ESI+): 467.0. HPLC (Condition B): Rt 3.55 min (HPLC purity 86.5%).

### Intermediate 59

### 4-{[[(3,4-dichlorophenyl)sulfonyl](pyridin-3-ylmethyl)amino]methyl}benzoic acid

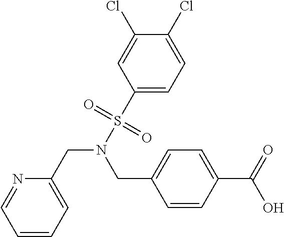

Following the general method as outlined for Intermediate 26, starting from methyl 4-{[[(3,4-dichlorophenyl)sulfonyl](pyridin-2-ylmethyl)amino]methyl}benzoate (Intermediate 58, 1 g, 2.15 mmol), the title compound was obtained as a white solid.

¹H NMR (DMSO-d6, 400 MHz): δ 8.30-8.31 (1H, d), 7.96 (1H, s), 7.78-7.96 (2H, m), 7.70-7.72 (2H, d), 7.67 (2H, s), 7.63-7.65 (1H, t), 7.17-7.20 (2H, t), 7.07-7.09 (2H, d), 4.49 (2H, s), 4.41 (2H, s). MS (ESI+): 453.0. HPLC (Condition B): Rt 2.98 min (HPLC purity 99.3%).

### Intermediate 60

### N-(pyridin-2-ylmethyl)-N-[4-(2H-tetrazol-5-yl)benzyl]amine

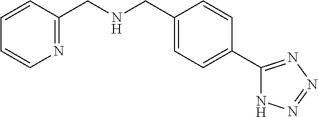

Following the general method as outlined in Example 17, starting from 4-{[(pyridin-2-ylmethyl)amino]methyl}benzonitrile (Intermediate 52, 1.00 g, 4.4 mmol), the title compound was obtained as off white solid.

¹H NMR (DMSO-d6, 400 MHz): δ8.61-8.62 (1H, d), 8.00-8.02 (2H, d), 7.83-7.85 (1H, d), 7.49-7.54 (3H, t), 7.39-7.40 (1H, d), 4.20 (2H, s), 4.13 (2H, s). MS (ESI+): 267.1. HPLC (Condition B): Rt 3.46 min (HPLC purity 96.2%).

### Intermediate 61

### 4-chloro-N-(4-cyano-3-fluorobenzyl)-N-(pyridin-2-ylmethyl)benzenesulfonamide

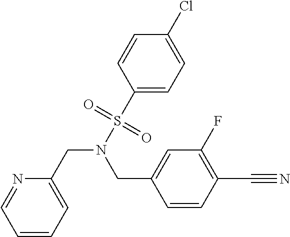

A solution of 4-chloro-N-(pyridin-2-ylmethyl)benzenesulfonamide (Intermediate 3, 1.00 g; 3.53 mmol) in anhydrous THF (20 ml) was treated with 4-(bromo methyl)-2-fluorobenzonitrile (0.760 g, 3.53 mmol) and cesium carbonate (2.3 g; 7.1 mmol) and heated to 65° C. for 2 h. The mixture was concentrated and diluted with DCM and extracted with brine. The organic phase was separated, dried over magnesium sulfate, filtered and concentrated to give solid. The crude product was purified by column chromatography over silica gel affording the title compound as yellow oil.

¹H NMR (DMSO-d6, 400 MHz): δ 829-8.30 (1H, m), 7.80-7.86 (2H, d), 7.76-7.78 (1H, t), 7.61-7.63 (3H, m), 7.16-7.23 (4H, m), 4.55 (2H, s), 4.47 (2H, s)

### Intermediate 62

### 4-chloro-N-(4-cyano-2-fluorobenzyl)-N-(pyridin-2-ylmethyl)benzene sulfonamide

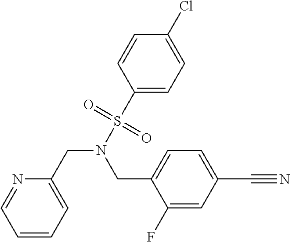

Following the general method as outlined for Intermediate 61, starting from 4-chloro-N-(pyridin-2-ylmethyl)benzenesulfonamide (Intermediate 3, 350 mg; 1.23 mmol) and 4-(bromo methyl)-3-fluorobenzonitrile (250 mg, 1.23 mmol), the title compound was obtained as off white solid in 95% yield.

¹H NMR (DMSO-d6, 400 MHz): δ 827-8.28 (1H, d), 7.82-7.84 (2H, d), 7.65-7.70 (1H, d), 7.61-7.62 (3H, m), 7.56-7.58 (1H, d), 7.46-7.49 (1H, t), 7.21-7.22 (1H, d), 7.14-7.18 (1H, t), 4.59 (2H, s), 4.47 (2H, s)

### Intermediate 63

### 4-chloro-N-(3,5-dimethylisoxazol-4-yl)methyl)-N-(4-cyanobenzyl)benzene sulfonamide

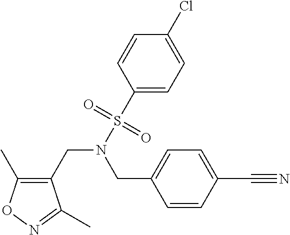

Following the general method as outlined for Intermediate 13, starting from 4-chloro-N-(4-cyanobenzyl)benzenesulfonamide (Intermediate 18, 300 mg; 0.97 mmol) and 4-chloromethyl 3,5-dimethylisoxazole (145 mg, 1.0 mmol), the title compound was obtained as off white solid in 87% yield.

¹H NMR (DMSO-d6, 400 MHz): δ7.92-7.94 (2H, d), 7.73-7.75 (2H, d), 7.68-7.70 (2H, d), 7.29-7.31 (2H, d), 4.34 (2H, s), 4.17 (2H, s), 2.02 (2H, s), 1.97 (2H, s). MS (ESI+): 415.9. HPLC (Condition B): Rt 3.85 min (HPLC purity 98.6%).

### Intermediate 64

### 4-chloro-N-(4-cyanobenzyl)-N-(1,3-oxazol-2-ylmethyl)benzenesulfonamide

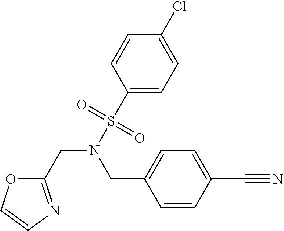

Following the general method as outlined for Intermediate 13, starting from 4-chloro-N-(4-cyanobenzyl)benzenesulfonamide (Intermediate 18, 300 mg; 0.97 mmol) and 2-chloromethyl oxazole (120 mg, 1.0 mmol), the title compound was obtained as white solid.

¹H NMR (DMSO-d6, 400 MHz): δ7.87 (1H, s), 7.82-7.84 (2H, t), 7.76-7.78 (2H, d), 7.64-7.67 (2H, t), 7.41-7.43 (2H, d), 6.98 (1H, s), 4.51 (2H, s), 4.49 (2H, s). MS (ESI+): 387.9. HPLC (Condition B): Rt 3.64 min (HPLC purity 98.1%).

### Intermediate 65

### 4-{[(2,4-difluorobenzyl)amino]methyl}benzonitrile hydrochloride

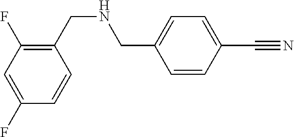

A solution of 2.4-difluorobenzylamine (500 mg, 3.4 mmol) in 10 ml of acetonitrile was treated with potassium carbonate (563 mg, 4.0 mmol) and 4-(bromomethyl)benzonitrile (685 mg, 3.4 mmol) and stirred for 2 h at RT. The reaction mixture was concentrated, dissolved in water and extracted with ethyl acetate. The organic layer was washed with brine solution, dried over sodium sulfate, concentrated under vacuum to get the crude mass. The crude was cooled in an ice bath and diluted with 10 ml of dioxane; then a solution of HCl in dioxane was added dropwise and stirred for 1 h. The reaction mixture was filtered, washed with chloroform, dried under vacuum to afford the title compound as a white solid.

¹H NMR (DMSO-d6, 400 MHz): δ9.75 (2H, s), 7.91-7.93 (2H, t), 7.70-7.76 (3H, m), 7.33-7.38 (1H, m), 7.17-7.22 (1H, m), 4.31 (2H, s), 4.18 (2H, s). MS (ESI+): 259.2. HPLC (Condition B): Rt 4.37 min (HPLC purity 96.2%).

### Intermediate 66

### 4-chloro-N-(4-cyanobenzyl)-N-(2,4-difluorobenzyl)benzenesulfonamide

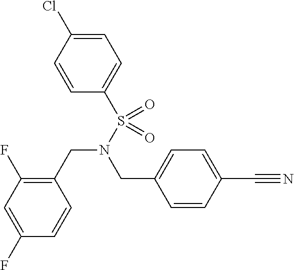

A cooled (0° C.) solution of 4-{[(2,4-difluorobenzyl)amino]methyl}benzonitrile hydrochloride (Intermediate 65, 250 mg, 0.84 mmol) in dry DCM (10 ml) was treated with triethylamine (255 mg, 2.5 mmol), stirred for 10 min and then treated with 4-chlorobenzenesulfonylchloride (214 mg, 1.0 mmol). The reaction mixture was stirred at RT for 16 h before being quenched with 10% sodium bicarbonate. The organic layer was separated, washed with water and saturated brine solution, dried over sodium sulphate and concentrated, The crude product was recrystallized with ethyl acetate and pet ether to afford the title compound as an off-white solid.

¹H NMR (DMSO-d6, 400 MHz): δ7.89-7.91 (2H, d), 7.69-7.71 (2H, d), 7.64-7.66 (2H, d), 7.26-7.28 (3H, d), 6.87-7.00 (2H, m), 4.41 (2H, s), 4.37 (2H, s). MS (ESI+): 433.0. HPLC (Condition B): Rt 4.11 min (HPLC purity 96.1%).

### Intermediate 67

### 4-chloro-N-(5-chloro-2-fluorobenzyl)-N-(4-cyanobenzyl)benzene sulfonamide

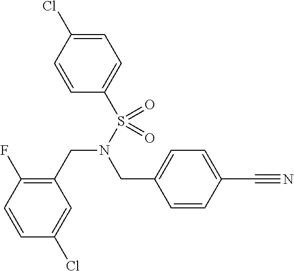

Following the general method as outlined for Intermediate 13, starting from 4-chloro-N-(4-cyanobenzyl)benzenesulfonamide (Intermediate 18, 300 mg; 0.97 mmol) and 2-(bromomethyl)-5-chloro-fluoro benzene (230 mg; 1.06 mmol), the title compound was obtained as white solid.

¹H NMR (DMSO-d6, 400 MHz) δ 7.90-7.92 (2H, d), 7.66-7.72 (4H, m), 7.31-7.33 (2H, d), 7.22-7.26 (1H, m), 7.07-7.09 (1H, m), 6.99-7.07 (1H, m), 4.46 (2H, s), 4.40 (2H, s). MS (ESI+): 449.1. HPLC (Condition B): Rt 4.20 min (HPLC purity 99.2%).

### Intermediate 68

### 4-chloro-N-(4-cyanobenzyl)-N-(2,6-difluorobenzyl)benzene sulfonamide

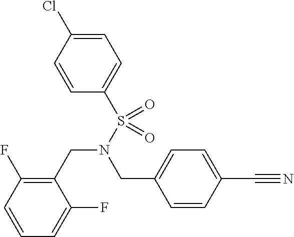

Following the general method as outlined for Intermediate 13, starting from 4-chloro-N-(4-cyanobenzyl)benzenesulfonamide (Intermediate 18, 300 mg; 0.97 mmol) and 2,6-diflouorobenzyl bromide (220 mg; 1.7 mmol), the title compound was obtained as white solid in 72% yield.

¹H NMR (DMSO-d₆, 400 MHz) δ 7.85-7.87 (2H, m), 7.65-7.70 (4H, m), 7.31-7.33 (2H, d), 7.24 (1H, s), 6.82-6.86 (2H, t), 4.41 (2H, s), 4.39 (2H, s). MS (ESI+): 433.1. HPLC (Condition B): Rt 4.09 min (HPLC purity 99.5%).

### Intermediate 69

### 4-chloro-N-(2-chlorobenzyl)-N-(4-cyanobenzyl)benzenesulfonamide

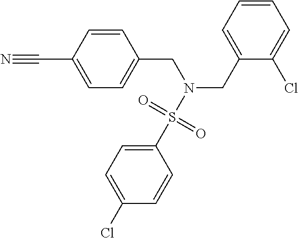

Following the general method as outlined for Intermediate 13, starting from 4-chloro-N-(4-cyanobenzyl)benzenesulfonamide (Intermediate 18, 300 mg; 0.97 mmol) and 2-chlorobenzyl bromide (200 mg; 0.97 mmol), the title compound was obtained as white solid.

¹H NMR (DMSO-d6, 400 MHz): δ7.91-7.93 (2H, d), 7.70-7.72 (2H, d), 7.60-7.62 (2H, d), 7.26-7.28 (3H, d), 7.15-7.20 (3H, m), 4.45 (2H, s), 4.42 (2H, s). MS (ESI+): 431.2. HPLC (Condition B): Rt 4.23 min (HPLC purity 98.9%).

### Intermediate 70

### Methyl 4-cyano-2-fluorobenzoate

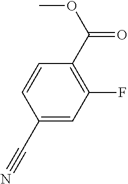

A solution of 4-cyano-2-fluoro benzoic acid (4.00 g, 24.2 mmol) in dry DMF (40 ml) was treated with methyl iodide (20.62 g, 14.5 mmol), potassium carbonate (5.02 g, 36.3 mmol) and the reaction mixture was stirred at RT for 12 h. The reaction mixture was filtered and filtrate was concentrated. The crude was diluted with ethyl acetate and extracted with water. The organic layer was washed with brine and dried over Na₂SO₄ and evaporated under vacuum. The title compound was obtained as a yellow solid.

¹H NMR (DMSO-d6, 400 MHz) δ 8.00-8.05 (2H, m) 7.80-7.82 (1H, d), 3.88 (3H, s). HPLC (Condition B): Rt 5.67 min (HPLC purity 99.0%).

### Intermediate 71

### Methyl 4-{[(tert-butoxycarbonyl)amino]methyl}-2-fluorobenzoate

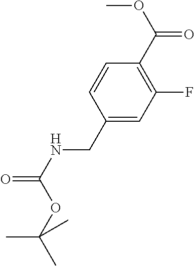

A solution of methyl 4-cyano-2-fluorobenzoate (intermediate 70, 4.00 g, 24.2 mmol) in dry THF (40 ml) was treated with Boc anhydride (3.65 g, 16.7 mmol), palladium on carbon 10% (1 g) and the reaction mixture was stirred at RT for 12 h under hydrogen atmosphere. The reaction mixture was filtered and the filtrate was concentrated. The title compound was obtained as colorless liquid (2.6 g), used without purification for the next step.

### Intermediate 72

### Methyl 4-(amino methyl)-2-fluorobenzoate hydrochloride

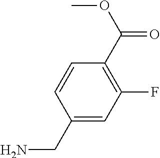

A cooled (0° C.) solution of crude methyl 4-{[(tert-butoxycarbonyl)amino]methyl}-2-fluorobenzoate (intermediate 71, 4.00 g, 24.2 mmol) in dry dioxane (40 ml) under nitrogen was treated with a solution of HCl in dioxane and the reaction mixture was stirred at 0° C. for 4 h. The reaction mixture was filtered and washed with dioxane and dried under vacuum. The crude obtained was neutralized with 7 ml of sat. aqueous ammonia solution and the solution was extracted with DCM, dried over Na₂SO₄ and evaporated under vacuum. The crude was purified by column chromatography to afford the title compound as a yellow solid.

¹H NMR (DMSO-d6, 400 MHz) δ 7.80-7.84 (1H, t) 7.30-7.34 (1H, d),) 7.26-7.28 (1H, d), 3.82 (3H, s), 3.79 (2H, s). MS (ESI+): 184.3. HPLC (Condition B): Rt 1.90 min (HPLC purity 95.2%).

### Intermediate 73

### Methyl 4-[(4-chloro-benzenesulfonylamino)-methyl]-2-fluoro-benzoate

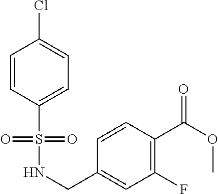

Following the general method as outlined for Intermediate 29, starting from methyl 4-(aminomethyl)-2-fluorobenzoate hydrochloride (intermediate 72, 300 mg, 1.36 mmol) and 4-chlorobenzenesulfonylchloride (315 mg, 1.36 mmol), the title compound was obtained as off-white solid in 92% yield.

¹H NMR (DMSO-d6, 400 MHz): δ 8.42-8.45 (1H, t), 7.74-7.80 (3H, m), 7.61-7.63 (2H, d), 7.16-7.19 (2H, m), 4.09-4.10 (2H, d), 3.82 (3H, s). MS (ESI+): 355.8. HPLC (Condition B): Rt 3.60 min (HPLC purity 98.7%).

### Intermediate 74

### Methyl 4-{[(4-chloro-benzenesulfonyl)-pyridin-2-ylmethyl-amino]-methyl}-2-fluoro-benzoate

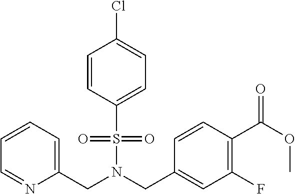

Following the general method as outlined for Intermediate 48, starting from methyl 4-[(4-Chloro-benzenesulfonylamino)-methyl]-2-fluoro-benzoate (intermediate 73, 240 mg, 0.67 mmol) and 2-picolyl amine (187 mg, 0.73 mmol), the title compound was obtained as white solid in 73% yield.

¹H NMR (DMSO-d6, 400 MHz) δ 8.30-8.31 (1H, d), 7.83-7.86 (2H, m), 7.72-7.75 (1H, t), 7.61-7.66 (3H, m), 7.20-7.22 (2H, d), 7.11-7.19 (1H, m), 7.04-7.07 (1H, d), 4.52 (2H, s), 4.46 (2H, s), 3.32 (3H, s). MS (ESI+): 449.1. HPLC (Condition B): Rt 3.28 min (HPLC purity 99.1%).

### Intermediate 75

### Methyl 4-{[(4-chloro-benzenesulfonyl)-(2-fluoro-benzyl)-amino]-methyl}-2-fluoro-benzoate

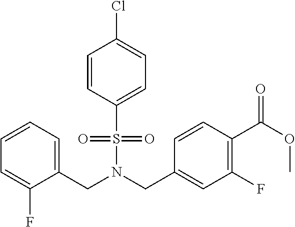

Following the general method as outlined for Intermediate 48, starting from methyl 4-[(4-chloro-benzenesulfonylamino)-methyl]-2-fluoro-benzoate (intermediate 73, 250 mg, 0.70 mmol) and 2-fluorobenzyl bromide (145 mg, 0.77 mmol), the title compound was obtained as white solid in 92% yield.

¹H NMR (DMSO-d6, 400 MHz): δ 7.89-7.91 (2H, d), 7.69-7.71 (2H, d), 7.19-7.25 (3H, m), 6.95-7.05 (4H, m), 4.41 (2H, s), 4.40 (2H, s), 3.80 (3H, s). MS (ESI+): 465.9. HPLC (Condition B): Rt 4.25 min (HPLC purity 91.0%).

### Intermediate 76

### Methyl 4-[(4-ethoxy-benzenesulfonylamino)-methyl]-2-fluoro-benzoate

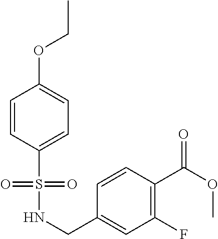

Following the general method as outlined for Intermediate 29, starting from methyl 4-(aminomethyl)-2-fluorobenzoate hydrochloride (intermediate 72, 300 mg, 1.3 mmol) and 4-ethoxybenzenesulfonylchloride (300 mg, 1.3 mmol), the title compound was obtained as off-white solid in 52% yield.

¹H NMR (DMSO-d6, 400 MHz): δ8.15 (1H, s), 7.75-7.79 (1H, m), 7.64-7.67 (2H, d), 7.13-7.19 (2H, m), 7.01-7.03 (2H, d), 4.03-4.10 (4H, m), 3.82-3.84 (3H, d), 1.30-1.34 (3H, t). MS (ESI+): 367.9. HPLC (Condition B): Rt 3.51 min (HPLC purity 88.9%).

### Intermediate 77

### Methyl 4-{[(4-ethoxy-benzenesulfonyl)-pyridin-2-ylmethyl-amino]-methyl}-2-fluoro-benzoate

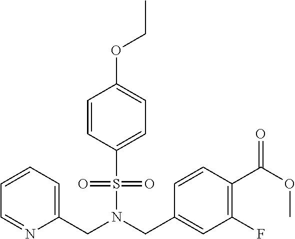

Following the general method as outlined for Intermediate 48, starting from methyl 4-[(4-ethoxy-benzenesulfonylamino)-methyl]-2-fluoro-benzoate (intermediate 76, 220 mg, 0.60 mmol) and 2-picolyl amine (166 mg; 0.65 mmol), the title compound was obtained as white solid in 91% yield.

¹H NMR (DMSO-d₆, 400 MHz) δ 8.32-8.33 (1H, m), 7.71-7.77 (2H, m), 7.62-7.69 (1H, m), 7.60 (1H, m), 7.18-7.22 (1H, m), 7.15-7.17 (1H, m), 7.01-7.11 (4H, m), 4.44 (2H, s), 4.39 (2H, s), 4.09-4.14 (2H, q), 3.81-3.82 (3H, s), 1.33-1.36 (3H, t). MS (ESI+): 459.2. HPLC (Condition B): Rt 5.60 min (HPLC purity 94.5%).

### Intermediate 78

### Methyl 4-({[(4-cyanophenyl)sulfonyl]amino}methyl)-2-fluorobenzoate

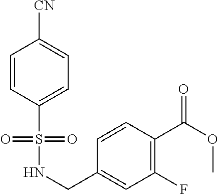

Following the general method as outlined for Intermediate 29, starting from methyl 4-(aminomethyl)-2-fluorobenzoate hydrochloride (intermediate 72, 300 mg, 1.36 mmol) and 4-cyanobenzene sulfonyl chloride (273 mg; 1.36 mmol), the title compound was obtained as off-white solid in 70% yield.

¹H NMR (DMSO-d6, 400 MHz): δ8.64 (1H, s), 8.01-8.03 (2H, m), 7.88-7.90 (2H, m), 7.74-7.78 (1H, t), 7.11-7.17 (2H, m), 4.14 (2H, s), 3.82 (3H, s). MS (ESI−): 346.9. HPLC (Condition B): Rt 3.27 min (HPLC purity 97.8%).

### Intermediate 79

### methyl 4{[[(4-cyanophenyl)sulfonyl](pyridin-2-ylmethyl)amino]methyl}-2-fluorobenzoate

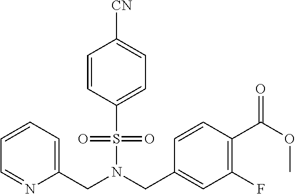

Following the general method as outlined for Intermediate 48, starting from methyl 4-({[(4-cyanophenyl)sulfonyl]amino}methyl)-2-fluorobenzoate (intermediate 78, 300 mg, 0.86 mmol) and 2-(bromomethyl)pyridine hydrobromide (239 mg, 0.94 mmol), the title compound was obtained as white solid.

¹H NMR (DMSO-d6, 400 MHz): δ8.26-8.28 (1H, t), 7.98-8.05 (4H, m), 7.73-7.77 (1H, t), 7.62-7.63 (1H, t), 7.06-7.22 (4H, m), 4.57 (2H, s), 4.50 (2H, s), 3.82 (3H, s). MS (ESI+): 440.0. HPLC (Condition B): Rt 3.07 min (HPLC purity 96.7%).

### Intermediate 80

### 4-Chloro-N-(4-cyano-benzyl)-N-(2-methyl-thiazol-4-ylmethyl)-benzenesulfonamide

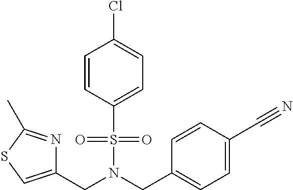

Following the general method as outlined for Intermediate 48, starting from 4-chloro-N-(4-cyanobenzyl)benzenesulfonamide (Intermediate 18, 350 mg; 1.14 mmol) and 4-chloromethyl-2-methyl-1,3-thiazole hydrochloride (231 mg; 1.25 mmol), the title compound was obtained as off white solid in 77% yield.

¹H NMR (DMSO-d6, 400 MHz): δ 7.75-7.80 (4H, m), 7.59-7.63 (2H, m), 7.40-7.42 (2H, d), 7.20 (1H, s), 4.50 (2H, s), 4.34 (2H, s), 2.41 (3H, s). MS (ESI+): 418.0. HPLC (Condition B): Rt 3.99 min (HPLC purity 96.7%).

### Intermediate 81

### 4-Chloro-N-(4-cyano-benzyl)-N-(5-tert-butyl-1,2,4-oxadiazol-3-ylmethyl)-benzenesulfonamide

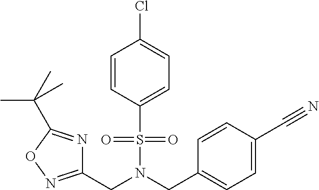

Following the general method as outlined for Intermediate 48, starting from 4-chloro-N-(4-cyanobenzyl)benzenesulfonamide (Intermediate 18, 350 mg; 1.14 mmol) and 3-chloromethyl-5-tert-butyl-1,2,4-oxadiazole (219 mg; 1.25 mmol), the title compound was obtained as white solid in 79% yield.

¹H NMR (DMSO-d6, 400 MHz): δ 7.81-7.85 (4H, m), 7.64-7.67 (2H, d), 7.46-7.49 (2H, d), 4.57 (2H, s), 4.48 (2H, s), 1.20 (9H, s). MS (ESI+): 444.9. HPLC (Condition B): Rt 4.25 min (HPLC purity 99.9%).

### Intermediate 82

### 4-Chloro-N-(4-cyano-benzyl)-N-(2-chloro-4-fluoro benzyl)-benzenesulfonamide

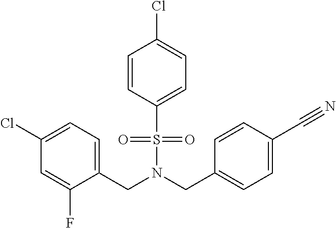

Following the general method as outlined for Intermediate 48, starting from 4-chloro-N-(4-cyanobenzyl)benzenesulfonamide (Intermediate 18, 400 mg; 1.30 mmol) and 2-chloro-4-fluoro benzyl bromide (320 mg; 1.43 mmol), the title compound was obtained as a white solid in 83% yield.

¹H NMR (DMSO-d6, 400 MHz): δ 7.89-7.91 (2H, d), 7.712-7.717 (2H, d), 7.69 (2H, d), 7.64-7.66 (2H, d), 7.30 (2H, d), 7.27 (1H, d), 7.23 (1H, d), 4.42 (2H, s), 4.38 (2H, s). HPLC (Condition B): Rt 4.32 min (HPLC purity 99.8%).

### Intermediate 83

### 4-Chloro-N-(4-cyano-benzyl)-N-pyridin-3-ylmethyl-benzenesulfonamide

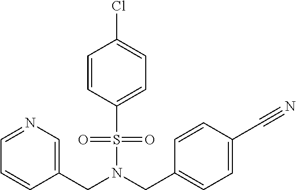

Following the general method as outlined for Intermediate 48, starting from 4-chloro-N-(4-cyanobenzyl)benzenesulfonamide (Intermediate 18, 400 mg; 1.30 mmol) and 3-picolyl chloride hydrochloride (299 mg; 1.82 mmol), the title compound was obtained as yellow solid in 97% yield.

¹H NMR (DMSO-d6, 400 MHz): δ 8.34 (1H, s), 8.25-8.27 (1H, d), 7.91-7.93 (2H, d), 7.70-7.72 (2H, d) 7.63-7.65 (2H, d), 7.47-7.48 (1H, d) 7.28-7.30 (2H, d), 7.15-7.17 (1H, d), 4.44 (2H, s), 4.453 (2H, s). MS (ESI+): 398.1. HPLC (Condition B): Rt 2.71 min (HPLC purity 90.7%).

### Intermediate 84

### 4-Chloro-N-(4-cyano-benzyl)-N-(5-methyl-1,2,4-oxadiazol-3-ylmethyl)-benzenesulfonamide

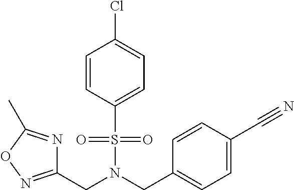

Following the general method as outlined for Intermediate 48, starting from 4-chloro-N-(4-cyanobenzyl)benzenesulfonamide (Intermediate 18, 400 mg; 1.34 mmol) and 3-chloromethyl-5-methyl-1,2,4-oxadiazole (189 mg; 1.43 mmol), the title compound was obtained as yellow solid in 72% yield.

¹H NMR (DMSO-d6, 400 MHz): δ 7.79-7.82 (4H, d), 7.64-7.66 (2H, d), 7.46-7.48 (2H, d), 4.56 (2H, s), 4.46 (2H, s), 2.41 (3H, s). MS (ESI+): 402.9. HPLC (Condition B): Rt 3.85 min (HPLC purity 99.7%).

### Intermediate 85

### 4-Chloro-N-(4-cyano-benzyl)-N-(isoquinolin-1-yl methyl)-benzenesulfonamide

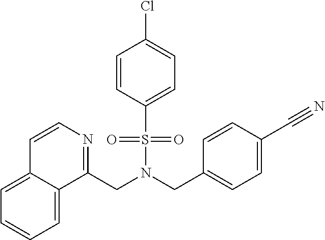

Following the general method as outlined for Intermediate 48, starting from 4-chloro-N-(4-cyanobenzyl)benzenesulfonamide (Intermediate 18, 400 mg; 1.30 mmol) and 1-bromomethyl-isoquinolin hydrobromide (588 mg; 1.95 mmol), the title compound was obtained as yellow solid in 86% yield.

¹H NMR (DMSO-d6, 400 MHz): δ 8.30-8.32 (1H, d), 8.13-8.15 (1H, d), 7.89-7.91 (2H, d), 7.83-7.85 (1H, d), 7.66-7.72 (4H, m), 7.59-7.60 (1H, d) 7.37-7.39 (2H, d) 7.06-7.08 (2H, d), 4.97 (2H, s), 4.47 (2H, s). MS (ESI+): 448.0. HPLC (Condition B): Rt 3.54 min (HPLC purity 99.2%).

### Intermediate 86

### 4-Chloro-N-(4-cyano-benzyl)-N-(quinolin-1-yl methyl)-benzenesulfonamide

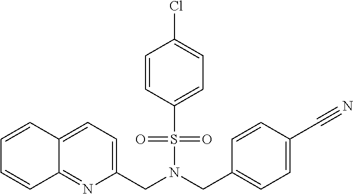

Following the general method as outlined for Intermediate 48, starting from 4-chloro-N-(4-cyanobenzyl)benzenesulfonamide (Intermediate 18, 350 mg; 1.4 mmol) and 1-bromomethyl-quinoline hydrobromide (269 mg; 1.25 mmol), the title compound was obtained as yellow solid in 91% yield.

¹H NMR (DMSO-d6, 400 MHz): δ 8.18 (1H, s), 7.86-7.90 (3H, m), 7.59-7.69 (6H, m), 7.54-7.56 (1H, t), 7.39-7.41 (2H, d), 7.31-7.33 (1H, d), 4.65 (2H, s), 4.60 (2H, s). MS (ESI+): 448.0. HPLC (Condition B): Rt 3.80 min (HPLC purity 96.7%).

### Intermediate 87

### 4-Chloro-N-(4-cyano-benzyl)-N-(isoquinolin-3-ylmethyl)-benzenesulfonamide

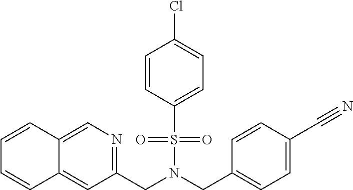

Following the general method as outlined for Intermediate 48, starting from 4-chloro-N-(4-cyanobenzyl)benzenesulfonamide (Intermediate 18, 200 mg; 0.65 mmol) and 3-bromomethyl-isoquinoline (159 mg; 0.71 mmol), the title compound was obtained as yellow solid in 85% yield.

¹H NMR (DMSO-d6, 400 MHz): δ 9.05 (1H, s), 8.02-8.04 (1H, d), 7.83-7.85 (2H, d), 7.72-7.80 (3H, m), 7.61-7.66 (3H, m), 7.50-7.55 (3H, t), 7.40-7.42 (2H, d), 4.60 (2H, s), 4.58 (2H, s). MS (ESI+): 447.9. HPLC (Condition B): Rt 3.52 min (HPLC purity 94.8%).

### Intermediate 88

### N-benzyl-2-fluoro-4-chloro-N-(4-cyanobenzyl)benzene sulfonamide

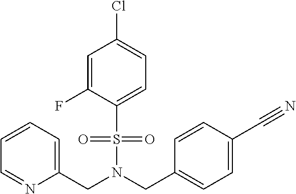

Following the general method as outlined for Intermediate 14, starting from 4-{([(pyridin-2-ylmethyl)amino]methyl}benzonitrile (Intermediate 52, 500 mg, 2.2 mmol) and 2-fluoro-4-chloro benzene sulfonyl chloride (562 mg, 2.4 mmol), the title compound was obtained as yellow solid in 76% yield.

¹H NMR (DMSO-d6, 400 MHz): δ8.25-8.27 (1H, d), 7.69-7.75 (4H, m), 7.80-7.81 (2H, t), 7.59-7.63 (2H, t), 7.38-7.42 (3H, m), 7.12-7.18 (2H, m), 4.66 (2H, s), 4.51 (2H, s). MS (ESI+): 416.0. HPLC (Condition B): Rt 4.46 min (HPLC purity 96.7%).

### Intermediate 89

### N-benzyl-2,4-dichloro-N-(4-cyanobenzyl)benzene sulfonamide

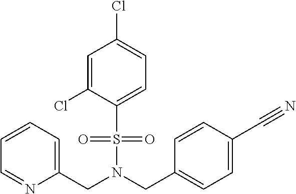

Following the general method as outlined for Intermediate 14, starting from 4-{[(pyridin-2-ylmethyl)amino]methyl}benzonitrile (Intermediate 52, 500 mg, 2.2 mmol) and 2,4-dichloro benzene sulfonyl chloride (602 mg, 2.46 mmol), the title compound was obtained as yellow solid in 83% yield.

¹H NMR (DMSO-d6, 400 MHz): δ8.34-8.35 (1H, d), 7.95-7.97 (1H, d), 7.83 (1H, s), 7.73-7.75 (2H, d), 7.60-7.63 (1H, t), 7.36-7.38 (1H, d), 7.18-7.21 (1H, m), 7.09-7.11 (2H, d) 4.71 (2H, s), 4.53 (2H, s). MS (ESI+): 432.1. HPLC (Condition B): Rt 4.69 min (HPLC purity 93.9%).

### Intermediate 90

### N-benzyl-2-fluoro-4-chloro-5-methyl-N-(4-cyanobenzyl)benzenesulfonamide

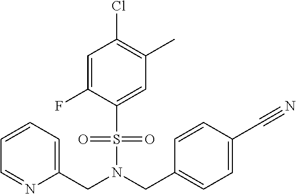

Following the general method as outlined for Intermediate 14, starting from 4-{[(pyridin-2-ylmethyl)amino]methyl}benzonitrile (Intermediate 52, 500 mg, 2.2 mmol) and 2-fluoro-4-chloro-5-methyl benzene sulfonyl chloride (602 mg, 2.47 mmol), the title compound was obtained as yellow solid in 73% yield.

¹H NMR (DMSO-d6, 400 MHz): δ8.27-8.28 (1H, d), 7.61-7.73 (5H, m), 7.39-7.41 (2H, d), 7.16-7.19 (2H, d), 4.66 (2H, s), 4.52 (2H, s) 2.28 (3H, s). MS (ESI+): 430.0. HPLC (Condition B): Rt 4.71 min (HPLC purity 95.8%).

### Intermediate 91

### 4-Ethoxy-N-(pyridin-2-ylmethyl)benzene sulfonamide

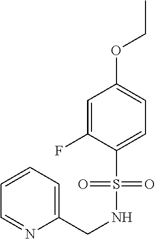

A cooled (0° C.) solution of 2-picolyl amine (300 mg; 2.77 mmol) in DCM (15 ml), was treated with triethylamine (0.98 ml; 8.31 mmol) followed by a solution of 4-ethoxybenzenesulfonyl chloride (670 mg; 3.07 mmol) in DCM (5 mL). The reaction mixture was allowed to warm to room temperature and stirred overnight. The reaction mixture was quenched with ice, diluted with DCM and washed with 10% aqueous sodium bicarbonate followed by brine. The organic layer was dried over sodium sulphate, concentrated and recrystallised from DCM/hexane to give the title compound as a white solid (0.28 g; 35% yield)

### Intermediate 92

### N-(4-cyano-3-fluorobenzyl)-4-ethoxy-N-(pyridin-2-ylmethyl)benzenesulfonamide

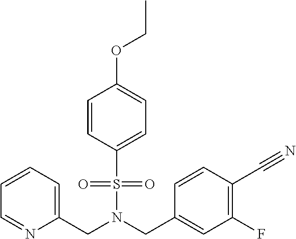

Following the general method as outlined for Intermediate 48, starting from 4-ethoxy-N-(pyridin-2-ylmethyl)benzene sulfonamide] (intermediate 91, 280 mg; 0.957 mmol) and 4-cyano-3-fluorobenzyl bromide (226 mg; 1.05 mmol), the title compound was obtained as yellow solid in 93% yield.

¹H NMR (DMSO-d6, 400 MHz): δ 8.32-8.33 (1H, d), 7.74-7.79 (3H, m), 7.61-7.65 (1H, t), 7.16-7.23 (4H, m), 7.06-7.09 (2H, d), 4.47 (2H, s), 4.41 (2H, s), 4.10-4.11 (2H, q), 1.33-1.37 (3H, t). MS (ESI+): 426.1. HPLC (Condition B): Rt 4.35 min (HPLC purity 95.6%).

### Intermediate 93

### 2-Fluoro-4-{[(2-fluorobenzyl)amino]methyl}benzonitrile

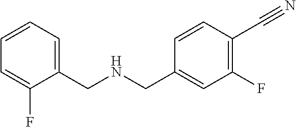

A solution of 2-fluorobenzylamine (300 mg, 2.39 mmol) in acetonitrile (20 ml) was treated with potassium carbonate (500 mg; 3.58 mmol) and 4-(bromomethyl)-3-fluorobenzonitrile (514 mg; 3.58 mmol) and refluxed for 3 h. The reaction mixture was evaporated under vacuum; the residue was dissolved in water and extracted with ethyl acetate. The combined organic layers were washed with brine (50 ml), dried over anhydrous sodium sulphate and concentrated under vacuum to afford the title compound as colorless oil.

¹H NMR (DMSO-d6, 400 MHz): δ 8.37 (1H, s), 7.83-7.87 (2H, t), 7.40-7.51 (1H, m) 7.30-7.38 (1H, m), 7.10-7.18 (2H, m), 3.79 (2H, s), 3.69 (2H, s). MS (ESI+): 259.2. HPLC (Condition B): Rt 2.79 min (HPLC purity 98.6%).

### Intermediate 94

### N-(2-fluorobenzyl)-N-[3-fluoro-4-(2H-tetrazol-5-yl)benzyl]amine

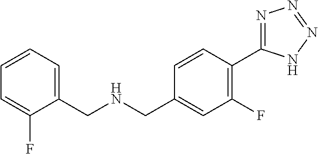

Following the general method as outlined in Intermediate 14, starting from 2-fluoro-4-{[(2-fluorobenzyl)amino]methyl}benzonitrile (Intermediate 93, 370 mg, 1.42 mmol), the title compound was obtained as white solid.

MS (ESI−): 299.8. HPLC (Condition B): Rt 3.85 min (HPLC purity 94.2%).

### Intermediate 95

### 4-chloro-N-(4-cyano-2-fluorobenzyl)-N-(2-fluorobenzyl)benzenesulfonamide

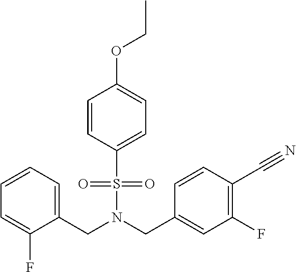

Following the general method as outlined for MC001_{—}129, starting from 2-fluoro-4-{[(2-fluorobenzyl)amino]methyl}benzonitrile (Intermediate 93, 400 mg; 1.54 mmol) and 4-chlorobenzenesulfonyl chloride (360 mg; 1.69 mmol), the title compound was obtained as white solid in 76% yield.

¹H NMR (DMSO-d6, 400 MHz): δ 7.89-7.90 (2H, d), 7.69-7.74 (3H, m), 7.56-7.58 (2H, m) 7.35-7.37 (2H, t), 7.03-7.19 (2H, d), 4.43 (2H, s), 4.42 (2H, s). MS (ESI+): 433.1. HPLC (Condition B): Rt 4.47 min (HPLC purity 85.1%).

### Intermediate 96

### 4-{[(2-fluorobenzyl)amino]methyl}benzonitrile

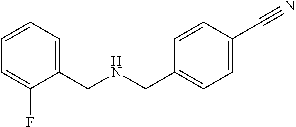

Following the general method as outlined for intermediate 93, starting from 2-fluoro-benzylamine (1.00 g; 7.99 mmol) and 4-(bromomethyl)benzonitrile (1.72 g; 8.78 mmol), the title compound was obtained as colorless oil (1.2 g, 63%).

¹H NMR (DMSO-d6, 400 MHz): δ 7.75-7.78 (2H, d), 7.53-7.55 (2H, d), 7.45-7.49 (1H, t) 7.25-7.30 (1H, m), 7.10-7.18 (2H, m), 3.77 (2H, s), 3.69 (2H, s). MS (ESI+): 241.0. HPLC (Condition B): Rt 4.20 min (HPLC purity 98.2%).

### Intermediate 97

### N-(2-fluorobenzyl)-N-[4-(2H-tetrazol-5-yl)benzyl]amine

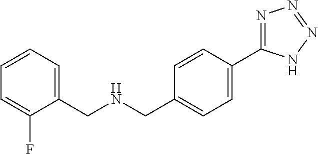

Following the general method as outlined in Intermediate 14, starting from 4-{[(2-fluorobenzyl)amino]methyl}benzonitrile (Intermediate 96, 1.00 g; 4.16 mmol), the title compound was obtained as white solid.

¹H NMR (DMSO-d6, 400 MHz): δ 7.99-8.01 (2H, d), 7.51-7.57 (3H, m), 7.38-7.42 (1H, t) 7.22-7.26 (2H, m), 4.07 (2H, s), 4.05 (2H, s). MS (ESI−): 282.0. HPLC (Condition B): Rt 3.92 min (HPLC purity 98.0%).

### Example 1

### 4-({benzyl[(4-chlorophenyl)sulfonyl]amino}methyl)-N-(3-chlorobenzyl)benzamide

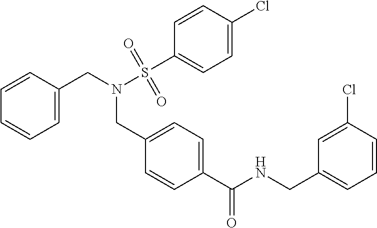

A mixture of 3-chlorobenzylamine (28.0 mg; 0.20 mmol), 4-([benzyl-(4-chloro-benzenesulfonyl)-amino]-methyl)-benzoic acid (Example 1, 91.5 mg; 0.22 mmol) and triethylamine (83 μl; 0.60 mmol) in DCM (2 ml) was treated with polymer-supported Mukaiyama reagent (320 mg; 0.40 mmol) and stirred for 16 hours. DCM was added to the reaction mixture and the solution was filtered through a SPE-NH₂ column (2 g). The DCM was evaporated in vacuo, to afford a residue which was purified by column chromatography (silica) eluting with chloroform containing increasing amounts of methanol, followed by crystallization from MeOH/Acetone to give the title compound as an off-white solid (16.8 mg, 15%.

¹H NMR (DMSO-d6, 300 MHz): δ 9.02 (1H, t, J=6.0 Hz), 7.91 (2H, d, J=8.5 Hz), 7.75-7.68 (4H, m), 7.39-7.10 (10H, m), 4.45 (2H, d, J=6.0 Hz), 4.37 (2H, s), 4.33 (2H, s). MS (ESI+): 538.9. HPLC (Condition A): Rt 5.50 min (HPLC purity 96.6%).

### Example 2

### 4-({benzyl[(4-chlorophenyl)sulfonyl]amino}methyl)-N-(cyclopropylmethyl)benzamide

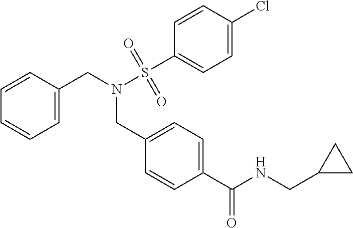

Following the general method as outlined in Example 2, starting from 4-([benzyl-(4-chloro-benzenesulfonyl)-amino]-methyl)-benzoic acid (Example 1, 100 mg; 0.24 mmol) and aminomethylcyclopropane (Aldrich, 20.5 mg; 0.29 mmol), the title compound was obtained as a white solid in 45% yield after crystallization from Et₂O.

¹H NMR (DMSO-d6, 300 MHz): δ 8.50 (1H, t, J=5.5 Hz), 7.91 (2H, d, J=8.5 Hz), 7.70 (4H, d, J=8.5 Hz), 7.24-7.10 (7H, m), 4.37 (2H, s), 4.33 (2H, s), 3.11 (2H, t, J=5.5 Hz), 1.01 (1H, m), 0.45-0.39 (2H, m), 0.24-0.19 (2H, m). MS (ESI+): 468.6. HPLC (Condition A): Rt 4.99 min (HPLC purity 97.7%).

### Example 3

### 4-({benzyl[(4-chlorophenyl)sulfonyl]amino}methyl)-N-[(2S)-tetrahydrofuran-2-ylmethyl]benzamide

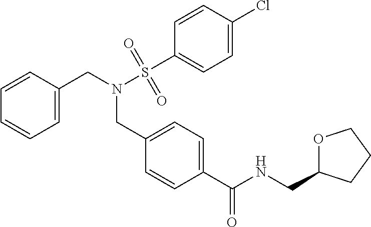

Following the general method as outlined in Example 2, starting from 4-([benzyl-(4-chloro-benzenesulfonyl)-amino]-methyl)-benzoic acid (Example 1, 125 mg; 0.30 mmol) and (S)-tetrahydrofurfurylamine (30.0 mg; 0.30 mmol), the title compound was obtained as a white solid in 31% yield after purification by column chromatography (silica) eluting with DCM.

¹H NMR (DMSO-d6, 300 MHz): δ 8.46 (1H, t, J=5.5 Hz), 7.90 (2H, d, J=8.5 Hz), 7.69 (4H, d, J=8.5 Hz), 7.24-7.09 (7H, m), 4.36 (2H, s), 4.32 (2H, s), 3.96 (1H, t, J=6.5 Hz), 3.76 (1H, m), 3.62 (1H, m), 3.30-3.26 (3H, m), 1.92-1.75 (3H, m), 1.58 (1H, m). MS (ESI+): 499.0. HPLC (Condition A): Rt 4.69 min (HPLC purity 98.9%).

### Example 4

### 4-({benzyl[(4-chlorophenyl)sulfonyl]amino}methyl)-N-[2-(3-nitrophenyl)ethyl]benzamide

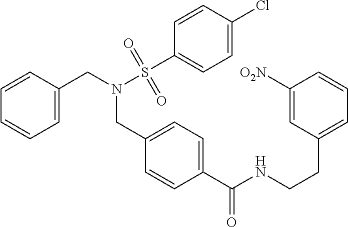

Following the general method as outlined in Example 2, starting from 4-([benzyl-(4-chloro-benzenesulfonyl)-amino]-methyl)-benzoic acid (Example 1, 410 mg; 0.99 mmol) and 3-nitrophenethylamine hydrochloride (200 mg; 0.99 mmol), the title compound was obtained as a white solid in 29% yield after slurrying in ethanol.

¹H NMR (DMSO-d6, 300 MHz): δ 8.49 (1H, t, J=5.5 Hz), 8.12 (1H, bs), 8.08 (1H, d, J=8.0 Hz), 7.90 (2H, d, J=8.5 Hz), 7.72-7.56 (6H, m), 7.21-7.19 (3H, m), 7.13-7.08 (4H, m), 4.35 (2H, s), 4.32 (2H, s), 3.52 (2H, q, J=6.5 Hz), 2.99 (2H, t, J=6.5 Hz). MS (ESI+): 564.3. HPLC (Condition A): Rt 5.28 min (HPLC purity 94.3%).

### Example 5

### 4-{[[(4-chlorophenyl)sulfonyl](pyridin-2-ylmethyl)amino]methyl}-N-(cyclopropylmethyl)benzamide

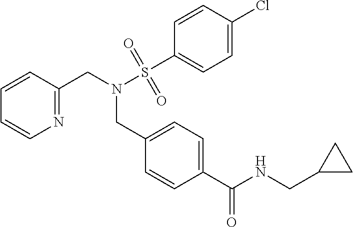

A mixture of 4-chloro-N-(pyridin-2-ylmethyl)benzenesulfonamide (100 mg; 0.35 mmol), 4-(bromomethyl)-N-(cyclopropylmethyl)benzamide (104 mg; 0.39 mmol), potassium carbonate (49.9 mg; 0.36 mmol), sodium iodide (1 mg; 0.01 mmol) in anhydrous DMF (1 ml) was heated to 100° C. for 4 h. The mixture was diluted with DCM and extracted with brine. The organic phase was separated, dried over magnesium sulfate, filtered and concentrated to give solid, which was which was purified by column chromatography (silica) eluting with DCM containing increasing amounts of methanol, followed by crystallisation from Et₂O to give the title compound as a pale yellow powder (71 mg, 43%).

¹H NMR (DMSO-d6, 300 MHz): δ 8.48 (1H, t, J=5.5 Hz), 8.32 (1H, m), 8.84 (2H, d, J=8.5 Hz), 7.72 (2H, d, J=8.5 Hz), 7.66-7.60 (3H, m), 7.25-7.16 (4H, m), 7.13-7.08 (4H, m), 4.50 (2H, s), 4.40 (2H, s), 3.11 (1H, t, J=6.0 Hz), 1.00 (1H, m), 0.44-0.38 (2H, m), 0.22-0.18 (2H, m). MS (ESI+): 470.3. HPLC (Condition A): Rt 3.31 min (HPLC purity 97.4%).

### Example 6

### N-(3-chlorobenzyl)-4-{[[(4-chlorophenyl)sulfonyl](pyridin-2-ylmethyl)amino]methyl}benzamide

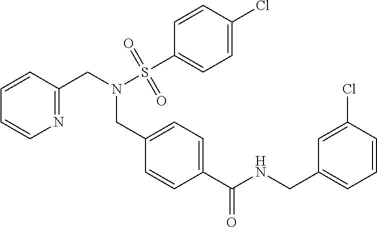

A mixture of 4-chloro-N-(pyridin-2-ylmethyl)benzenesulfonamide (90 mg; 0.32 mmol), N-(3-chlorobenzyl)-4-(chloromethyl)benzamide (103 mg; 0.35 mmol), potassium carbonate (44.9 mg; 0.36 mmol), sodium iodide (1 mg; 0.01 mmol) in anhydrous DMF (0.5 ml) was heated to 100° C. for 2.5 h. The mixture was diluted with DCM and extracted with brine. The organic phase was separated, dried over magnesium sulfate, filtered and concentrated to give solid, which was which was purified by crystallisation from Et₂O to give the title compound as a pale yellow powder (89 mg, 51%).

¹H NMR (DMSO-d6, 300 MHz): δ 9.03 (1H, t, J=6.0 Hz), 8.33 (1H, m), 7.85 (2H, d, J=8.5 Hz), 7.77 (2H, d, J=8.5 Hz), 7.67-7.61 (3H, m), 7.39-7.25 (6H, m), 7.21-7.17 (2H, m), 4.52 (2H, s), 4.46 (2H, d, J=6.0 Hz), 4.43 (2H, s). MS (ESI+): 540.5. HPLC (Condition A): Rt 4.11 min (HPLC purity 97.1%).

### Example 7

### 4-({benzyl[(4-chlorophenyl)sulfonyl]amino}methyl)-N-(2-thienylmethyl)benzamide

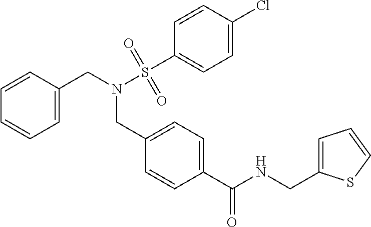

A solution of 4-([benzyl-(4-chloro-benzenesulfonyl)-amino]-methyl)-benzoic acid (Example 1, 100 mg; 0.24 mmol) and thiophene-2-methylamine (32.7 mg; 0.29 mmol) in DCM (5 mL) was treated with 1-(3-dimethylaminopropyl)-3-ethylcarbodiimide hydrochloride (55.3 mg; 0.29 mmol) and 1-hydroxybenzotriazole (39.0 mg; 0.29 mmol). After stirring for 20 h, the mixture was diluted with DCM and extracted with brine. The organic phase was separated, dried over magnesium sulfate, filtered and concentrated to give solid, which was which was purified by crystallisation from EtOH/Et₂O to give the title compound as a white powder (76.8 mg, 62%).

¹H NMR (DMSO-d6, 300 MHz): δ 9.08 (1H, t, J=6.0 Hz), 7.90 (2H, d, J=8.5 Hz), 7.73-7.67 (4H, m), 7.38 (1H, dd, J=5.0 Hz, J=1.5 Hz), 7.24-7.19 (3H, m), 7.16-7.09 (4H, m), 7.00 (1H, dd, J=3.0 Hz, J=1.0 Hz), 6.95 (1H, dd, J=5.0 Hz, J=3.0 Hz), 4.60 (2H, d, J=6.0 Hz), 4.37 (2H, s), 4.33 (2H, s). MS (ESI+): 511.2. HPLC (Condition A): Rt 5.20 min (HPLC purity 98.8%).

### Example 8

### 6-({benzyl[(4-chlorophenyl)sulfonyl]amino}methyl)-N-(3-chlorobenzyl)nicotinamide

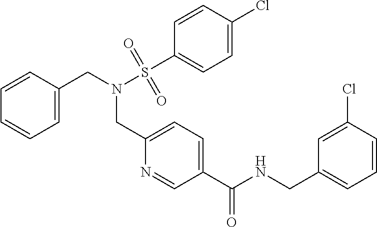

Following the general method as outlined in Example 8, starting from 6-({(benzyl[(4-chlorophenyl)sulfonyl]amino}methyl)nicotinic acid (Intermediate 6, 100 mg; 0.24 mmol) and 3-chlorobenzylamine (40.8 mg; 0.29 mmol), the title compound was obtained as a white powder in 52% yield after slurrying in Et₂O.

¹H NMR (DMSO-d6, 300 MHz): δ 9.19 (1H, t, J=6.0 Hz), 8.80 (1H, d, J=1.5 Hz), 8.07 (1H, dd, J=8.0 Hz, J=2.5 Hz), 7.86 (2H, d, J=8.5 Hz), 7.64 (2H, d, J=8.5 Hz), 7.40-7.17 (10H, m), 4.47 (2H, d), 4.47 (4H, s). MS (ESI+): 540.2. HPLC (Condition A): Rt 5.18 min (HPLC purity 99.3%).

### Example 9

### N-(3-chlorobenzyl)-4-{[[(4-methoxyphenyl)sulfonyl](pyridin-3-ylmethyl)amino]methyl}benzamide

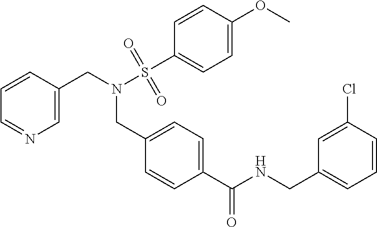

Following the general method as outlined in Example 8, starting from 4-{[[(4-methoxyphenyl)sulfonyl](pyridin-3-ylmethyl)amino]methyl}benzoic acid (Intermediate 9, 110 mg; 0.27 mmol) and 3-chlorobenzylamine (45.3 mg; 0.32 mmol), the title compound was obtained as an off-white solid in 88% yield after slurrying in Et₂O.

¹H NMR (DMSO-d6, 300 MHz): δ 9.03 (1H, t, J=6.0 Hz), 8.34 (1H, d, J=4.5 Hz), 8.26 (1H, bs), 7.86 (2H, d, J=8.5 Hz), 7.73 (2H, d, J=8.0 Hz), 7.48 (1H, d, J=8.0 Hz), 7.39-7.26 (4H, m), 7.21-7.14 (5H, m), 4.45 (2H, d, J=6.0 Hz), 4.35 (4H, s), 4.32 (4H, s), 3.88 (4H, s). MS (ESI+): 536.3. HPLC (Condition A): Rt 3.55 min (HPLC purity 97.4%).

### Example 10

### 4-({benzyl[(4-chlorophenyl)sulfonyl]amino}methyl)-N-(1-phenylcyclopropyl)benzamide

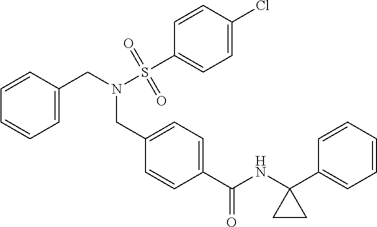

Following the general method as outlined in Example 8, starting from 4-([benzyl-(4-chloro-benzenesulfonyl)-amino]-methyl)-benzoic acid (Example 1, 100 mg; 0.24 mmol) and 1-phenyl-cyclopropylamine (38.4 mg; 0.29 mmol), the title compound was obtained as a white powder in 10% yield after purification by column chromatography (silica) eluting with chloroform containing increasing amounts of EtOAc.

¹H NMR (DMSO-d6, 300 MHz): δ 9.11 (1H, bs), 7.90 (2H, d, J=8.5 Hz), 7.74 (2H, d, J=8.0 Hz), 7.69 (2H, d, J=8.5 Hz), 7.29-7.23 (5H, m), 7.18-7.11 (7H, m), 4.37 (2H, s), 4.33 (2H, s), 1.25 (4H, s). MS (ESI+): 531.3. HPLC (Condition A): Rt 5.41 min (HPLC purity 89.9%).

### Example 11

### N-(benzylsulfonyl)-4-{[[(4-chlorophenyl)sulfonyl](pyridin-2-ylmethyl)amino]methyl}benzamide

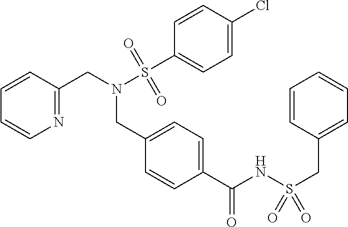

A solution of 4-{[[(4-chlorophenyl)sulfonyl](pyridin-2-ylmethyl)amino]methyl}benzoic acid (Intermediate 3, 70 mg; 0.17 mmol) and alpha-toluenesulfonamide (30.2 mg; 0.18 mmol) in DCM (2 mL) was treated with 1-(3-dimethylaminopropyl)-3-ethylcarbodiimide hydrochloride (64.4 mg; 0.34 mmol) and 4-dimethylaminopyridine (41.0 mg; 0.34 mmol). After stirring for 4 h, the mixture was diluted with DCM and extracted with brine. The organic phase was separated, dried over magnesium sulfate, filtered and concentrated to give solid, which was which was purified by slurrying from EtOH/Et₂O to give the title compound as a white powder (30.1 mg, 31%).

¹H NMR (DMSO-d6, 400 MHz): δ 8.3 (1H, d, J=4.5 Hz), 7.83 (2H, d, J=8.5 Hz), 7.76 (2H, d, J=8.0 Hz), 7.65-7.58 (3H, m), 7.36-7.34 (3H, m), 7.31-7.28 (4H, m), 7.17 (2H, d, J=7.5 Hz), 4.83 (2H, s), 4.53 (2H, s), 4.42 (2H, s). MS (ESI+): 570.3. HPLC (Condition A): Rt 4.02 min (HPLC purity 95.6%).

### Example 12

### N-[(3-chlorophenyl)sulfonyl]-4-{[benzyl[(4-chlorophenyl)sulfonyl]amino]methyl}benzamide

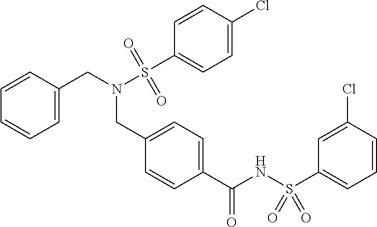

Following the general method as outlined in Example 12, starting from 4-([benzyl-(4-chloro-benzenesulfonyl)-amino]-methyl)-benzoic acid (Example 1, 100 mg; 0.24 mmol) and 3-chlorobenzenesulphonamide (48.4 mg; 0.25 mmol), the title compound was obtained as an off-white powder in 20% yield after purification by column chromatography (silica) eluting with DCM containing increasing amounts of AcOH.

MS (ESI+): 589.3. HPLC (Condition A): Rt 5.96 min (HPLC purity >99.8%).

### Example 13

### N-[(3-chlorophenyl)sulfonyl]-4{[[(4-chlorophenyl)sulfonyl](pyridin-2-ylmethyl)amino]methyl}benzamide

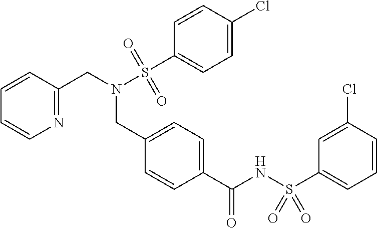

Following the general method as outlined in Example 12, starting from 4-{[[(4-chlorophenyl)sulfonyl](pyridin-2-ylmethyl)amino]methyl}benzoic acid (Intermediate 3, 50.0 mg; 0.12 mmol) and 3-chlorobenzenesulphonamide (24.1 mg; 0.13 mmol), the title compound was obtained as a white solid in 24% yield after slurrying in Et₂O.

¹H NMR (DMSO-d6, 400 MHz): δ 8.30 (1H, dd, J=5.0 Hz, J=2.0 Hz), 7.95-7.88 (2H, m), 7.85-7.79 (2H, d, J=8.0 Hz), 7.78-7.71 (3H, m), 7.67-7.58 (4H, m), 7.25 (2H, d, J=8.0 Hz), 7.19-7.14 (2H, m), 4.51 (2H, s), 4.40 (2H, s). MS (ESI+): 590.2. HPLC (Condition A): Rt 4.12 min (HPLC purity 94.4%).

### Example 14

### 4-{[[(4-chlorophenyl)sulfonyl](pyridin-2-ylmethyl)amino]methyl}-N-[(4-methoxyphenyl)sulfonyl]benzamide

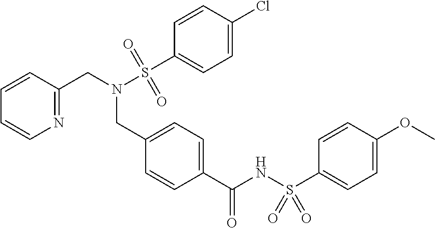

Following the general method as outlined in Example 12, starting from 4-{[[(4-chlorophenyl)sulfonyl](pyridin-2-ylmethyl)amino]methyl}benzoic acid (Intermediate 3, 50.0 mg; 0.12 mmol) and 4-methoxybenzenesulphonamide (23.6 mg; 0.13 mmol), the title compound was obtained as a white solid in 6% yield after slurrying in ethanol. MS (ESI+): 586.3. HPLC (Condition A): Rt 3.79 min (HPLC purity 97.7%).

### Example 15

### 4-({benzyl[(4-chlorophenyl)sulfonyl]amino}methyl)-N-[(3-nitrobenzyl)sulfonyl]benzamide

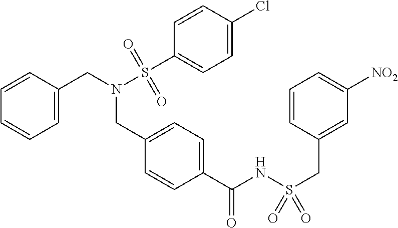

Following the general method as outlined in Example 12, starting from 4-([benzyl-(4-chloro-benzenesulfonyl)-amino]-methyl)-benzoic acid (100 mg; 0.24 mmol) and 1-(3-nitrophenyl)methanesulfonamide (Intermediate 8, 54.6 mg; 0.25 mmol), the title compound was obtained as an ivory powder in 29% yield after slurrying in ethanol.

MS (ESI−): 612.4. HPLC (Condition A): Rt 5.70 min (HPLC purity 93.9%).

### Example 16

### 4-chloro-N-(pyridin-2-ylmethyl)-N-[4-(1H-tetrazol-5-yl)benzyl]benzenesulfonamide

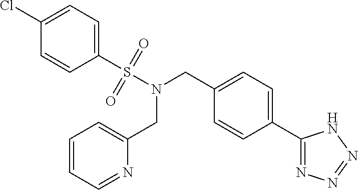

A solution of 4-chloro-N-(4-cyanobenzyl)-N-(pyridin-2-ylmethyl)benzenesulfonamide (83.0 mg; 0.21 mmol) in toluene (7 ml) was treated with azidotrimethylsilane (72.1 mg; 0.63 mmol) and dibutyltin oxide (31.2 mg; 0.13 mmol). After heating at 90° C. for 18 h, the mixture was diluted with DCM and extracted with a solution of NaOH (0.1 N) in water. The aqueous phase was cautiously acidified with an HCl solution (5 N). The resulting precipitate was filtered, washed with water and dried in vacuo to give the title compound as a brown powder (68.8 mg, 75%).

¹H NMR (DMSO-d6, 400 MHz): δ 8.33 (1H, d, J=5.0 Hz), 7.94-7.89 (5H, m), 7.68 (2H, d, J=8.5 Hz), 7.42-7.38 (4H, m), 4.57 (2H, s), 4.54 (2H, s). MS (ESI+): 441.2. HPLC (Condition A): Rt 3.14 min (HPLC purity 96.8%).

### Example 17

### 4-{[Benzyl-(4-methoxy-benzenesulfonyl)-amino]-methyl}-N-cyclopropylmethylbenzamide

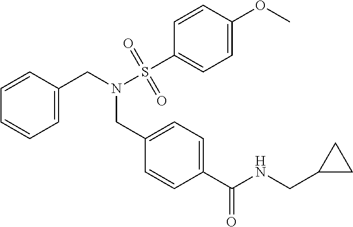

Following the general method as outlined in Example 2, starting from 4-{[benzyl (4-methoxysulfonyl)amino]}benzoic acid (Intermediate 15, 100 mg, 0.24 mmol), the title compound was obtained as an off-white solid in 79% yield after purification by column chromatography (silica) eluting with chloroform containing increasing amounts of EtOAc.

¹H NMR (DMSO-d6, 400 MHz): δ 8.48-8.45 (1H, m), 7.83 (2H, d, J=9.0 Hz), 7.67 (2H, d, J=8.0 Hz), 7.21-7.18 (3H, m), 7.14-7.08 (6H, m), 4.29 (2H, s), 4.26 (2H, s), 3.86 (3H, s), 3.10-3.08 (2H, m), 1.02-0.9 (1H, m), 0.41-0.38 (2H, m), 0.23-0.17 (2H, m). MS (ESI+): 465.2. HPLC (Condition B): Rt 3.89 min (HPLC purity 99.9%).

### Example 18

### 4-{[Benzyl-(4-ethoxy-benzenesulfonyl)-amino]-methyl}-N-cyclopropylmethylbenzamide

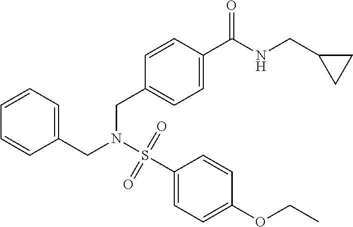

Following the general method as outlined in Example 2, starting from 4-{[Benzyl-(4-ethoxy-benzenesulfonyl)-amino]-methyl}benzoic acid (Example 18, 100 mg 0.23 mmol), the title compound was obtained as a white solid.

¹H NMR (DMSO-d6, 400 MHz): δ 8.47 (1H, m), 7.81 (2H, d, J=9.0 Hz), 7.67 (2H, d, J=8.0 Hz) 7.21-7.18 (3H, m) 7.12-7.07 (6H, m), 4.28 (2H, s), 4.25 (2H, s), 4.13 (2H, q, J=7.0 Hz), 3.08-3.11 (2H, m), 1.36 (3H, t, J=7.0 Hz), 1.01-0.97 (1H, m), 0.43-0.40 (2H, m), 0.23-0.20 (2H, m). MS (ESI+): 479.2. HPLC (Condition B): Rt 4.03 min (HPLC purity 94.7%).

### Example 19

### 4-chloro-N-[4-(5-hydroxy-1,3,4-oxadiazol-2-yl)benzyl]-N-(pyridin-2-ylmethyl)benzene sulfonamide

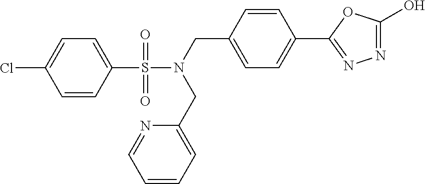

A cooled (0° C.) solution of 4-chloro-N-[4-(hydrazinomethyl)benzyl]-N-(pyridin-2-ylmethyl)benzenesulfonamide (Intermediate 17, 150 mg, 0.34 mmol) in DMF (10 ml) was treated with 1,1-carbonyldiimidazole (112 mg, 0.69 mol) and triethylamine (70 mg, 0.69 mmol). The reaction mixture was stirred for 4 h at 0° C., and then at room temperature for 14 hrs. The solvent was removed under reduced pressure and the crude was purified by column chromatography to afford the title compound as an off-white solid (120 mg, 77%).

¹H NMR (DMSO-d6, 400 MHz): 812.6 (1H, bs), 8.31 (1H, m), 7.83 (2H, d, J=8.5 Hz), 7.67-7.61 (5H, m) 7.34 (2H, d, J=8.5 Hz), 7.19-7.16 (2H, m), 4.52 (2H, s), 4.43 (2H, s). MS (ESI−) δ 455.0. HPLC (Condition B): Rt 2.75 min (HPLC purity 99.8%).

### Example 20

### 4-chloro-N-(2-fluorobenzyl)-N-[4-(1H-tetrazol-5-yl)benzyl]benzene sulfonamide

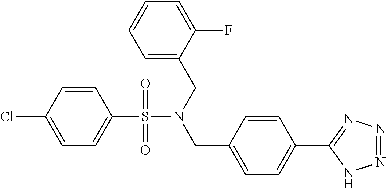

Following the general method as outlined in Example 17, starting from 4-chloro-N-(4-cyanobenzyl)-N-(2-fluorobenzyl)benzene sulfonamide (Intermediate 19; 100 mg; 0.24 mmol), the title compound was obtained as a white solid.

¹H NMR (DMSO-d6, 400 MHz) δ 7.89 (2H, d, J=8.5 Hz), 7.85 (2H, d, J=8.0 Hz), 7.69 (2H, d, J=8.5 Hz), 7.31 (2H, d, J=8.0 Hz), 7.25-7.17 (2H m), 7.03-6.94 (2H, m), 4.42 (2H, s), 4.41 (2H, s). MS (ESI−): 455.8. HPLC (Condition B): Rt 3.74 min (HPLC purity 99.5%).

### Example 21

### 4-chloro-N-(3-chlorobenzyl)-N-[4-(1H-tetrazol-5-yl)benzyl]benzenesulfonamide

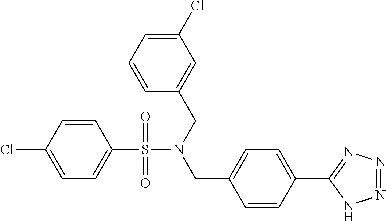

Following the general method as outlined in Example 17, starting from 4-Chloro-N-(3-chloro-benzyl)-N-(4-cyano-benzyl)-benzenesulfonamide (Intermediate 20, 100 mg; 0.24 mmol), the title compound was obtained as a white solid.

¹H NMR (DMSO-d6, 400 MHz): δ 7.92 (2H, d, J=8.5 Hz), 7.86 (2H, d, J=8.0 Hz), 7.70 (2H, d, J=8.5 Hz), 7.32 (2H, d, J=8.0 Hz), 7.22-7.17 (2H, m), 7.10-7.06 (1H, m), 7.01 (1H, s), 4.40 (2H, s), 4.36 (2H, s). MS (ESI−): 471.9. HPLC (Condition B): Rt 3.90 min (HPLC purity 97.4%).

### Example 22

### 4-chloro-N-(4-fluorobenzyl)-N-[4-(1H-tetrazol-5-yl)benzyl]benzenesulfonamide

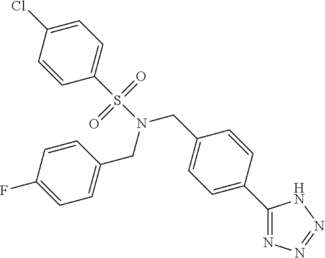

Following the general method as outlined in Example 17, starting from 4-chloro-N-(4-cyanobenzyl)-N-(4-fluorobenzyl)benzenesulfonamide (Intermediate 21, 250 mg, 0.6 mmol), the title compound was obtained as a white solid.

¹H NMR (DMSO-d6, 400 MHz): δ7.91 (2H, d, J=8.5 Hz), 7.85 (2H, d, J=8.0 Hz), 7.69 (2H, d, J=8.5 Hz), 7.28 (2H, d, J=8.0 Hz), 7.18-7.14 (2H, m), 7.02-6.98 (2H, t, J=8.5 Hz), 4.40 (2H, s), 4.34 (2H, s). MS (ESI−): 455.8. HPLC (Condition B): Rt 3.77 min (HPLC purity 99.8%).

### Example 23

### 4-chloro-N-(3-methoxybenzyl)-N-[4-(1H-tetrazol-5-yl)benzyl]benzene sulfonamide

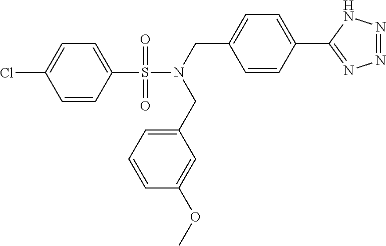

Following the general method as outlined in Example 17, starting from 4-chloro-N-(4-cyanobenzyl)-N-(3-methoxybenzyl)benzenesulfonamide (Intermediate 22; 100 mg: 0.24 mmol), the title compound was obtained as an off-white solid.

¹H NMR (DMSO-d6, 400 MHz) δ 7.92-7.86 (4H, m), 7.69 (2H, d, J=8.5 Hz), 7.30 (2H, d, J=8.0 Hz), 7.11 (1H, t, J=8.0 Hz), 6.73-7.68 (2H, m), 6.56 (1H, s), 4.39 (2H, s), 4.32 (2H, s), 3.57 (3H, s). MS (ESI−): 470.1. HPLC (Condition B): Rt 3.77 min (HPLC purity 99.5%).

### Example 24

### 4-Chloro-N-(4-methoxy-benzyl)-N-[4-(1H-tetrazol-5-yl)-benzyl]-benzenesulfonamide

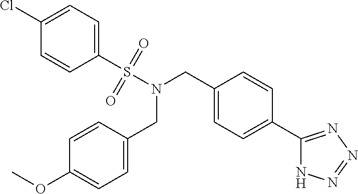

Following the general method as outlined in Example 17, starting from 4-chloro-N-(4-cyanobenzyl)-N-(4-methoxybenzyl)benzenesulfonamide (Intermediate 23; 600 mg, 1.4 mmol), the title compound was obtained as an off-white solid.

¹H NMR (DMSO-d6, 400 MHz): δ 7.90-7.87 (4H, m), 7.68 (2H, d, J=8.5 Hz), 7.27 (2H, d, J=8.0 Hz), 7.02 (2H, d, J=8.5 Hz), 6.74 (2H, d, J=8.5 Hz), 4.35 (2H, s), 4.27 (2H, s), 3.64 (3H, s). MS (ESI−): 468.0. HPLC (Condition B): Rt 3.76 min (HPLC purity 98.8%).

### Example 25

### 4-chloro-N-(4-chlorobenzyl)-N-[4-(1H-tetrazol-5-yl)benzyl]benzenesulfonamide

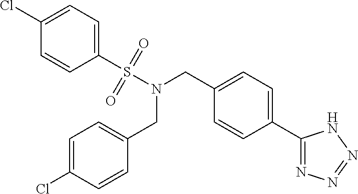

Following the general method as outlined in Example 17, starting from 4-chloro-N-(4-chlorobenzyl)-N-(4-cyanobenzyl)benzenesulfonamide (Intermediate 24; 150 mg, 0.35 mmol), the title compound was obtained as a white solid.

¹H NMR (DMSO-d6, 400 MHz): δ 7.93-7.90 (2H, m), 7.86-7.84 (2H, m), 7.70-7.68 (2H, m), 7.29 (2H, d, J=8.0 Hz), 7.25-7.23 (2H, m), 7.15-7.13 (2H, m), 4.40 (2H, s), 4.34 (2H, s). MS (ESI−): 471.9. HPLC (Condition B): Rt 3.93 min (HPLC purity 98.9%).

### Example 26

### 4-{[Benzyl({[4-(trifluoromethoxy)phenyl]sulfonyl)amino]methyl}-N-(cyclopropyl methyl)benzamide

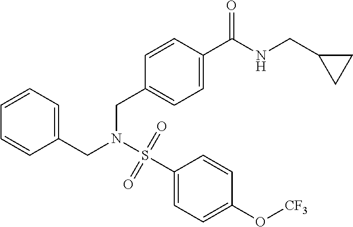

Following the general method as outlined for Example 8, starting from 4[(benzyl)[4(trifluoromethoxy)phenyl]sulfonyl}amino)methyl]benzoic acid (Intermediate 26, 50 mg, 0.107 mmol) and cyclopropane methylamine hydrochloride, the title compound was obtained as a white solid.

¹H NMR (DMSO-d6, 400 MHz): δ 8.49-8.46 (1H, m), 8.02-8.00 (2H, d), 7.69-7.67 (2H, d), 7.59-7.57 (2H, d), 7.20-7.19 (2H, d), 7.14-7.12 (3H, m), 7.09-7.07 (4H, m), 4.38 (2H, s), 4.34 (2H, s), 3.32 (2H, m), 1.0 (1H, m), 0.42-0.39 (2H, m), 0.21-0.20 (2H, m). MS (ESI+): 518.8. HPLC (Condition B): Rt 4.18 min (HPLC purity 96.9%).

### Example 27

### N-Benzyl-4-{[benzyl[3,4-dichlorobenzene]sulfonyl)amino]methyl}benzamide

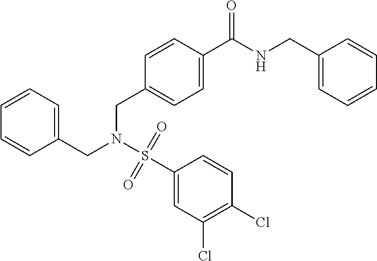

A solution of 4-({benzyl[(3,4-dichlorophenyl)sulfonylurea]amino}methyl)benzoic acid (Intermediate 28; 100 mg, 0.22 mmol) in THF (10 ml) was treated with triethylamine (66 mg, 6.6 mmol), EDC.HCl (84 mg, 0.44 mmol) and benzylamine (28.2 mg, 0.264 mmol) and stirred at RT for 16 h. The reaction mixture was concentrated under vacuum, water was added and extracted with ethyl acetate (3×20 ml). The combined organic layer was washed with brine and then dried over anhydrous sodium sulphate and concentrated under vacuum. The crude obtained was dissolved in DCM, passed through an SCX column and the solvent evaporated to yield the title compound as white solid.

¹H NMR (CDCl₃, 400 MHz): δ7.82-7.83 (1H, d), 7.67-7.69 (2H, d) 7.58-7.61 (2H, d), 7.37-7.38 (4H, d), 7.24-7.27 (3H, m), 7.16-7.18 (2H, d), 7.06-7.07 (2H, d), 6.31 (1H, s), 4.65-4.67 (2H, d), 4.38 (2H, s), 4.34 (2H, s), 2.18 (2H, s). MS (ESI+): 538.7. HPLC (Condition B): Rt 4.38 min (HPLC purity 95.3%).

### Example 28

### 4-({trifluoromethyl benzyl[(4-chlorophenyl)sulfonyl]amino}methyl)-N-(benzyl)benzamide

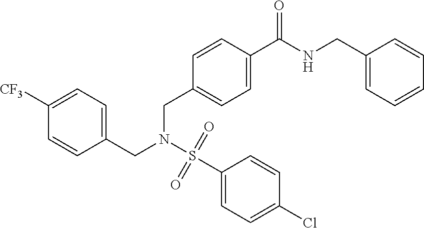

A solution of 4-({[(4-chlorophenyl)sulfonyl][4-(trifluoromethyl)benzyl]amino}methyl)benzoic acid (Intermediate 31, 50 mg 0.1 mmol) in DMF (5 mL) was treated with triethylamine (30 mg; 0.3 mmol) and EDC.HCl (39.6 mg; 0.2 mmol), benzylamine (11.6 mg, 0.2 mmol) and HOBt (27 mg, 0.2 mmol) and stirred at room temperature for 16 h. The reaction mixture was concentrated under vacuum, and then water was added and extracted with ethyl acetate. The combined organic layer was washed with brine and then dried over anhydrous sodium sulphate and concentrated under vacuum. The crude obtained was purified by SCX column to yield the title compound as white solid.

¹H NMR (DMSO-d6, 400 MHz): δ7.93-7.96 (1H, t), 7.89-7.93 (2H, t), 7.67-7.71 (4H, t), 7.52-7.54 (2H, d), 7.20-7.32 (7H, m), 7.15-7.17 (2H, d), 4.40-4.44 (6H, m). MS (ESI+): 572.7. HPLC (Condition B): Rt 4.33 min (HPLC purity 98.0%).

### Example 29

### 4-{[(4-Chloro-benzenesulfonyl)-(2-fluoro-benzyl)-amino]-methyl}-N-cyclopropyl methyl benzamide

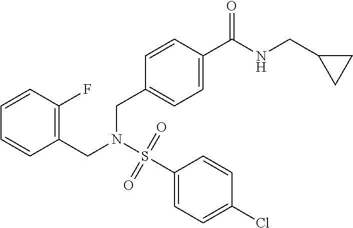

A solution of 4-{[[(4-chlorophenyl)sulfonyl](2-fluorobenzyl)amino]methyl}benzoic acid (Intermediate 33, 75 mg, 0.174 mmol) in dry DMF (8 ml) was treated with cyclopropyl methyl amine hydrochloride (66 mg, 0.35 mmol), triethylamine (0.11 ml, 0.872 mmol), EDC.HCl (66 mg, 0.35 mmol) and DMAP (4.5 mg). The reaction mixture was stirred at RT for 15 h. The reaction mixture was quenched into water and extracted with dichloromethane. The organic layer was washed with water, and brine solution and dried over Na₂SO₄ and evaporated under vacuum. The crude mass was purified by column chromatography to afford the title compound as a white solid.

¹H NMR (DMSO-d6, 400 MHz) δ 8.45-8.48 (1H, m), 7.87-7.89 (2H, d), 7.67-7.69 (4H, d), 7.15-7.25 (4H, m), 6.97-6.99 (2H, m), 4.37-4.38 (4H, d), 3.08-3.11 (2H, t), 0.96-1.01 (1H, m), 0.39-0.41 (2H, m) 0.41-0.42 (2H, m). MS (ESI+): 486.9. HPLC (Condition B): Rt 4.11 min (HPLC purity 99.8%).

### Example 30

### 4-{[(4-Chloro-benzenesulfonyl)-(3-chlorobenzyl)-amino]-methyl}-N-cyclopropyl methyl benzamide

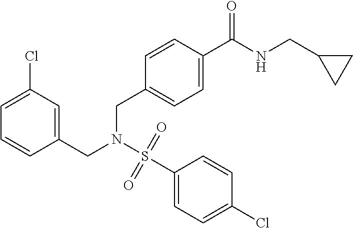

Following the general method as outlined for Example 29, starting from 4-{[[(4-chlorophenyl)sulfonyl](3-chlorobenzyl)amino]methyl}benzoic acid (Intermediate 35, 100 mg, 0.22 mmol) and cyclopropyl methyl amine hydrochloride (35 mg, 0.33 mmol), the title compound was obtained as a white solid.

¹H NMR (DMSO-d6, 400 MHz): δ 8.47 (1H, s), 7.70-7.91 (2H, m), 7.68-7.69 (4H, m), 7.21-7.22 (2H, m), 7.16-7.18 (2H, m), 7.04-7.06 (1H, m), 7.01 (1H, s), 4.39 (2H, s), 4.32 (2H, s), 3.09-3.32 (2H, t), 1.0 (1H, s), 0.38-0.42 (2H, m), 0.19-0.21 (2H, m). MS (ESI+): 502.6. HPLC (Condition B): Rt 4.23 min (HPLC purity 99.3%).

### Example 31

### N-benzyl-4-{[benzyl[4-methoxy phenyl]sulfonyl)amino]methyl}benzamide

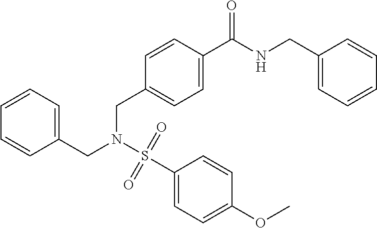

Following the general method as outlined in Example 8, starting from 4-([benzyl-(4-methoxy-benzenesulfonyl)-amino]-methyl)-benzoic acid (Intermediate 37, 100 mg; 0.24 mmol) and benzylamine (Aldrich, 0.031 ml; 0.29 mmol), the title compound was obtained as a white solid.

¹H NMR (DMSO-d6, 400 MHz): δ 8.95 (1H, s), 7.81-7.83 (2H, d), 7.70-7.72 (2H, d), 7.27-7.31 (4H, m), 7.19-7.22 (4H, t), 7.08-7.14 (6H, m), 4.43-4.44 (2H, d), 4.29 (2H, s), 4.26 (2H, s), 3.85 (3H, s). MS (ESI+): 500.9. HPLC (Condition B): Rt 4.00 min (HPLC purity 99.5%).

### Example 32

### N-benzyl-4-{[benzyl[4-fluoro phenyl]sulfonyl)amino]methyl}benzamide

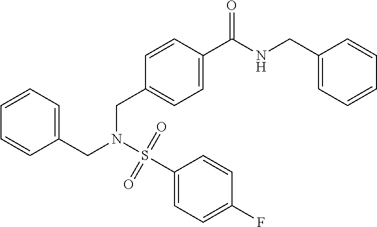

Following the general method as outlined in Example 35, starting from 4-([benzyl-(4-fluoro-benzene sulfonyl)-amino]-methyl)-benzoic acid (Intermediate 39, 100 mg; 0.25 mmol) and benzyl amine (Aldrich, 31 mg; 0.30 mmol), the title compound was obtained as off white.

¹H NMR (DMSO-d6, 400 MHz): δ 8.95-8.98 (1H, t), 7.94-7.97 (2H, m), 7.71-7.73 (2H, d) 7.42-7.47 (2H, t), 7.27-7.33 (4H, m), 7.20-7.24 (4H, m), 7.08-7.17 (4H, m), 4.43-4.45 (2H, d), 4.35 (2H, s), 3.31 (2H, s). MS (ESI+): 488.5. HPLC (Condition B): Rt 4.06 min (HPLC purity 97.9%).

### Example 33

### 4-{[Benzyl-(4-ethoxy-benzenesulfonyl)-amino]-methyl}-N-benzylmethyl-benzamide

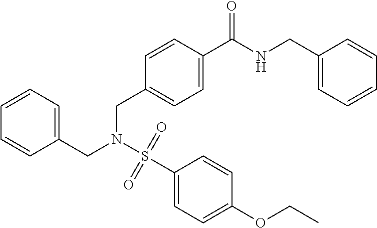

A solution of 4-({benzyl[(4-ethoxyphenyl) sulfonylurea]amino}methyl)benzoic acid (Intermediate 41; 100 mg; 0.23 mmol) in DMF (5 mL) was treated with triethylamine (72 mg; 0.7 mmol), TBTU (150.8 mg; 0.47 mmol) and benzylamine (30 mg, 0.28 mmol) and stirred at room temperature for 16 h. The reaction mixture was concentrated under vacuum, then water was added and extracted with ethyl acetate. The combined organic layer was washed with brine and then dried over anhydrous sodium sulphate and concentrated under vacuum. The crude obtained was purified by SCX column to yield the title compound as white solid.

¹H NMR (DMSO-d6, 400 MHz): δ8.96 (1H, s), 7.79-7.81 (2H, m), 7.70-7.72 (2H, d) 7.27-7.31 (4H, m) 7.18-7.23 (4H, m), 7.08-7.13 (6H, m), 4.43-4.44 (2H, d), 4.29 (2H, s), 4.26 (2H, s), 4.10-4.15 (2H, q), 1.33-1.37 (3H, t). MS (ESI+): 515.3. HPLC (Condition B): Rt 4.13 min (HPLC purity 96.1%).

### Example 34

### 4-{[[(4-chlorophenyl)sulfonyl](4-fluorobenzyl)amino]methyl}-N-phenyl methyl benzamide

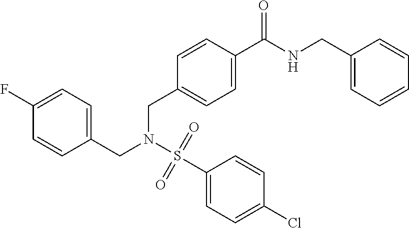

A solution of 4-{[[(4-chlorophenyl)sulfonyl](4-fluorobenzyl)amino]methyl}benzoic acid (Intermediate 41, 100 mg, 0.23 mmol) in DCM (20 ml) was treated with TBTU (150 mg, 4.8 mmol), triethylamine (0.1 ml) and benzyl amine (0.029 ml, 0.28 mmol) and stirred at room temperature for 16 h. The reaction mixture was quenched with ice and extracted with ethyl acetate. The combined organic layer was washed with brine and then dried over anhydrous sodium sulphate and concentrated under vacuum. The crude mass was purified by column chromatography to afford the title compound as off white solid.

¹H NMR (DMSO-d6, 400 MHz): δ 8.96 (1H, t), 7.88-7.91 (2H, d), 7.73-7.71 (2H, d), 7.67-7.73 (4H, m), 7.22-7.29 (4H, m), 7.12-7.15 (5H, m), 6.99-7.04 (2H, t), 4.43-4.45 (2H, d), 4.36 (2H, s), 4.30 (2H, s). MS (ESI+): 523.0. HPLC (Condition B): Rt 4.19 min (HPLC purity 99.3%).

### Example 35

### 4-{[(4-Chloro-benzenesulfonyl)-(4-methoxybenzyl)-amino]-methyl}-N-phenyl methyl benzamide

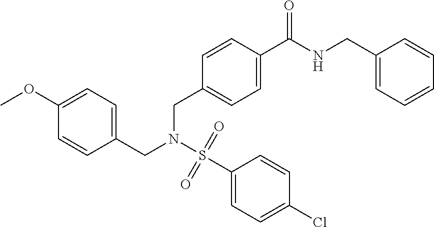

Following the general method as outlined for Example 36, starting from 4-{[[(4-chlorophenyl)sulfonyl](4-methoxybenzyl)amino]methyl}benzoic acid (Intermediate 45, 100 mg, 0.24 mmol) and benzyl amine (0.029 ml, 0.28 mmol), the title compound was obtained as off white solid.

¹H NMR (DMSO-d6, 400 MHz): δ 8.96-8.98 (1H, d), 7.86-7.89 (2H, m), 7.72-7.74 (2H, d), 7.66-7.68 (2H, m), 7.27-7.33 (4H, m), 7.20-7.24 (1H, m), 7.12-7.14 (2H, d), 6.99-7.01 (2H, d), 6.74-6.76 (2H, d), 4.44-4.45 (2H, d), 4.32 (2H, s), 4.24 (2H, s), 3.32 (3H, s). MS (ESI+): 535.2. HPLC (Condition B): Rt 4.16 min (HPLC purity 99.7%).

### Example 36

### N-benzyl-4-{[benzyl[2-chloro pyridin-3-yl]sulfonyl)amino]methyl}benzamide

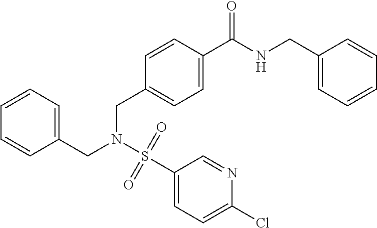

Following the general method as outlined in Example 8, starting from 4-({benzyl[(4-chloropyridin-3-yl)sulfonyl]amino}methyl)benzoic acid (Intermediate 47, 50 mg; 0.106 mmol) and benzylamine (Aldrich, 0.013 ml; 0.106 mmol), the title compound was obtained as a white solid.

¹H NMR (DMSO-d6, 400 MHz): δ 8.97 (1H, m), 8.84-8.85 (1H, d), 8.26-8.29 (1H, d), 7.72-7.75 (3H, m), 7.21-7.31 (4H, m), 7.18-7.20 (2H, m), 7.13-7.18 (2H, m), 4.44-4.45 (2H, d), 4.39 (2H, s). MS (ESI+): 506.1. HPLC (Condition B): Rt 3.90 min (HPLC purity 99.8%).

### Example 37

### 4-{[benzyl({[2-chloropyridine)l]sulfonyl)amino]methyl}-N(cyclopropylmethyl)benzamide

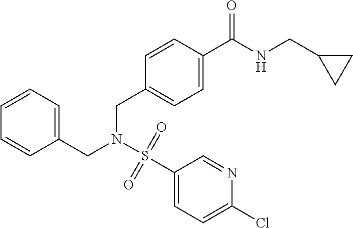

Following the general method as outlined in Example 8, starting from 4-({benzyl[(4-chloropyridin-3-yl)sulfonyl]amino}methyl)benzoic acid (Intermediate 47, 50 mg; 0.106 mmol) and cyclopropane methylamine (Aldrich, 0.014 ml; 0.106 mmol), the title compound was obtained as a white solid.

¹H NMR (DMSO-d6, 400 MHz): δ 8.85-8.86 (1H, d), 8.47-8.48 (1H, t), 8.27-8.30 (1H, d), 7.69-7.75 (3H, m), 7.12-7.19 (7H, m), 4.43 (2H, s), 4.38 (2H, s), 3.09-3.12 (2H, t), 1.00-1.16 (1H, m), 0.38-0.43 (2H, m), 0.18-0.22 (2H, m). MS (ESI+): 470.1. HPLC (Condition B): Rt 6.58 min (HPLC purity 95.1%).

### Example 38

### 4-{[(4-Chloro-benzenesulfonyl)-(3-methoxybenzyl)-amino]-methyl}-N-phenyl methyl benzamide

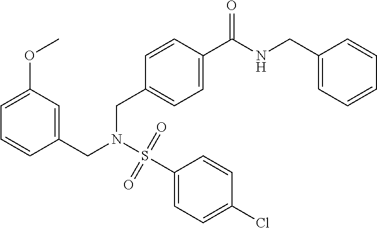

Following the general method as outlined for Example 36, starting from 4-{[[(4-chlorophenyl) sulfonyl] (3-methoxybenzyl)amino]methyl}benzoic acid (Intermediate 49; 100 mg, 0.24 mmol) and benzyl amine (0.025 ml, 0.23 mmol), the title compound was obtained as white solid.

¹H NMR (DMSO-d6, 400 MHz): δ 8.95-8.98 (1H, t), 7.89-7.91 (2H, t), 7.74-7.76 (2H, d), 7.67-7.69 (2H, t), 7.28-7.33 (4H, m), 7.11-7.25 (4H, m), 6.73-6.74 (1H, d), 6.66-6.68 (1H, d), 6.54 (1H, s), 4.44-4.46 (2H, d), 4.36 (2H, s), 4.29 (2H, s), 3.59 (3H, s). MS (ESI+): 535.2. HPLC (Condition B): Rt 4.16 min (HPLC purity 99.9%).

### Example 39

### 4-{[[(4-chlorophenyl)sulfonyl](4-chlorobenzyl)amino]methyl}-N-phenyl methyl benzamide

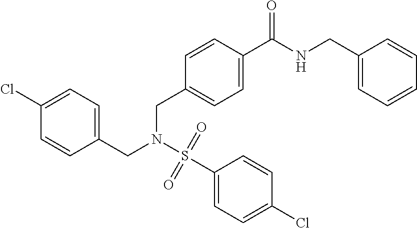

Following the general method as outlined for Example 36, starting from 4-{[[(4-chlorophenyl)sulfonyl](4-chlorobenzyl)amino]methyl}benzoic acid (Intermediate 51, 100 mg, 0.22 mmol) and benzyl amine (0.027 ml, 0.22 mmol), the title compound was obtained as an off white solid.

¹H NMR (DMSO-d6, 400 MHz): δ 8.95-8.98 (1H, t), 7.89-7.91 (2H, d), 7.73-7.71 (2H, d), 7.67-7.73 (4H, m), 7.21-7.33 (7H, m), 7.10-7.20 (4H, m), 4.40-4.45 (2H, d), 4.36 (2H, s), 4.31 (2H, s). MS (ESI+): 539.0. HPLC (Condition B): Rt 4.35 min (HPLC purity 91.9%).

### Example 40

### 4-{[(4-Chloro-benzenesulfonyl)-pyridin-2-ylmethyl-amino]-methyl}-N-(1-phenyl-cyclopropyl)-benzamide

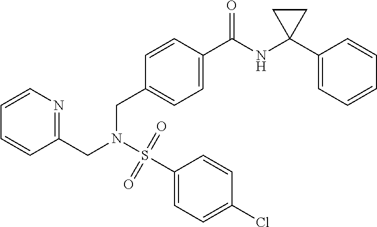

Following the general method as outlined for Example 36, starting from 4-({[(4-chlorophenyl) sulfonyl](pyridin-2-ylmethyl)amino}methyl)benzoic acid (Intermediate 5, 100 mg, 0.24 mmol) and 1-phenyl cyclopropylamine (44 mg, 0.26 mmol), the title compound was obtained as a white solid.

¹H NMR (DMSO-d6, 400 MHz): δ 9.11 (1H, s), 8.32-8.34 (1H, d), 7.82-7.84 (2H, d), 7.76-7.80 (2H, d), 7.61-7.63 (3H, m), 7.24-7.27 (4H, m), 7.12-7.121 (5H, m), 4.51 (2H, s), 4.41 (2H, s), 1.24 (4H, s). MS (ESI+): 532.0. HPLC (Condition B): Rt 3.23 min (HPLC purity 99.3%).

### Example 41

### N-benzyl-3,4-dichloro-N-[4-(2H-tetrazol-5-yl)benzyl]benzenesulfonamide

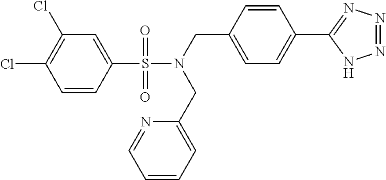

Following the general method as outlined in Example 17, starting from N-benzyl-3,4-dichloro-N-(4-cyanobenzyl)benzene sulfonamide (Intermediate 53, 150 mg, 0.34 mmol), the title compound was obtained as a white solid.

¹H NMR (DMSO-d6, 400 MHz): δ8.30-8.32 (1H, t), 7.92-7.96 (3H, m), 7.81 (2H, s), 7.63-7.67 (1H, t), 7.42-7.44 (2H, d), 7.22-7.24 (1H, d), 7.17-7.20 (1H, m), 4.61 (2H, s), 4.51 (2H, s). MS (ESI+): 451.2. HPLC (Condition B): Rt 2.82 min (HPLC purity 96.8%).

### Example 42

### 4-Ethoxy-N-(pyridin-2-ylmethyl)-N-[4-(2H-tetrazol-5-yl)benzyl]benzenesulfonamide

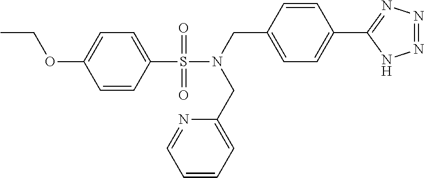

Following the general method as outlined in Example 17, starting from N-(4-cyanobenzyl)-4-ethoxy-N-(pyridin-2-ylmethyl)benzene sulfonamide (Intermediate 54, 500 mg, 1.20 mmol), the title compound was obtained as a white solid in 62% yield.

¹H NMR (DMSO-d6, 400 MHz): δ8.30-8.34 (1H, t), 7.86-7.88 (2H, d), 7.76-7.78 (2H, d), 7.59-7.63 (1H, m), 7.35-7.37 (2H, d), 7.14-7.21 (2H, m), 7.06-7.08 (2H, d), 4.46 (2H, s), 4.39 (2H, s), 4.09-4.14 (2H, m), 1.33-1.36 (3H, t). MS (ESI+): 451.2. HPLC (Condition B): Rt 4.82 min (HPLC purity 97.5%).

### Example 43

### 4-{[[(4-cyanophenyl)sulfonyl](pyridin-2-ylmethyl)amino]}-N-(cyclopropylmethyl)benzamide

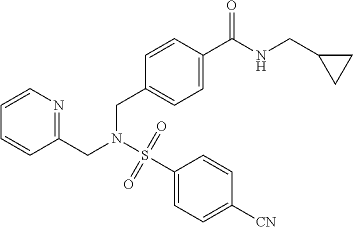

Following the general method as outlined in Example 35, starting from 4-{[[(4-cyanophenyl)sulfonyl](pyridin-2-ylmethyl)amino]methyl}benzoic acid (Intermediate 57, 60 mg, 0.15 mmol) and cyclopropyl methylamine hydrochloride (Aldrich, 0.018 ml; 0.16 mmol), the title compound was obtained as a white solid.

¹H NMR (DMSO-d6, 400 MHz): δ 8.49-8.52 (1H, t), 8.28-8.29 (1H, d), 7.98-8.04 (4H, m), 7.72-7.74 (2H, d), 7.62-7.66 (1H, m), 7.23-7.25 (2H, d), 7.16-7.20 (2H, d), 4.55 (2H, s), 4.44 (2H, s), 3.09-3.12 (2H, m), 1.0 (1H, m), 0.42-0.39 (2H, m), 0.20-0.22 (2H, m). MS (ESI+): 461.0. HPLC (Condition B): Rt 3.75 min (HPLC purity 99.4%).

### Example 44

### 4-{[[(4-cyanophenyl)sulfonyl](pyridin-2-ylmethyl)amino]}-N-(1-phenyl cyclopropyl methyl)benzamide

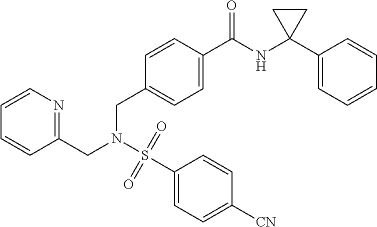

Following the general method as outlined in Example 35, starting from 4-{[[(4-cyanophenyl)sulfonyl]pyridin-2-ylmethyl)amino]methyl}benzoic acid (Intermediate 57, 60 mg, 0.15 mmol) and 1-phenyl cyclopropyl amine hydrochloride (Aldrich, 28 mg; 0.16 mmol), the title compound was obtained as a white solid.

¹H NMR (DMSO-d6, 400 MHz): δ 9.13 (1H, s), 8.29 (1H, d), 7.97-8.03 (4H, m), 7.77-7.79 (2H, d), 7.63-7.67 (1H, t), 7.24-7.26 (4H, m), 7.12-7.19 (5H, m), 4.56 (2H, s), 4.45 (2H, s), 1.24 (4H, s). MS (ESI+): 523.0. HPLC (Condition B): Rt 4.18 min (HPLC purity 98.0%).

### Example 45

### 4-{[[(3,4-dichlorophenyl)sulfonyl](pyridin-2-ylmethyl)amino]}-N-(cyclopropyl methyl)benzamide

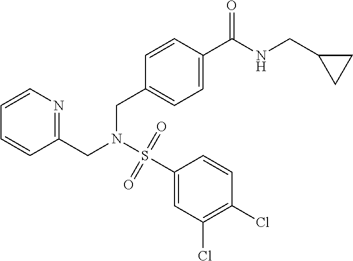

Following the general method as outlined in Example 35, starting from 4-{[[(3,4-dichlorophenyl)sulfonyl](pyridin-2-ylmethyl)amino]methyl}benzoic acid (Intermediate 59, 100 mg, 0.22 mmol) and cyclopropyl methylamine hydrochloride (0.028 ml; 0.28 mmol), the title compound was obtained as a white solid.

¹H NMR (DMSO-d6, 400 MHz): δ 8.49-8.52 (1H, t), 8.30-8.32 (1H, m), 7.95 (1H, d), 7.74-7.80 (4H, m), 7.64-7.68 (1H, m), 7.27-7.29 (2H, d), 7.18-7.22 (2H, m), 4.57 (2H, s), 4.46 (2H, s), 3.10-3.13 (2H, t), 0.99-1.02 (1H, m), 0.39-0.43 (2H, m), 0.19-0.39 (2H, m). MS (ESI+): 504.0. HPLC (Condition B): Rt 4.34 min (HPLC purity 99.5%).

### Example 46

### 4-{[[(4-cyanophenyl)sulfonyl](pyridin-2-ylmethyl)amino]}-N-(1-phenyl cyclopropyl methyl)benzamide

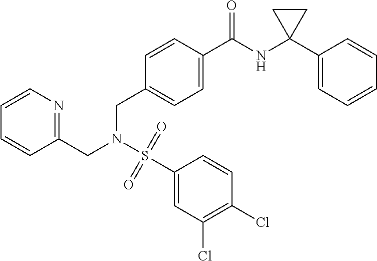

Following the general method as outlined in Example 35, starting from 4-{[[(3,4-dichlorophenyl)sulfonyl](pyridin-2-ylmethyl)amino]methyl}benzoic acid (Intermediate 59, 100 mg, 0.22 mmol) and 1-phenylcyclopropyl amine hydrochloride (75 mg, 0.44 mmol), the title compound was obtained as a white solid.

¹H NMR (DMSO-d6, 400 MHz): δ 9.13 (1H, s), 8.32-8.33 (1H, d), 7.93 (1H, s), 7.79-7.81 (4H, m), 7.65-7.70 (1H, m), 7.28-7.30 (2H, d), 7.19-7.25 (4H, m), 7.12-7.17 (3H, m), 4.57 (2H, s), 4.46 (2H, s), 1.24 (4H, s). MS (ESI+): 566.0. HPLC (Condition B): Rt 4.72 min (HPLC purity 94.2%).

### Example 47

### 4-cyano-N-(pyridin-2-ylmethyl)-N-[4-(2H-tetrazol-5-yl)benzyl]benzenesulfonamide

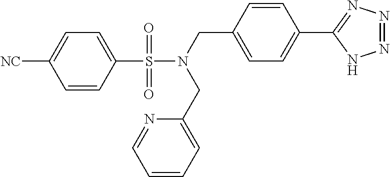

Following the general method as outlined for Intermediate 14, starting from N-(pyridin-2-ylmethyl)-N-[4-(2H-tetrazol-5-yl)benzyl]amine (Intermediate 60, 100 mg, 0.37 mmol) and 4-cyanobenzenesulfonylchloride (75 mg, 0.37 mmol), the title compound was obtained as off white solid.

¹H NMR (DMSO-d6, 400 MHz): δ 8.28 (1H, s), 7.99-8.04 (4H, m), 7.89-7.91 (2H, d), 7.61-7.64 (1H, t), 7.36-7.38 (2H, d), 7.18-7.20 (2H, d), 4.58 (2H, s), 4.48 (2H, s). MS (ESI−): 429.9. HPLC (Condition B): Rt 3.52 min (HPLC purity 90.3%).

### Example 48

### N-(methanesulfonyl)-4{[[(4-chlorophenyl)sulfonyl](pyridin-2-ylmethyl)amino]methyl}benzamide

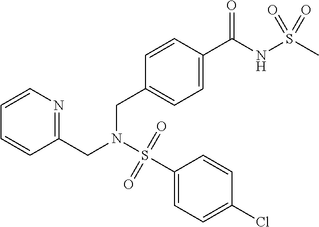

Following the general method as outlined in Example 12, starting from 4-{[[(4-chlorophenyl)sulfonyl](pyridin-2-ylmethyl)amino]methyl}benzoic acid (Intermediate 5, 200 mg; 0.479 mmol) and methane sulphonamide (50 mg; 0.52 mmol), the title compound was obtained as yellow solid.

¹H NMR (DMSO-d6, 400 MHz): δ 8.32-8.34 (1H, d), 7.72-7.77 (4H, m), 7.64-7.68 (1H, t), 7.34-7.49 (2H, d), 7.19-7.27 (4H, m), 4.54 (2H, s), 4.53 (2H, s), 3.25 (3H, s). MS (ESI+): 493.9. HPLC (Condition B): Rt 3.79 min (HPLC purity 95.8%).

### Example 49

### N-(cyclopropanesulfonyl)-4-{[[(4-chlorophenyl)sulfonyl](pyridin-2-ylmethyl)amino]methyl}benzamide

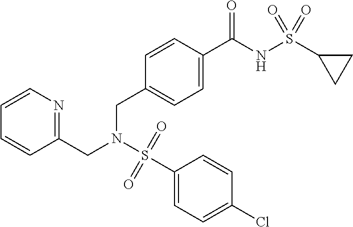

Following the general method as outlined in Example 12, starting from 4-{[[(4-chlorophenyl)sulfonyl](pyridin-2-ylmethyl)amino]methyl}benzoic acid (Intermediate 5, 200 mg; 0.479 mmol) and cyclopropane sulfonamide (63 mg, 0.52 mmol), the title compound was obtained as yellow solid.

¹H NMR (DMSO-d6, 400 MHz): δ 12.04 (1H, s), 8.31-8.32 (1H, t), 7.79-7.85 (4H, m), 7.61-7.65 (3H, m), 7.27-7.29 (2H, d), 7.17-7.27 (2H, m), 4.53 (2H, s), 4.42 (2H, s), 3.06-3.10 (1H, m), 1.02-1.09 (4H, m). MS (ESI+): 520.0. HPLC (Condition B): Rt 4.01 min (HPLC purity 93.3%).

### Example 50

### N-(3-nitrophenylmethanesulfonyl)-4-{[[(4-chlorophenyl)sulfonyl](pyridin-2-yl-methyl)amino]methyl}benzamide

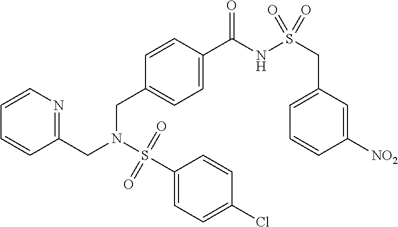

Following the general method as outlined in Example 12, starting from 4-{[[(4-chlorophenyl)sulfonyl](pyridin-2-ylmethyl)amino]methyl}benzoic acid (Intermediate 5, 200 mg; 0.479 mmol) and (3-nitrophenyl)-methane sulfonamide (114 mg, 0.53 mmol), the title compound was obtained as off white solid.

¹H NMR (DMSO-d6, 400 MHz): δ 829-8.31 (1H, m), 821-8.24 (2H, m), 7.82-7.84 (2H, d), 7.74-7.76 (3H, m), 7.65-7.69 (1H, d), 7.59-7.65 (3H, m), 727-7.29 (2H, d), 7.16-7.18 (2H, d), 5.05 (2H, s), 4.52 (2H, s), 4.42 (2H, s). MS (ESI+): 615.0. HPLC (Condition B): Rt 4.48 min (HPLC purity 99.7%).

### Example 51

### N-(3-fluorobenzenesulfonyl)-4-{[[(4-chlorophenyl)sulfonyl](pyridin-2-yl-methyl)amino]methyl}benzamide

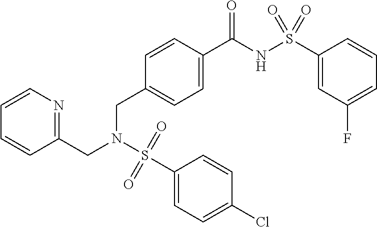

Following the general method as outlined in Example 12, starting from 4-{[[(4-chlorophenyl)sulfonyl](pyridin-2-ylmethyl)amino]methyl}benzoic acid (Intermediate 5, 200 mg; 0.479 mmol) and 3-fluorobenzenesulfonamide (92 mg, 0.53 mmol), the title compound was obtained as a white solid.

¹H NMR (DMSO-d6, 400 MHz): δ 8.29-8.31 (1H, d), 7.82-7.84 (2H, d), 7.73-7.75 (3H, d), 7.66 7.69 (5H, m), 7.46-7.50 (1H, m) 7.16-7.19 (4H, d), 4.48 (2H, s), 4.39 (2H, s). MS (ESI+): 574.0. HPLC (Condition B): Rt 4.45 min (HPLC purity 98.8%).

### Example 52

### N-(3-pyridylsulfonyl)-4-{[[(4-chlorophenyl)sulfonyl](pyridin-2-ylmethyl)amino]methyl}benzamide

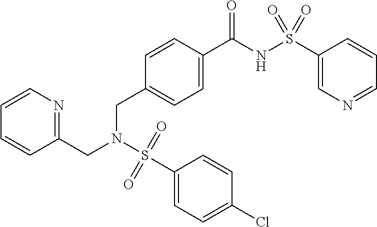

Following the general method as outlined in Example 12, starting from 4-{[[(4-chlorophenyl)sulfonyl](pyridin-2-ylmethyl)amino]methyl}benzoic acid (Intermediate 5, 200 mg; 0.479 mmol) and pyridine-3-sulfonamide (88 mg; 0.52 mmol), the title compound was obtained as a white powder.

¹H NMR (DMSO-d6, 400 MHz): δ 9.06-9.07 (1H, d), 8.81-8.82 (1H, d), 8.29-8.32 (2H, m), 7.81-7.83 (2H, d), 7.73-7.75 (2H, d), 7.59-7.65 (4H, m), 7.22-7.25 (2H, d), 7.15-7.18 (2H, m), 4.54 (2H, s), 4.49 (2H, s). MS (ESI+): 556.9. HPLC (Condition B): Rt 3.84 min (HPLC purity 90.1%).

### Example 53

### N-(1-methylsulfonyl-3-propylsulfonyl)-4-{[[(4-chlorophenyl)sulfonyl](pyridin-2-yl-methyl)amino]methyl}benzamide

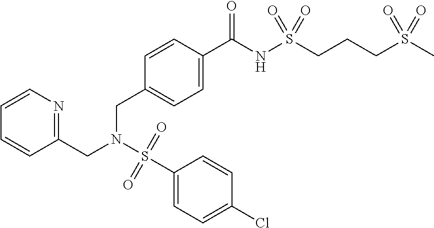

Following the general method as outlined in Example 12, starting from 4-{[[(4-chlorophenyl)sulfonyl](pyridin-2-ylmethyl)amino]methyl}benzoic acid (Intermediate 5, 200 mg; 0.479 mmol) and 3-methyl sulfonyl-1-propane sulfonamide (105 mg, 0.52 mmol), the title compound was obtained as a yellow solid.

¹H NMR (DMSO-d6, 400 MHz): δ 8.30-8.31 (1H, t), 7.80-7.85 (4H, m), 7.64-7.60 (3H, m), 7.30-7.32 (2H, d), 7.16-7.19 (2H, m), 4.53 (2H, s), 4.42 (2H, s), 3.63-3.67 (2H, t), 3.27-3.32 (2H, t), 2.97 (3H, s), 2.61-2.76 (1H, m), 2.46-2.49 (1H, m). MS (ESI+): 600.0. HPLC (Condition B): Rt 2.64 min (HPLC purity 93.1%).

### Example 54

### N-(3-methoxy-propane-1-sulfonyl)-4-{[[(4-chlorophenyl)sulfonyl](pyridin-2-yl-methyl)amino]methyl}benzamide

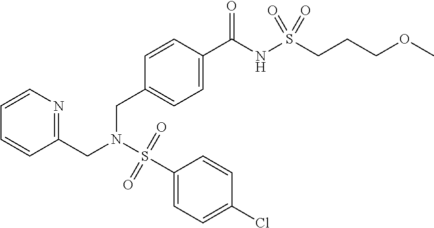

Following the general method as outlined in Example 12, starting from 4-{[[(4-chlorophenyl)sulfonyl](pyridin-2-ylmethyl)amino]methyl}benzoic acid (Intermediate 5, 200 mg; 0.479 mmol) and 3-methoxy propane sulfonamide (80 mg, 0.53 mmol), the title compound was obtained as an orange gum.

¹H NMR (DMSO-d6, 400 MHz): δ 12.05 (1H, brs), 8.30-8.31 (1H, d), 7.79-7.84 (4H, m), 7.61-7.63 (3H, m), 7.29-7.31 (2H, d), 7.16-7.19 (2H, d), 4.53 (2H, s), 4.42 (2H, s), 3.7 (2H, m), 3.45 (2H, t), 3.15 (3H, s), 1.88 (2H, t). MS (ESI+): 552. HPLC (Condition B): Rt 2.85 min (HPLC purity 95.3%).

### Example 55

### N-(ethanesulfonyl)-4-{[[(4-chlorophenyl)sulfonyl](pyridin-2-yl-methyl)amino]methyl}benzamide

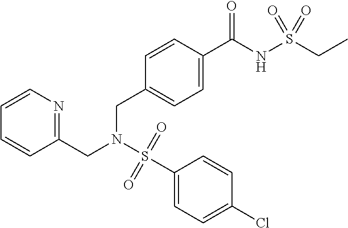

Following the general method as outlined in Example 12, starting from 4-{[[(4-chlorophenyl)sulfonyl](pyridin-2-ylmethyl)amino]methyl}benzoic acid (Intermediate 5, 200 mg; 0.479 mmol) and ethane sulfonamide (57 mg, 0.53 mmol), the title compound was obtained as off white solid.

¹H NMR (DMSO-d6, 400 MHz): δ 8.30-8.31 (1H, d), 7.80-7.84 (4H, m), 7.60-7.65 (3H, m), 7.29-7.31 (2H, d), 7.16-7.19 (2H, d), 4.53 (2H, s), 4.42 (2H, s) 3.46-3.51 (2H, q) 1.21-1.25 (3H, t). MS (ESI+): 507.9. HPLC (Condition B): Rt 5.20 min (HPLC purity 98.2%).

### Example 56

### 4-chloro-N-[3-fluoro-4-(1H-tetrazol-5-yl)benzyl]-N-(pyridin-2 yl methyl)benzene sulfonamide

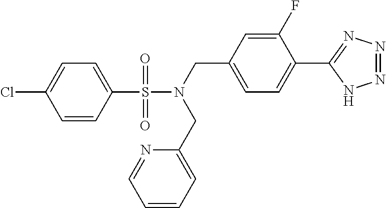

Following the general method as outlined in Example 17, starting from 4-chloro-N-(4-cyano-3-fluorobenzyl)-N-(pyridine-2-yl-methyl)benzene sulfonamide (Intermediate 61, 500 mg, 1.20 mmol), the title compound was obtained as a white solid.

¹H NMR (DMSO-d6, 400 MHz): δ 8.42-8.44 (1H, d), 7.85-7.93 (4H, m), 7.70-7.69 (2H, d) 7.67-7.69 (2H, d), 7.35-7.39 (2H, m), 4.56 (2H, s), 4.58 (2H, s). MS (ESI+): 458.9. HPLC (Condition B): Rt 5.05 min (HPLC purity 93.1%).

### Example 57

### 4-chloro-N-[2-fluoro-4-(1H-tetrazol-5-yl)benzyl]-N-(pyridin-2 yl methyl)benzene sulfonamide

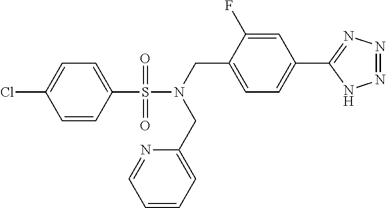

Following the general method as outlined in Example 17, starting from 4-chloro-N-(4-cyano-2-fluorobenzyl)-N-(pyridine-2-yl-methyl)benzene sulfonamide (Intermediate 62, 370 mg, 0.89 mmol), the title compound was obtained as a white solid.

¹H NMR (DMSO-d6, 400 MHz): δ 8.36-8.37 (1H, d), 7.85-7.87 (2H, d), 7.66-7.68 (2H, m), 7.66-7.64 (3H, m), 7.51-7.53 (1H, t), 6.98-7.02 (1H, m), 7.35-7.49 (1H, d), 7.27-7.33 (1H, m), 4.60 (2H, s), 4.54 (2H, s). MS (ESI−): 456.8. HPLC (Condition B): Rt 3.91 min (HPLC purity 97.2%).

### Example 58

### 4-chloro-N-[(3,5-dimethylisoxazol-4-yl)methyl]-N-[4-(2H-tetrazol-5-yl)benzyl]benzene sulfonamide

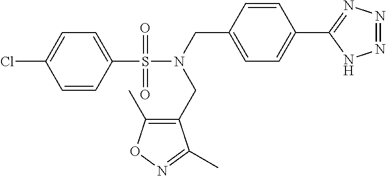

Following the general method as outlined in Example 17, starting from 4-chloro-N-(4-cyanobenzyl)-N-[(3,5-dimethylisoxazol-4-ylmethyl]benzenesulfonamide (Intermediate 63, 250 mg, 0.6 mmol), the title compound was obtained as a white solid.

¹H NMR (DMSO-d6, 400 MHz): δ7.92-7.95 (2H, m), 7.86-7.88 (2H, d), 7.73-7.75 (2H, m), 7.30-7.32 (2H, d), 4.34 (2H, s), 4.19 (2H, s), 2.13 (3H, s), 2.03 (3H, s). MS (ESI−): 456.8. HPLC (Condition B): Rt 3.23 min (HPLC purity 98.2%).

### Example 59

### 4-chloro-N-(1,3-oxazol-2-ylmethyl)-N-[4-(2H-tetrazol-5-yl)benzyl]benzene sulfonamide

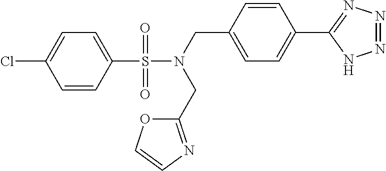

Following the general method as outlined in Example 17, starting from 4-chloro-N-(4-cyanobenzyl)-N-[(1,3-oxazol-2-yl)methyl]benzenesulfonamide (Intermediate 64, 160 mg, 0.40 mmol), the title compound was obtained as a white solid.

¹H NMR (DMSO-d6, 400 MHz): δ7.96-7.98 (2H, d), 7.88 (1H, s), 7.82-7.84 (2H, t), 7.64-7.67 (2H, t), 7.43-7.45 (2H, d), 7.00 (1H, s), 4.51 (2H, s), 4.49 (2H, s). MS (ESI−): 430.9. HPLC (Condition B): Rt 3.19 min (HPLC purity 98.2%).

### Example 60

### 4-chloro-N-(2,4-difluorobenzyl)-N-[4-(2H-tetrazol-5-yl)benzyl]benzenesulfonamide

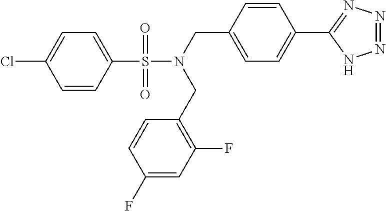

Following the general method as outlined in Example 17, starting from 4-chloro-N-(4-cyanobenzyl)-N-(4-methoxybenzyl)benzene sulfonamide (Intermediate 66, 200 mg, 0.46 mmol), the title compound was obtained as a white solid.

¹H NMR (DMSO-d6, 400 MHz): δ7.89-7.91 (2H, d), 7.84-7.86 (2H, d), 7.69-7.71 (2H, d), 7.28-7.32 (3H, t), 6.89-7.01 (2H, m), 4.42 (2H, s), 4.38 (2H, s). MS (ESI−): 474.0. HPLC (Condition B): Rt 3.74 min (HPLC purity 99.5%).

### Example 61

### 4-chloro-N-(5-chloro-2-fluorobenzyl)-N-[4-(2H-tetrazol-5-yl)benzyl]benzene sulfonamide

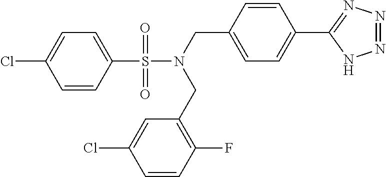

Following the general method as outlined in Example 17, starting from 4-chloro-N-(5-chloro-2-fluorobenzyl)-N-(4-cyanobenzyl)benzene sulfonamide (Intermediate 67, 200 mg; 0.44 mmol), the title compound was obtained as a white solid in 60% yield.

¹H NMR (DMSO-d₆, 400 MHz) δ 7.90-7.94 (2H, m), 7.85-7.87 (2H, d), 7.69-7.72 (2H, m), 7.34-7.36 (2H, d), 7.19-7.23 (1H, m), 7.09-7.11 (1H, m), 6.98-7.03 (1H, t), 4.46 (2H, s), 4.41 (2H, s). MS (ESI−): 491.8. HPLC (Condition B): Rt 3.79 min (HPLC purity 97.5%).

### Example 62

### 4-chloro-N-(2,6-difluorobenzyl)-N-[4-(2H-tetrazol-5-yl)benzyl]benzene sulfonamide

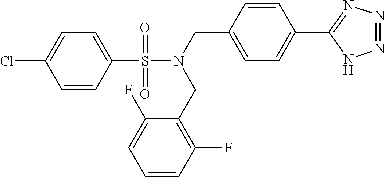

Following the general method as outlined in Example 17, starting from 4-chloro-N-(4-cyanobenzyl)-N-(2,6-difluorobenzyl)benzene sulfonamide (Intermediate 68, 200 mg; 0.46 mmol), the title compound was obtained as a white solid.

¹H NMR (DMSO-d₆, 400 MHz) δ 7.84-7.86 (2H, d), 7.67-7.69 (1H, d), 7.34-7.36 (1H, d), 7.20-7.26 (1H, m), 6.83-6.87 (1H, t), 4.42 (2H, s), 4.40 (2H, s). MS (ESI−): 474.0. HPLC (Condition B): Rt min (HPLC purity %).

### Example 63

### 4-chloro-N-(2-chlorobenzyl)-N-[4-(2H-tetrazol-5-yl)benzyl]benzenesulfonamide

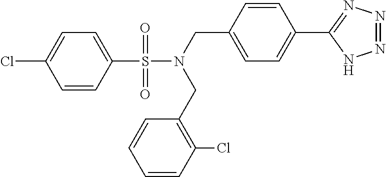

Following the general method as outlined in Example 17, starting from 4-chloro-N-(2-chlorobenzyl)-N-(4-cyanobenzyl)benzenesulfonamide (Intermediate 69, 300 mg; 0.69 mmol), the title compound was obtained as a white solid.

¹H NMR (DMSO-d6, 400 MHz): δ7.92-7.94 (2H, d), 7.80-7.82 (2H, d), 7.70-7.72 (2H, d), 7.28-7.32 (3H, t), 7.14-7.24 (3H, m), 4.47 (2H, s), 4.44 (2H, s). MS (ESI−): 471.9. HPLC (Condition B): Rt 3.84 min (HPLC purity 98.0%).

### Example 64

### 4-{[(4-Chloro-benzenesulfonyl)-pyridin-2-ylmethyl-amino]-methyl}-2-fluoro-benzoic acid

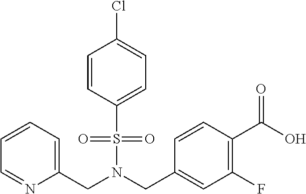

Following the general method as outlined for Intermediate 41, starting from methyl 4-{[(4-chloro-benzenesulfonyl)-pyridin-2-ylmethyl-amino]-methyl}-2-fluoro-benzoate (intermediate 74, 140 mg; 0.31 mmol), the title compound was obtained as white solid in 89% yield.

¹H NMR (DMSO-d6, 400 MHz): δ 13.17 (1H, brs), 8.30-8.31 (1H, d), 7.83-7.86 (2H, m), 7.69-7.73 (1H, t), 7.61-7.65 (3H, m), 7.16-7.22 (2H, m), 7.08-7.10 (1H, m), 7.00-7.03 (1H, d), 4.51 (2H, s), 4.45 (2H, s). MS (ESI−): 432.6. HPLC (Condition B): Rt 2.74 min (HPLC purity 99.5%).

### Example 65

### 4-{[(4-Chloro-benzenesulfonyl)-(2-fluoro-benzyl)-amino]-methyl}-2-fluorobenzoic acid

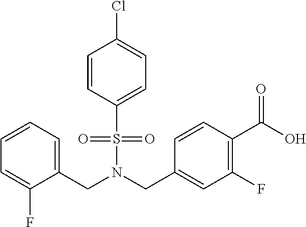

Following the general method as outlined for Intermediate 41, starting from methyl 4-{[(4-Chloro benzene sulfonyl)-(2-fluoro-benzyl)-amino]-methyl}-2-fluoro-benzoate (intermediate 75, 200 mg; 0.43 mmol), the title compound was obtained as white solid in 70% yield.

¹H NMR (DMSO-d6, 400 MHz): δ 7.88-7.89 (2H, t), 7.68-7.70 (2H, m), 7.60-7.64 (1H, t), 7.19-7.25 (2H, m), 6.95-7.04 (3H, m), 6.88-6.91 (1H, d), 4.40 (2H, s), 4.38 (2H, s). MS (ESI−): 449.8. HPLC (Condition B): Rt 3.83 min (HPLC purity 99.0%).

### Example 66

### 4-{[(4-Ethoxy-benzenesulfonyl)-pyridin-2-ylmethyl-amino]-methyl}-2-fluoro-benzoic acid

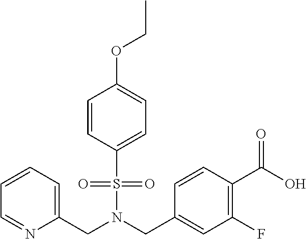

Following the general method as outlined for Intermediate 41, starting from methyl 4-{[(4-Ethoxy-benzenesulfonyl)-pyridin-2-ylmethyl-amino]-methyl}-2-fluoro-benzoate (intermediate 77, 250 mg; 0.54 mmol), the title compound was obtained as white solid in 71% yield.

¹H NMR (DMSO-d6, 400 MHz): δ 13.18 (1H, brs), 8.33-8.34 (1H, t), 7.75-7.78 (2H, d), 7.64-7.68 (1H, m), 7.60-7.63 (1H, m), 7.15-7.22 (2H, m), 7.06-7.08 (3H, d), 6.97-7.00 (1H, d), 4.43 (2H, s), 4.39 (2H, s), 4.09-4.14 (2H, q), 1.33-1.36 (3H, t). MS (ESI−): 443.0. HPLC (Condition B): Rt 5.0 min (HPLC purity 97.7%).

### Example 67

### 4-{[[(4-cyanophenyl)sulfonyl](pyridin-2-ylmethyl)amino]methyl}-2-fluorobenzoic acid

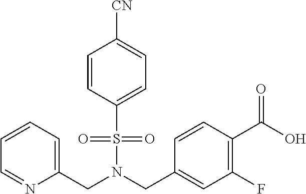

Following the general method as outlined for Intermediate 41, starting from methyl 4-{[[(4-cyanophenyl)sulfonyl](pyridin-2-ylmethyl)amino]methyl}-2-fluorobenzoate (intermediate 79, 200 mg; 0.45 mmol), the title compound was obtained as white solid.

¹H NMR (DMSO-d6, 400 MHz): 813.23 (1H, s), 8.26-8.28 (1H, m), 7.98-8.05 (4H, m), 7.70-7.74 (1H, t), 7.61-7.66 (1H, m), 7.11-7.21 (2H, m), 7.08-7.10 (1H, d), 7.01-7.04 (1H, d), 4.56 (2H, s), 4.50 (2H, s). MS (ESI+): 426.0. HPLC (Condition B): Rt 4.84 min (HPLC purity 98.5%).

### Example 68

### 4-chloro-N-(2-methyl-thiazol-4-ylmethyl)-N-[4-(2H-tetrazol-5yl)benzyl]benzenesulfonamide

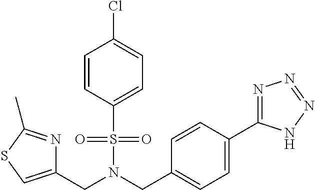

Following the general method as outlined in Intermediate 14, starting from 4-Chloro-N-(4-cyano-benzyl)-N-(2-methyl-thiazol-4-ylmethyl)-benzenesulfonamide (intermediate 80, 200 mg, 0.47 mmol), the title compound was obtained as a white solid.

¹H NMR (DMSO-d6, 400 MHz): δ 7.95-7.97 (2H, s), 7.77-7.79 (2H, m), 7.59-7.62 (2H, m), 7.44-7.46 (2H, d), 7.21 (1H, s), 4.52 (2H, s), 4.35 (2H, s), 2.41 (3H, s). MS (ESI−): 458.9. HPLC (Condition B): Rt 3.40 min (HPLC purity 98.1%).

### Example 69

### 4-chloro-N-(5-tert-butyl-1,2,4-oxadiazol-3-ylmethyl)-N-[4-(2H-tetrazol-5yl)benzyl]benzene sulfonamide

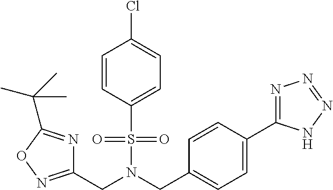

Following the general method as outlined in Intermediate 14, starting from 4-Chloro-N-(4-cyano-benzyl)-N-(5-tert-butyl-1,2,4-oxadiazol-3-ylmethyl)benzenesulfonamide (intermediate 81, 300 mg, 0.68 mmol), the title compound was obtained as off white solid.

¹H NMR (DMSO-d6, 400 MHz): δ 7.98-8.00 (2H, d), 7.84-7.86 (2H, d), 7.64-7.66 (2H, d) 7.49-7.51 (2H, d), 4.58 (2H, s), 4.48 (2H, s), 1.18 (9H, s). MS (ESI−): 485.9. HPLC (Condition B): Rt 3.83 min (HPLC purity 99.9%).

### Example 70

### 4-chloro-N-(2-fluoro-4-chloro benzyl)-N-[4-(2H-tetrazol-5yl)benzyl]benzene sulfonamide

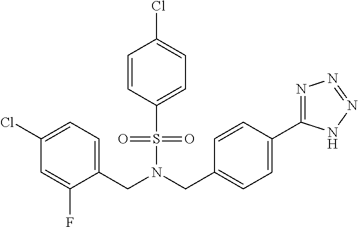

Following the general method as outlined in Intermediate 14, starting from 4-Chloro-N-(4-cyano-benzyl)-N-(5-tert-butyl-1,2,4-oxadiazol-3-ylmethyl)benzenesulfonamide (intermediate 82, 300 mg, 0.67 mmol), the title compound was obtained as white solid in 78% yield.

¹H NMR (DMSO-d6, 400 MHz): δ 7.90-7.91 (2H, d), 7.84-7.89 (2H, d), 7.70-7.71 (2H, d) 7.31-7.33 (2H, d), 7.24-7.28 (1H, d), 7.15-7.18 (1H, d), 7.09-7.12 (1H, d), 4.43 (2H, s), 4.39 (2H, s). MS (ESI−): 491.8. HPLC (Condition B): Rt 3.90 min (HPLC purity 99.7%).

### Example 71

### 4-chloro-N-(pyridin-3-ylmethyl)-N-[4-(2H-tetrazol-5yl)benzyl]benzene sulfonamide

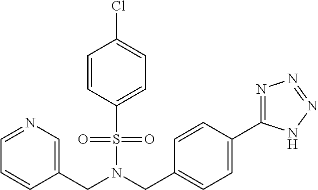

Following the general method as outlined in Intermediate 14, starting from 4-Chloro-N-(4-cyano-benzyl)-N-pyridin-3-ylmethyl-benzenesulfonamide (intermediate 83, 500 mg, 1.25 mmol), the title compound was obtained as brown solid.

¹H NMR (DMSO-d6, 400 MHz): δ 8.31-8.32 (1H, d), 8.27 (1H, s), 7.93-7.95 (2H, d), 7.84-7.86 (2H, d) 7.70-7.72 (2H, d), 7.49-7.51 (1H, d) 7.31-7.33 (2H, d), 7.15-7.19 (1H, m), 4.43 (2H, s), 4.39 (2H, s). MS (ESI−): 439. HPLC (Condition B): Rt 4.71 min (HPLC purity 98.3%).

### Example 73

### 4-chloro-N-(5-methyl-1,2,4-oxadiazol-3-ylmethyl)-N-[4-(2H-tetrazol-5yl)benzyl]benzene sulfonamide

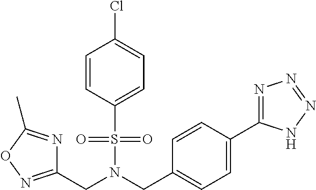

Following the general method as outlined in Intermediate 14, starting from 4-Chloro-N-(4-cyano-benzyl)-N-(5-methyl-1,2,4-oxadiazol-3-ylmethyl)benzenesulfonamide (intermediate 84, 400 mg, 0.99 mmol), the title compound was obtained as yellow solid in 68% yield.

¹H NMR (DMSO-d6, 400 MHz): δ 7.97-7.99 (2H, d), 7.80-7.83 (2H, d), 7.64-7.66 (2H, d) 7.48-7.50 (2H, d), 4.56 (2H, s), 4.46 (2H, s). 2.40 (3H, s). MS (ESI−): 444.0. HPLC (Condition B): Rt 4.41 min (HPLC purity 99.1%).

### Example 74

### 4-chloro-N-(isoquinolin-1-yl methyl)-N-[4-(2H-tetrazol-5yl)benzyl]benzene sulfonamide

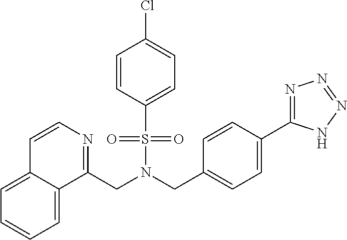

Following the general method as outlined in Intermediate 14, starting from 4-Chloro-N-(4-cyano-benzyl)-N-(isoquinolin-1-ylmethyl)-benzenesulfonamide (intermediate 85, 400 mg, 0.89 mmol), the title compound was obtained as green solid in 90% yield.

¹H NMR (DMSO-d6, 400 MHz): δ 8.30-8.33 (1H, d), 8.16-8.17 (1H, d), 7.88-7.90 (2H, d) 7.78-7.80 (1H, d), 7.59-7.71 (7H, m), 7.12-7.14 (2H, d) 4.99 (2H, s), 4.51 (2H, s). MS (ESI−): 488.8. HPLC (Condition B): Rt 3.92 min (HPLC purity 94.0%).

### Example 75

### 4-chloro-N-(quinolin-1-yl methyl)-N-[4(2H-tetrazol-5yl)benzyl]benzene sulfonamide

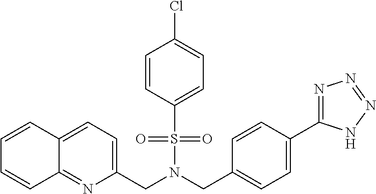

Following the general method as outlined in Intermediate 14, starting from 4-Chloro-N-(4-cyano-benzyl)-N-(quinolin-1-yl methyl)-benzenesulfonamide (intermediate 86, 540 mg, 1.20 mmol), the title compound was obtained as green solid.

¹H NMR (DMSO-d6, 400 MHz): δ 8.30 (1H, s), 8.19-8.30 (1H, m), 7.85-7.91 (5H, m), 7.66-7.67 (2H, d), 7.59-7.61 (2H, d), 7.49-7.53 (1H, m), 7.42-7.44 (2H, d), 7.33-7.35 (1H, d), 4.65 (2H, s), 4.61 (2H, s). MS (ESI−): 489.0. HPLC (Condition B): Rt 3.0 min (HPLC purity 97.3%).

### Example 76

### 4-chloro-N-(isoquinolin-3-yl methyl)-N-[4-(2H-tetrazol-5yl)benzyl]benzene sulfonamide

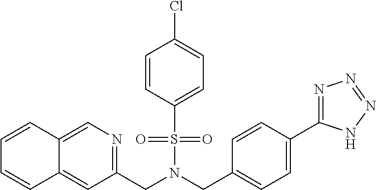

Following the general method as outlined in Intermediate 14, starting from 4-Chloro-N-(4-cyano-benzyl)-N-(isoquinolin-3-yl methyl)-benzenesulfonamide (intermediate 87, 300 mg, 0.67 mmol), the title compound was obtained as green solid.

¹H NMR (DMSO-d6, 400 MHz): δ 8.00-8.02 (2H, d), 7.83-7.89 (1H, m), 7.78-7.80 (4H, d), 7.68-7.72 (1H, m), 7.60-7.62 (1H, m), 7.52-7.59 (3H, m), 7.43-7.46 (2H, d), 4.62 (2H, s), 4.59 (2H, s). MS (ESI−): 488.8. HPLC (Condition B): Rt 3.83 min (HPLC purity 98.8%).

### Example 77

### N-benzyl-2-fluoro-4-chloro-N-[4-(2H-tetrazol-5-yl)benzyl]benzenesulfonamide

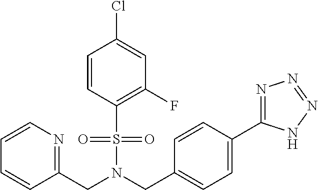

Following the general method as outlined in Intermediate 14, starting from N-benzyl-2-fluoro-4-chloro-N-(4-cyanobenzyl)benzene sulfonamide (intermediate 88, 500 mg, 1.20 mmol), the title compound was obtained as pale brown solid.

¹H NMR (DMSO-d6, 400 MHz): δ8.28-8.29 (1H, d), 7.92-7.94 (2H, d), 7.79-7.81 (1H, t), 7.69-7.71 (1H, d), 7.60-7.64 (1H, t), 7.39-7.42 (3H, d), 7.15-7.18 (2H, d), 4.65 (2H, s), 4.51 (2H, s). MS (ESI−): 457.0. HPLC (Condition B): Rt 3.66 min (HPLC purity 99.1%).

### Example 78

### N-benzyl-2,4-dichloro-N-[4-(2H-tetrazol-5-yl)benzyl]benzenesulfonamide

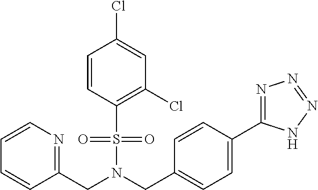

Following the general method as outlined in Intermediate 14, starting from N-benzyl-2,4-dichloro-N-(4-cyanobenzyl)benzene sulfonamide (intermediate 89, 500 mg, 1.16 mmol), the title compound was obtained as green solid.

¹H NMR (DMSO-d6, 400 MHz): δ8.37-8.38 (1H, d), 7.98-8.0 (1H, d), 7.91-7.93 (2H, d), 7.83-7.84 (1H, d), 7.62-7.66 (1H, t), 7.54-7.56 (1H, d), 7.35-7.37 (2H, d), 7.20-7.22 (1H, d),), 7.11-7.13 (1H, d), 4.68 (2H, s), 4.54 (2H, s). MS (ESI+): 474.8. HPLC (Condition B): Rt 3.88 min (HPLC purity 98.0%).

### Example 79

### N-benzyl-2-fluoro-4-chloro-5-methyl-N-[4-(2H-tetrazol-5-yl)benzyl]benzenesulfonamide

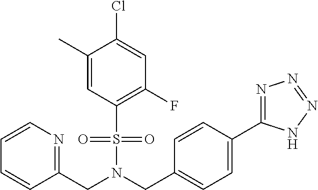

Following the general method as outlined in Intermediate 14, starting from N-benzyl-2-fluoro-4-chloro-5-methyl-N-(4-cyanobenzyl)benzene sulfonamide (intermediate 90, 500 mg, 1.16 mmol), the title compound was obtained as pale green solid.

¹H NMR (DMSO-d6, 400 MHz): δ8.29-8.30 (1H, d), 7.91-7.93 (2H, d), 7.61-7.69 (3H, m), 7.40-7.42 (2H, d), 7.15-7.20 (2H, m), 4.66 (2H, s), 4.53 (2H, s). 2.26 (3H, s). MS (ESI−): 471.0. HPLC (Condition B): Rt 3.87 min (HPLC purity 98.7%).

### Example 80

### 4-Ethoxy-N-(pyridin-2-ylmethyl)-3-fluoro-N-[4-(2H-tetrazol-5-yl)benzyl]benzene sulfonamide

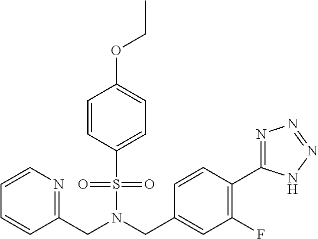

Following the general method as outlined in Intermediate 14, starting from N-(4-cyano-3-fluoro benzyl)-4-ethoxy-N(pyridin2ylmethyl)benzenesulfonamide (intermediate 92, 370 mg; 0.87 mmol), the title compound was obtained as white solid.

¹H NMR (DMSO-d6, 400 MHz): δ 8.34-8.35 (1H, d), 7.80-7.89 (1H, t), 7.76-7.79 (2H, d) 7.61-7.65 (1H, m), 7.10-7.25 (4H, m), 7.07-7.09 (2H, d) 4.47 (2H, s), 4.42 (2H, s), 4.09-4.11 (2H, q), 1.32-1.36 (3H, t). MS (ESI−): 466.8. HPLC (Condition B): Rt 3.46 min (HPLC purity 97.8%).

### Example 81

### 4-cyano-N-(2-fluorobenzyl)-N-[3-fluoro-4-(2H-tetrazol-5-yl)benzyl]benzene sulfonamide

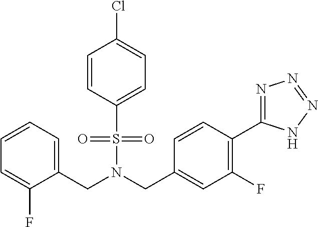

A cooled (0° C.) solution of N-(2-fluorobenzyl)-N-[3-fluoro-4-(2H-tetrazol-5-yl)benzyl]amine (intermediate 93; 160 mg, 0.53 mmol) in dry DMF (15 ml) was treated with triethylamine (0.23 ml; 1.593 mmol) followed by a solution of 4-cyanobenzenesulfonyl chloride (118 mg; 0.58 mmol) in dry DMF (2 mL). The reaction mixture was allowed to warm to room temperature and stirred overnight, quenched with ice, diluted with DCM and washed with 10% aqueous sodium bicarbonate solution and brine. The organic layer was dried over sodium sulphate, concentrated and the crude recrystallized with DCM/hexane to give the title compound as an off-white solid.

¹H NMR (DMSO-d6, 400 MHz): δ 8.05-8.11 (4H, m), 7.80-7.94 (1H, t), 7.18-7.27 (2H, m), 6.95-7.08 (4H, m), 4.47 (2H, s), 4.46 (2H, s). MS (ESI−): 465.0. HPLC (Condition B): Rt 4.58 min (HPLC purity 90.6%).

### Example 82

### 4-chloro-N-(2-fluorobenzyl)-N-[3-fluoro-4-(2H-tetrazol-5-yl)benzyl]benzenesulfonamide

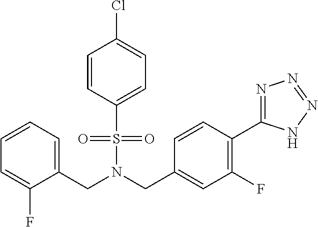

Following the general method as outlined in Intermediate 14, starting from 4-chloro-N-(4-cyano-2-fluorobenzyl)-N-(2-fluorobenzyl)benzenesulfonamide (intermediate 95, 500 mg; 1.15 mmol), the title compound was obtained as white solid.

¹H NMR (DMSO-d6, 400 MHz): δ 7.90-7.92 (2H, d), 7.83-7.87 (1H, t), 7.69-7.71 (2H, d) 7.25-7.29 (1H, t), 7.13-7.19 (3H, m), 6.99-7.10 (2H, m), 4.44 (4H, s). MS (ESI−): 473.9. HPLC (Condition B): Rt 3.76 min (HPLC purity 97.0%).

### Example 83

### 4-cyano-N-(2-fluorobenzyl)-N-[4-(2H-tetrazol-5-yl)benzyl]benzenesulfonamide

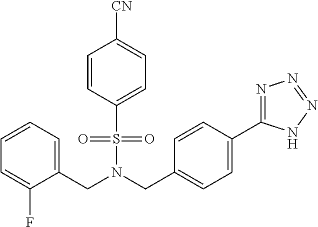

Following the general method as outlined 94, starting from 3 N-(2-fluorobenzyl)-N-[4-(2H-tetrazol-5-yl)benzyl]amine (intermediate 97, 500 mg, 1.76 mmol) and 4-cyanobenzene sulfonyl chloride (392 mg; 1.94 mmol), the title compound was obtained as yellow solid.

¹H NMR (DMSO-d6, 400 MHz): δ 8.04-8.10 (2H, m), 7.94 (2H, d), 7.83-7.85 (2H, d) 7.25-7.27 (4H, m), 7.18-7.24 (1H, m), 6.94-7.04 (1H, m), 4.45 (2H, s), 4.44 (2H, s). MS (ESI−): 446.8. HPLC (Condition B): Rt 4.58 min (HPLC purity 92.0%).

### Example 89

### rac-6-(4-{[(4-Ethoxy-benzenesulfonyl)-pyridin-2-ylmethyl-amino]-methyl}-benzoylamino)-3-aza-bicyclo[3.1.0]hexane-3-carboxylic acid tert-butyl ester

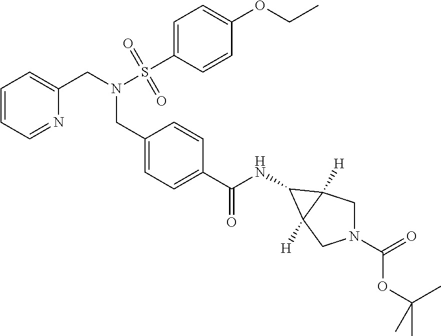

A solution of 4-{[(4-Ethoxy-benzenesulfonyl)-pyridin-2-ylmethyl-amino]-methyl}-benzoic acid (intermediate 5a; 150 mg; 0.35 mmol) and 70 mg (0.35 mmol) rac-6-Amino-3-aza-bicyclo[3.1.0]hexane-3-carboxylic acid tert-butyl ester in DMF (2 mL) was treated with 1-(3-dimethylaminopropyl)-3-ethylcarbodiimide hydrochloride (74.2 mg; 0.38 mmol), 1-hydroxy-benzotriazole (59.2 mg; 0.38 mmol) and N-methylmorpholine (116 μl; 1.0 mmol). After stirring for 12 h, the mixture was diluted with water and extracted with EtOAc. The organic phase was separated, dried over magnesium sulfate, filtered and concentrated to give solid, which was purified by column chromatography (silica; benzene/EtOAc: 2/1) to give the title compound as a white powder (141.0 mg, 64.2%). (MS: m/z: 607).

### Example 117

### 4-{[(4-Ethoxy-benzenesulfonyl)-pyridin-2-ylmethyl-amino]-methyl}-N-[(1S,5R,6S)-3-(4-trifluoromethoxy-benzoyl)-3-aza-bicyclo[3.1.0]hex-6-yl]-benzamide

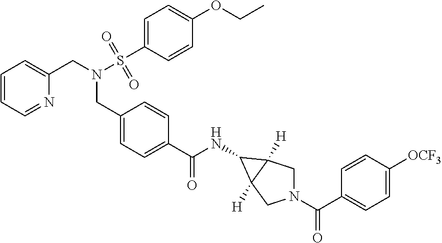

Following the general method as outlined in Example 30b, starting from the amine 28a (43 mg; 0.08 mmol) and 4-Trifluoromethoxy-benzoyl chloride (21 mg; 0.093 mmol) the title compound was obtained as a white solid in 27% yield. (MS: m/z: 695).

### Example 134

### 4-{[(4-Chloro-benzenesulfonyl)-(1-oxy-pyridin-2-ylmethyl)-amino]-methyl}-N-cyclopropylmethyl-benzamide

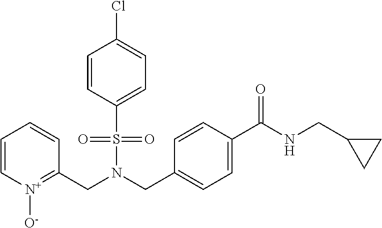

4-{[(4-Chloro-benzenesulfonyl)-pyridin-2-ylmethyl-amino]-methyl}-N-cyclopropylmethyl-benzamide (0.09 mmol) is dissolved in dichloromethane (0.5 ml) and 3-Chloroperbenzoicacid (1.1 eq) is added. The reaction is stirred 1 day at room temperature and extracted with saturated NaHCO₃ and brine. The organic phase is dried over MgSO₄ and the solvent removed in vacuo. 4-{[(4-Chloro-benzenesulfonyl)-(1-oxy-pyridin-2-ylmethyl)-amino]-methyl}-N-cyclopropylmethyl-benzamide is obtained as colorless solid (72% yield). HPLC (condition D): 3.11, LCMS: 486.1 m/z. ¹H NMR (400 MHz, DMSO) δ 8.50 (t, J=5.7, 1H), 8.17-8.07 (m, 1H), 7.93-7.84 (m, 2H), 7.77-7.65 (m, 4H), 7.33 (d, J=8.3, 2H), 7.30-7.16 (m, 3H), 4.59 (s, 2H), 4.46 (s, 2H), 3.13-3.08 (m, 2H), 1.05-0.96 (m, 3H), 0.50-0.34 (m, 2H), 0.25-0.14 (m, 2H).

### Example 149

### (4-{[(4-Chloro-benzenesulfonyl)-pyridin-2-ylmethyl-amino]-methyl}-benzoylamino)-(3,4-difluoro-phenyl)-acetic acid

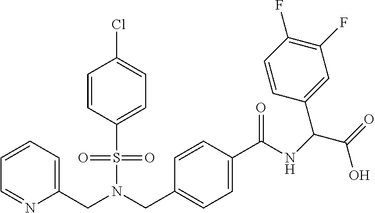

(4-{[(4-Chloro-benzenesulfonyl)-pyridin-2-ylmethyl-amino]-methyl}-benzoylamino)-(3,4-difluoro-phenyl)-acetic acid methyl ester (0.18 mmol) (prepared following the protocols described above) is dissolved in THF (5 ml) and LiOH (7 eq) in water (2 ml) is added. The reaction solution is stirred 19 hours at room temperature and acidified with citric acid. The reaction solution is extracted with ethylacetate and the combined organic phases are dried over MgSO₄. (4-{[(4-Chloro-benzenesulfonyl)-pyridin-2-ylmethyl-amino]-methyl}-benzoylamino)-(3,4-difluoro-phenyl)-acetic acid is obtained as colorless solid (82% yield).

HPLC (condition D): 3.12, LCMS: 585.8 m/z; ¹H NMR (500 MHz, DMSO) δ 13.06 (s, 1H), 9.00 (d, J=7.6, 1H), 8.38-8.28 (m, 1H), 7.87-7.82 (m, 2H), 7.79 (d, J=8.3, 2H), 7.67-7.61 (m, 3H), 7.57 (ddd, J=11.5, 7.7, 2.0, 1H), 7.43 (dt, J=10.6, 8.5, 1H), 7.35 (br, 1H), 7.27 (d, J=8.3, 2H), 7.22-7.16 (m, 2H), 5.62 (d, J=7.6, 1H), 4.52 (s, 2H), 4.41 (s, 2H).

### Example 162

### 1-Phenyl-cyclopropanecarboxylic acid (4-{[(4-chloro-benzenesulfonyl)-pyridin-2-ylmethyl-amino]-methyl}-phenyl)-amide

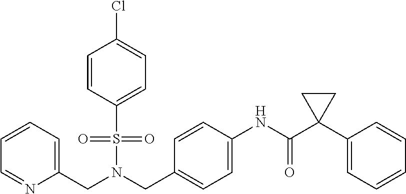

a) 0.50 g (3.08 mmol) 1-phenylcyclopropanecarboxylic acid, 2.24 ml (30.83 mmol) thionyl chloride and 1 drop of dimethylformamide are placed in 25 ml dichloromethane, and then refluxed for 3 hours with stirring. Then the reaction mixture is concentrated by evaporation, taken up in toluene and evaporated to dryness. 1-phenyl-cyclopropanecarbonyl chloride (0.553 g; 99.9% yield) was obtained as pale oil, which was used in the next step without additional purification.

b) A cold (0° C.) solution of the amine 4c (200 mg; 0.52 mmol) in anhydrous DCM (10 ml) was treated with triethylamine (0.215 ml; 1.55 mmol) followed by the addition of 1-phenyl-cyclopropanecarbonyl chloride (93.1 mg; 0.52 mmol). The reaction mixture was allowed to warm to room temperature and stirred for 2 h, and after quenching with water and separating of the DCM layer, the aqueous layer was extracted with EtOAc. The combined organic layer were dried over sodium sulfate, concentrated and purified by column chromatography (Condition B) to give the Title compound (195 mg, 71%). (MS: m/z: 533).

### Example 202

### 4-{[(4-Ethoxy-benzenesulfonyl)-pyridin-2-ylmethyl-amino]-methyl}-N-((1S,2S)-2-hydroxy-1-hydroxymethyl-2-phenyl-ethyl)-benzamide

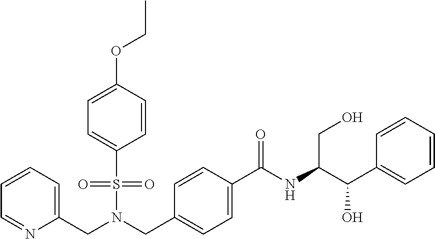

Following the general method as outlined in Example 28, starting from 4-{[(4-Ethoxy-benzenesulfonyl)-pyridin-2-ylmethyl-amino]-methyl}-benzoic acid (intermediate 5a; 100 mg, 0.23 mmol) and (1S,2S)-2-Amino-1-phenyl-propane-1,3-diol (39.2 mg, 0.23 mmol) the title compound was obtained as a yellow solid (120 mg; 89% yield). (MS: m/z: 576).

### Example 210

### rac-N-3-Aza-bicyclo[3.1.0]hex-6-yl-4-{[(4-ethoxy-benzenesulfonyl)-pyridin-2-ylmethyl-amino]-methyl}-benzamide

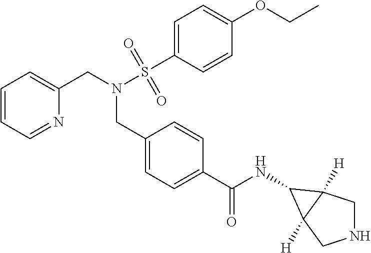

A solution of rac-6-(4-{[(4-Ethoxy-benzenesulfonyl)-pyridin-2-ylmethyl-amino]-methyl}-benzoylamino)-3-aza-bicyclo[3.1.0]hexane-3-carboxylic acid tert-butyl ester (130 mg; 0.21 mmol) in 1 ml 4N HCl in dioxane was stirred for 12 h. and then evaporated to dryness. The remained residue was purified by column chromatography (Condition B) to give the Title compound as a pale brown solid (113 mg, 97% yield). (MS: m/z: 507).

### Example 279

### 4-Chloro-N-(4-cyano-benzyl)-N-(pyridine-2-carbonyl)-benzenesulfonamide

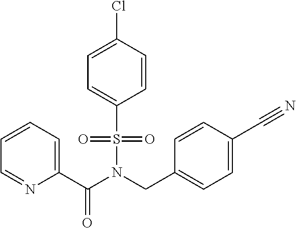

4-Chloro-N-(4-cyano-benzyl)-benzenesulfonamide (0.3 mmol) is dissolved in dichloromethane (1 ml) and Triethylamine is added (2.3 eq). After addition of Picolinoyl chloride HCl (1 eq), the reaction is stirred 1 day at room temperature. Afterwards Picolinoyl chloride HCl (1 eq) and Triethylamine (2.3 eq) is added and the reaction is stirred 3 days at room temperature. The reaction solution is diluted with dichloromethane and extracted with water and brine. The organic phase is dried over MgSO₄ and the solvent removed in vacuo. The residue is suspended in water and filtrated. 4-Chloro-N-(4-cyano-benzyl)-N-(pyridine-2-carbonyl)-benzenesulfonamide is obtained as an offwhite solid (64% yield). HPLC (condition D): 3.35, LCMS: 412 m/z; ¹H NMR (500 MHz, DMSO) δ 8.54-8.49 (m, 1H), 8.04-7.97 (m, 2H), 7.96-7.91 (m, 1H), 7.80-7.71 (m, 4H), 7.66 (d, J=7.8, 1H), 7.57 (ddd, J=7.7, 4.8, 1.1, 1H), 7.40 (d, J=8.4, 2H), 5.30 (s, 2H).

### Example 281

### 4-Chloro-N-(4-cyano-benzoyl)-N-pyridin-2-ylmethyl-benzenesulfonamide

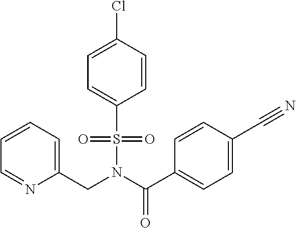

4-Chloro-N-pyridin-2-ylmethyl-benzenesulfonamide (0.5 mmol) is dissolved in DMF and NaH (60% suspension in paraffin oil, 1.1 eq) added. After 30 min, Ethyl-4-cyanobenzoylchloride (1 eq) is added and the reaction solution is stirred 1 day at room temperature. The reaction solution is poured on water and diluted with methanol and acetonitrile. The precipitate is filtered and dried in vacuo at 40° C. 4-Chloro-N-(4-cyano-benzoyl)-N-pyridin-2-ylmethyl-benzenesulfonamide is obtained as colorless solid (61% yield). HPLC (condition D): 3.16, LCMS: 412 m/z; ¹H NMR (400 MHz, DMSO) δ 8.38-8.32 (m, 1H), 7.90-7.84 (m, 2H), 7.82-7.76 (m, 2H), 7.73 (td, J=7.7, 1.8, 1H), 7.71-7.67 (m, 2H), 7.65-7.60 (m, 2H), 7.32-7.21 (m, 2H), 5.13 (s, 2H).

### Example 286

### 4-Chloro-N-[2-(4-cyano-phenyl)-ethyl]-N-pyridin-2-ylmethyl-benzenesulfonamide

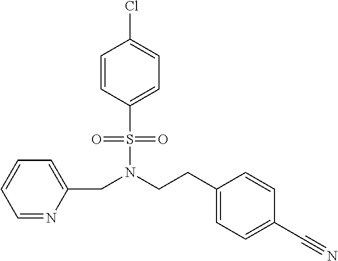

4-Chloro-N-pyridin-2-ylmethyl-benzenesulfonamide (1 mmol) is dissolved in THF (2 ml) and triphenylphosphine (1.3 eq) is added at 0° C. After 10 min, diethylazodicarboxylate (1.3 eq) in toluene (0.7 ml) is added and the reaction solution is stirred 4 hours at 0° C. The solvent is removed in vacuo and the crude product dissolved in diethylether and filtrated over celite. 4-Chloro-N-[2-(4-cyano-phenyl)-ethyl]-N-pyridin-2-ylmethyl-benzenesulfonamide is obtained after column chromatography (heptane/ethylacetate) as colorless solid (4% yield). HPLC (condition C): 1.76, LCMS: 412 m/z; ¹H NMR (500 MHz, DMSO) δ 8.45 (d, J=4.0, 1H), 7.83-7.72 (m, 3H), 7.67 (d, J=8.3, 2H), 7.63-7.58 (m, 2H), 7.35 (d, J=7.8, 1H), 7.32-7.26 (m, 3H), 4.49 (s, 2H), 3.49-3.45 (m, 2H), 2.83-2.77 (m, 2H).

### Example 355

### 1-(4-{[(4-Chloro-benzenesulfonyl)-pyridin-2-ylmethyl-amino]-methyl}-benzoylamino)-cyclopropanecarboxylic acid

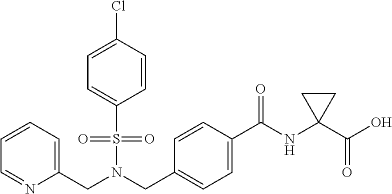

1-(4-{[(4-Chloro-benzenesulfonyl)-pyridin-2-ylmethyl-amino]-methyl}-benzoylamino)-cyclopropanecarboxylic acid ethyl ester (0.08 mmol) is dissolved in ethanol (1 ml) and 1N NaOH (5 eq) is added. The reaction solution is stirred at room temperature over night and acidified with citric acid. The reaction solution is extracted with ethylacetate and the combined organic phases are dried over MgSO₄. 1-(4-{[(4-Chloro-benzenesulfonyl)-pyridin-2-ylmethyl-amino]-methyl}-benzoylamino)-cyclopropanecarboxylic acid is obtained as colorless (65% yield). HPLC (condition D): 2.88, LCMS: 499.9 m/z.

### Example 347

### 3-Amino-N-pyridin-2-ylmethyl-N-[4-(1H-tetrazol-5-yl)-benzyl]-benzenesulfonamide

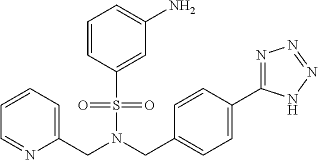

3-Nitro-N-pyridin-2-ylmethyl-N-[4-(1H-tetrazol-5-yl)-benzyl]-benzenesulfonamide (0.12 mmol) is dissolved in methanol and is hydrogenated over Pd/C at room temperature for 1 hour. After filtration over celite, the solvent is removed in vacuo. 3-Amino-N-pyridin-2-ylmethyl-N-[4-(1H-tetrazol-5-yl)-benzyl]-benzenesulfonamide is obtained after reversed phase column chromatography as colorless solid (64% yield). HPLC (condition D): 2.63, LCMS: 421.85 m/z.; ¹H NMR (500 MHz, DMSO) δ 8.38 (d, J=4.1, 1H), 7.86 (d, J=8.2, 2H), 7.70-7.60 (m, 1H), 7.35 (d, J=8.2, 2H), 7.22 (ddd, J=17.3, 10.1, 4.8, 3H), 7.10 (t, J=2.0, 1H), 6.97 (d, J=7.6, 1H), 6.84 (dd, J=8.1, 1.5, 1H), 4.45 (s, 2H), 4.40 (s, 2H), 4.20 (s, 3H).

### Example 359

### (S)-2-(4-{[(4-Chloro-benzenesulfonyl)-pyridin-2-ylmethyl-amino]-methyl}-benzoylamino)-3-phenyl-propionic acid

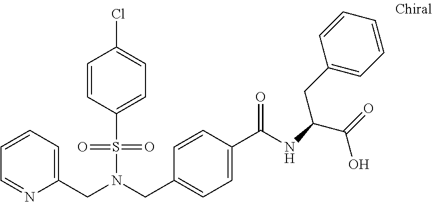

(S)-2-(4-{[(4-Chloro-benzenesulfonyl)-pyridin-2-ylmethyl-amino]-methyl}-benzoylamino)-3-phenyl-propionic acid tert-butyl ester (0.1 mmol) is dissolved in 4M HCl in Dioxan (1.7 ml) and stirred over night at room temperature. The reaction solution is diluted with water and extracted with dichloromethane. The combined organic phases are dried over MgSO₄ and the solvent removed in vacuo. (S)-2-(4-{[(4-Chloro-benzenesulfonyl)-pyridin-2-ylmethyl-amino]-methyl}-benzoylamino)-3-phenyl-propionic acid is obtained after reversed phase column chromatography as colorless solid (69% yield). HPLC (condition D): 3.09, LCMS: 563.8 m/z. ¹H NMR (500 MHz, DMSO) δ 12.72 (br, 1H), 8.60 (d, J=8.2, 1H), 8.34 (dd, J=5.3, 1.8, 1H), 7.87-7.82 (m, 2H), 7.70-7.61 (m, 5H), 7.33-7.12 (m, 9H), 4.59 (ddd, J=10.7, 8.2, 4.5, 1H), 4.50 (s, 2H), 4.42 (s, 2H), 3.17 (dd, J=13.8, 4.4, 1H), 3.05 (dd, J=13.8, 10.6, 1H).

The following compounds have been synthesised according to the procedure hereabove described:

| | | | RT | | | |
|---|---|---|---|---|---|---|
| | | MS | HPLC | | amount | |
| Ex number | Synthesisroute | (M + H) | (Method) | Yield | (μmol) | NMR |
| 84 | 1 | 546 | | 40.6 | 95 | |
| 85 | 1 | 567 | | 96 | 225 | |
| 86 | 1 | 544 | | 44.7 | 105 | |
| 87 | 1 | 560 | | 95.6 | 224 | |
| 88 | 1 | 542 | | 98.3 | 231 | |
| 90 | 1 | 565 | | 58.9 | 138 | |
| 91 | 1 | 546 | | 92.8 | 218 | |
| 92 | 1 | 544 | | 44.7 | 105 | |
| 93 | 1 | 542 | | 57.4 | 135 | |
| 94 | 1 | 607 | | 82.9 | 291 | |
| 95 | 1 | 609 | | 49.7 | 117 | |
| 96 | 1 | 588 | | 78.5 | 289 | |
| 97 | 1 | 546 | | 29.9 | 73 | |
| 98 | 1 | 525 | | 24.8 | 61 | |
| 99 | 1 | 590 | | 61.1 | 150 | |
| 100 | 1 | 554 | | 45.1 | 111 | |
| 101 | 1 | 548 | | 71.4 | 175 | |
| 102 | 1 | 527 | | 65.3 | 160 | |
| 103 | 2 + 7 | 516.1 | 3.52 min. | 36.9 | 158 | |
| | | | (D) | | | |
| 104 | 2 + 7 | 550 | 3.56 min. | 40.3 | 142 | |
| | | | (D) | | | |
| 105 | 1 | 598 | | 98 | 470 | |
| 106 | 1 | 597 | | 86.8 | 417 | |
| 107 | 1 | 549 | | 63.5 | 56 | |
| 108 | 1 | 549 | | 73.8 | 73 | |
| 109 | 1 | 527 | | 24.7 | 46 | |
| 110 | 1 | 546 | | 22.9 | 42 | |
| 111 | 1 | 548 | | 24.3 | 45 | |
| 112 | 1 | 525 | | 16.3 | 30 | |
| 113 | 1 | 523 | | 23.9 | 44 | |
| 114 | 1 | 557 | | 56.5 | 104 | |
| 115 | 2 + 7 | 489.95 | 2.07 min. | 34.7 | 254 | |
| | | | (C) | | | |
| 116 | 1 | 695 | | 12.6 | 13 | |
| 118 | 1 | 539 | | 69.8 | 87 | |
| 119 | 1 | 530 | | 54.7 | 47 | |
| 120 | 2 + 7 | 490.95 | 1.69 min. | 9.3 | 83 | |
| | | | (C) | | | |
| 121 | 2 + 7 | 440 | 3.33 min. | 27.5 | 89 | |
| | | | (D) | | | |
| 122 | 1 | 567 | | 81.7 | 196 | |
| 123 | 1 | 558 | | 58.5 | 144 | |
| 124 | 1 | 577 | | 68.8 | 161 | |
| 125 | 1 | 567 | | 58.9 | 141 | |
| 126 | 1 | 558 | | 54.1 | 133 | |
| 127 | 1 | 577 | | 82.9 | 194 | |
| 128 | 1 | 577 | | 49.9 | 120 | |
| 129 | 1 | 567 | | 43.1 | 106 | |
| 130 | 1 | 586 | | 40 | 94 | |
| 131 | 1 | 617 | | 29.1 | 70 | |
| 132 | 1 | 565 | | 64.6 | 310 | |
| 133 | 1 | 551 | | 58.1 | 154 | |
| 135 | 1 | 498 | | 26.2 | 64 | |
| 136 | 1 | 498 | | 79.7 | 196 | |
| 137 | 1 | 498 | | 88.6 | 217 | |
| 138 | 1 | 497 | | 90.6 | 222 | |
| 139 | 3b + 7 | 421.1 | 2.81 min. | 10.3 | 44 | |
| | | | (D) | | | |
| 140 | 3b + 7 | 451.8 | 2.91 min. | 15.5 | 64 | |
| | | | (D) | | | |
| 141 | 3b + 7 | 462.9 | 3.12 min. | 50.2 | 239 | |
| | | | (D) | | | |
| 142 | 1 | 502.8 | 2.76 min. | 6.7 | 17 | |
| | | | (D) | | | |
| 143 | 1 | 550.9 | 2.81 min. | 4.7 | 12 | |
| | | | (D) | | | |
| 144 | 1 | 518.9 | 2.92 min. | 4.8 | 12 | |
| | | | (D) | | | |
| 145 | 1 | 577.8 | 3.2 min. | 54 | 194 | |
| | | | (D) | | | |
| 146 | 1 | 593.8 | 3.04 min. | 22.2 | 80 | |
| | | | (D) | | | |
| 147 | 1 | 599.8 | 3.25 min. | 64.4 | 231 | ¹H NMR (500 MHz, DMSO) δ |
| | | | (D) | | | 9.15 (d, J = 7.3, 1H), 8.36 (dd, |
| | | | | | | J = 5.5, 1.6, 1H), |
| | | | | | | 7.90-7.83 (m, 2H), 7.79 (d, J = 8.2, 2H), |
| | | | | | | 7.69 (td, J = 7.7, 1.6, 1H), |
| | | | | | | 7.65-7.62 (m, 2H), 7.61-7.55 (m, |
| | | | | | | 1H), 7.45 (dt, J = 10.6, 8.5, |
| | | | | | | 1H), 7.39-7.32 (m, 1H), |
| | | | | | | 7.28 (d, J = 8.3, 2H), |
| | | | | | | 7.25-7.19 (m, 2H), 5.72 (d, J = 7.3, 1H), |
| | | | | | | 4.52 (s, 2H), 4.43 (s, 2H), |
| | | | | | | 3.67 (s, 3H). |
| 148 | 1 | 540.9 | 2.72 min. | 22 | 52 | |
| | | | (D) | | | |
| 150 | 3b | 555.85 | 3.11 min. | 29 | 59 | |
| | | | (D) | | | |
| 151 | 1 + 13 | 549.8 | 3.05 min. | 71.4 | 22 | ¹H NMR (500 MHz, DMSO) δ |
| | | | (D) | | | 8.94 (d, J = 7.5, 1H), 8.34 (dd, |
| | | | | | | J = 5.2, 1.8, 1H), |
| | | | | | | 7.87-7.83 (m, 2H), 7.80 (d, J = 8.3, 2H), |
| | | | | | | 7.70-7.59 (m, 3H), |
| | | | | | | 7.51-7.46 (m, 2H), 7.40-7.35 (m, |
| | | | | | | 2H), 7.35-7.31 (m, 1H), |
| | | | | | | 7.25 (d, J = 8.3, 2H), |
| | | | | | | 7.22-7.17 (m, 2H), 5.58 (d, J = 7.5, 1H), |
| | | | | | | 4.52 (s, 2H), 4.42 (s, 2H), |
| | | | | | | 3.65 (br, 1H). |
| 152 | 1 + 13 | 549.8 | 3.05 min. | 43.6 | 73 | |
| | | | (D) | | | |
| 153 | 1 + 13 | 563.8 | 3.07 min. | 74.2 | 110 | |
| | | | (D) | | | |
| 154 | 1 + 13 | 579.8 | 2.92 min. | 92.6 | 59 | ¹H NMR (500 MHz, DMSO) δ |
| | | | (D) | | | 12.63 (s, 1H), 12.19 (s, 1H), |
| | | | | | | 9.12 (s, 1H), 8.51 (d, J = 8.2, |
| | | | | | | 1H), 8.32 (d, J = 4.2, 1H), |
| | | | | | | 7.87-7.82 (m, 2H), 7.68 (d, J = 8.3, |
| | | | | | | 2H), 7.65-7.59 (m, 2H), |
| | | | | | | 7.23 (d, J = 8.3, 2H), |
| | | | | | | 7.21-7.14 (m, 2H), 7.07 (d, J = 8.5, |
| | | | | | | 2H), 6.63 (d, J = 8.5, 2H), |
| | | | | | | 4.55-4.45 (m, 3H), 4.41 (s, 2H), |
| | | | | | | 3.04 (dd, J = 13.9, 4.4, 1H), |
| | | | | | | 2.92 (dd, J = 13.8, 10.3, 1H). |
| 155 | 2 | 455 | | 95.9 | 3282 | |
| 156 | 2 | 436 | | 89.7 | 3280 | |
| 157 | 2 | 427 | | 96.9 | 3196 | |
| 158 | 2a | 409 | | 80.2 | 2935 | |
| 159 | 2 | 445 | | 88.9 | 4715 | |
| 160 | 1 | 541 | | 71.1 | 458 | |
| 161 | 1 | 532 | | 58 | 307 | |
| 163 | 1 | 523 | | 94 | 497 | |
| 164 | 1 | 547 | | 92 | 229 | |
| 165 | 3b + 7 | 499.2 | 3.07 min. | 45.4 | 355 | |
| | | | (D) | | | |
| 166 | 3b + 7 | 483.2 | 3.05 min. | 21 | 172 | |
| | | | (D) | | | |
| 167 | 1 | 537 | | 41 | 104 | |
| 168 | 1 | 547 | | 57.9 | 108 | |
| 171 | 3b + 7 | 454.05 | 3.37 min. | 46.2 | 399 | |
| | | | (D) | | | |
| 172 | 3b | 408.95 | 2.97 min. | 39.9 | 406 | |
| | | | (D) | | | |
| 173 | 3b | 409.05 | 2.97 min. | 7.1 | 72 | |
| | | | (D) | | | |
| 174 | 3b | 378.2 | 2.97 min. | 50.8 | 516 | |
| | | | (D) | | | |
| 175 | 3b | 378.2 | 2.96 min. | 39.6 | 402 | |
| | | | (D) | | | |
| 177 | 3b | 419.9 | 3.29 min. | 55.5 | 564 | |
| | | | (D) | | | |
| 178 | 3b | 393.9 | 3 min. | 41.8 | 425 | |
| | | | (D) | | | |
| 179 | 3b + 7 | 499.8 | 3.63 min. | 38 | 15 | ¹H NMR (400 MHz, CDCl₃) δ |
| | | | (D) | | | 7.86 (ddd, J = 9.2, 8.4, 5.4, |
| | | | | | | 4H), 7.58-7.52 (m, 2H), |
| | | | | | | 7.29-7.23 (m, 3H), 7.11 (s, 1H), |
| | | | | | | 6.95 (dd, J = 8.1, 1.9, 1H), |
| | | | | | | 6.52 (d, J = 8.2, 1H), 5.54 (dd, |
| | | | | | | J = 8.3, 6.3, 1H), 4.36 (d, J = 16.4, |
| | | | | | | 1H), 4.13 (d, J = 16.4, |
| | | | | | | 1H), 2.72-2.64 (m, 2H), |
| | | | | | | 2.29 (dtd, J = 14.1, 8.5, 5.6, 1H), |
| | | | | | | 1.75 (ddd, J = 13.7, 8.6, 7.1, |
| | | | | | | 1H). |
| 180 | 1 | 622 | | 11 | 28 | |
| 181 | 1 | 522 | | 66.3 | 107 | |
| 182 | 1 | 537 | | 94.6 | 122 | |
| 184 | 1 | 557 | | 13.5 | 17 | |
| 185 | 1 | 564 | | 97.9 | 77 | |
| 186 | 3b | 397.8 | 3.4 min. | 19.3 | 196 | |
| | | | (D) | | | |
| 187 | 1 | 534 | | 58.8 | 197 | |
| 188 | 2 | 470.9 | 1.72 min. | 58.7 | 293 | |
| | | | (C) | | | |
| 189 | 1 | 571.9 | 1.89 min. | 48.7 | 47229 | |
| | | | (C) | | | |
| 190 | 2 | 492 | | 100 | 3319 | ¹H NMR (400 MHz, DMSO) δ |
| | | | | | | 8.38-8.32 (m, 1H), |
| | | | | | | 7.83-7.75 (m, 4H), 7.63 (td, J = 7.7, |
| | | | | | | 1.8, 1H), 7.31 (d, J = 8.3, 2H), |
| | | | | | | 7.23-7.14 (m, 2H), |
| | | | | | | 7.12-7.05 (m, 2H), 4.47 (s, 2H), |
| | | | | | | 4.38 (s, 2H), 4.35-4.22 (m, |
| | | | | | | 2H), 4.14 (q, J = 7.0, 2H), |
| | | | | | | 1.37 (t, J = 7.0, 3H), 1.30 (q, J = 7.4, |
| | | | | | | 3H). |
| 192 | 2 | 436 | | 74.6 | 2730 | |
| 194 | 2 | 427 | | 97.8 | 3186 | |
| 195 | 2 | 398 | 1.73 min. | 77.1 | 683 | |
| | | | (C) | | | |
| 196 | 2 | 398 | 1.72 min. | 44.3 | 422 | |
| | | | (C) | | | |
| 197 | 1 | 565 | | 43.1 | 101 | |
| 198 | 3b | 425.2 | 3.67 min. | 22 | 101 | |
| | | | (D) | | | |
| 199 | 3b | 416.2 | 3.55 min. | 29.6 | 136 | |
| | | | (D) | | | |
| 200 | 1 | 609 | | 72.1 | 169 | |
| 201 | 1 | 573 | | 41.7 | 98 | |
| 203 | 2 | 408 | | 35.1 | 1128 | |
| 204 | 3b + 7 | 454 | 3.43 min. | 11.5 | 36 | |
| | | | (D) | | | |
| 205 | 1 | 567 | | 71.4 | 167 | |
| 206 | 1 | 544 | | 90.1 | 211 | |
| 207 | 1 | 573 | | 67.7 | 159 | |
| 208 | 1 | 576 | | 37.7 | 89 | |
| 209 | 1 | 560 | | 94.9 | 223 | |
| 211 | 1 | 507 | | 95.4 | 238 | |
| 212 | 3b | 459.1 | 3.37 min. | 23.4 | 75 | |
| | | | (D) | | | |
| 214 | 2 + 7 | 441 | 1.61 min. | 21.5 | 156 | |
| | | | (D) | | | |
| 215 | 3b | 439 | 3.69 min. | 73.4 | 651 | |
| | | | (D) | | | |
| 216 | 1 | 541 | | 48.2 | 118 | |
| 217 | 1 | 557 | | 58.2 | 143 | |
| 219 | 2 | 445 | | 97.3 | 5164 | |
| 220 | 2a | 418 | | 93.3 | 4949 | |
| 221 | 3b + 7 | 468 | 3.47 min. | 56.5 | 502 | |
| | | | (D) | | | |
| 222 | 3b + 7 | 455 | 2.91 min. | 16.8 | 110 | |
| | | | (D) | | | |
| 223 | 3b + 7 | 455 | 2.92 min. | 31.4 | 158 | |
| | | | (D) | | | |
| 224 | 3b + 7 | 482.1 | 3.49 min. | 40.2 | 125 | |
| | | | (D) | | | |
| 225 | 1 | 379 | | 98 | 2640 | |
| 226 | 2 | 417 | | 89.9 | 4242 | |
| 227 | 3b + 7 | 468 | 3.47 min. | 44.8 | 321 | |
| | | | (D) | | | |
| 228 | 2 + 7 | 441 | 1.61 min. | 15.9 | 94 | |
| | | | (C) | | | |
| 229 | 2 + 7 | 439.95 | 3.35 min. | 68.9 | 915 | |
| | | | (D) | | | |
| 230 | 2 | 396.95 | 3.53 min. | 62.7 | 2187 | |
| | | | (D) | | | |
| 231 | 2a | 388 | | 89.9 | 4301 | |
| 233 | 2 + 7 | 508.1 | 1.65 min. | 67.6 | 352 | |
| | | | (C) | | | |
| 234 | 2 + 7 | 439.95 | 3.36 min. | 53.8 | 271 | |
| | | | (D) | | | |
| 235 | 1 | 555 | | 87 | 216 | |
| 236 | 3b + 7 | 482.2 | 3.51 min. | 15.6 | 102 | |
| | | | (D) | | | |
| 237 | 3b + 7 | 499.2 | 3.03 min. | 45.8 | 365 | |
| | | | (D) | | | |
| 238 | 3b + 7 | 454.95 | 2.93 min. | 19.3 | 105 | |
| | | | (D) | | | |
| 239 | 3b + 7 | 454.95 | 2.96 min. | 5.3 | 27 | |
| | | | (D) | | | |
| 240 | 1 | 546 | | 33.1 | 84 | |
| 241 | 1 | 555 | | 29 | 54 | |
| 242 | 1 | 488 | | 93 | 215 | |
| 243 | 1 | 588 | | 85.9 | 316 | |
| 244 | 3b + 7 | 455.1 | 2.92 min. | 11 | 44 | |
| | | | (D) | | | |
| 245 | 1 | 598 | | 97.3 | 467 | |
| 246 | 1 | 546 | | 12.6 | 39 | |
| 247 | 1 | 537 | | 17.7 | 54 | |
| 248 | 1 | 541 | | 25.1 | 46 | |
| 250 | 1 | 590 | | 49.7 | 91 | |
| 251 | 1 | 554 | | 52.9 | 97 | |
| 253 | 3b + 7 | 483.2 | 3.04 min. | 10.3 | 88 | |
| | | | (D) | | | |
| 254 | 1 | 498 | | 98.4 | 420 | |
| 255 | 1 | 498 | | 96.7 | 424 | |
| 256 | 1 | 497 | | 82.9 | 314 | |
| 257 | 3b + 7 | 411.1 | 3.59 min. | 47 | 951 | |
| | | | (D) | | | |
| 258 | 3b + 7 | 411.1 | 3.57 min. | 51 | 1165 | |
| | | | (D) | | | |
| 259 | 1 | 488 | | 81.3 | 221 | |
| 260 | 3 | 459 | | 41.8 | 159 | |
| 261 | 1 | 540 | | 64.5 | 91 | |
| 262 | 1 | 540 | | 53.2 | 76 | |
| 263 | 3 | 543 | | 19.1 | 72 | |
| 264 | 3 | 503 | | 55.4 | 175 | |
| 265 | 1 | 530 | | 55.2 | 45 | |
| 266 | 3b + 7 | 407.2 | 2.76 min. | 9.2 | 51 | |
| | | | (D) | | | |
| 268 | 1 | 467 | | 75.2 | 281 | |
| 269 | 1 | 468 | | 94.1 | 250 | |
| 270 | 1 | 476 | | 98.5 | 256 | |
| 271 | 3b + 7 | 454.05 | 3.36 min. | 11.4 | 122 | |
| | | | (D) | | | |
| 272 | 3b + 7 | 454.2 | 3.39 min. | 8 | 31 | |
| | | | (D) | | | |
| 273 | 1 | 600 | | 62.6 | 147 | |
| 274 | 1 | 591 | | 38.1 | 91 | |
| 275 | 3b | 432.1 | 3.79 min. | 19.6 | 832 | |
| | | | (D) | | | |
| 276 | 3b | 409.1 | 2.97 min. | 42.2 | 428 | |
| | | | (D) | | | |
| 279 | 10 | 412 | 3.35 min. | 63.7 | 197 | ¹H NMR (500 MHz, DMSO) δ |
| | | | (D) | | | 8.54-8.49 (m, 1H), |
| | | | | | | 8.04-7.97 (m, 2H), 7.96-7.91 (m, |
| | | | | | | 1H), 7.80-7.71 (m, 4H), |
| | | | | | | 7.66 (d, J = 7.8, 1H), 7.57 (ddd, J = 7.7, |
| | | | | | | 4.8, 1.1, 1H), 7.40 (d, J = 8.4, |
| | | | | | | 2H), 5.30 (s, 2H),. |
| 280 | 2 | 506.2 | 3.27 min. | 19.1 | 63 | |
| | | | (D) | | | |
| 281 | 10 | 412 | 3.16 min. | 60.5 | 311 | ¹H NMR (400 MHz, DMSO) δ |
| | | | (D) | | | 8.38-8.32 (m, 1H), |
| | | | | | | 7.90-7.84 (m, 2H), 7.82-7.76 (m, |
| | | | | | | 2H), 7.73 (td, J = 7.7, 1.8, 1H), |
| | | | | | | 7.71-7.67 (m, 2H), |
| | | | | | | 7.65-7.60 (m, 2H), 7.32-7.21 (m, |
| | | | | | | 2H), 5.13 (s, 2H). |
| 282 | 1 | 607 | | 37.6 | 92 | |
| 283 | 1 | 626 | | 72.3 | 169 | |
| 284 | 14 | 350.2 | 3.47 min. | 3.7 | 15 | |
| | | | (D) | | | |
| 285 | 12 + 7 | 455 | 1.57 min. | 21.3 | 127 | |
| | | | (C) | | | |
| 286 | 11 | 412 | 1.76 min. | 3.5 | 35 | ¹H NMR (500 MHz, DMSO) δ |
| | | | (C) | | | 8.45 (d, J = 4.0, 1H), |
| | | | | | | 7.83-7.72 (m, 3H), 7.67 (d, J = 8.3, |
| | | | | | | 2H), 7.63-7.58 (m, 2H), |
| | | | | | | 7.35 (d, J = 7.8, 1H), |
| | | | | | | 7.32-7.26 (m, 3H), 4.49 (s, 2H), |
| | | | | | | 3.49-3.45 (m, 2H), 2.83-2.77 (m, |
| | | | | | | 2H). |
| 287 | 3b | 378.2 | 2.97 min. | 35.5 | 361 | |
| | | | (D) | | | |
| 288 | 3 | 443 | | 94.7 | 1513 | |
| 289 | 3 | 453 | | 100 | 1414 | |
| 290 | 3 | 434 | | 4.7 | 52 | |
| 291 | 1 | 559 | | 51.6 | 116 | |
| 292 | 1 | 567 | | 78.2 | 177 | |
| 293 | 1 | 557 | | 29.5 | 67 | |
| 294 | 1 | 568 | | 65.4 | 144 | |
| 295 | 1 | 577 | | 57.3 | 127 | |
| 296 | 12 | 486.1 | 3.11 min. | 72 | 61 | ¹H NMR (400 MHz, DMSO) δ |
| | | | (D) | | | 8.50 (t, J = 5.7, 1H), |
| | | | | | | 8.17-8.07 (m, 1H), 7.93-7.84 (m, |
| | | | | | | 2H), 7.77-7.65 (m, 4H), |
| | | | | | | 7.33 (d, J = 8.3, 2H), |
| | | | | | | 7.30-7.16 (m, 3H), 4.59 (s, 2H), 4.46 (s, |
| | | | | | | 2H), 3.13-3.08 (m, 2H), |
| | | | | | | 1.05-0.96 (m, 3H), 0.50-0.34 (m, |
| | | | | | | 2H), 0.25-0.14 (m, 2H). |
| 297 | 1 | 558 | | 12.7 | 39 | |
| 298 | 3b + 7 | 452.1 | 2.83 min. | 49.2 | 176 | |
| | | | (D) | | | |
| 299 | 3b | 457 | 3.64 min. | 4.1 | 53 | ¹H NMR (500 MHz, DMSO) δ |
| | | | (D) | | | 8.00-7.94 (m, 2H), |
| | | | | | | 7.76-7.70 (m, 2H), 7.65 (d, J = 8.3, |
| | | | | | | 2H), 7.30 (d, J = 8.3, 2H), |
| | | | | | | 7.20 (s, 1H), 6.98 (dd, J = 8.2, 1.9, |
| | | | | | | 1H), 6.64 (d, J = 8.2, 1H), |
| | | | | | | 5.55 (dd, J = 8.4, 5.9, 1H), 4.32 (d, |
| | | | | | | J = 17.0, 1H), 4.22 (d, J = 17.1, |
| | | | | | | 1H), 2.85-2.75 (m, 1H), |
| | | | | | | 2.74-2.65 (m, 1H), |
| | | | | | | 2.22-2.12 (m, 1H), 1.61 (ddd, J = 20.0, |
| | | | | | | 9.2, 6.1, 1H). |
| 300 | 1 | 489 | | 97.2 | 257 | |
| 309 | 1 | 558 | | 14 | 18 | |
| 311 | 3b + 7 | 421.15 | 2.83 min. | 49.5 | 157 | |
| | | | (D) | | | |
| 312 | 3b | 405.9 | 3.59 min. | 17.3 | 176 | |
| | | | (D) | | | |
| 313 | 3b | 397.8 | 3.45 min. | 43.5 | 442 | |
| | | | (D) | | | |
| 314 | 1 | 544 | | 44.5 | 107 | |
| 315 | 1 | 543 | | 69.1 | 166 | |
| 316 | 3b | 421.1 | 2.85 min. | 50.1 | 146 | |
| | | | (D) | | | |
| 318 | 3b + 7 | 436.9 | 2.85 min. | 48.4 | 172 | |
| | | | (D) | | | |
| 319 | 3b | 418 | | 0.5 | 10 | |
| 320 | 3b | 393.9 | 2.95 min. | 41.3 | 420 | |
| | | | (D) | | | |
| 321 | 3b + 7 | 449.2 | 3.05 min. | 32 | 63 | |
| | | | (D) | | | |
| 322 | 3b + 7 | 440.8 | 2.93 min. | 45.5 | 171 | |
| | | | (D) | | | |
| 323 | 3b + 7 | 440.8 | 2.88 min. | 19.2 | 49 | |
| | | | (D) | | | |
| 324 | 2 | 519.8 | 3.57 min. | 4.9 | 20 | |
| | | | (D) | | | |
| 325 | 2 | 519.1 | 3.49 min. | 10.4 | 97 | |
| | | | (D) | | | |
| 326 | 14 | 441.8 | 3.69 min. | 9.1 | 54 | |
| | | | (D) | | | |
| 327 | 2 | 468 | | 10.8 | 36 | |
| 328 | 2 | 478 | | 100 | 314 | |
| 330 | 1 | 584 | | 29.6 | 21 | |
| 331 | 1 | 594 | | 54.5 | 148 | |
| 333 | 3b + 7 | 436.85 | 2.79 min. | 47.1 | 168 | |
| | | | (D) | | | |
| 334 | 14 | 427.8 | 3.47 | 70 | 183 | |
| | | | (D) | | | |
| 335 | 1 | 598.8 | 3.64 | 42.7 | 329 | |
| | | | (D) | | | |
| 336 | 2 | 584.9 | 3.45 min. | 41.5 | 116 | |
| | | | (D) | | | |
| 337 | 1 | 654.9 | 3.21 min. | 80.1 | 101 | |
| | | | (D) | | | |
| 339 | 1 | 619.8 | 3.41 min. | 40.6 | 146 | |
| | | | (D) | | | |
| 342 | 1 | 563.8 | 3.17 min. | 65.6 | 235 | |
| | | | (D) | | | |
| 343 | 1 | 605.8 | 3.37 min. | 13.5 | 49 | |
| | | | (D) | | | |
| 345 | 1 | 593.8 | 3.03 min. | 54.3 | 195 | |
| | | | (D) | | | |
| 346 | 2 + 13 | 456.85 | 2.92 min. | 83.1 | 217 | |
| | | | (D) | | | |
| 347 | 2a | 421.85 | 2.63 min. | 63.8 | 79 | ¹H NMR (500 MHz, DMSO) δ |
| | | | (D) | | | 8.38 (d, J = 4.1, 1H), 7.86 (d, J = 8.2, |
| | | | | | | 2H), 7.70-7.60 (m, |
| | | | | | | 1H), 7.35 (d, J = 8.2, 2H), |
| | | | | | | 7.22 (ddd, J = 17.3, 10.1, 4.8, 3H), |
| | | | | | | 7.10 (t, J = 2.0, 1H), 6.97 (d, J = 7.6, |
| | | | | | | 1H), 6.84 (dd, J = 8.1, |
| | | | | | | 1.5, 1H), 4.45 (s, 2H), 4.40 (s, |
| | | | | | | 2H), 4.20 (s, 3H). |
| 349 | 2 | 546.8 | 1.99 min. | 3.1 | 15550 | |
| | | | (C) | | | |
| 351 | 3b | 541.8 | 2.97 min. | 30.8 | 86 | |
| | | | (D) | | | |
| 352 | 1 | 527.8 | 3 min. | 76.2 | 110 | |
| | | | (D) | | | |
| 353 | 3b | 456.85 | 1.81 min. | 4.6 | 11829 | |
| | | | (C) | | | |
| 354 | 1 + 13 | 579.8 | 2.93 min. | 51.5 | 76 | |
| | | | (D) | | | |
| 355 | 1 + 13 | 499.9 | 2.88 min. | 64.7 | 50 | |
| | | | (D) | | | |
| 356 | 3b | 584 | 2.99 min. | 56.8 | 127 | |
| | | | (D) | | | |
| 357 | 3b | 575.9 | 3 min. | 11.9 | 26724 | |
| | | | (D) | | | |
| 358 | 3b | 569 | 3.08 min. | 42.9 | 96 | |
| | | | (D) | | | |
| 359 | 1 + 13 | 563.8 | 3.09 min. | 68.7 | 67 | ¹H NMR (500 MHz, DMSO) δ |
| | | | (D) | | | 12.72 (br, 1H), 8.60 (d, J = 8.2, |
| | | | | | | 1H), 8.34 (dd, J = 5.3, 1.8, |
| | | | | | | 1H), 7.87-7.82 (m, 2H), |
| | | | | | | 7.70-7.61 (m, 5H), 7.33-7.12 (m, |
| | | | | | | 9H), 4.59 (ddd, J = 10.7, 8.2, |
| | | | | | | 4.5, 1H), 4.50 (s, 2H), 4.42 (s, |
| | | | | | | 2H), 3.17 (dd, J = 13.8, 4.4, |
| | | | | | | 1H), 3.05 (dd, J = 13.8, 10.6, |
| | | | | | | 1H). |

### Biological Assays

### Cell Culture

Human chinese hamster ovary (CHO) cell line stably expressing hCXCR3 was purchased from Euroscreen (Belgium) and culture in HAM's F12 (Invitrogen) containing 10% heat inactivated fetal calf serum (Cancerra, Australia), 50 units ml−1 penicillin, 50 μml−1 streptomycin, (Invitrogen, USA) and 400 μg/ml geneticin (G418) (Calbiochem, San Diego), according to the manufacturer.

Human CXCR3 cDNA was amplified by PCR from a human cells cDNA library (Clontech) and subcloned into pcDNA3.1 (Invitrogen). Murine pre-B L1.2 cells were transfected with hCXCR3-pcDNA3.1 and were grown at 37° C., 5% CO2 in RPMI 1640 medium (Invitrogen, USA) supplemented with 5% heat inactivated fetal calf serum (Cancerra, Australia), 2 mM glutamine (Invitrogen), 50 units ml−1 penicillin, 50 μml−1 streptomycin. Stable transfectant clonal populations were selected using 800 μg/ml geneticin.

### Membrane Preparation

CHO cells expressing the human CXCR3 were disrupted by nitrogen cavitation (Parr Instruments, USA) at 4° C., 800 p.s.i. for 30 min in 50 mM Tris-HCl pH 7.5, 2 mM EDTA, 250 mM Sucrose and protease inhibitors (Roche). Cell membranes were prepared by differential centrifugation (200×g for 10 min, then 100000×g for 60 min). Membranes pellets were re-suspended in 50 mM Tris-HCL pH 7.4, 1 mM EDTA, 10 mM MgCl2, 250 mM sucrose and inhibitor of proteases. Purified CHO-CXCR3 cell membranes were frozen in liquid nitrogen and stored at −80° C.

### Experiment A

### Radioligand Binding

A scintillation proximity assay was used for radioligand competition and saturation binding assays. For each assay point, 1 to 5 μg of human CXCR3 cell membranes were incubated in a final volume of 100 μl in 96 well plates (Corning, USA) for 120 minutes with shaking at room temperature in presence of 100 μg of wheat germ agglutinin-coated scintillation proximity assay beads (WGA-SPA, RPNQ0001, GE Healthcare), 0.05-0.1 nM [125I]I-TAC (Perkin Elmer, 1366Cie/mmol) or 0.1 nM [125I]IP-10 (Perkin Elmer, 2200 Cie/mmol) in binding buffer (50 mM HEPES/KOH pH 7.4, 10 mM MgCl2, 1 mM CaCl2, 0.1% bovine serum albumin (BSA), 100 mM NaCl with protease inhibitor cocktail tablets (Roche). Assay was performed in presence of 1% dimethylsulphoxide (Me2SO). Binding activity was determined using a 1450 Micro-beta scintillation counter (Wallac, UK). Ki values were calculated using the Cheng-Prusoff equation (Cheng and Prusoff, 1973) and represent the average of at least three independent dose response experiments.

### Experiment B

### In Vitro Assays Chemotaxis Assay IP10

### Culture of L1.2 Cells

L1.2 recombinant cells expressing the receptor hCXCR3 were maintained in culture in RPMI 1640 (invitrogen), 5% Foetal Bovine Serum (invitrogen), 2 mM glutamine(invitrogen), 50 u/ml penicillin/streptomycin (invitrogen), at 37° C.-5% CO₂—in an H₂O saturated incubator. In order to stimulate the expression of the receptor, cells were incubated overnight with 5 mM butyric acid (Sigma)

Chemotaxis Assay:

CXCR3 chemokine was diluted with a serial dilution of compounds in the chemotaxis medium (white RPMI 1640 (invitrogen), 5% Foetal Bovine Serum (invitrogen) at 1% DMSO final (chemotaxis medium). The concentration of the ligand IP10 was determined according to the EC80 to be around 0.3 nM. The chemokine/compounds solution was then added in the lower chamber of a chemotaxis system (neuroprobe). A framed filter (8 uM pore size) was placed on the lower chamber. L1.2 cells were centrifuged and resuspended in chemotaxis medium at 3×10⁶ cells/ml and then diluted with the same serial dilution of compounds at 1% DMSO final. This mix of cells/compounds was then incubated at 37° C.-5% CO₂—in an H₂O saturated incubator during 30 minutes. Cells were then dispensed as a drop on each corresponding wells of the chemotaxis system. Cell migration was then induced at 37° C.-5% CO₂—in an H₂O incubator during 4 hours. Filters were then removed and cells that had migrated were transferred in a black plate (Costar). The plates were stored overnight at −80° C. Cell migration ratio was calculated using the cyquant dye (Molecular Probes-C7026).

### Experiment C

### In Vitro Assays Chemotaxis Assay ITAC

L1.2-CXCR3 cells were grown for 24 hours at 0.5×10⁶ cells/ml in chemotaxis medium containing 5 mM butyric acid (Sigma). Compounds were on one hand mixed with 1 nM of CXCL11 (I-TAC) in phenol-red free RPMI 1640 (invitrogen) supplemented with 5% fetal bovine serum in presence of 1% DMSO. The CXCL11/compounds mixture was then added to lower chambers of chemotaxis 96 well microplates (neuroprobe), and framed filters (8 μM pore size) were put on top of the lower chambers. Compounds were on the other hand mixed with L1.2 CXCR3 cells and incubated in chemotaxis medium at 1.5×106 cells/ml in presence of 1% DMSO at 37° C., 5% CO2 for 30 minutes. Cells/compounds mixture was then added on top of the frame filters and migration was performed at 37° C., 5% CO2 for 4 hours. The number of migrated cells in the bottom chamber was determined using the CyQuant GR dye (Molecular Probe), according to the manufacturer.

### Experiment D

### In Vitro Assays CXCR3 Ca²⁺ Mobilization

CXCR3Ca²⁺ mobilization was measured using a stable hCXCR3—CHO cell line and a microtiter-plate based assay using FLIPR^{TETRA}™ (Molecular Devices). In more detail, cells were harvested and plated into black 384-well plates (Becton-Dickinson) at a density of 15 000 cells per well and grown in the incubator for 18 hours. On the next day the media was aspirated and replaced with the cell loading buffer (HBSS—(Invitrogen) based buffer containing calcium indicator and signal enhancer from a commercial Ca2+ assay kit (Becton Dickinson). The plates were incubated for 60 minutes in the incubator, the test compounds were added and the plates equilibrated for 20 minutes at room temperature.

Plates were placed into FLIPR and the CXCR3 agonist (I-TAC, 100 nM) stimulated fluorescence change was quantitated. The activity of CXCR3 antagonists was determined as percent of the CXCR3 ligand I-TAC in the absence of the test compounds (=100% activity). For antagonist potencies, the IC₅₀ is defined as the molar concentration of an antagonist that reduces the I-TAC-induced response to 50%.

The following results have been obtained:

| | | | | L1.2 | |
|---|---|---|---|---|---|
| | | | Binding | chemotaxis | |
| | | Ca- | Ki | IC50 | Chemotaxis/ |
| | | ITAC | (μM) | (μM) | ITAC |
| structure | Ex | (μM) | IP10 | IP10 | (μM) |
| 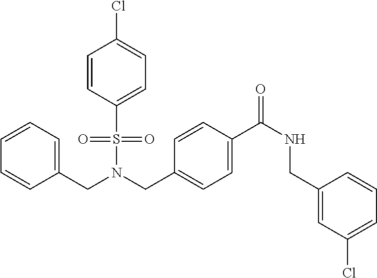 | 1 | — | — | 1.295 | — |
| 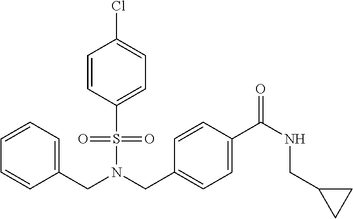 | 2 | — | — | 1.985 | — |
| 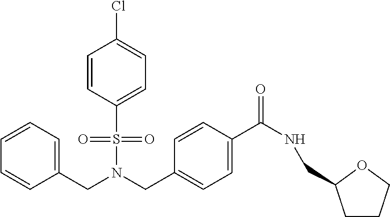 | 3 | — | — | 2.275 | — |
| 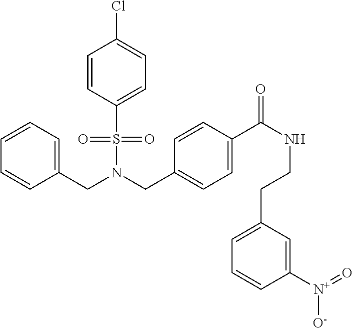 | 4 | — | — | 2.450 | — |
| 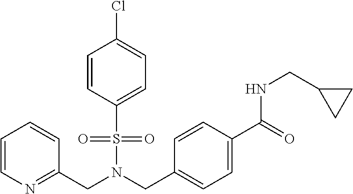 | 5 | — | 0.168 | 0.200 | — |
| 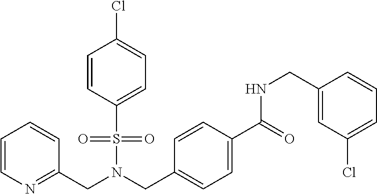 | 6 | — | 0.123 | 0.085 | — |
| 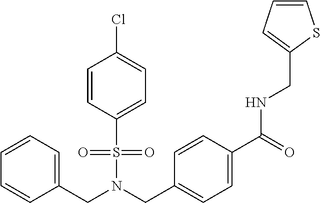 | 7 | — | 0.299 | 0.192 | — |
| 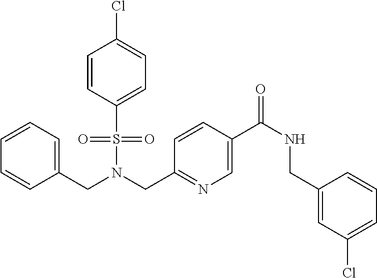 | 8 | — | 0.640 | 0.369 | — |
| 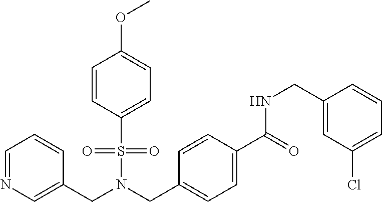 | 9 | — | — | 1.152 | — |
| 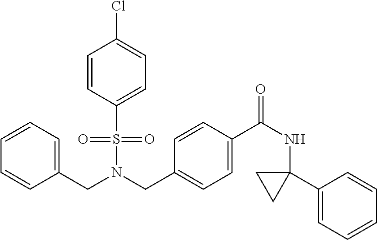 | 10 | — | 0.155 | 0.238 | — |
| 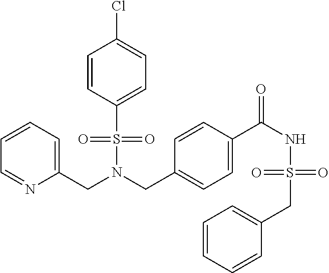 | 11 | — | 0.100 | 0.404 | — |
| 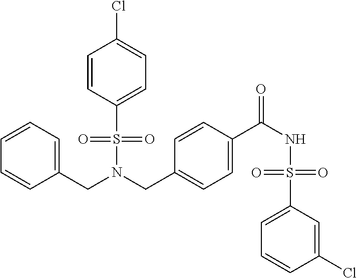 | 12 | — | 1.650 | 0.865 | — |
| 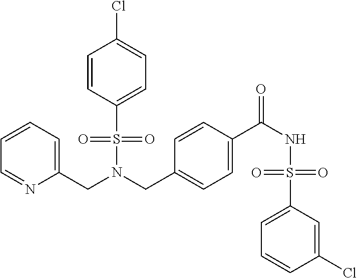 | 13 | — | 0.066 | 0.138 | — |
| 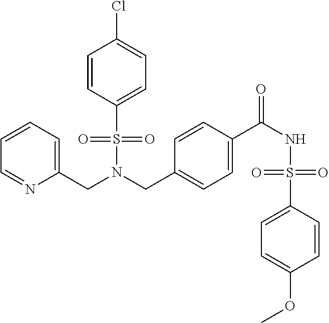 | 14 | — | — | 3.865 | — |
| 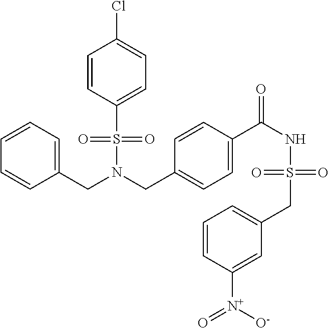 | 15 | — | — | 2.420 | — |
| 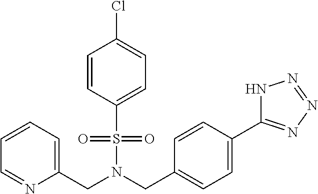 | 16 | — | 0.092 | 0.496 | — |
| 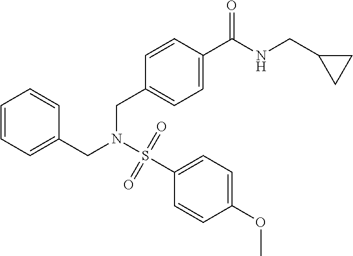 | 17 | — | — | 0.697 | — |
| 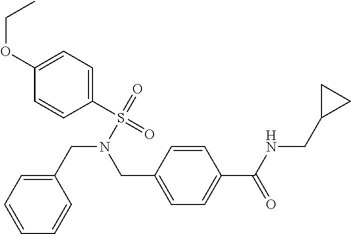 | 18 | — | 0.78 | 0.673 | 1.685 |
| 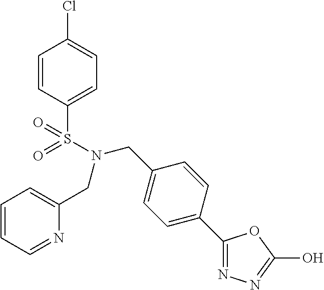 | 19 | — | — | 0.695 | — |
| 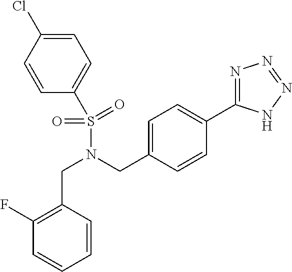 | 20 | — | — | 0.470 | — |
| 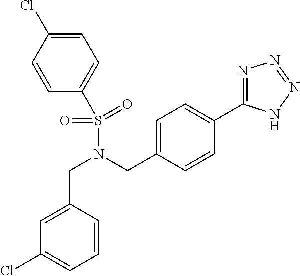 | 21 | — | — | 1.295 | — |
| 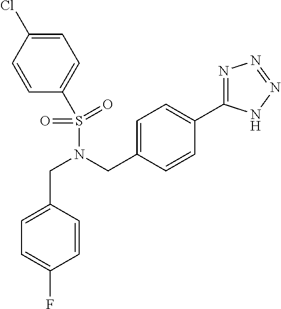 | 22 | — | — | 2.560 | — |
| 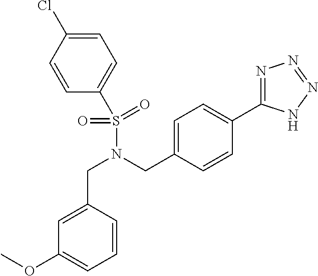 | 23 | — | — | 1.460 | — |
| 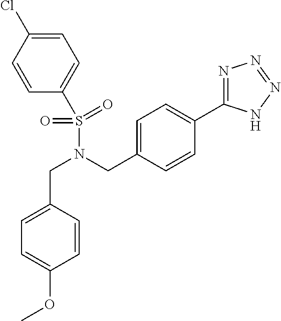 | 24 | — | — | 0.885 | — |
| 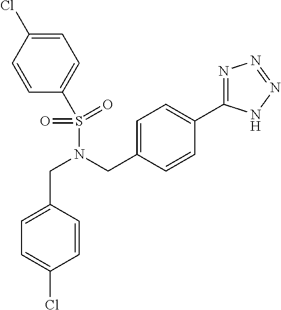 | 25 | — | — | 0.882 | — |
| 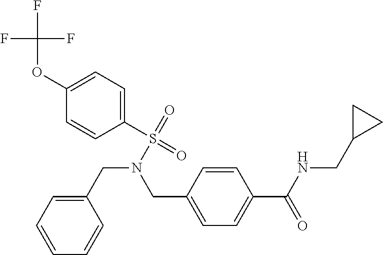 | 26 | — | — | 5.58 | — |
| 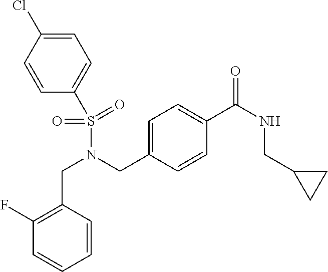 | 29 | 1 | — | 0.51 | — |
| 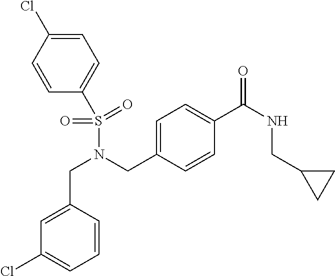 | 30 | — | — | 0.94 | — |
| 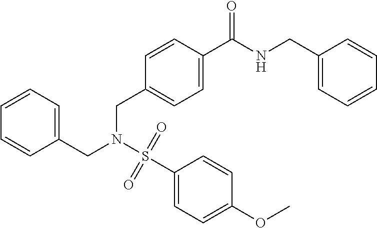 | 31 | 1.9 | — | 0.347 | — |
| 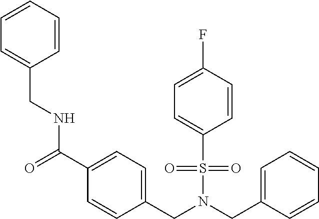 | 32 | 3.9 | — | — | — |
| 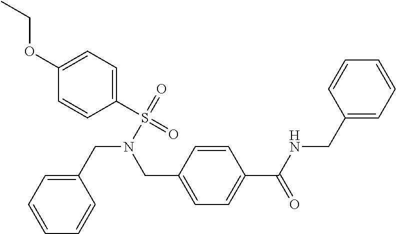 | 33 | 0.53 | 0.197 | — | — |
| 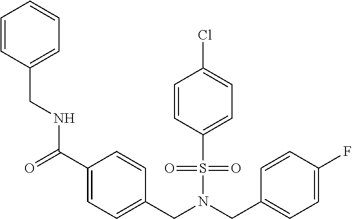 | 34 | 1.8 | — | — | — |
| 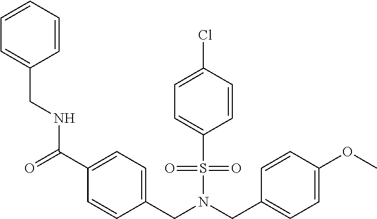 | 35 | 6 | — | — | — |
| 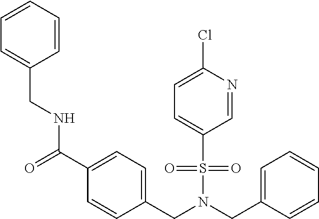 | 36 | 0.15 | — | — | — |
| 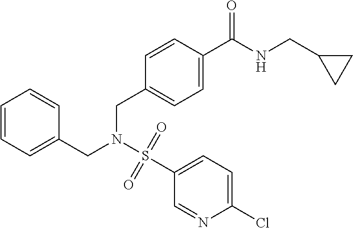 | 37 | 3.35 | — | 0.328 | — |
| 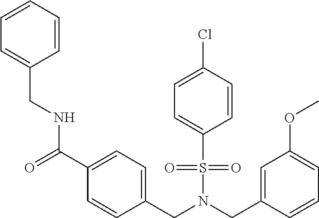 | 38 | 10 | — | — | — |
| 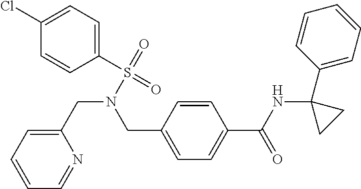 | 40 | 0.09 | 0.015 | 0.025 | 0.053 |
| 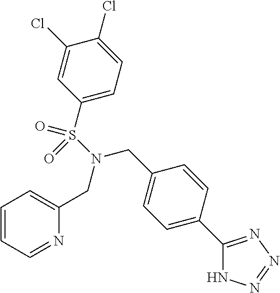 | 41 | 1.375 | 0.373 | 1.15 | — |
| 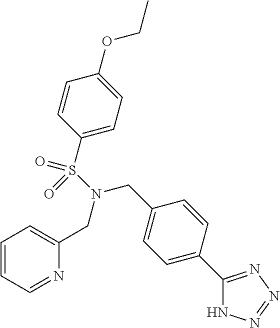 | 42 | 0.52 | 0.161 | 0.54 | 1.035 |
| 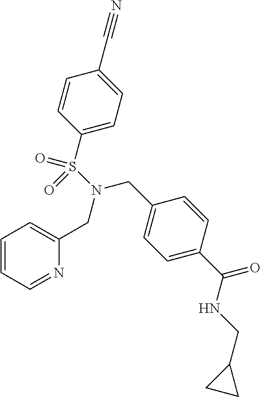 | 43 | 4.75 | — | 0.36 | — |
| 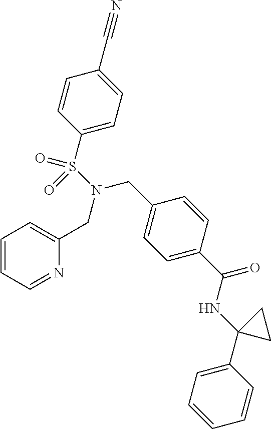 | 44 | 0.268 | 0.01 | 0.013 | 0.033 |
| 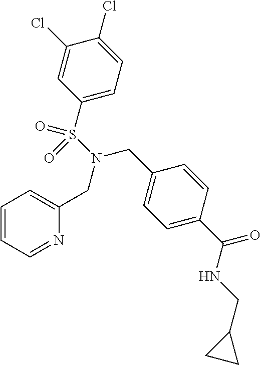 | 45 | 13 | — | — | — |
| 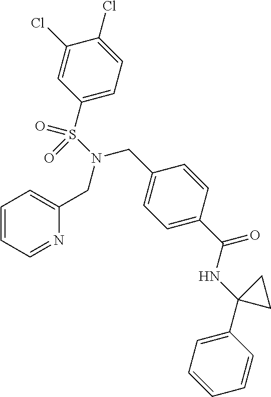 | 46 | 0.705 | — | — | — |
| 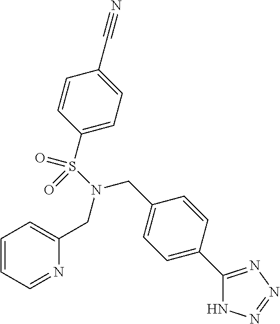 | 47 | 1.155 | — | 0.233 | — |
| 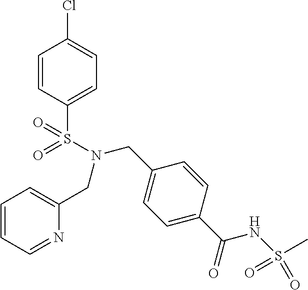 | 48 | 8.3 | — | — | — |
| 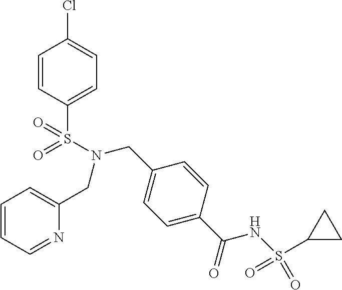 | 49 | 5.6 | — | — | — |
| 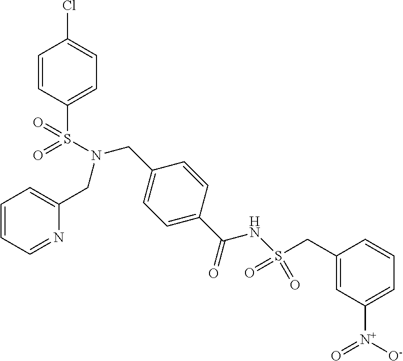 | 50 | 0.175 | — | 0.207 | — |
| 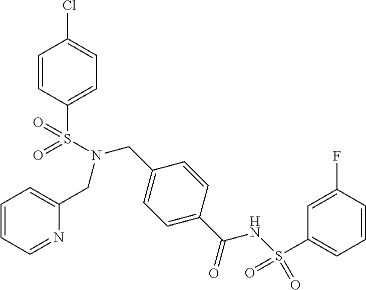 | 51 | 0.131 | — | 0.339 | — |
| 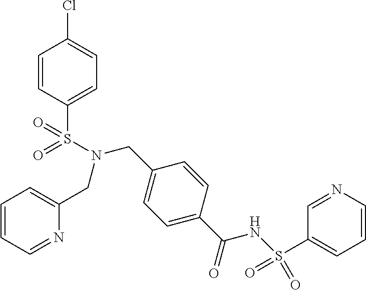 | 52 | 0.285 | — | — | — |
| 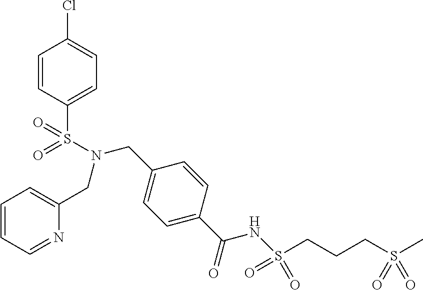 | 53 | 1.7 | — | 1.317 | — |
| 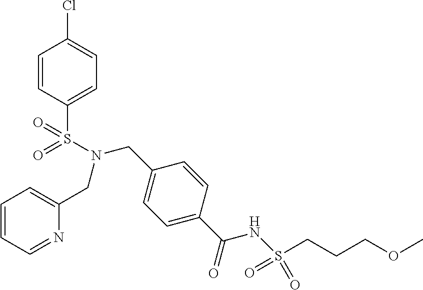 | 54 | 5.6 | — | 3.83 | — |
| 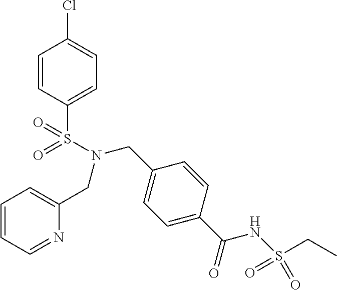 | 55 | 11 | — | — | — |
| 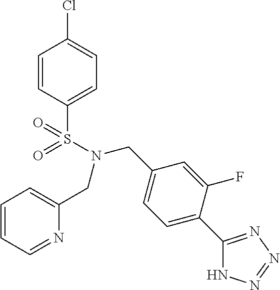 | 56 | 5.3 | — | 1.16 | — |
| 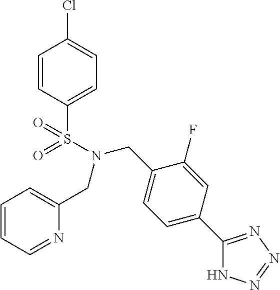 | 57 | 5 | — | 0.44 | 1.78 |
| 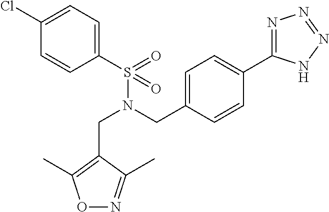 | 58 | — | — | 1.428 | — |
| 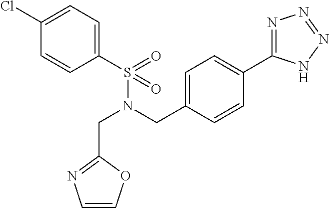 | 59 | — | — | 3.67 | — |
| 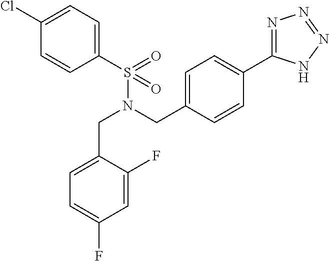 | 60 | 1.45 | — | 0.681 | — |
| 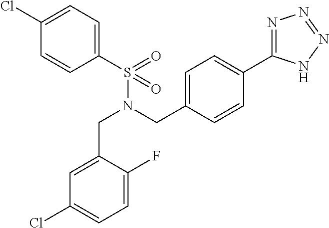 | 61 | 0.2 | — | — | — |
| 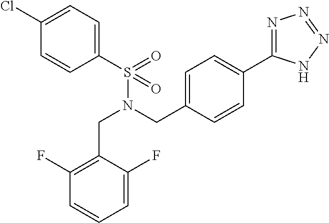 | 62 | 0.044 | — | 0.183 | 0.212 |
| 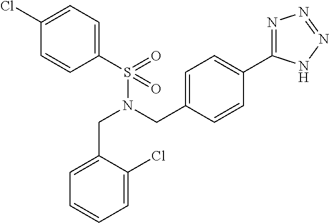 | 63 | 0.415 | — | 0.548 | — |
| 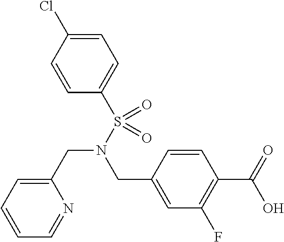 | 64 | 3.038 | — | 1.132 | 3.15 |
| 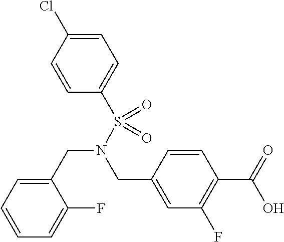 | 65 | 1.005 | — | 0.513 | 2.295 |
| 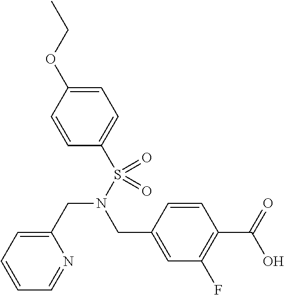 | 66 | 2 | — | — | 4.6 |
| 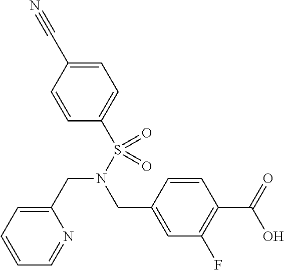 | 67 | 0.505 | — | 1.08 | 3.065 |
| 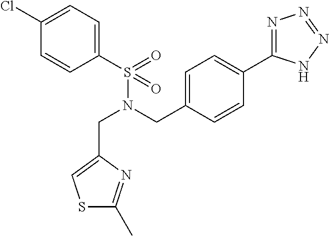 | 68 | 2 | — | — | 0.13 |
| 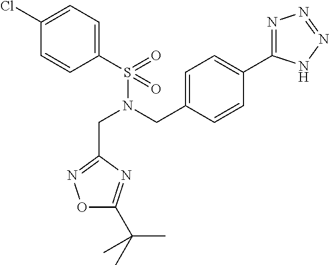 | 69 | 0.056 | — | 0.254 | 0.5605 |
| 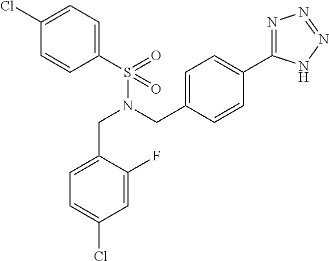 | 70 | 3 | — | — | — |
| 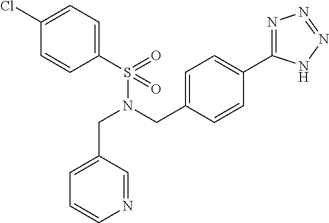 | 71 | 16 | — | — | 1.54 |
| 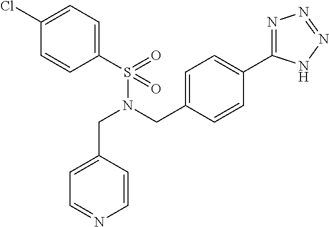 | 72 | — | — | — | — |
| 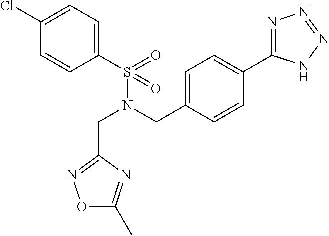 | 73 | 12 | — | — | 3.24 |
| 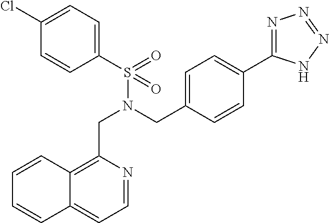 | 74 | 2 | — | — | 0.666 |
| 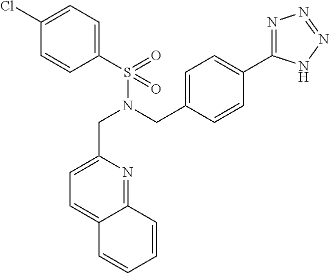 | 75 | 2 | — | — | 2.525 |
| 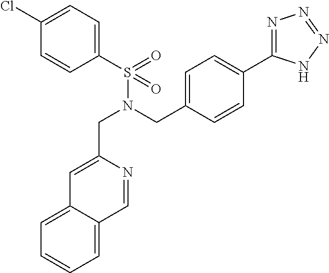 | 76 | 0.3 | — | 0.911 | — |
| 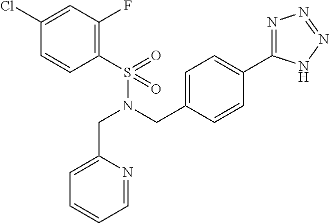 | 77 | 0.5 | — | 0.409 | 0.409 |
| 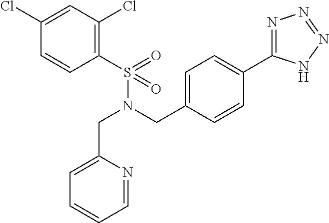 | 78 | 3 | — | — | — |
| 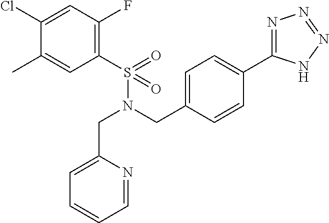 | 79 | 4 | — | — | — |
| 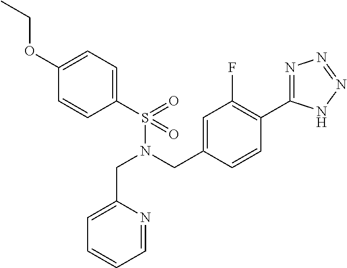 | 80 | 0.055 | — | 0.158 | 0.909 |
| 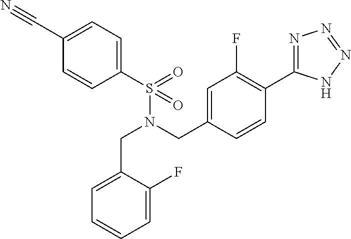 | 81 | 0.079 | — | 0.1925 | 0.5655 |
| 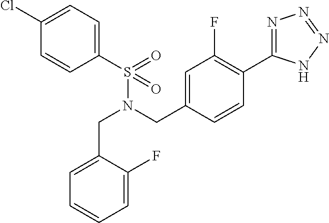 | 82 | 0.15 | — | 0.157 | — |
| 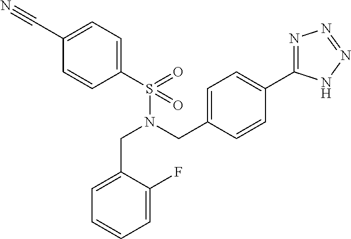 | 83 | 0.01 | — | 0.236 | 1.04 |
| 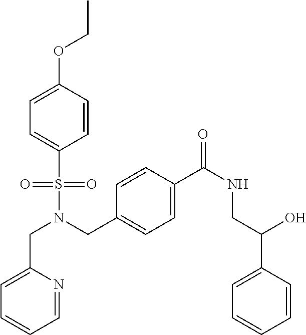 | 84 | 0.02 | — | 0.01 | 0.02 |
| 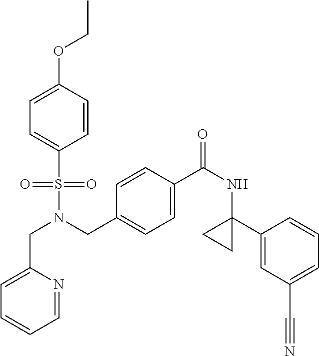 | 85 | 4 | — | — | — |
| 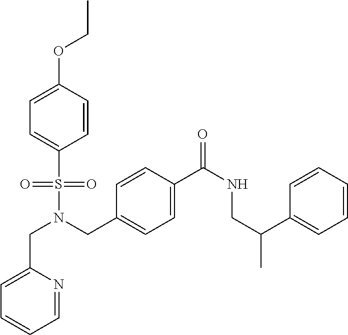 | 86 | 1.7 | — | — | — |
| 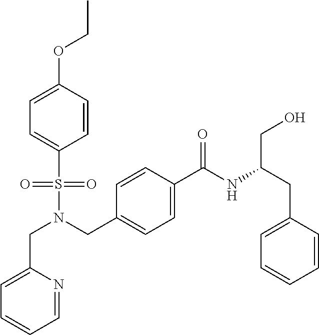 | 87 | 17 | — | — | 0.76 |
| 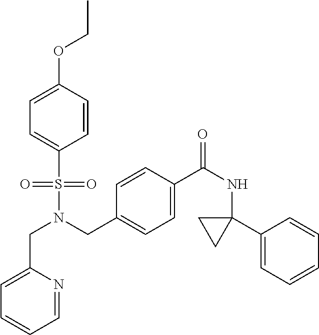 | 88 | 0.01 | — | 0.01 | 0.03 |
| 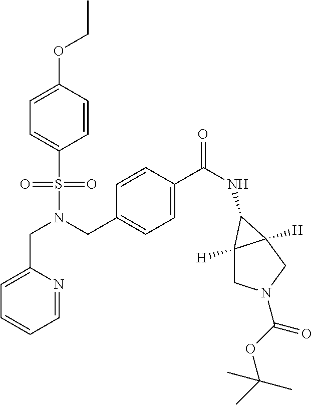 | 89 | 0.05 | — | 0.01 | — |
| 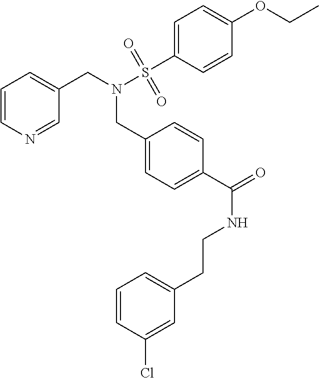 | 90 | 1.9 | — | — | — |
| 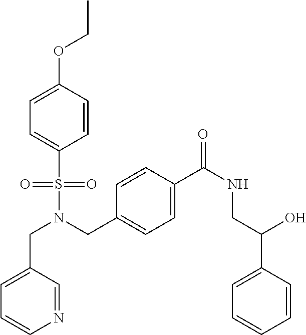 | 91 | 0.03 | — | 0.05 | 0.17 |
| 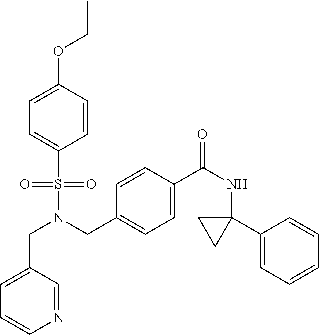 | 93 | 0.03 | — | 0.04 | — |
| 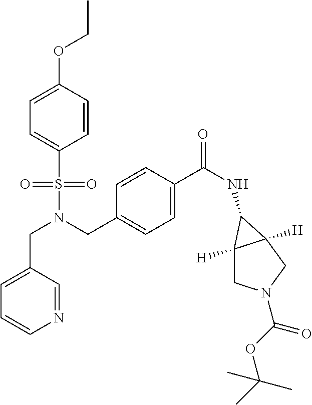 | 94 | 2 | — | — | 0.26 |
| 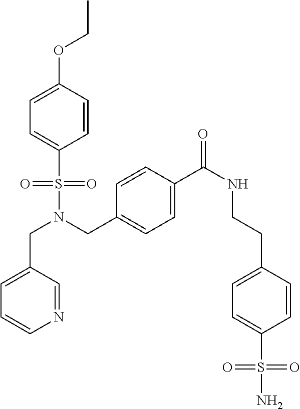 | 95 | 2 | — | — | — |
| 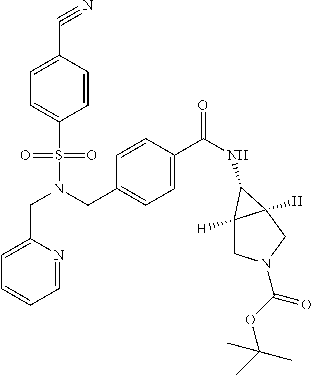 | 96 | 0.01 | — | 0.01 | — |
| 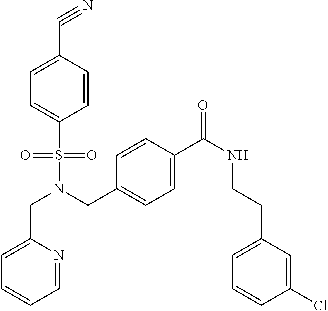 | 97 | 0.07 | — | 0.05 | — |
| 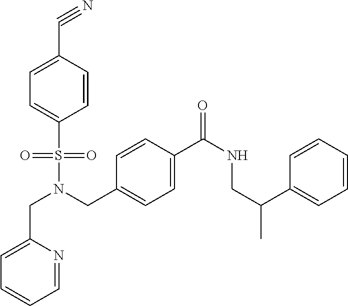 | 98 | 0.4 | — | 0.16 | — |
| 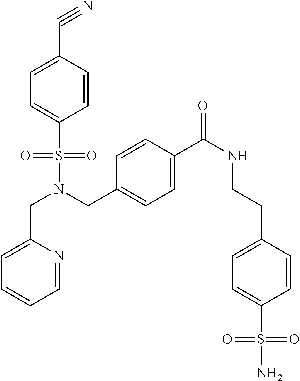 | 99 | 1.45 | — | — | 1.03 |
| 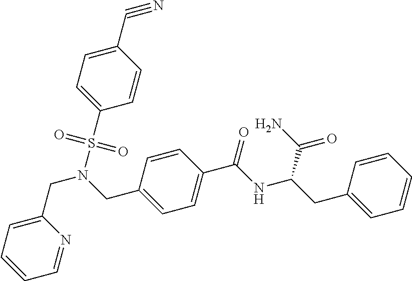 | 100 | 0.2 | — | — | 0.63 |
| 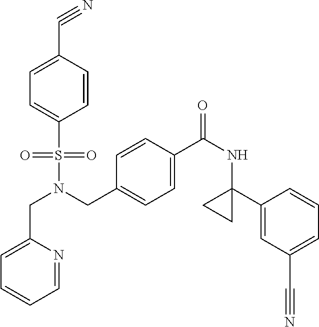 | 101 | 0.06 | — | 0.19 | — |
| 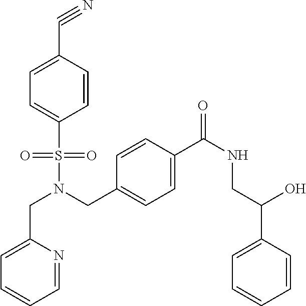 | 102 | 0.02 | — | 0.01 | 0.02 |
| 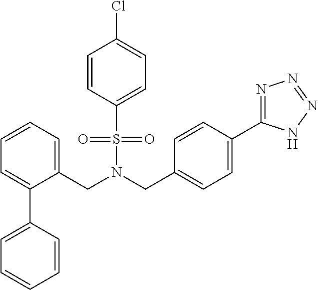 | 103 | 3.45 | — | — | — |
| 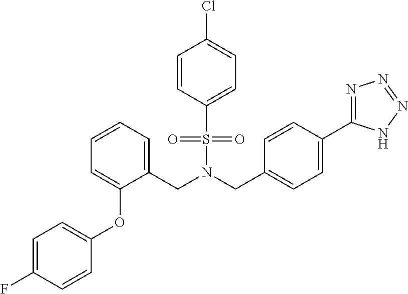 | 104 | 1 | — | — | — |
| 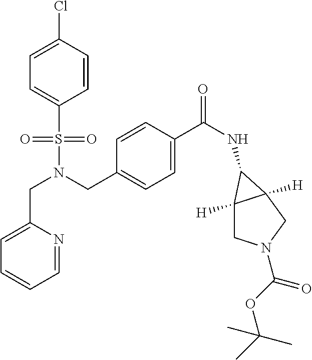 | 105 | 0.02 | — | — | 0.07 |
| 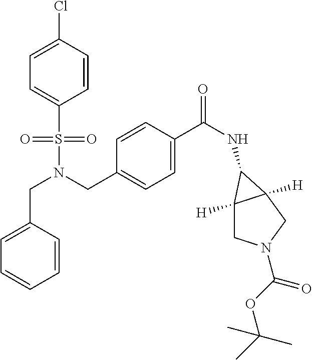 | 106 | 0.3 | — | — | 0.51 |
| 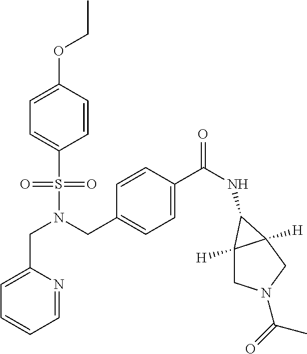 | 107 | 5.5 | — | — | 2.07 |
| 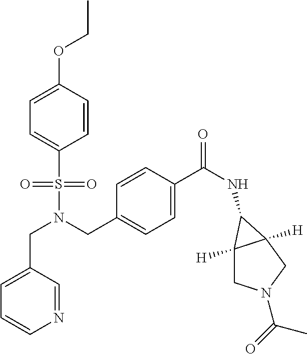 | 108 | 1.8 | — | — | — |
| 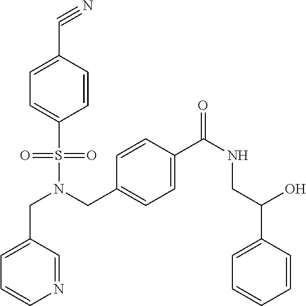 | 109 | 0.75 | — | — | 0.13 |
| 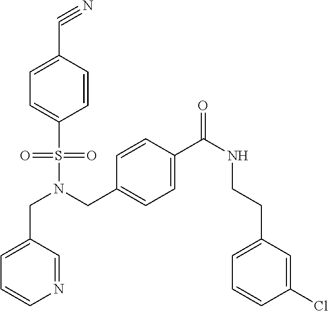 | 110 | 0.75 | — | — | 0.39 |
| 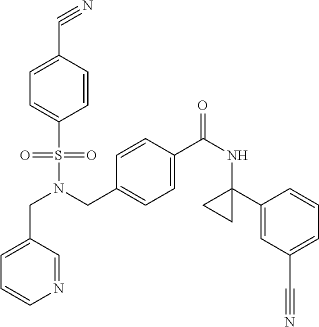 | 111 | 1.5 | — | — | — |
| 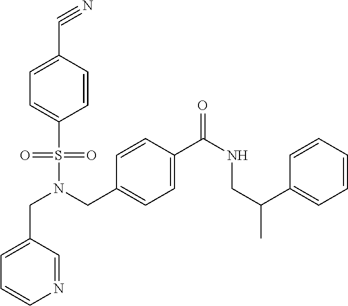 | 112 | 1.25 | — | — | — |
| 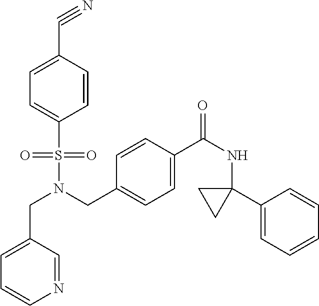 | 113 | 0.32 | — | — | 1.3 |
| 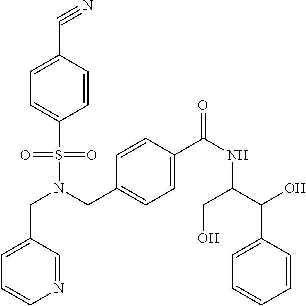 | 114 | 2 | — | — | 2.5 |
| 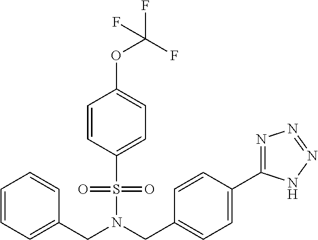 | 115 | 2 | — | — | — |
| 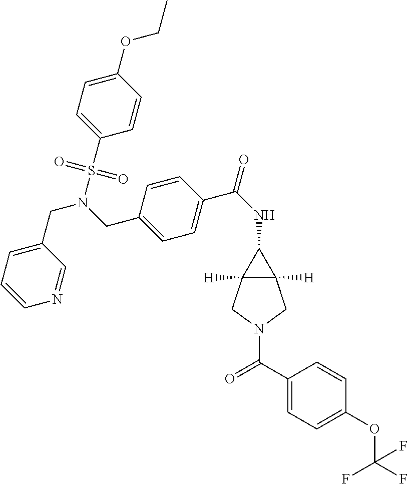 | 116 | 2 | — | — | — |
| 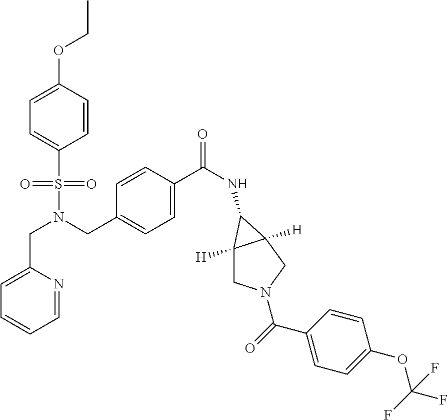 | 117 | 0.8 | — | — | — |
| 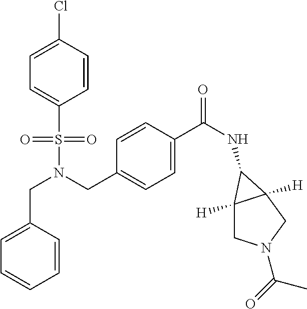 | 118 | 3 | — | — | 2.61 |
| 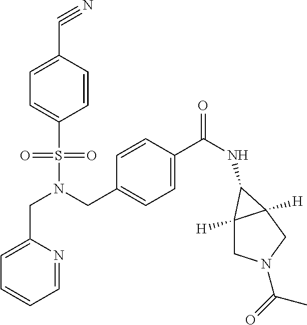 | 119 | 0.87 | — | — | 2.14 |
| 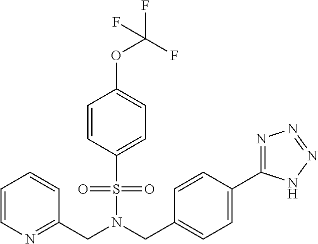 | 120 | 0.2 | — | — | 4.47 |
| 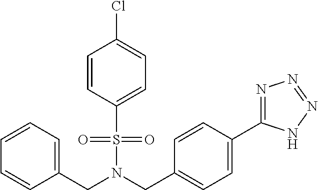 | 121 | 3 | — | — | 3.89 |
| 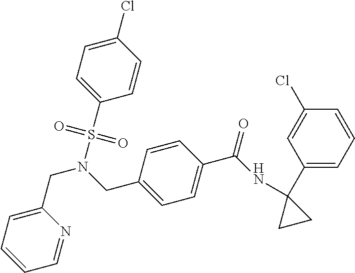 | 122 | 0.6 | — | — | 0.24 |
| 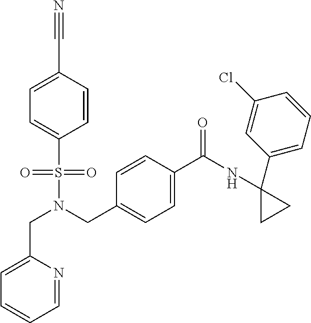 | 123 | 0.3 | — | — | 0.06 |
| 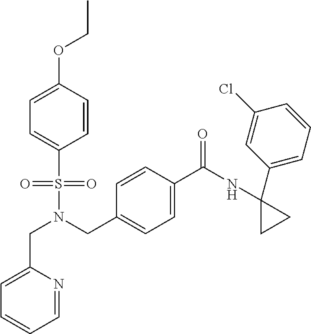 | 124 | 0.5 | — | — | 0.16 |
| 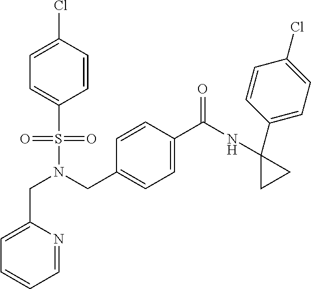 | 125 | 0.4 | — | — | 0.35 |
| 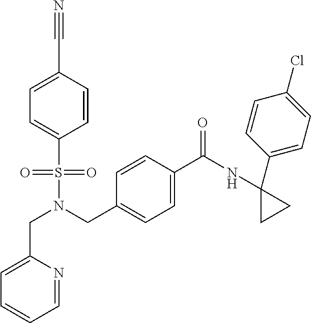 | 126 | 0.7 | — | — | 0.17 |
| 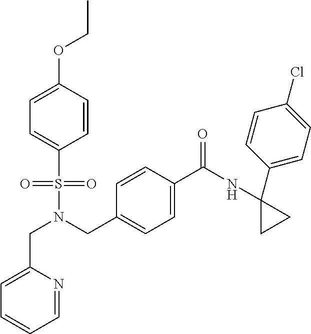 | 127 | 0.5 | — | — | 0.38 |
| 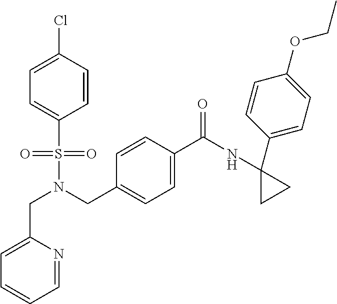 | 128 | 0.85 | — | — | — |
| 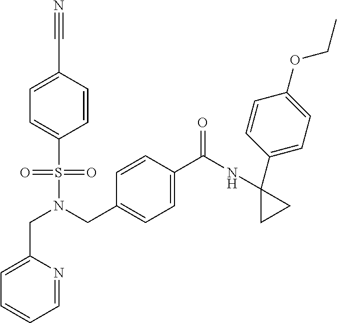 | 129 | 2.5 | — | — | — |
| 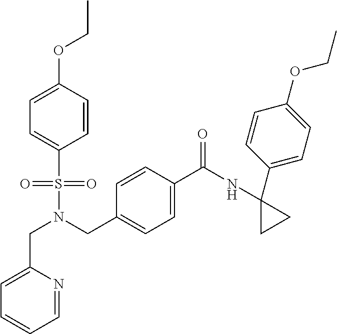 | 130 | 3 | — | — | — |
| 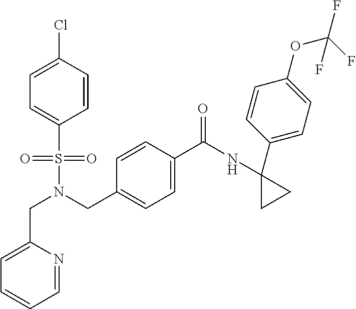 | 131 | 5 | — | — | — |
| 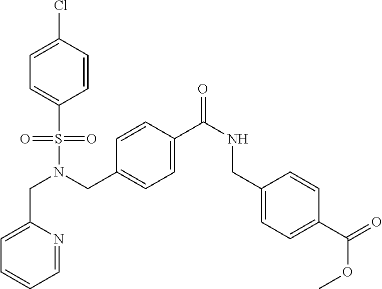 | 132 | 0.9 | — | — | 0.27 |
| 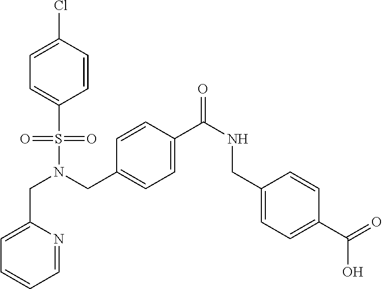 | 133 | 2 | — | — | — |
| 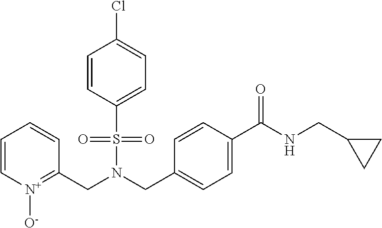 | 134 | — | — | — | 2.98 |
| 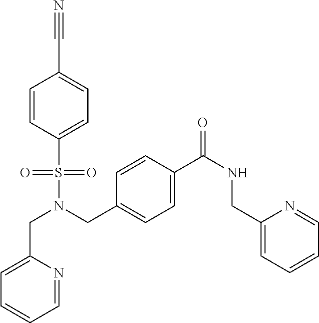 | 135 | 0.9 | — | — | 0.75 |
| 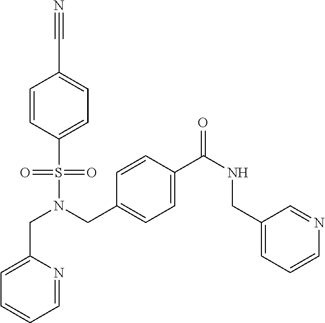 | 136 | 8 | — | — | 0.93 |
| 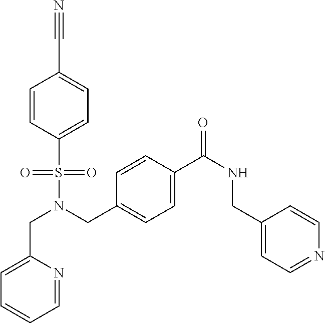 | 137 | 3 | — | — | 4.31 |
| 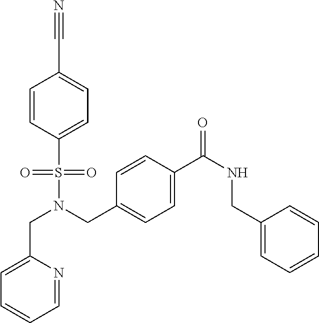 | 138 | 0.1 | — | — | 0.06 |
| 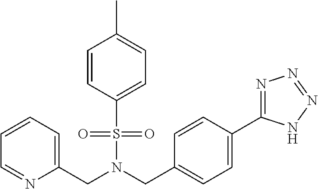 | 139 | 2 | — | — | — |
| 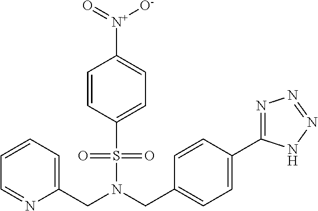 | 140 | 2 | — | — | — |
| 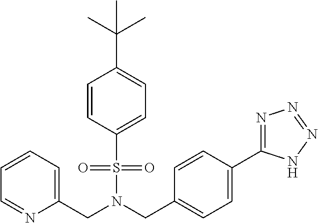 | 141 | 3 | — | — | — |
| 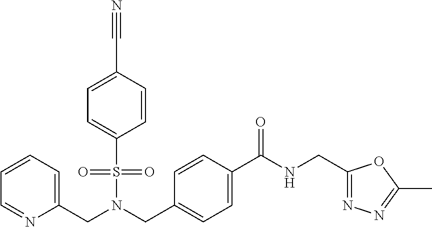 | 142 | 10 | — | — | 4.18 |
| 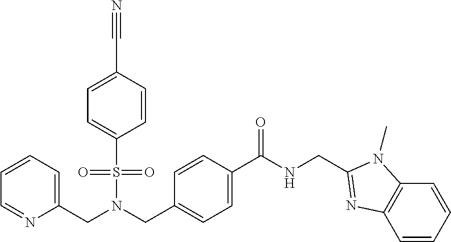 | 143 | 0.4 | — | — | 1 |
| 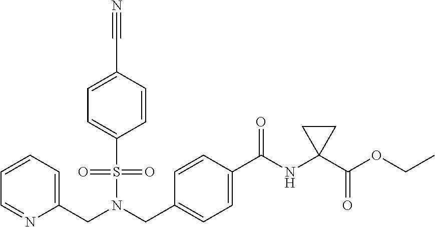 | 144 | 0.14 | — | — | — |
| 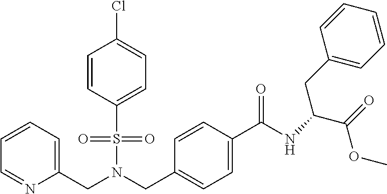 | 145 | 2 | — | — | 0.4 |
| 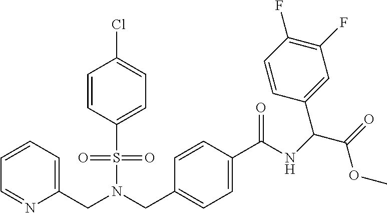 | 147 | 0.4 | — | — | 0.082 |
| 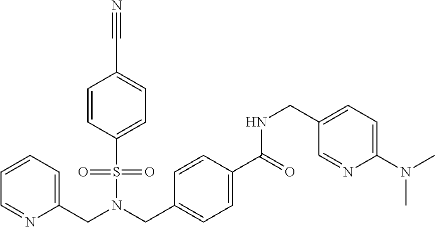 | 148 | 3 | — | — | — |
| 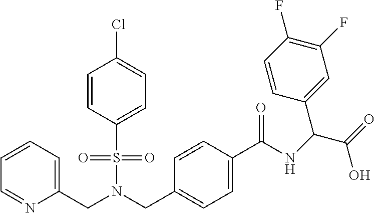 | 149 | 0.1 | — | — | 0.073 |
| 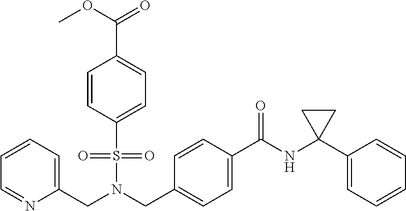 | 150 | 0.3 | — | — | 0.01 |
| 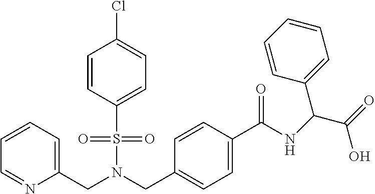 | 151 | 0.15 | — | — | — |
| 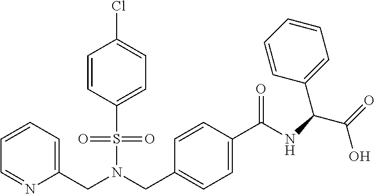 | 152 | 0.75 | — | — | — |
| 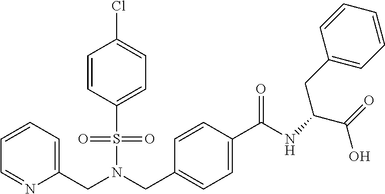 | 153 | 2 | — | — | — |
| 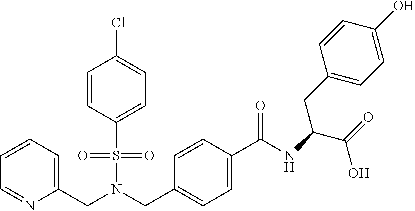 | 154 | 4 | — | — | — |
| 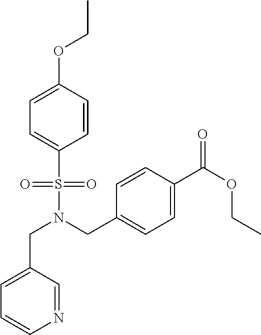 | 155 | 2 | — | — | — |
| 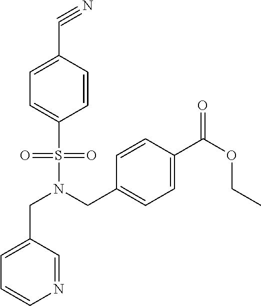 | 156 | 1.9 | — | — | — |
| 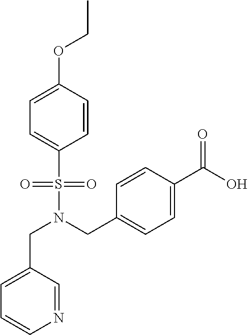 | 157 | 1.62 | — | 4.18 | — |
| 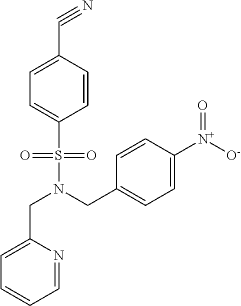 | 158 | 1 | — | — | 4.8 |
| 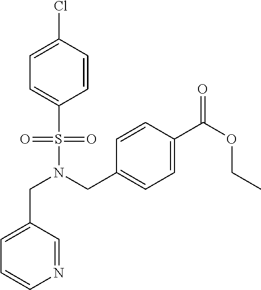 | 159 | 4 | — | — | — |
| 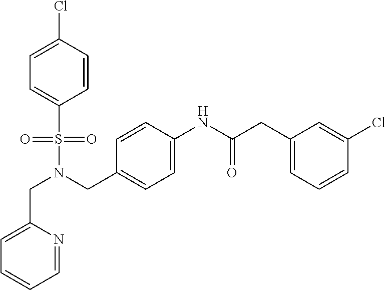 | 160 | 0.1 | — | 0.12 | — |
| 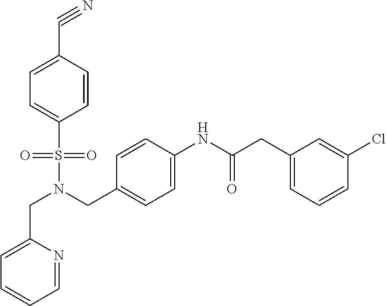 | 161 | 0.02 | — | 0.06 | — |
| 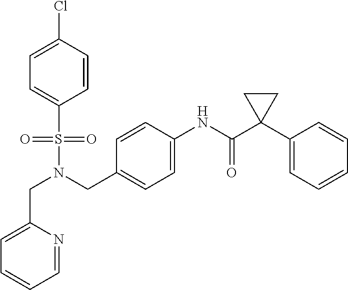 | 162 | 0.17 | — | 0.07 | 0.17 |
| 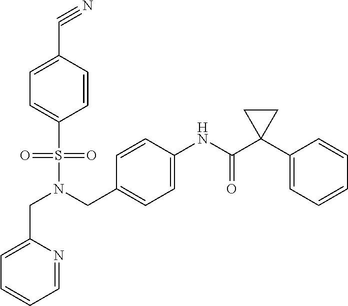 | 163 | 0.02 | — | 0.02 | 0.12 |
| 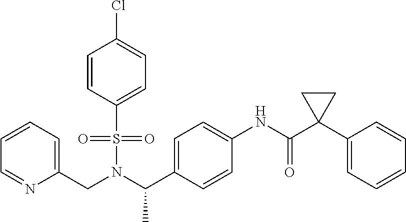 | 164 | — | — | — | 4.27 |
| 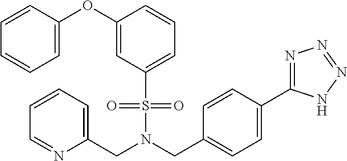 | 165 | 1.1 | — | — | — |
| 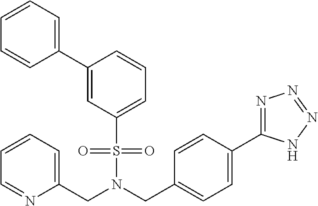 | 166 | 1.43 | — | — | 9.05 |
| 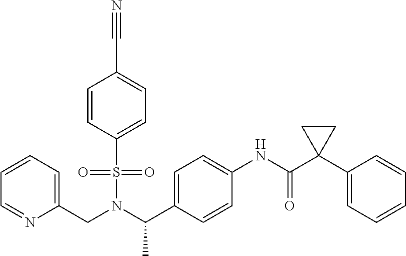 | 167 | 0.25 | — | — | 1.96 |
| 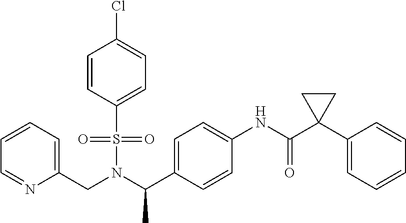 | 168 | 3.33 | — | — | — |
| 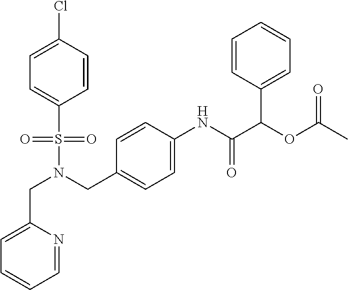 | 169 | 0.7 | — | — | 0.01 |
| 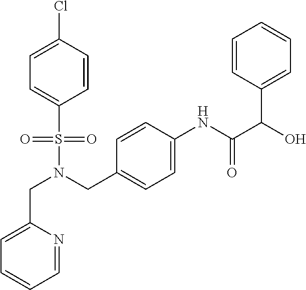 | 170 | 0.4 | — | — | 0.05 |
| 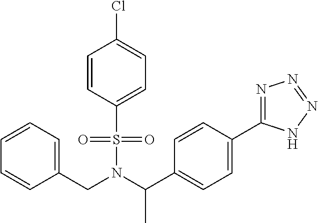 | 171 | 1.95 | — | — | — |
| 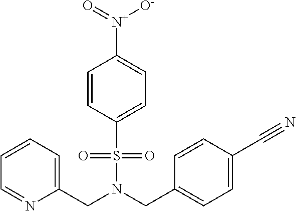 | 172 | 0.7 | — | — | — |
| 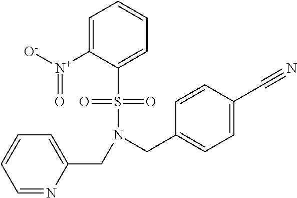 | 173 | 0.4 | — | — | — |
| 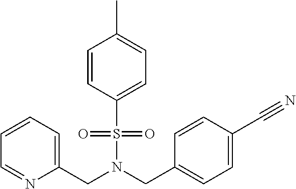 | 174 | 1.5 | — | — | — |
| 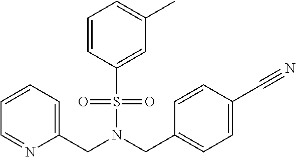 | 175 | 3.5 | — | — | — |
| 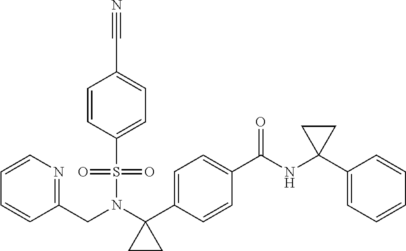 | 176 | — | — | — | 4.45 |
| 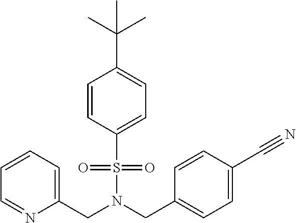 | 177 | 1.45 | — | — | — |
| 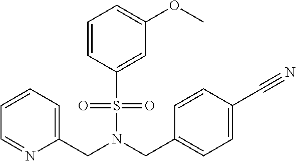 | 178 | 2 | — | — | — |
| 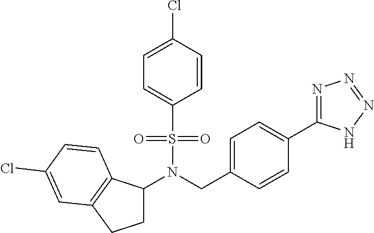 | 179 | 0.35 | — | — | 8.86 |
| 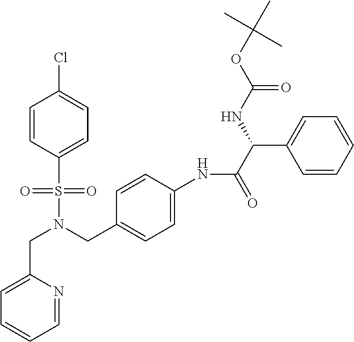 | 180 | 1 | — | — | — |
| 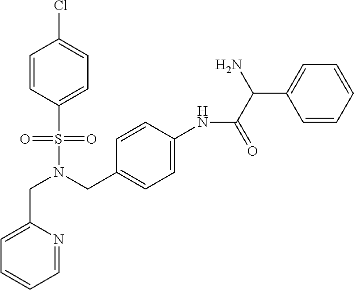 | 181 | 0.15 | — | — | 0.094 |
| 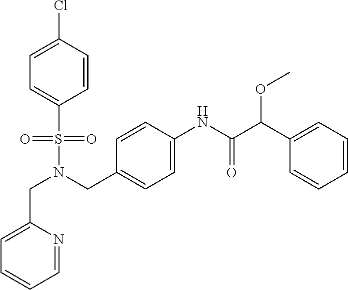 | 182 | 0.04 | — | — | — |
| 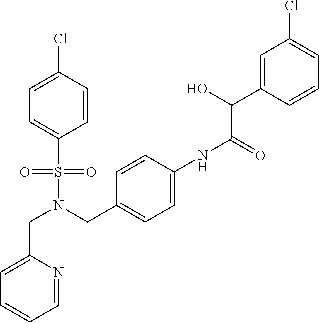 | 184 | 1 | — | — | — |
| 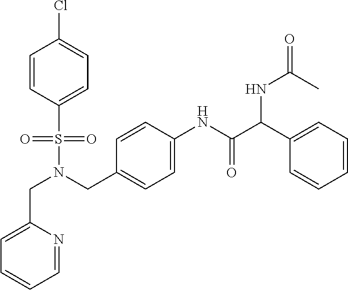 | 185 | 0.3 | — | — | 0.03 |
| 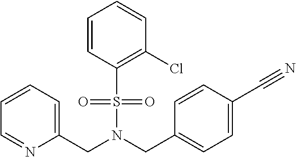 | 186 | 5 | — | — | — |
| 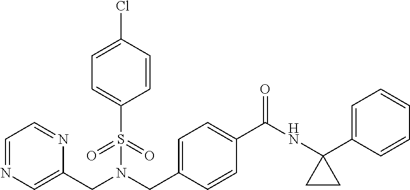 | 187 | 4 | — | — | — |
| 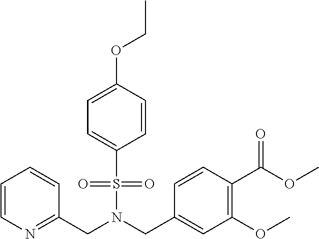 | 188 | 10.5 | — | — | 3.8 |
| 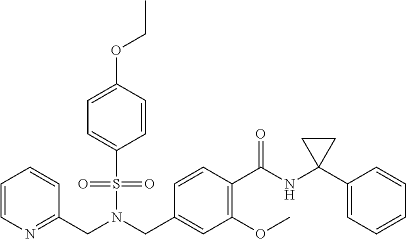 | 189 | 2 | — | — | 0.28 |
| 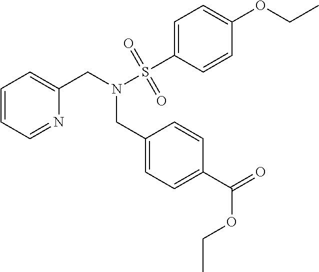 | 191 | 13 | — | — | — |
| 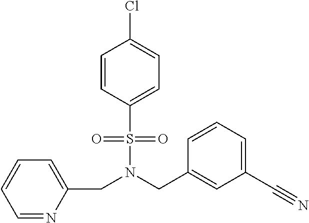 | 195 | 4.2 | — | — | 6.81 |
| 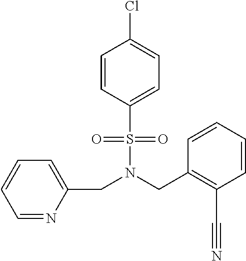 | 196 | 13 | — | — | — |
| 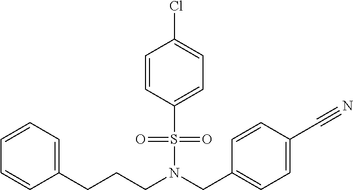 | 198 | 3.1 | — | — | — |
| 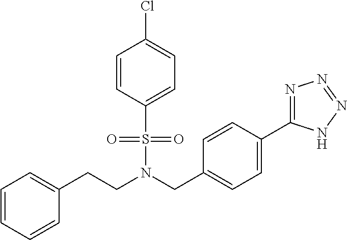 | 204 | 2.01 | — | — | — |
| 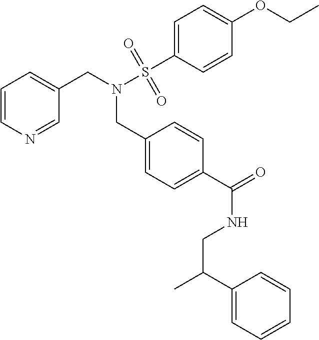 | 206 | 4.5 | — | — | — |
| 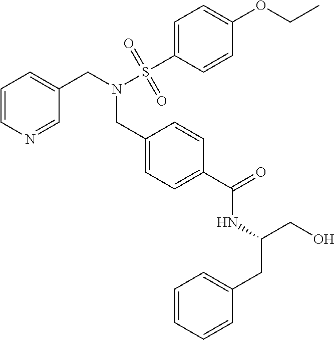 | 209 | 30 | — | — | — |
| 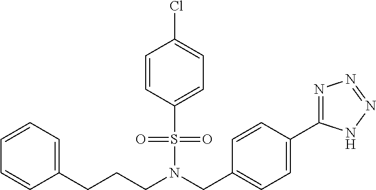 | 221 | 1.7 | — | — | — |
| 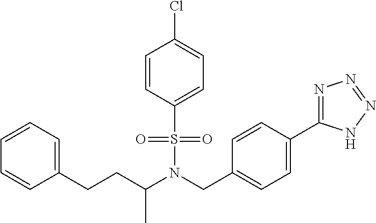 | 224 | 1.5 | — | — | — |
| 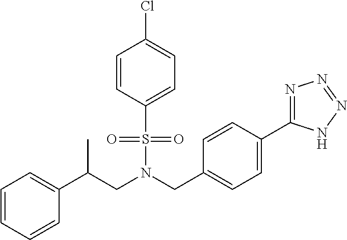 | 227 | 5 | — | — | — |
| 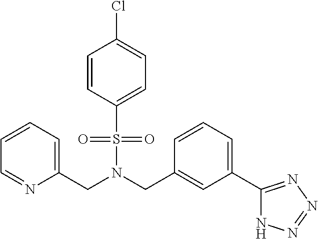 | 228 | 2.8 | — | — | — |
| 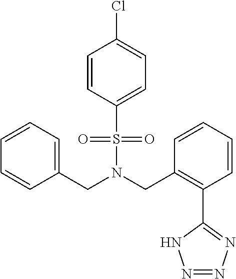 | 229 | 1.6 | — | — | — |
| 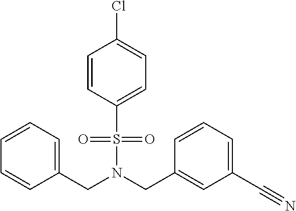 | 230 | 1.2 | — | — | — |
| 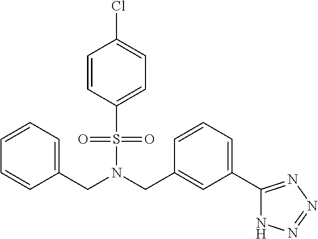 | 234 | 1.5 | — | — | — |
| 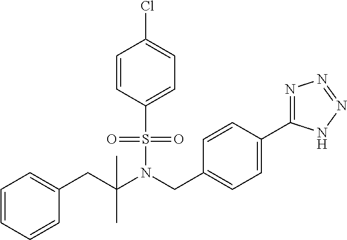 | 236 | 3 | — | — | — |
| 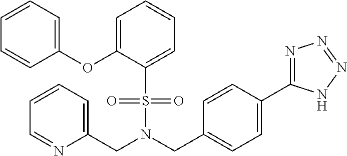 | 237 | 25 | — | — | — |
| 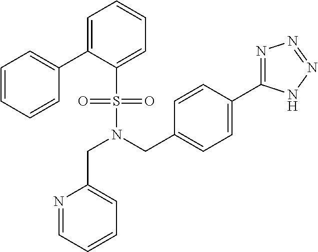 | 253 | 9.5 | — | — | — |
| 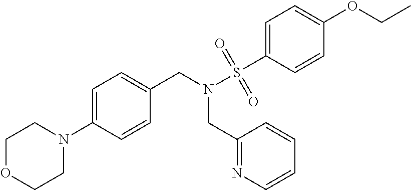 | 269 | 9 | — | — | — |
| 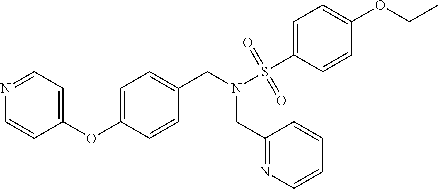 | 270 | 6 | — | — | — |
| 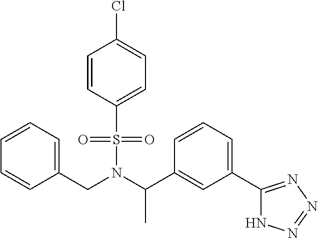 | 271 | 5 | — | — | — |
| 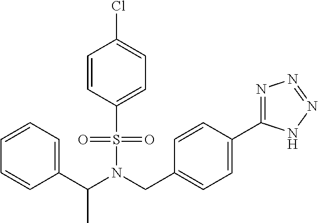 | 272 | 30 | — | — | — |
| 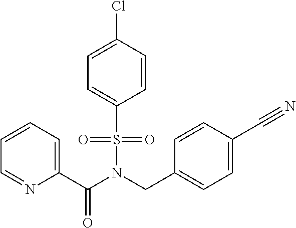 | 279 | 10 | — | — | — |
| 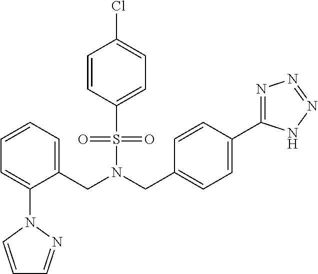 | 280 | 20 | — | — | — |
| 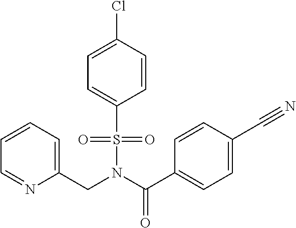 | 281 | 7 | — | — | — |
| 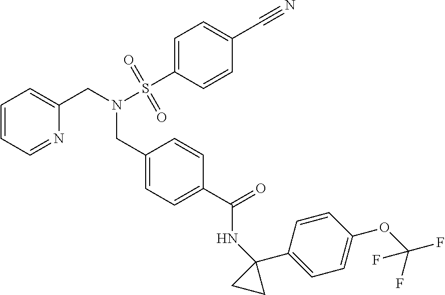 | 282 | 5.5 | — | — | — |
| 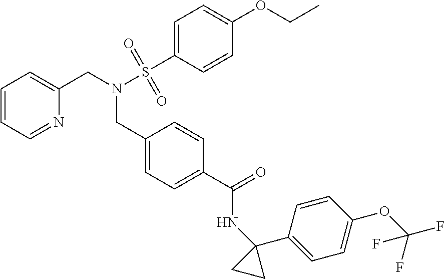 | 283 | 7.5 | — | — | — |
| 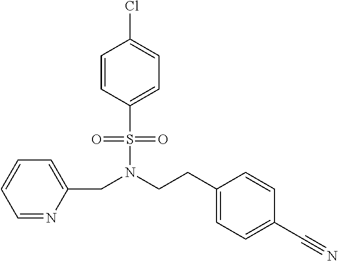 | 286 | 4 | — | — | — |
| 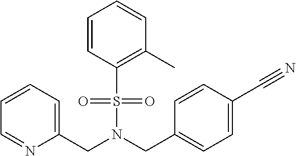 | 287 | 7 | — | — | — |
| 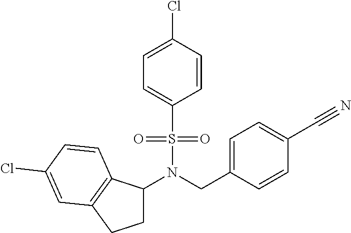 | 299 | 5.5 | — | — | — |
| 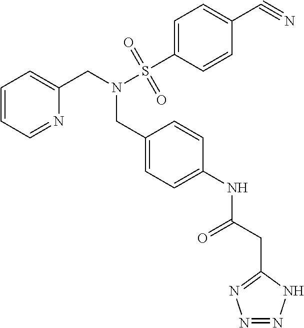 | 300 | 12 | — | — | — |
| 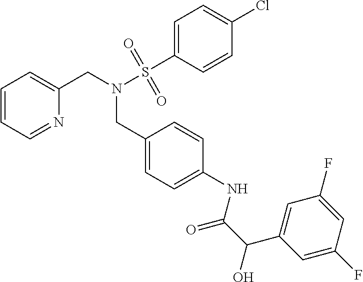 | 309 | 30 | — | — | — |
| 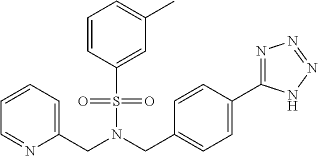 | 311 | 10 | — | — | — |
| 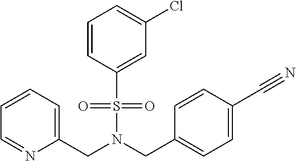 | 313 | 15 | — | — | — |
| 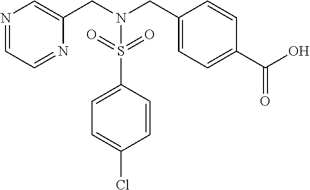 | 319 | 20 | — | — | — |
| 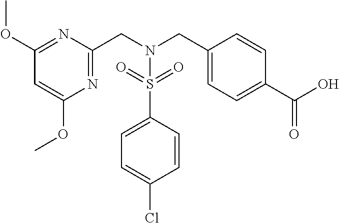 | 328 | 13 | — | — | — |
| 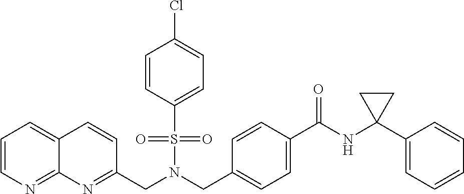 | 330 | 10 | — | — | — |
| 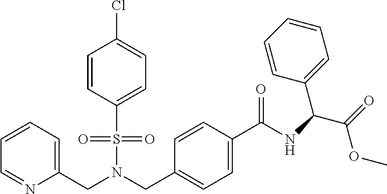 | 342 | 0.8 | — | — | 0.054 |
| 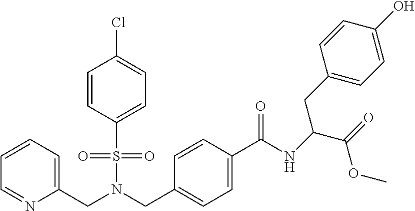 | 345 | 18 | — | — | — |
| 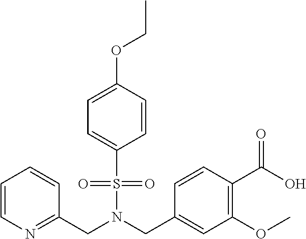 | 346 | 7 | — | — | — |
| 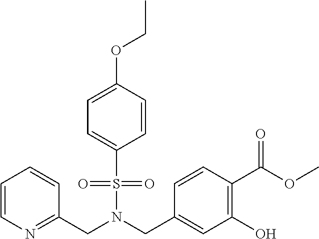 | 353 | 6 | — | — | — |
| 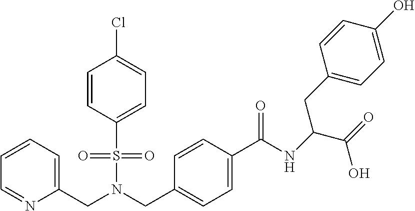 | 354 | 6 | -- | — | — |

### Preparation of a Pharmaceutical Formulation

### Formulation 1

### Tablets

A compound of formula (I) is admixed as a dry powder with a dry gelatin binder in an approximate 1:2 weight ratio. A minor amount of magnesium stearate is added as a lubricant. The mixture is formed into 240-270 mg tablets (80-90 mg of active compound according to the invention per tablet) in a tablet press.

### Formulation 2

### Capsules

A compound of formula (I) is admixed as a dry powder with a starch diluent in an approximate 1:1 weight ratio. The mixture is filled into 250 mg capsules (125 mg of active compound according to the invention per capsule).

### Formulation 3

### Liquid

A compound of formula (I) (1250 mg), sucrose (1.75 g) and xanthan gum (4 mg) are blended, passed through a No. 10 mesh U.S. sieve, and then mixed with a previously prepared solution of microcrystalline cellulose and sodium carboxymethyl cellulose (11:89, 50 mg) in water. Sodium benzoate (10 mg), flavor, and color are diluted with water and added with stirring. Sufficient water is then added to produce a total volume of 5 mL.

### Formulation 4

### Tablets

A compound of formula (I) is admixed as a dry powder with a dry gelatin binder in an approximate 1:2 weight ratio. A minor amount of magnesium stearate is added as a lubricant. The mixture is formed into 450-900 mg tablets (150-300 mg of active compound according to the invention) in a tablet press.

### Formulation 5

### Injection

A compound of formula (I) is dissolved in a buffered sterile saline injectable aqueous medium to a concentration of approximately 5 mg/mL.

1-15. (canceled)

16. A compound of formula (I): 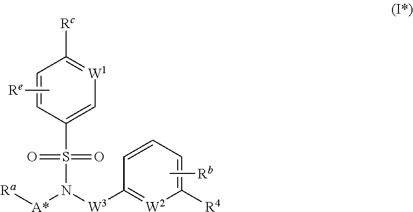
wherein:
A* represents V, a C-1 to C-6 alkylene group that is unsubstituted or substituted by R^{f}, R^{g}, carbonyl (═O), or by a group —C(O)—OR^{f} or —C(O)NR^{f}R^{g};
V represents a —CO— group, a linear or branched (C1-C6)-alkylene group, or a bond;
W¹, W² are independently of one another N or CH;
W³ represents CR¹R² or a C-1 to C-6 alkylene group that is unsubstituted or substituted by R^{f}, R^{g}, carbonyl (═O), or by a group —C(O)—OR^{f} or —C(O)NR^{f}R^{g};
R^{a} denotes Ar or Het;
R^{b} denotes Hal, Ar, CN, Het, —NO₂, —N(R³)₂, —NH—C(O)A, —COOR^{S}, —COOA, —C(O)—NHSO₂A, —C(O)—NHSO₂Het, —C(O)—NHSO₂Ar, Cyc, CONHZ, OR^{f} or a group —C(O)—NHQR^{d}, —NH—C(O)QR^{d}, —COOH or tetrazolyl or oxadiazolyl, or hydroxyl-substituted oxadiazolyl, which may all be unsubstituted or substituted by alkyl having 1 to 8 carbon atoms or if R^{a} is substituted Ar or substituted Het, also H;
or, if R^{a} is Het or substituted Ar, or if R^{c} is H, F, Br, I, CN, CF₃, OCF₃, NO₂, Het, tetrazol, alkyl having 1 to 6 carbon atoms, or alkoxy having 1 to 6 carbon atoms, or if W² is N, or if W¹ is N, or if R¹ and R² are alkyl having 1 to 3 carbon atoms, or R¹ and R² build together with the atom to which they are attached a carbocyclic or heterocyclic ring having 3 to 7 atoms, or if V represents a CO or a linear or branched (C2-C6)-alkylene group, or a bond, or if W3 represents a C-2 to C-5 alkylene group that is unsubstituted or substituted by R^{f}, R^{g}, carbonyl (═O), or by a group —C(O)—OR^{f} or —C(O)NR^{f}R^{g};
or if A* represents C-2 to C-5 alkylene group that is unsubstituted or substituted by R^{f}, R^{g}, carbonyl (═O), or by a group —C(O)—OR^{f}, or —C(O)NR^{f}R^{g};
then R^{b} also denotes a group —C(O)—NHA, —C(O)—NHHet, —C(O)—NHQR^{d} or —C(O)—NHAr;
Z denotes one of the following groups: 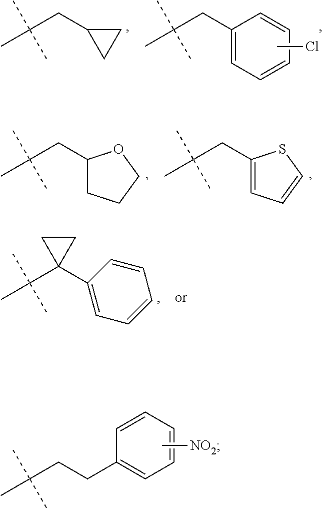
A denotes a branched or linear alkylene having 1 to 12 carbon atoms wherein one or more, H atoms may be replaced by Hal, OR³, N(R³)₂, Het, Ar, NHCOOR³, COOR^{S}, —CON(R³)₂, and wherein one or more CH₂-groups may be replaced by O, NR³, OCO, NHCO, SO₂, and/or by —CH═CH—, —C≡C—, or denotes cycloalkyl, cycloalkylene or cycloalkylalkylene having 3 to 7 ring C-atoms;
R³ denotes H or alkyl having 1 to 6 carbon atoms wherein 1 or more H atom may be replaced by Ar;
R^{c} denotes H, Hal, CN, CF₃, OCF₃, Het, NO₂, tetrazol, or alkyl having 1 to 6 carbon atoms or alkoxy having 1 to 6 carbon atoms;
Q is (CR¹R²)ₚ, (CH₂)ₚ, (CH₂)ₚSO₂(CH₂)_{p′}, or 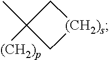
R^{d} denotes H, Ar, Het or cycloalkyl having 3 to 7 carbon atoms;
denotes H, Hal, NH₂, NO₂, Ar, O—Ar, Het or cycloalkyl having 3 to 7 carbon atoms, or R^{f};
R^{f} and R^{g} are independently of one another H, Ar, Het, or lower alkyl or R^{f} and R^{g} build together with the atom or atoms at which they are attached a carbocyclic or heterocyclic ring having 3 to 7 atoms;
R¹ and R² are independently of one another H, alkyl, alkyloxy, hydroxy, hydroxyalkyl, amino, aminoalkyl, alkylamino, alkylanimoalkyl, carboxy, alkyloxycarbonyl, aminocarbonyl or alkylaminocarbonyl, or R¹ and R² build together with the atom or atoms at which they are attached a carbocyclic or heterocyclic ring having 3 to 7 atoms or R¹ and R² are independently of one another H, alkyl having 1 to 3 carbon atoms, or R¹ and R² build together with the atom to which they are attached a carbocyclic or heterocyclic ring having 3 to 7 atoms, or R¹ is a (C1-C6)-alkylene linked to R^{a};
R⁴ denotes H or OR³;
Hal denotes F, Cl, Br, or I;
Ar denotes a monocyclic or bicyclic, saturated, unsaturated or aromatic carbocyclic ring having 6 to 14 carbon atoms, which is unsubstituted or monosubstituted, disubstituted or trisubstituted by alkyl having 1 to 8 carbon atoms, alkoxy having 1 to 8 carbon atoms, Hal, CF₃, OCF₃, NO₂, N(R³)₂, COOR³, COR³, SO₂N(R³)₂, COHet, Het, OHet, OR³, CONH(CH₂)ₚN(R³)₂, Cyc, SO₂N(R³)₂, CN, and/or acyl;
Het denotes a monocyclic or bicyclic, saturated, unsaturated or aromatic heterocyclic ring having 1 to 4 N, O and/or S atoms or one CO function, which is unsubstituted or monosubstituted, disubstituted or trisubstituted by alkyl having 1 to 8 carbon atoms, alkoxy having 1 to 8 carbon atoms, Hal, CF₃, OCF₃, NO₂CN, N(R³)₂, COOR³, COR³, SO₂N(R³)₂, COAr, OR³, Ar, CONH(CH₂)ₚN(R³)₂, Cyc, SO₂N(R³)₂, Ar, OAr, and/or acyl;
Cyc denotes a cycloalkyl having 3 to 12 carbon atoms, which is unsubstituted or monosubstituted, disubstituted, trisubstituted by OR³, Hal, CN;
p and p′ are each independently of one another 0, 1, 2, 3, 4, 5 or 6;
s is 0, 1, 2, 3 or 4;
and pharmaceutically acceptable solvates, tautomers, salts and stereoisomers and mixtures thereof.

17. The compound according to claim 16, wherein R^{a} denotes one of the following groups:

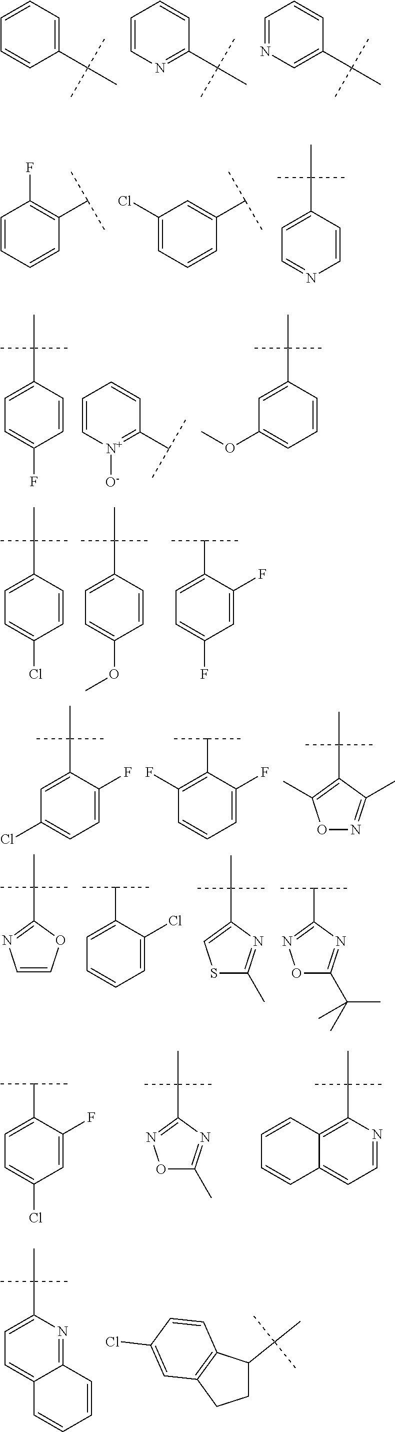

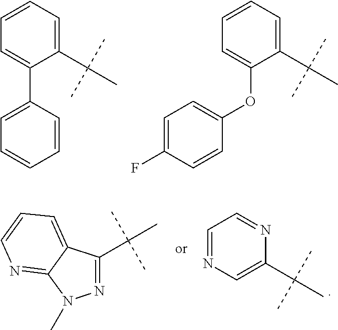

18. The compound according to claim 16, wherein R^{b} denotes one of the following groups:

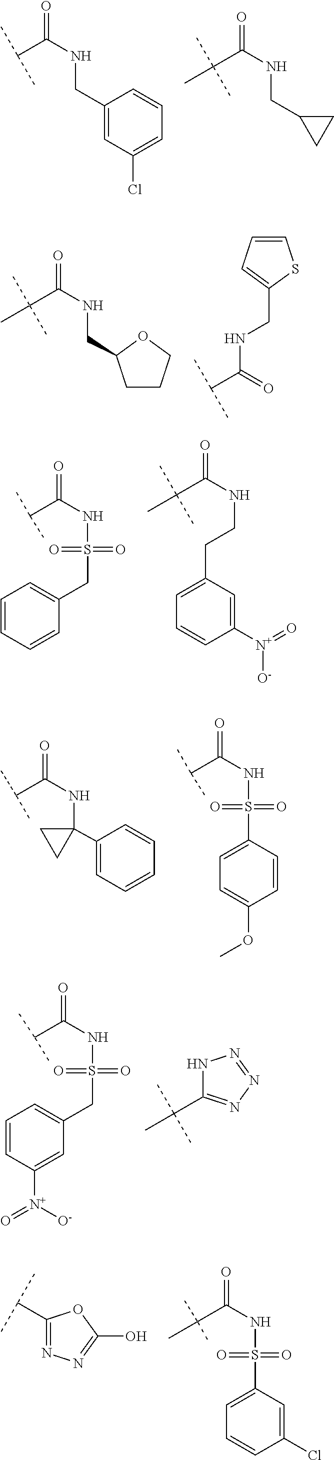

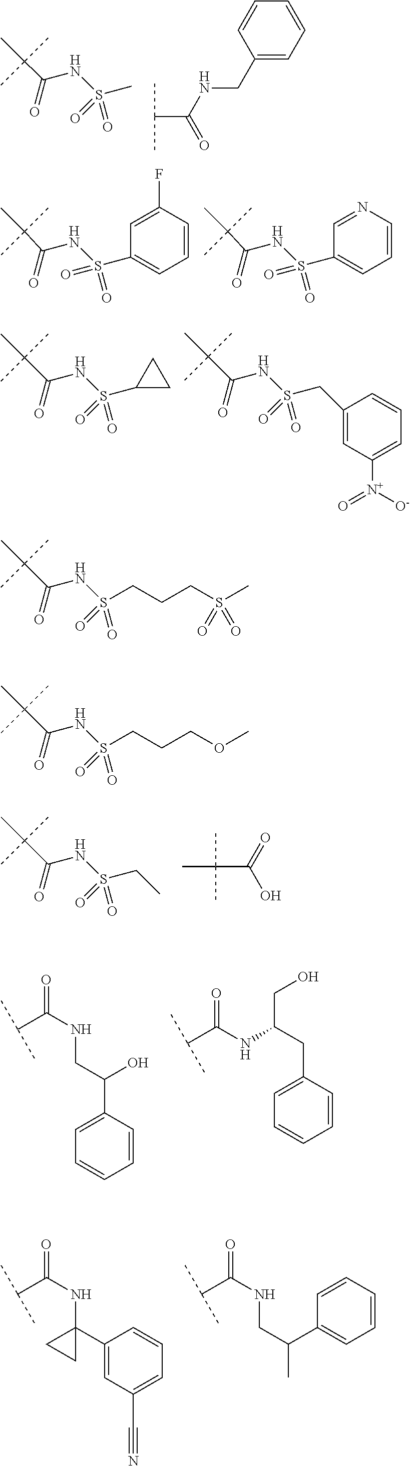

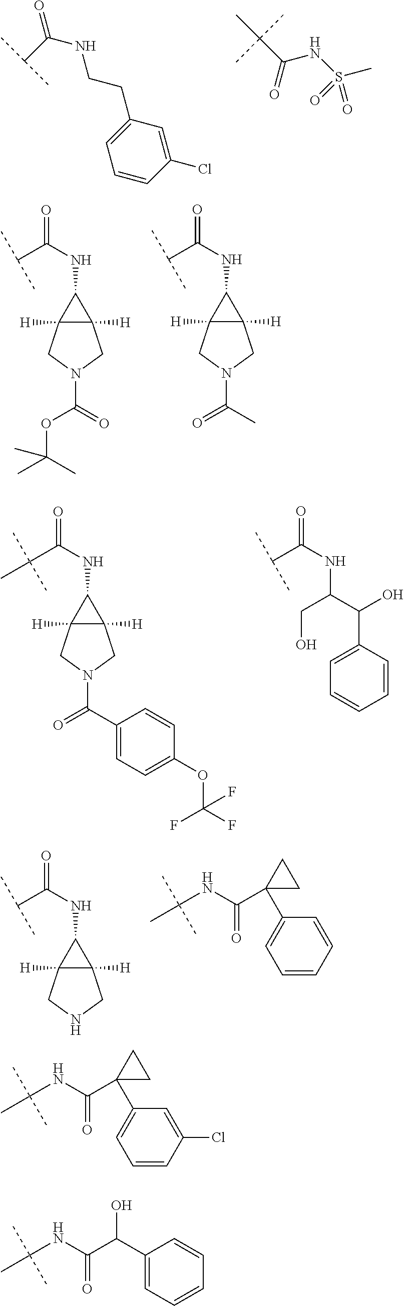

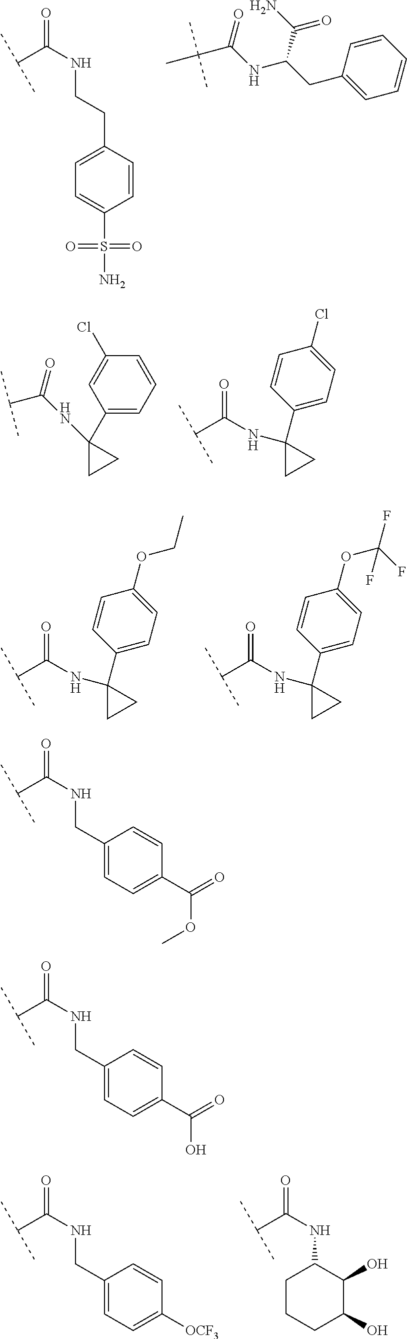

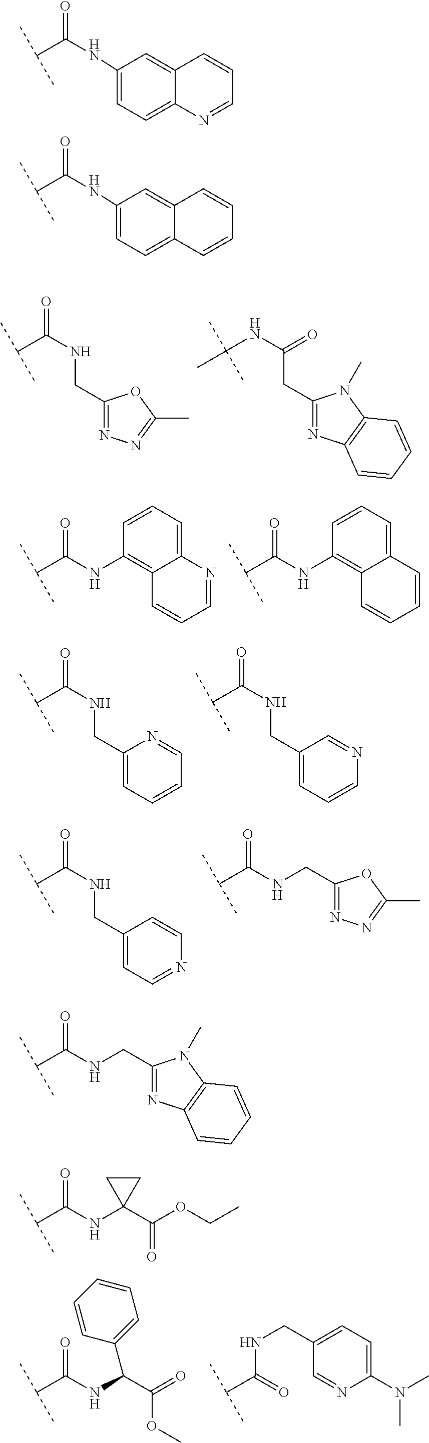

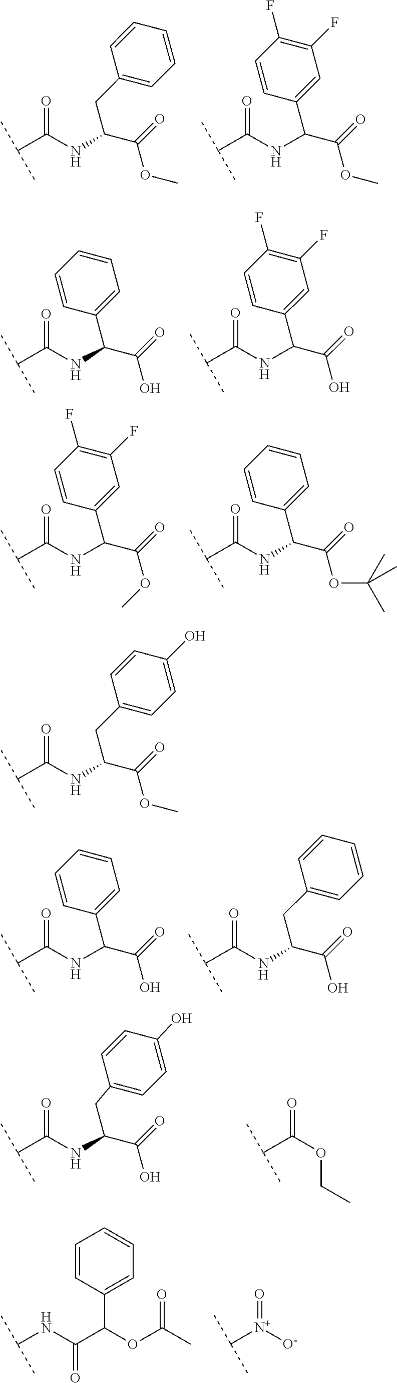

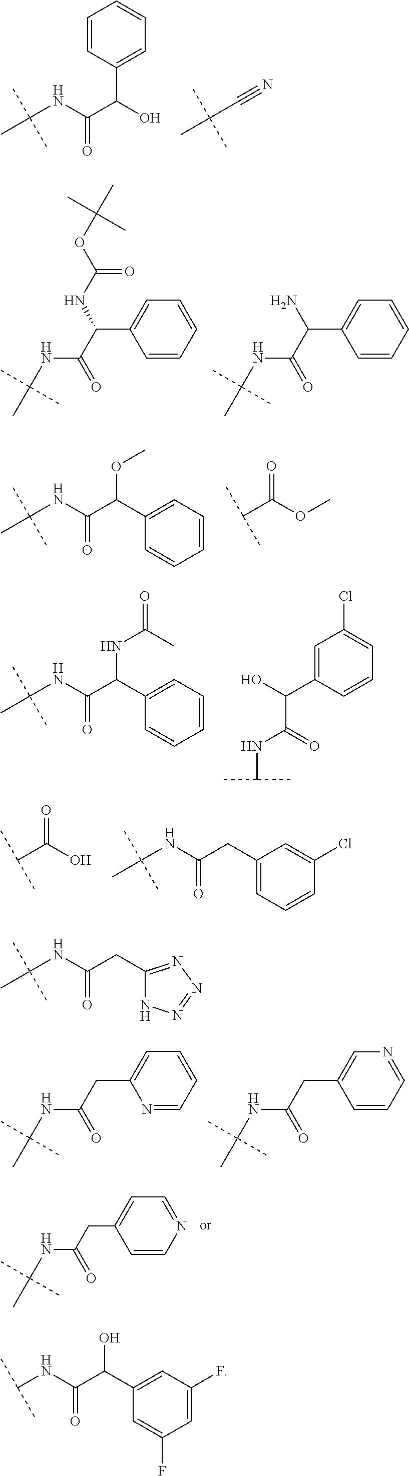

19. The compound according to claim 16, wherein R^{c} denotes Hal, CN, or alkoxy having 1 to 6 carbon atoms.

20. The compound according to claim 16, wherein W₂ denotes CH.

21. The compound according to claim 16, said compound being selected from:

| structure | Ex |
|---|---|
| 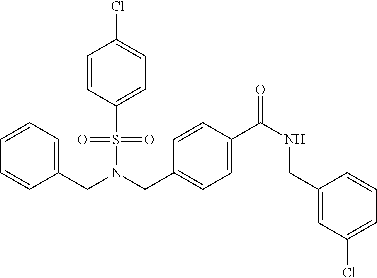 | 1 |
| 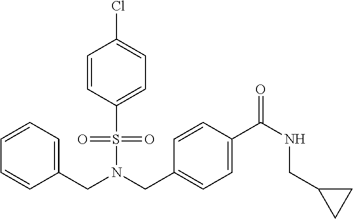 | 2 |
| 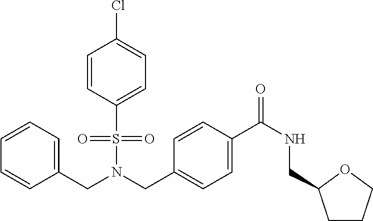 | 3 |
| 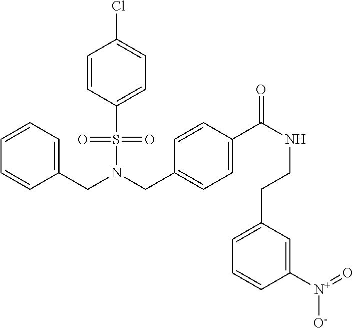 | 4 |
| 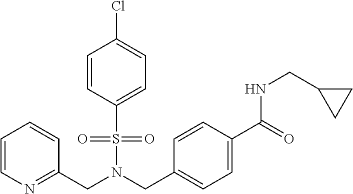 | 5 |
| 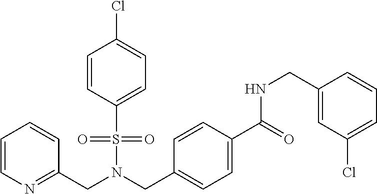 | 6 |
| 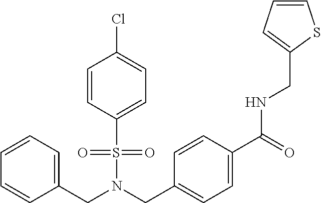 | 7 |
| 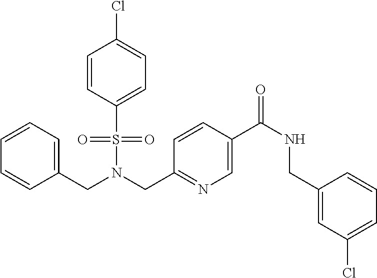 | 8 |
| 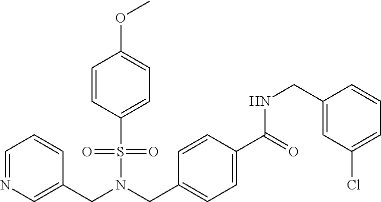 | 9 |
| 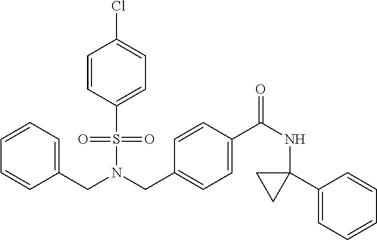 | 10 |
| 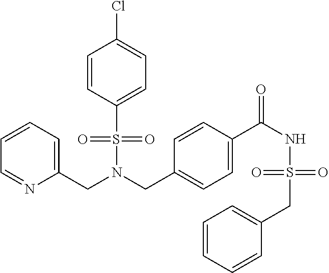 | 11 |
| 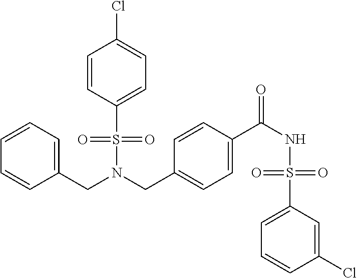 | 12 |
| 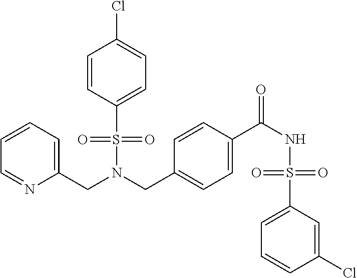 | 13 |
| 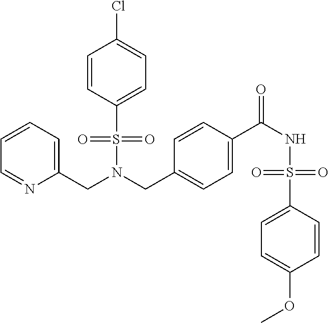 | 14 |
| 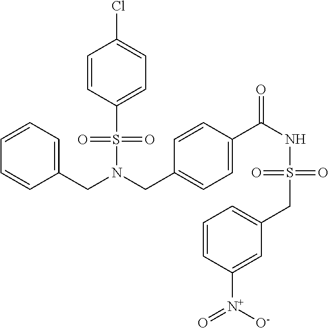 | 15 |
| 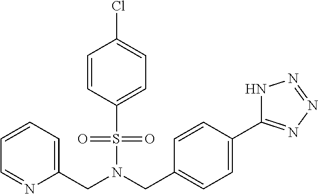 | 16 |
| 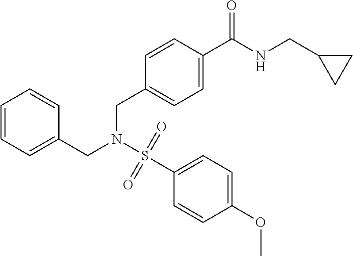 | 17 |
| 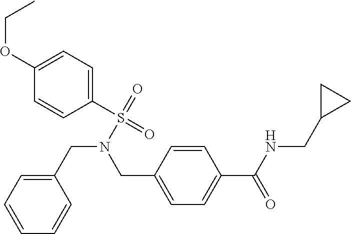 | 18 |
| 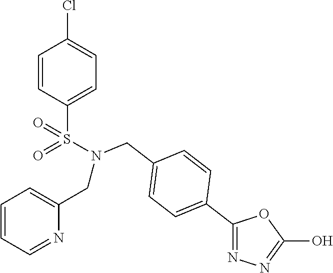 | 19 |
| 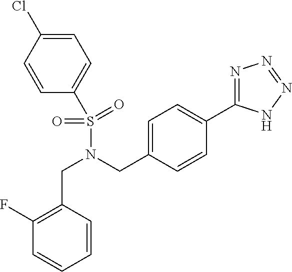 | 20 |
| 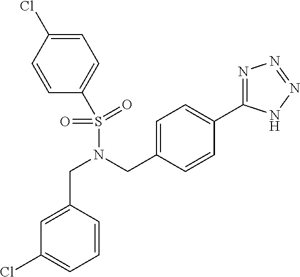 | 21 |
| 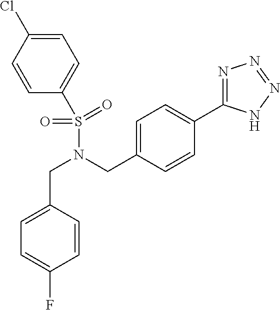 | 22 |
| 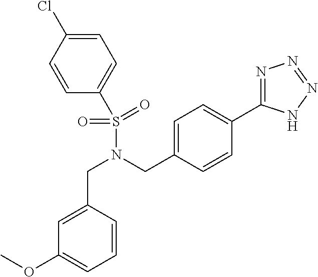 | 23 |
| 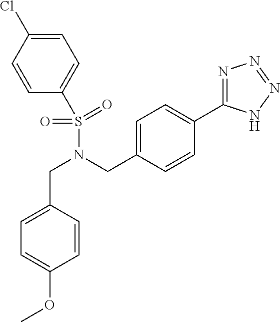 | 24 |
| 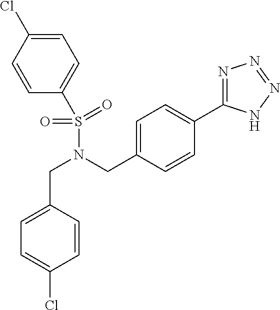 | 25 |
| 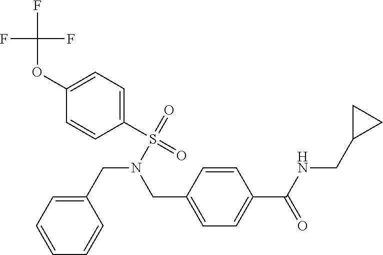 | 26 |
| 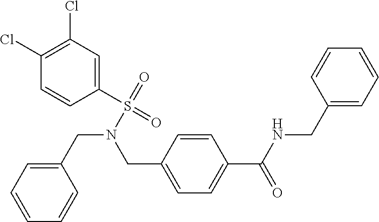 | 27 |
| 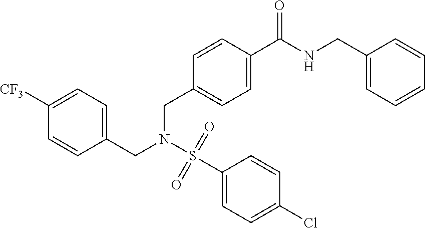 | 28 |
| 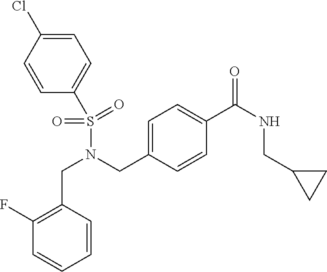 | 29 |
| 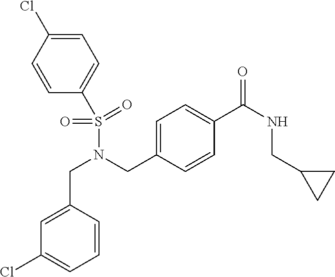 | 30 |
| 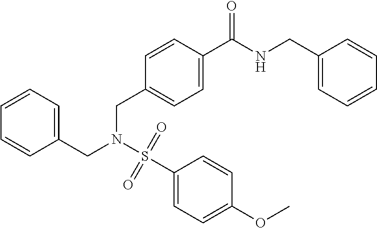 | 31 |
| 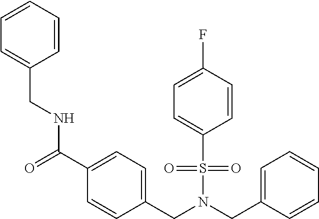 | 32 |
| 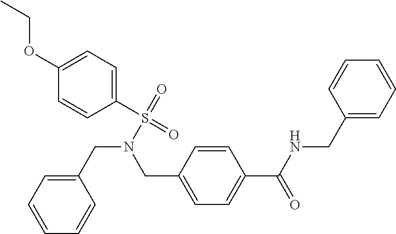 | 33 |
| 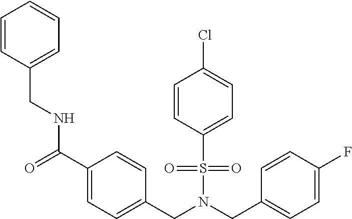 | 34 |
| 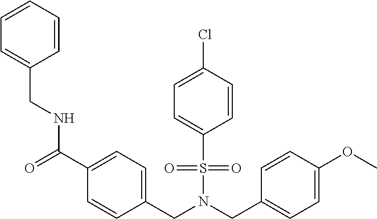 | 35 |
| 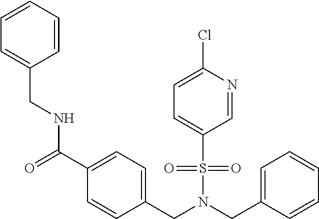 | 36 |
| 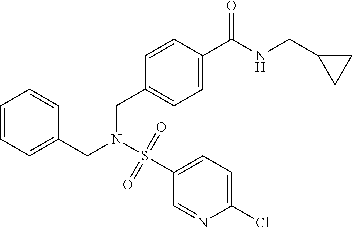 | 37 |
| 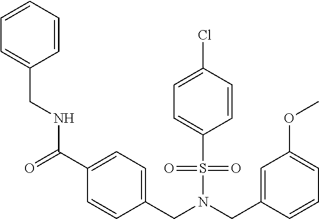 | 38 |
| 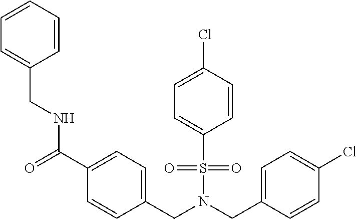 | 39 |
| 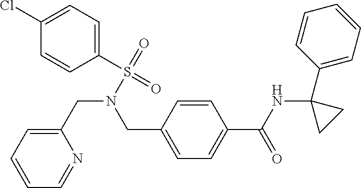 | 40 |
| 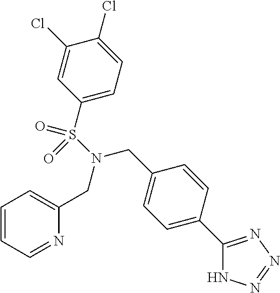 | 41 |
| 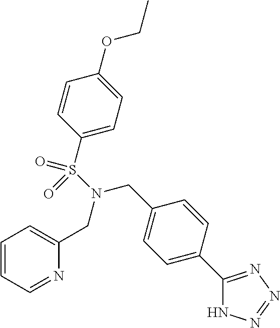 | 42 |
| 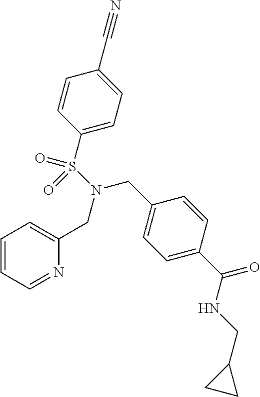 | 43 |
| 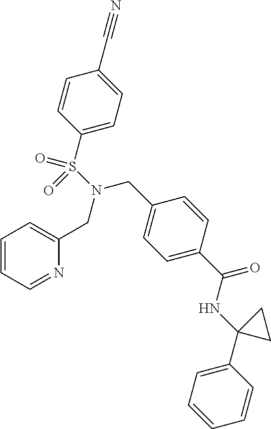 | 44 |
| 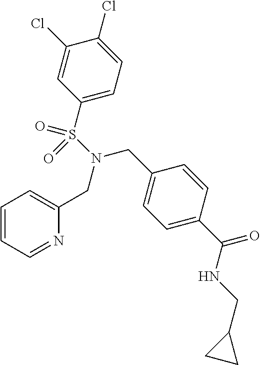 | 45 |
| 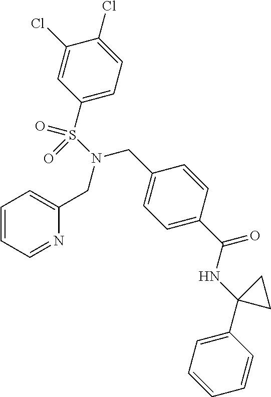 | 46 |
| 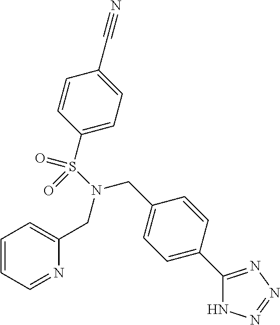 | 47 |
| 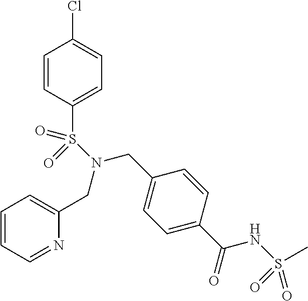 | 48 |
| 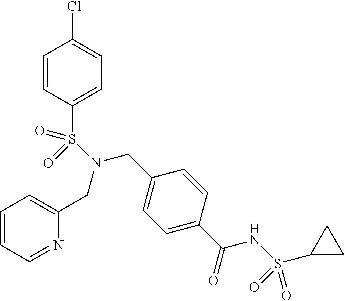 | 49 |
| 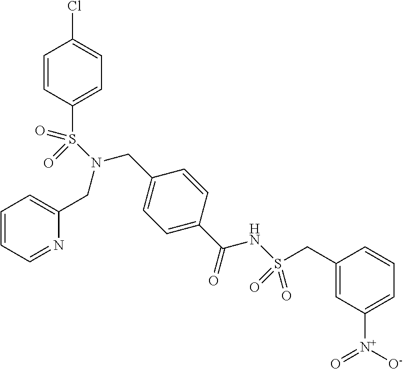 | 50 |
| 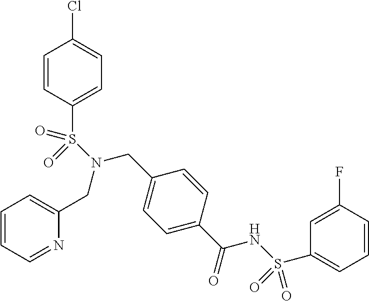 | 51 |
| 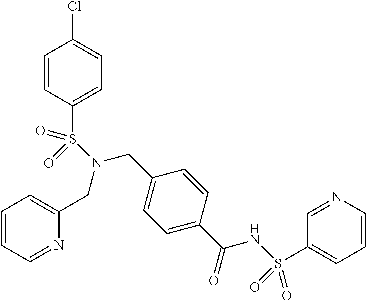 | 52 |
| 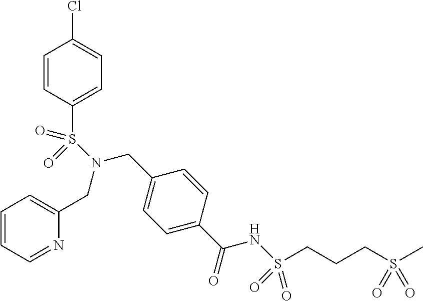 | 53 |
| 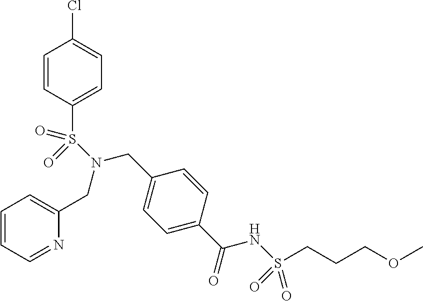 | 54 |
| 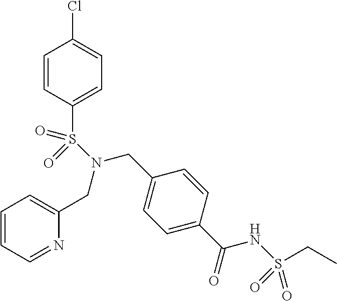 | 55 |
| 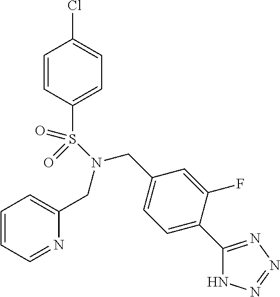 | 56 |
| 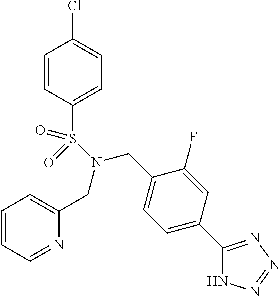 | 57 |
| 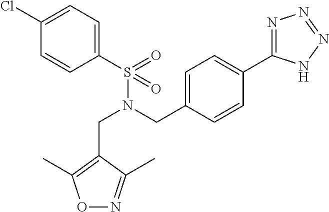 | 58 |
| 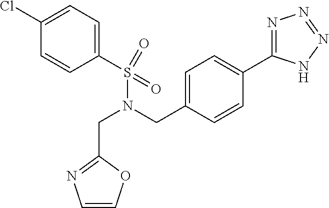 | 59 |
| 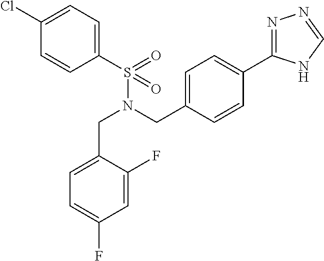 | 60 |
| 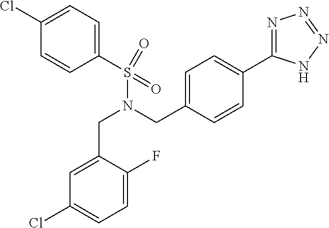 | 61 |
| 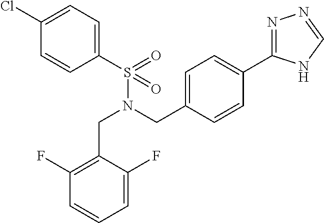 | 62 |
| 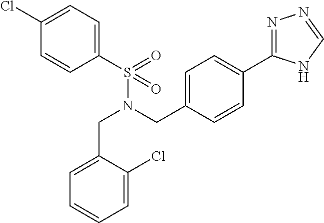 | 63 |
| 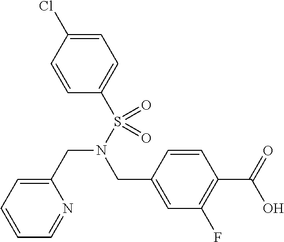 | 64 |
| 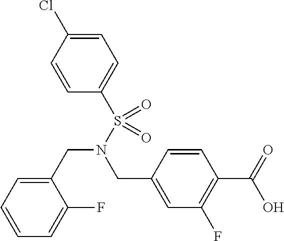 | 65 |
| 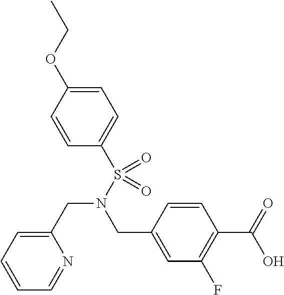 | 66 |
| 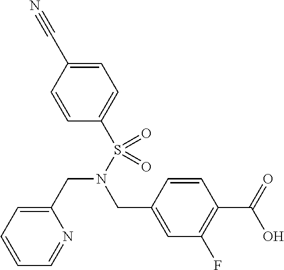 | 67 |
| 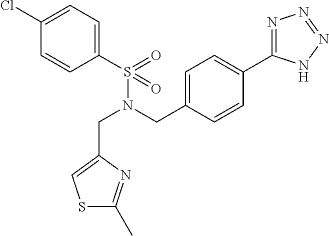 | 68 |
| 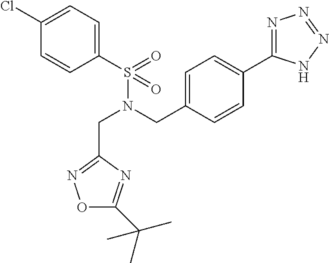 | 69 |
| 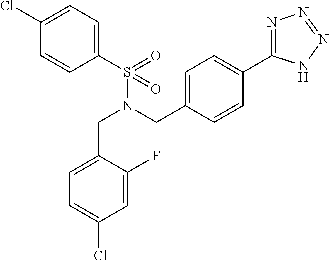 | 70 |
| 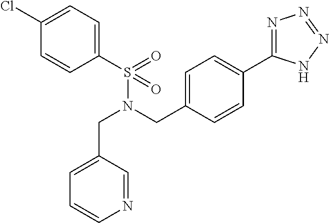 | 71 |
| 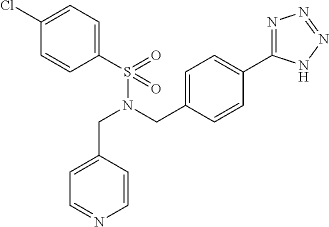 | 72 |
| 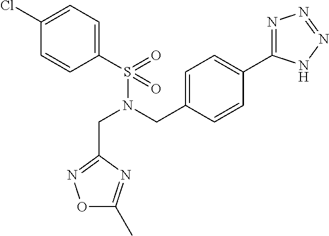 | 73 |
| 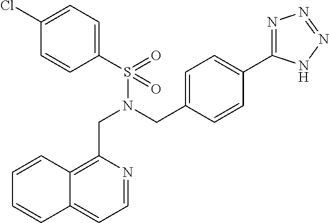 | 74 |
| 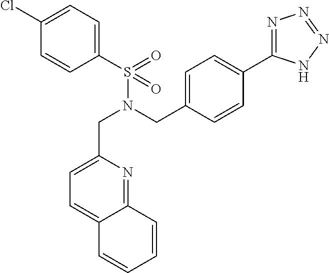 | 75 |
| 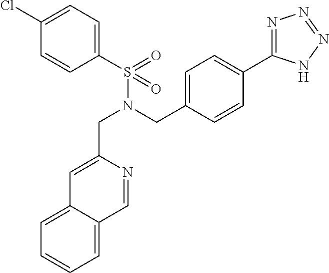 | 76 |
| 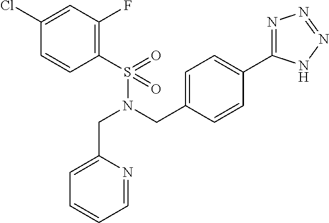 | 77 |
| 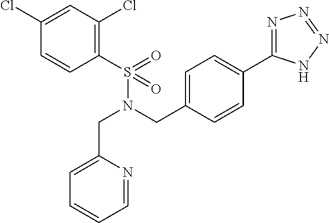 | 78 |
| 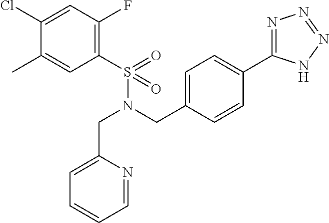 | 79 |
| 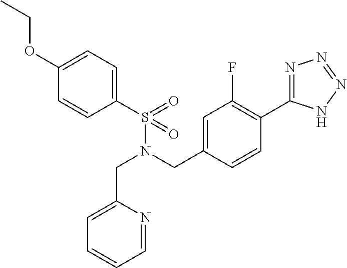 | 80 |
| 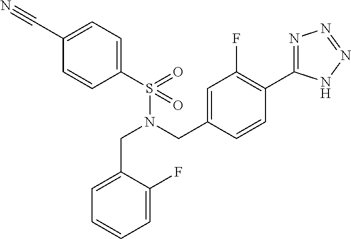 | 81 |
| 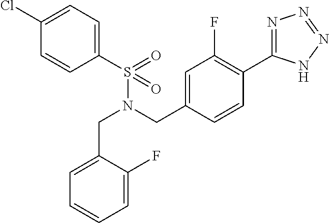 | 82 |
| 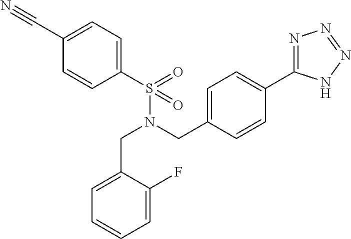 | 83 |
| 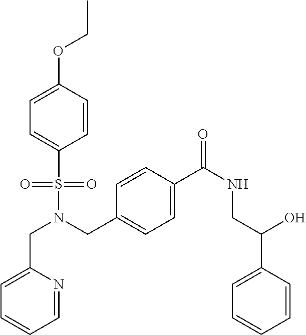 | 84 |
| 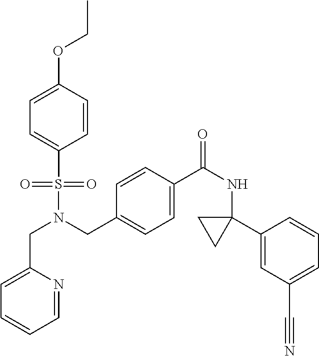 | 85 |
| 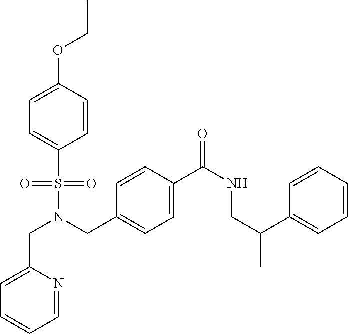 | 86 |
| 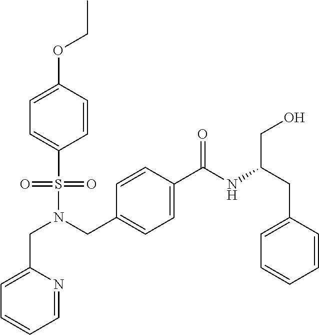 | 87 |
| 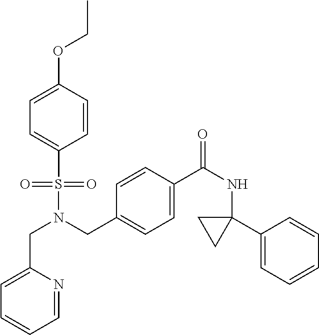 | 88 |
| 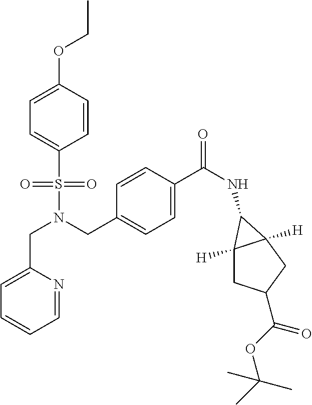 | 89 |
| 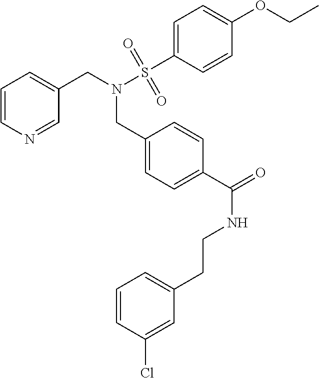 | 90 |
| 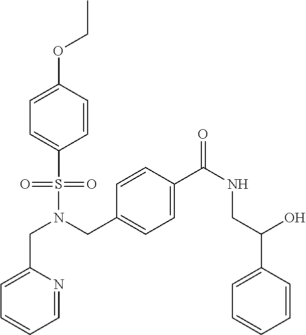 | 91 |
| 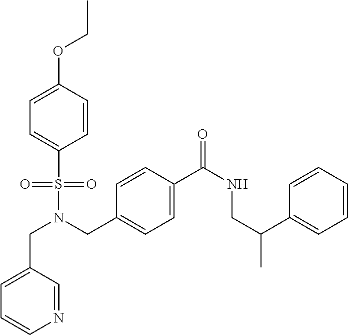 | 92 |
| 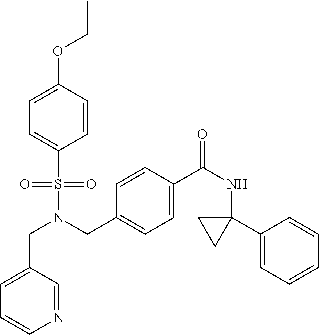 | 93 |
| 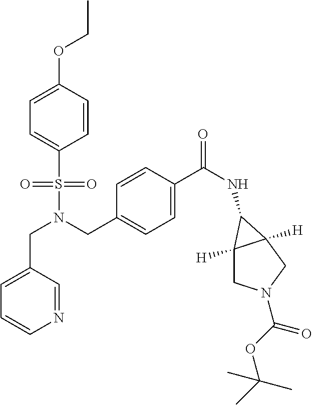 | 94 |
| 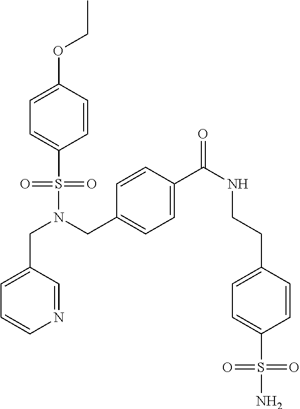 | 95 |
| 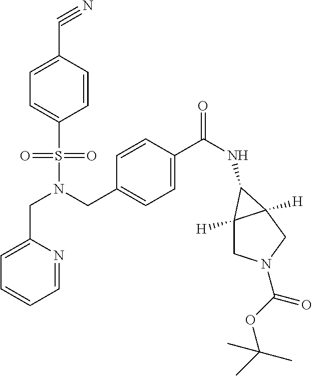 | 96 |
| 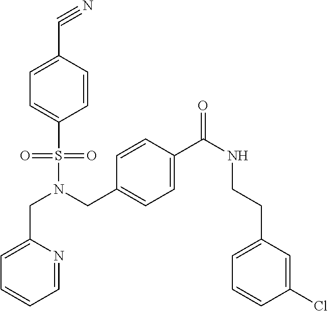 | 97 |
| 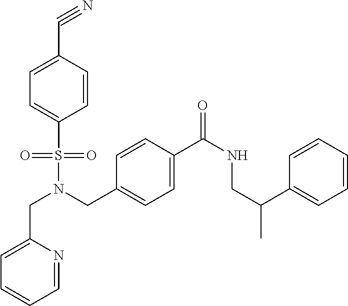 | 98 |
| 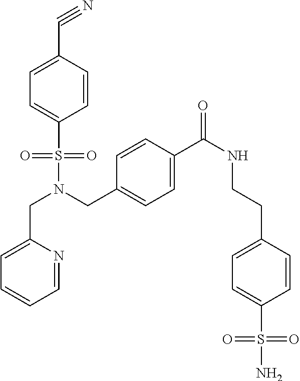 | 99 |
| 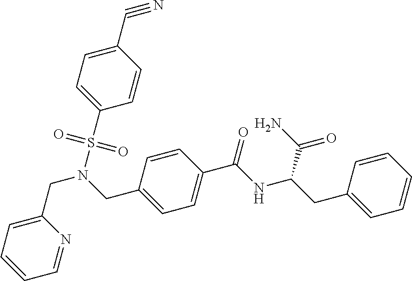 | 100 |
| 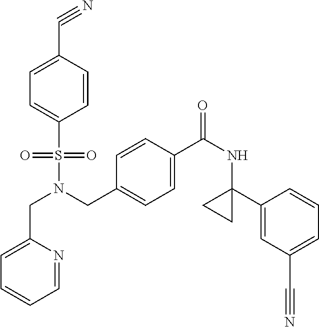 | 101 |
| 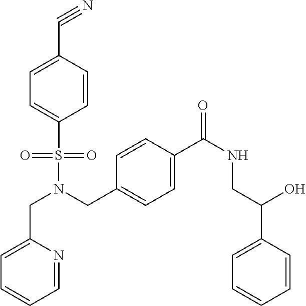 | 102 |
| 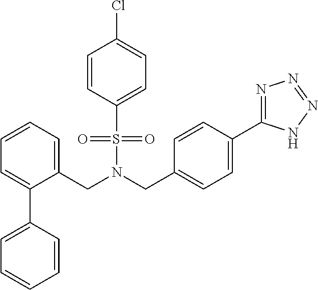 | 103 |
| 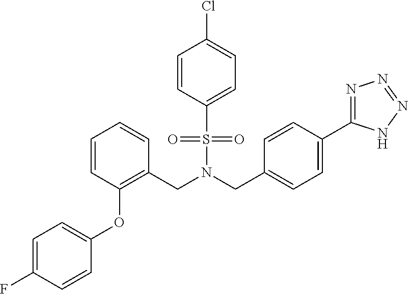 | 104 |
| 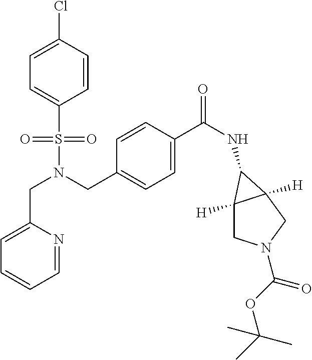 | 105 |
| 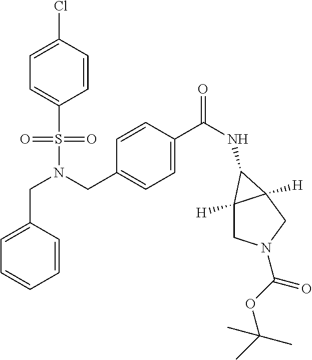 | 106 |
| 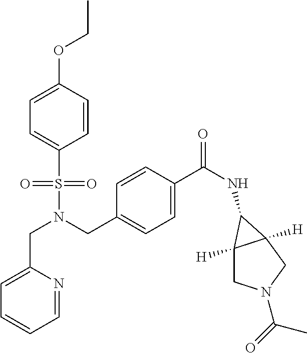 | 107 |
| 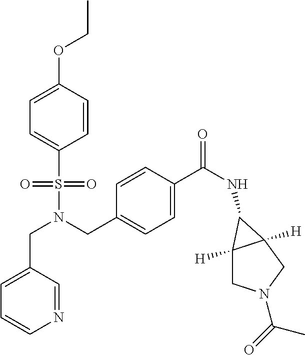 | 108 |
| 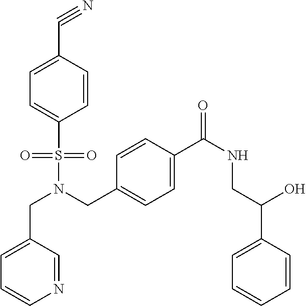 | 109 |
| 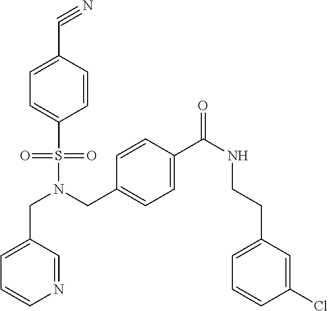 | 110 |
| 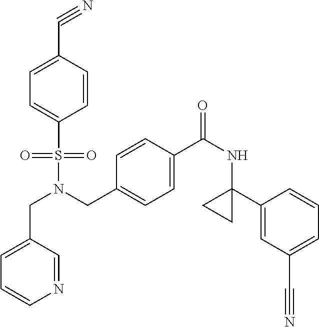 | 111 |
| 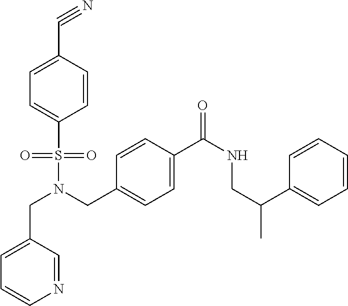 | 112 |
| 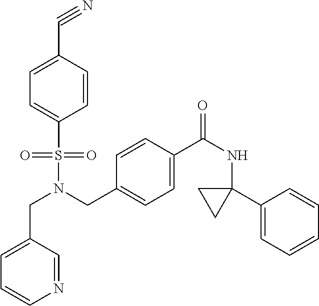 | 113 |
| 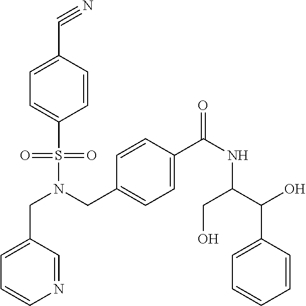 | 114 |
| 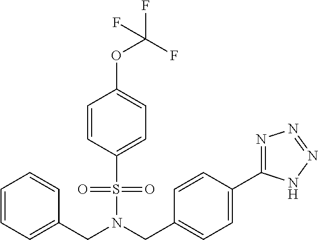 | 115 |
| 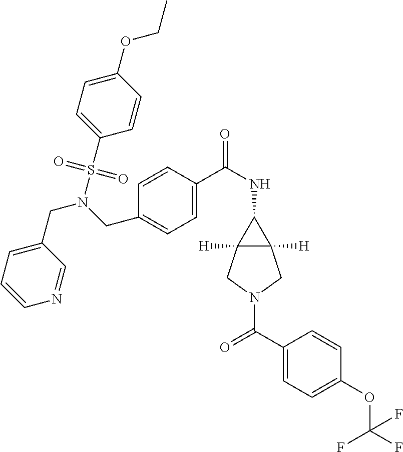 | 116 |
| 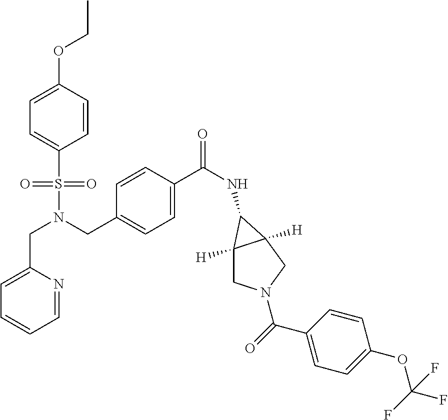 | 117 |
| 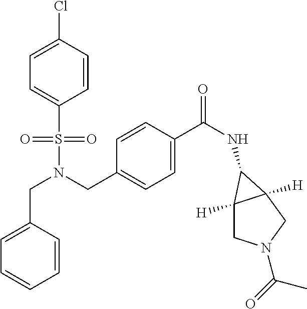 | 118 |
| 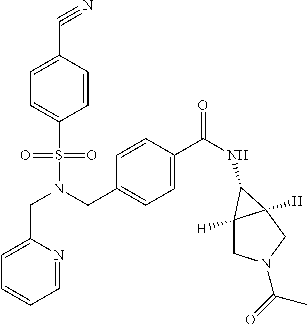 | 119 |
| 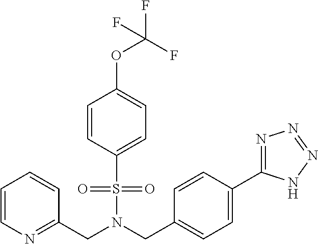 | 120 |
| 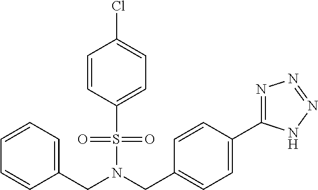 | 121 |
| 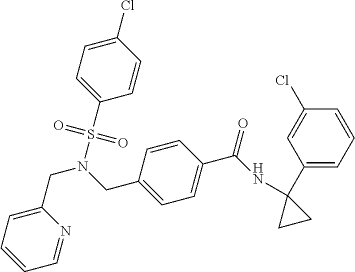 | 122 |
| 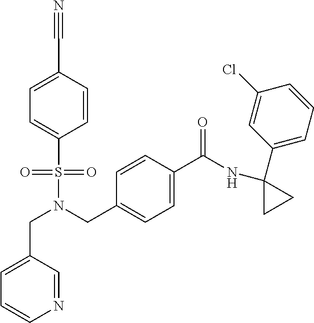 | 123 |
| 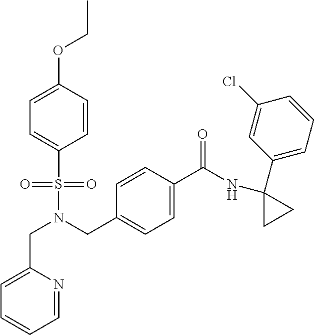 | 124 |
| 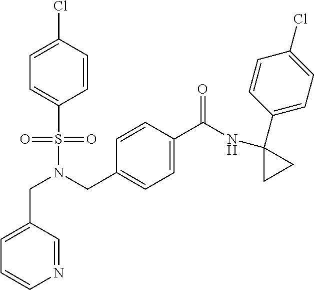 | 125 |
| 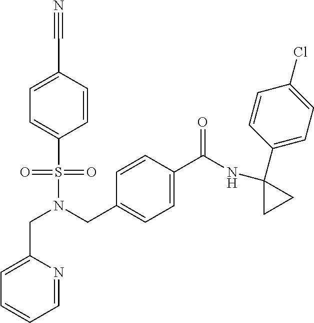 | 126 |
| 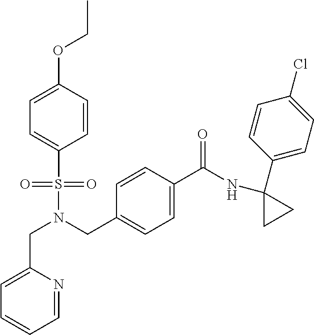 | 127 |
| 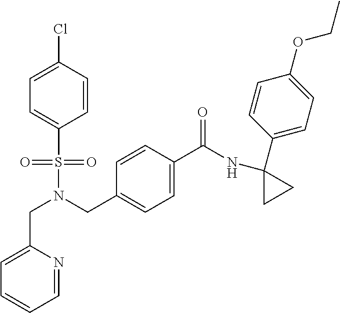 | 128 |
| 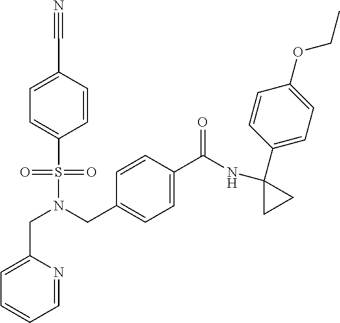 | 129 |
| 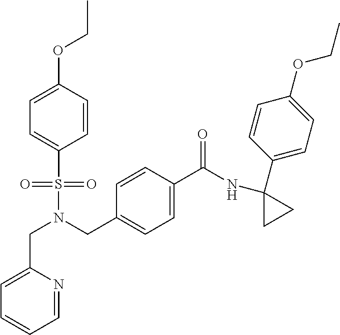 | 130 |
| 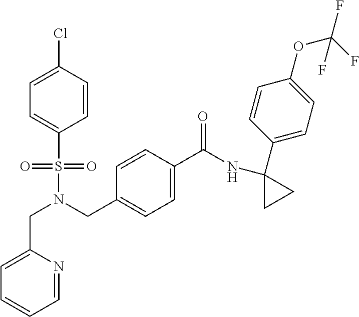 | 131 |
| 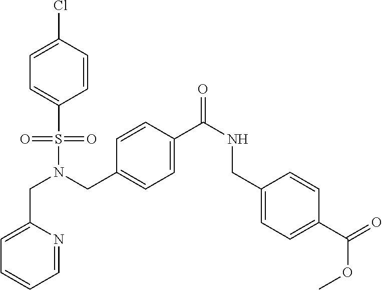 | 132 |
| 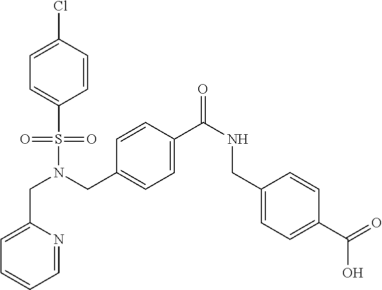 | 133 |
| 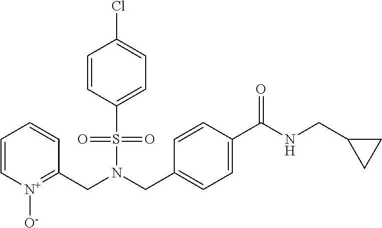 | 134 |
| 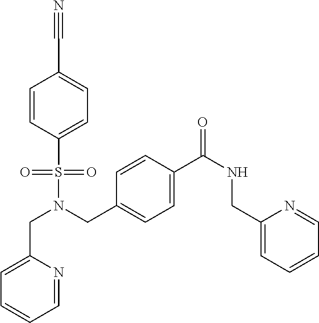 | 135 |
| 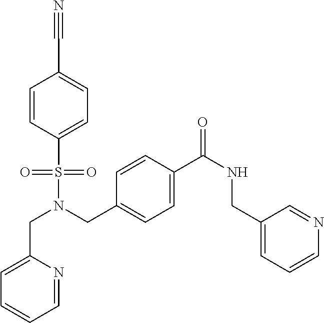 | 136 |
| 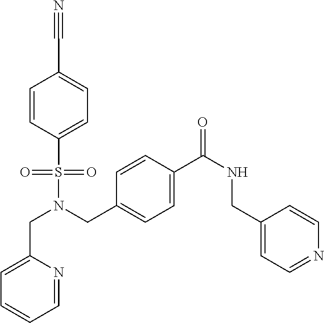 | 137 |
| 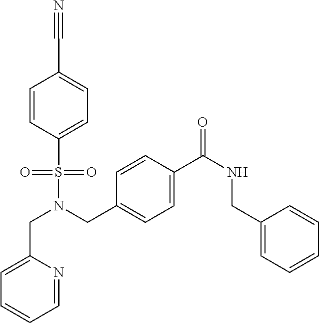 | 138 |
| 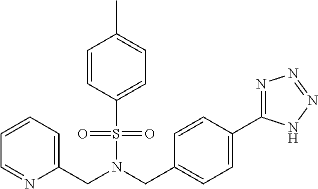 | 139 |
| 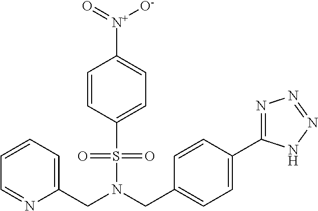 | 140 |
| 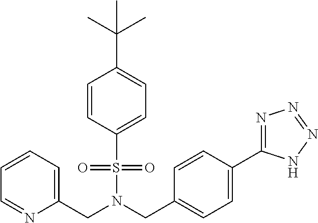 | 141 |
| 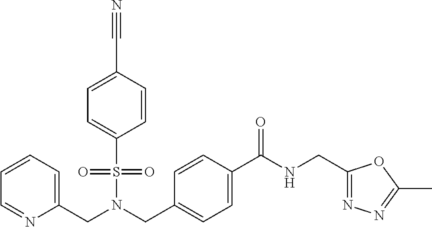 | 142 |
| 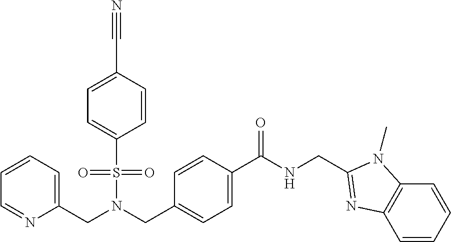 | 143 |
| 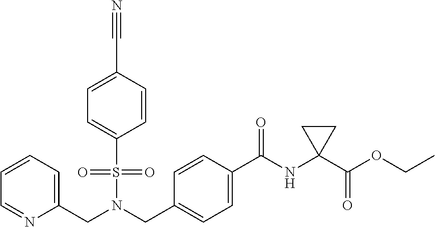 | 144 |
| 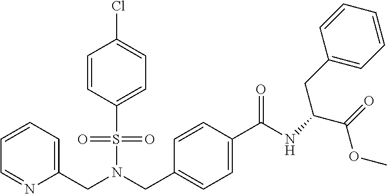 | 145 |
| 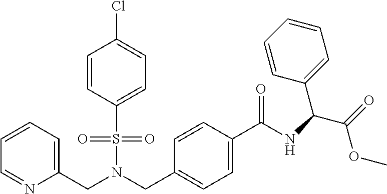 | 146 |
| 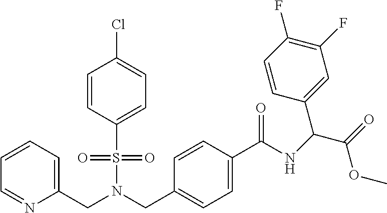 | 147 |
| 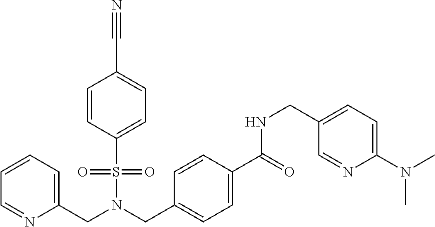 | 148 |
| 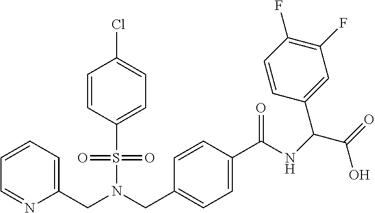 | 149 |
| 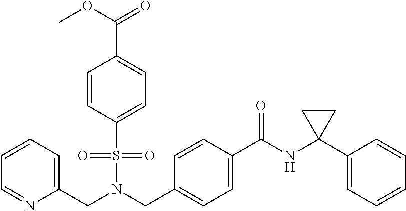 | 150 |
| 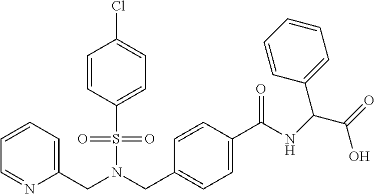 | 151 |
| 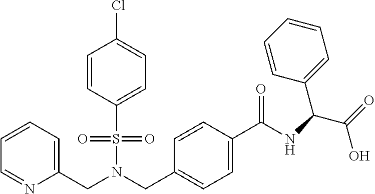 | 152 |
| 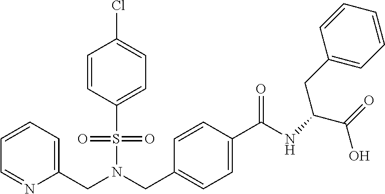 | 153 |
| 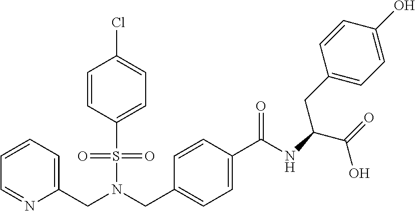 | 154 |
| 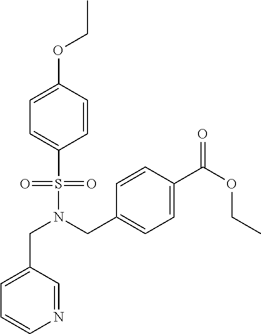 | 155 |
| 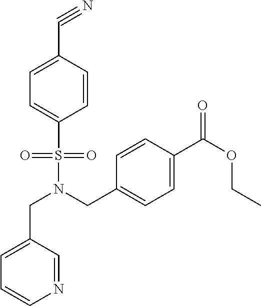 | 156 |
| 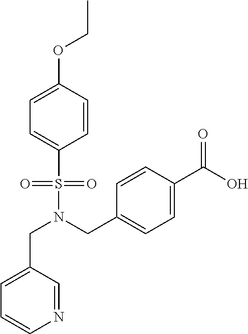 | 157 |
| 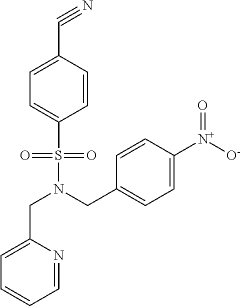 | 158 |
| 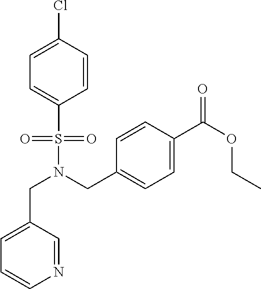 | 159 |
| 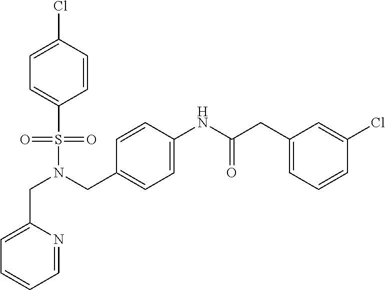 | 160 |
| 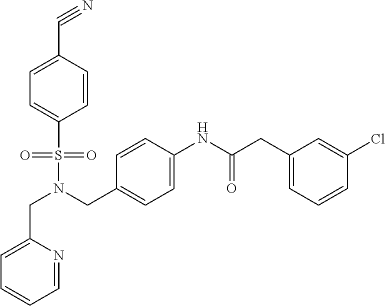 | 161 |
| 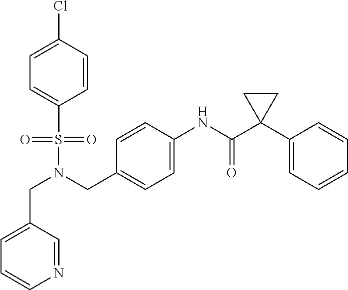 | 162 |
| 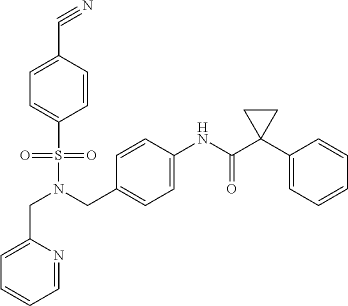 | 163 |
| 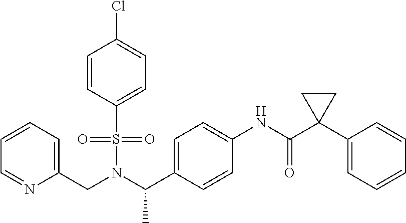 | 164 |
| 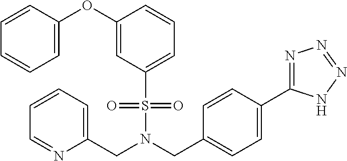 | 165 |
| 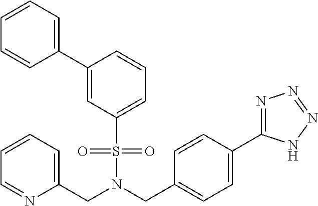 | 166 |
| 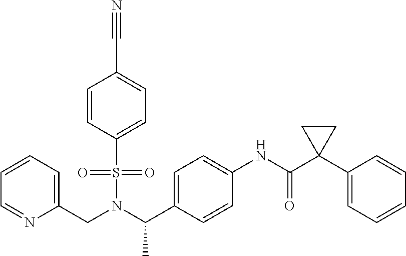 | 167 |
| 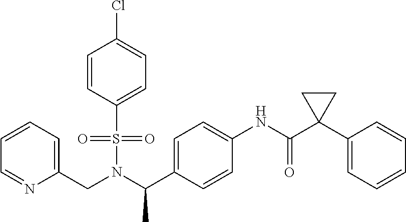 | 168 |
| 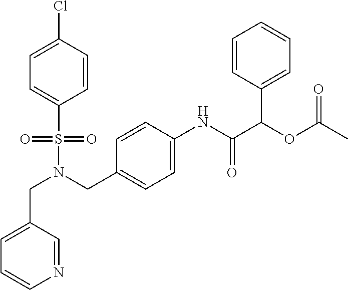 | 169 |
| 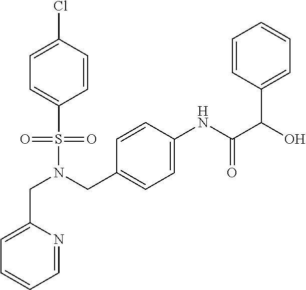 | 170 |
| 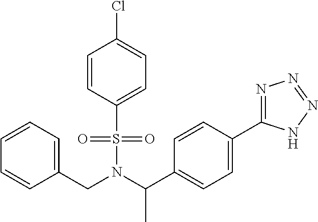 | 171 |
| 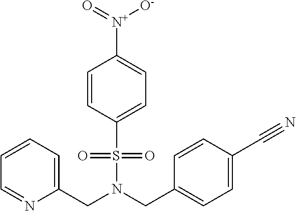 | 172 |
| 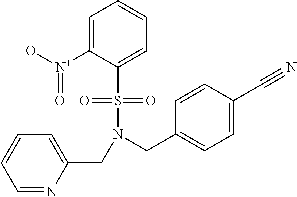 | 173 |
| 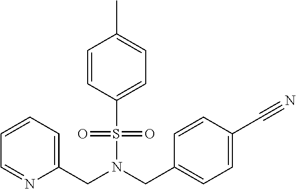 | 174 |
| 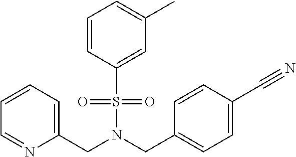 | 175 |
| 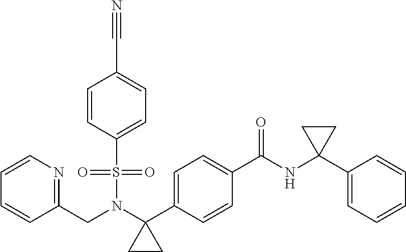 | 176 |
| 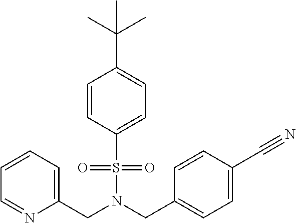 | 177 |
| 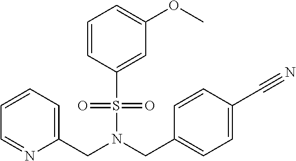 | 178 |
| 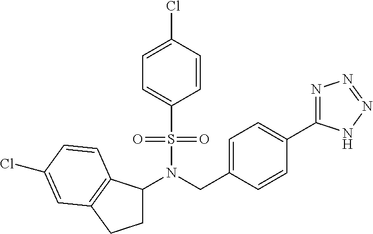 | 179 |
| 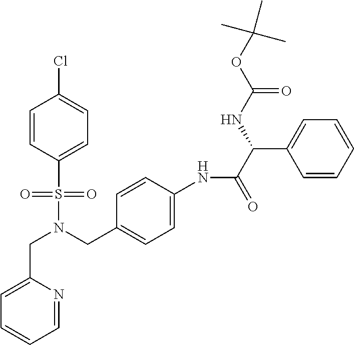 | 180 |
| 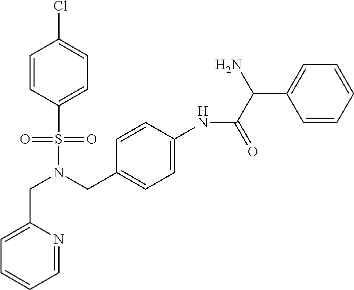 | 181 |
| 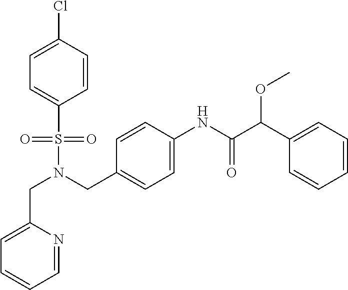 | 182 |
| 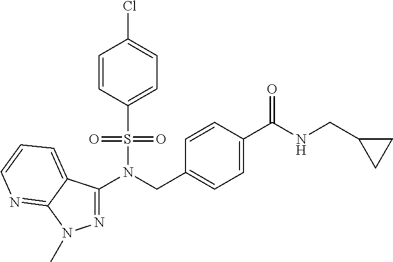 | 183 |
| 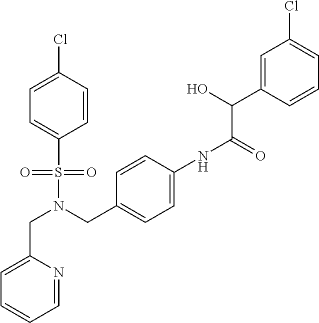 | 184 |
| 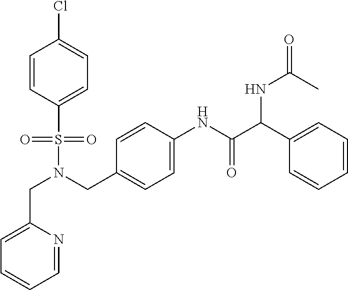 | 185 |
| 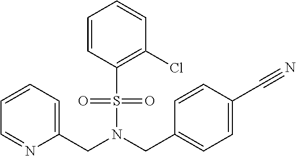 | 186 |
| 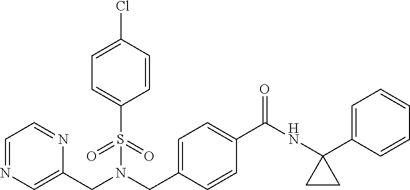 | 187 |
| 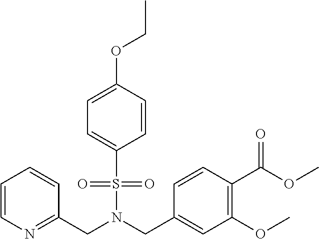 | 188 |
| 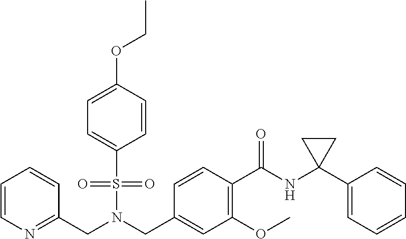 | 189 |
| 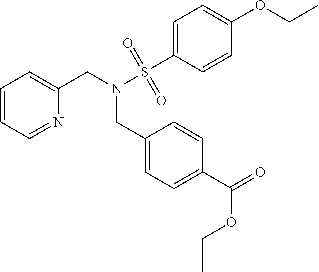 | 190 |
| 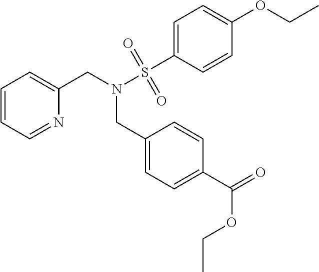 | 191 |
| 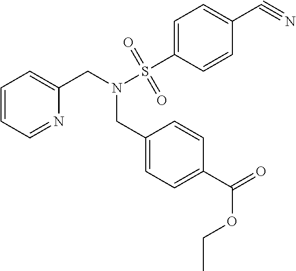 | 192 |
| 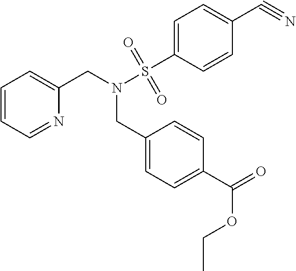 | 193 |
| 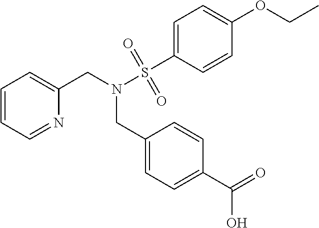 | 194 |
| 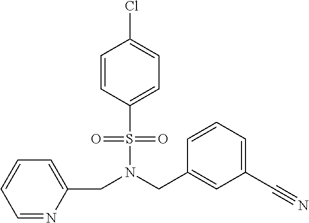 | 195 |
| 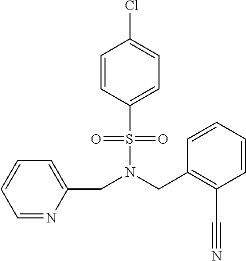 | 196 |
| 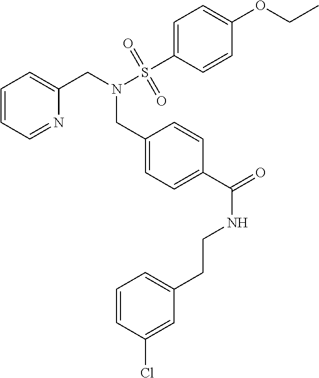 | 197 |
| 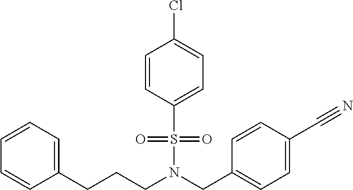 | 198 |
| 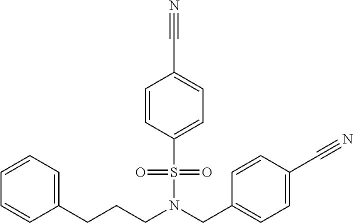 | 199 |
| 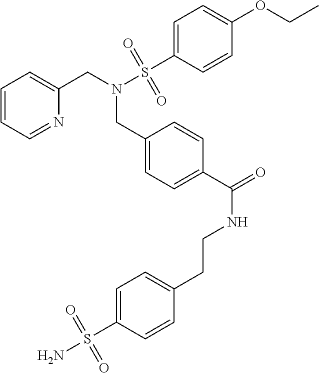 | 200 |
| 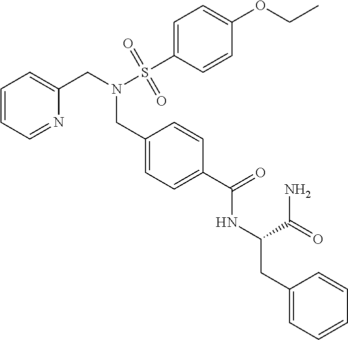 | 201 |
| 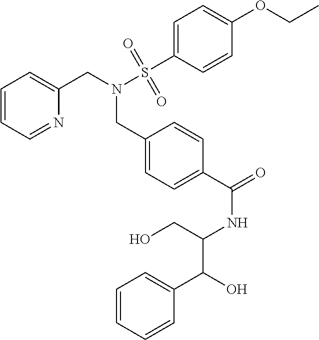 | 202 |
| 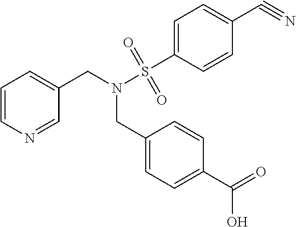 | 203 |
| 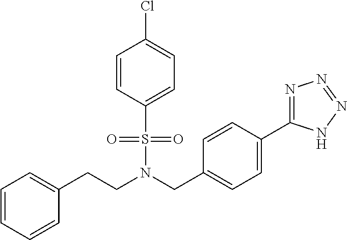 | 204 |
| 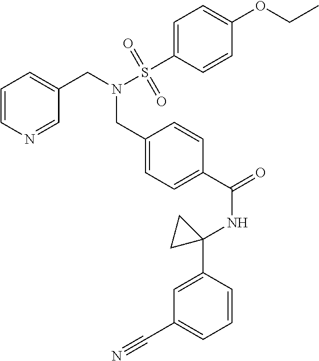 | 205 |
| 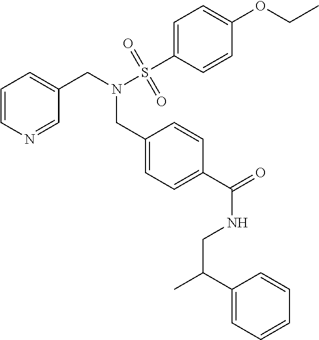 | 206 |
| 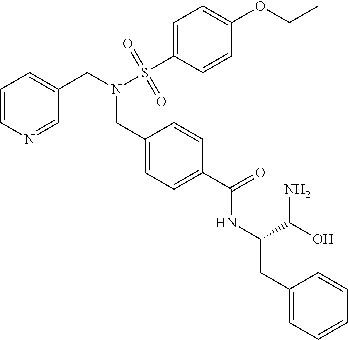 | 207 |
| 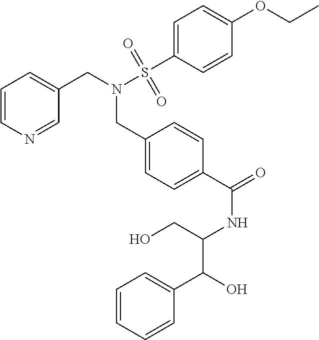 | 208 |
| 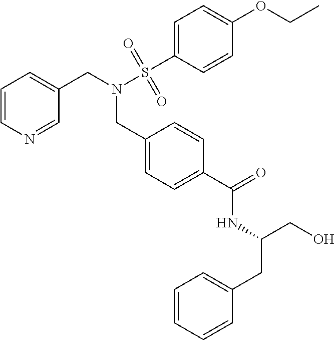 | 209 |
| 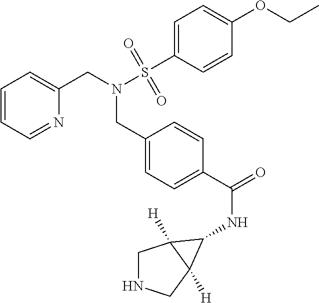 | 210 |
| 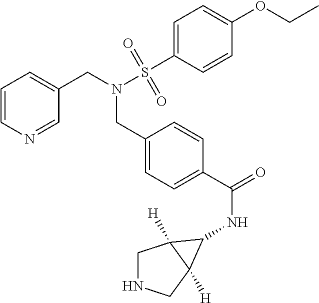 | 211 |
| 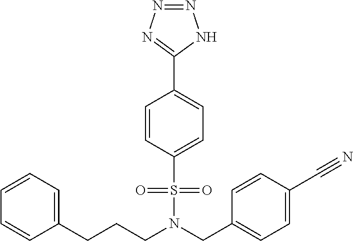 | 212 |
| 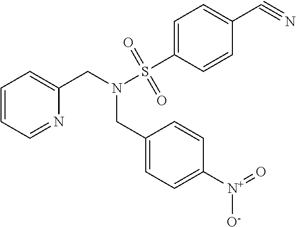 | 213 |
| 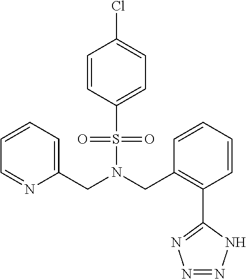 | 214 |
| 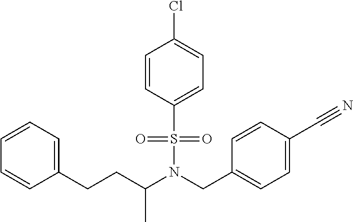 | 215 |
| 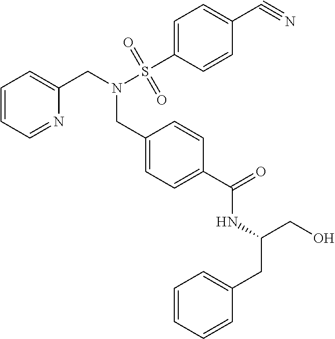 | 216 |
| 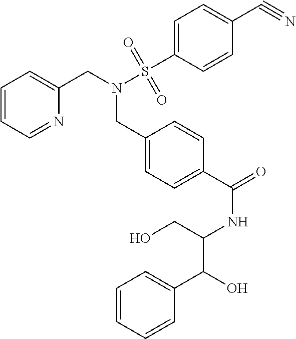 | 217 |
| 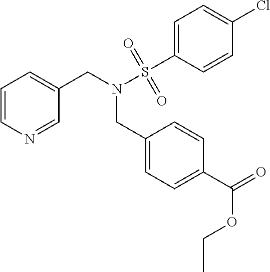 | 218 |
| 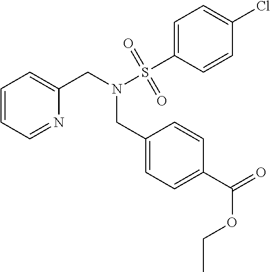 | 219 |
| 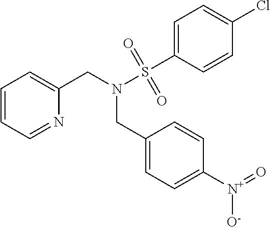 | 220 |
| 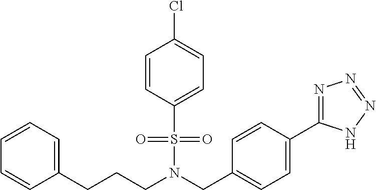 | 221 |
| 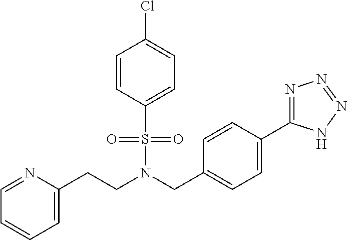 | 222 |
| 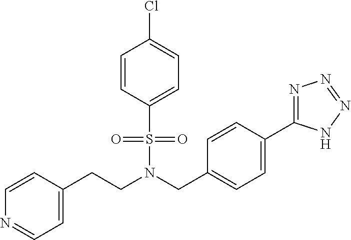 | 223 |
| 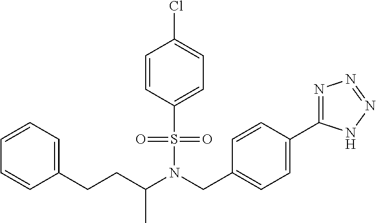 | 224 |
| 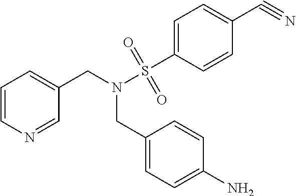 | 225 |
| 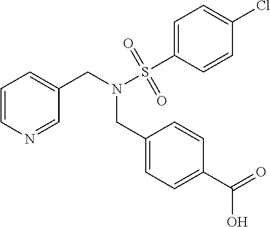 | 226 |
| 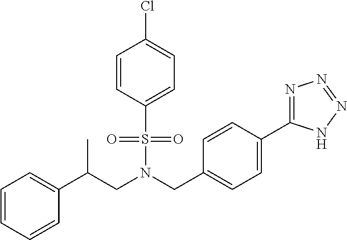 | 227 |
| 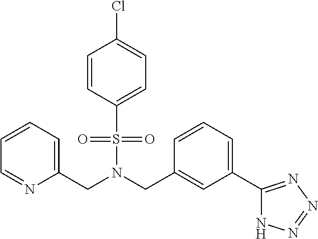 | 228 |
| 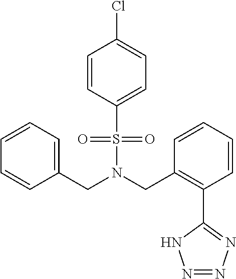 | 229 |
| 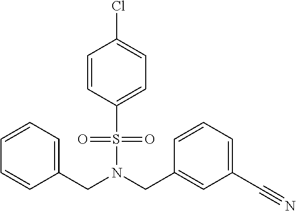 | 230 |
| 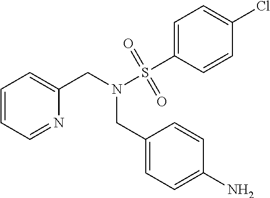 | 231 |
| 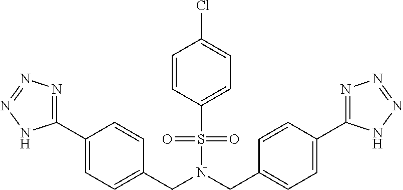 | 233 |
| 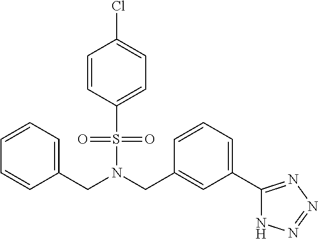 | 234 |
| 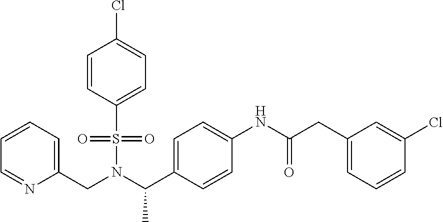 | 235 |
| 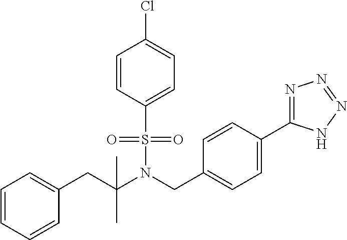 | 236 |
| 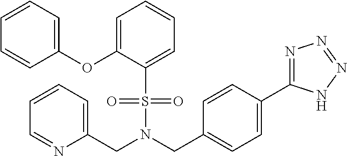 | 237 |
| 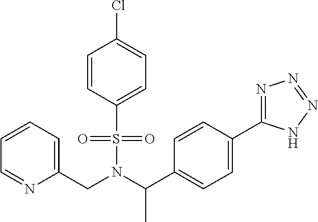 | 238 |
| 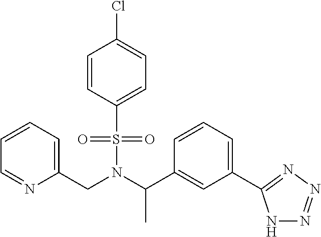 | 239 |
| 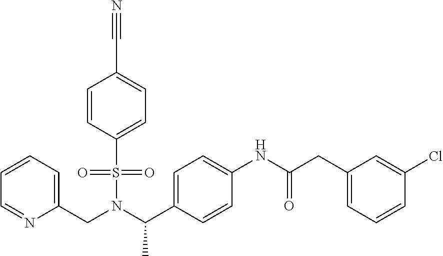 | 240 |
| 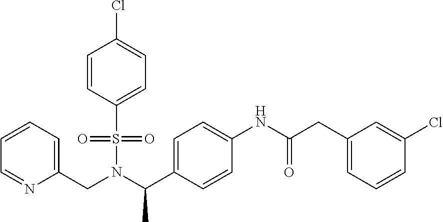 | 241 |
| 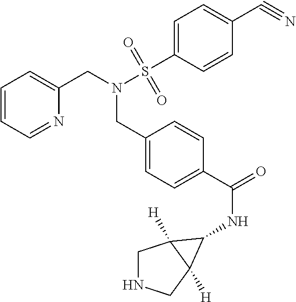 | 242 |
| 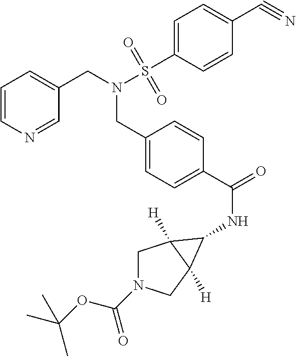 | 243 |
| 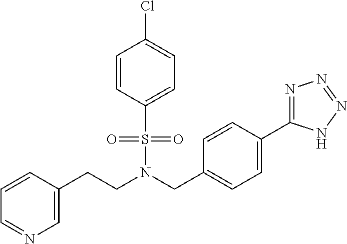 | 244 |
| 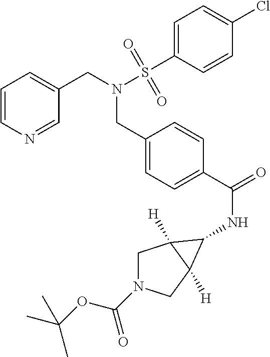 | 245 |
| 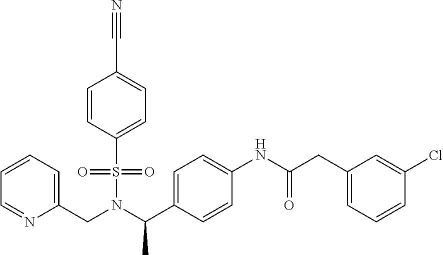 | 246 |
| 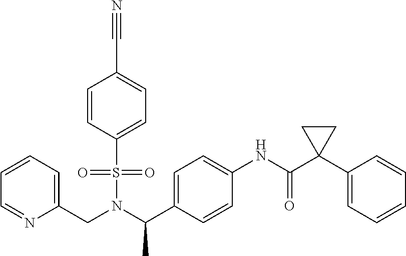 | 247 |
| 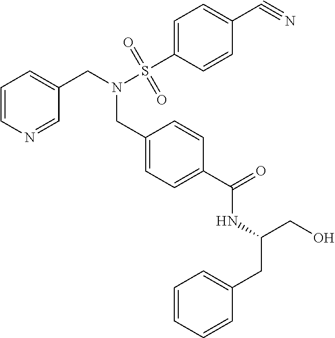 | 248 |
| 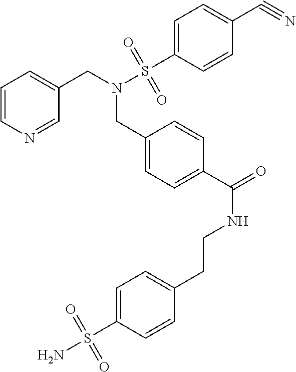 | 250 |
| 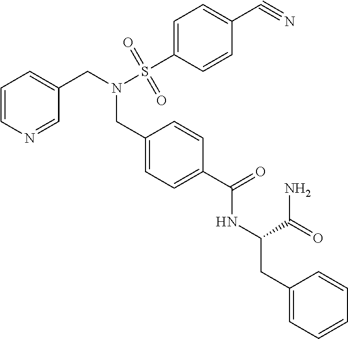 | 251 |
| 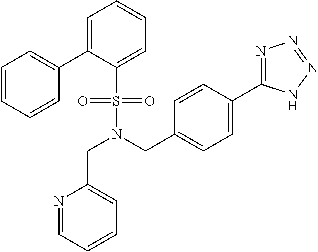 | 253 |
| 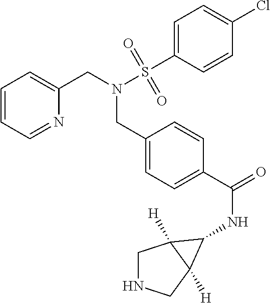 | 254 |
| 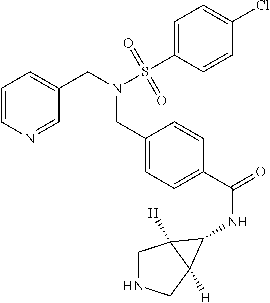 | 255 |
| 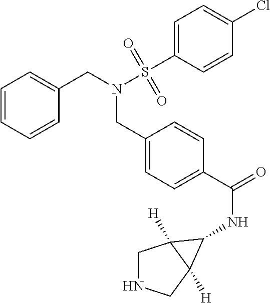 | 256 |
| 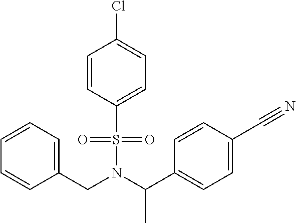 | 257 |
| 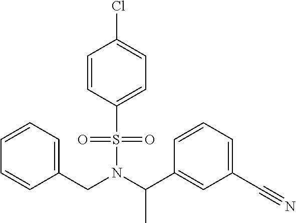 | 258 |
| 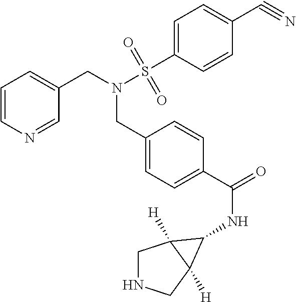 | 259 |
| 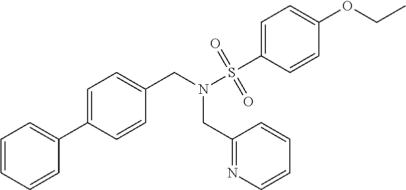 | 260 |
| 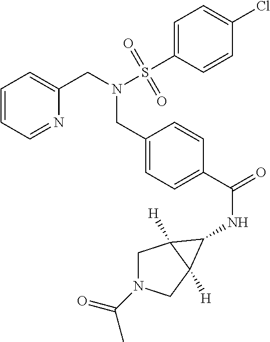 | 261 |
| 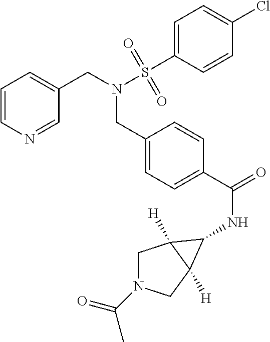 | 262 |
| 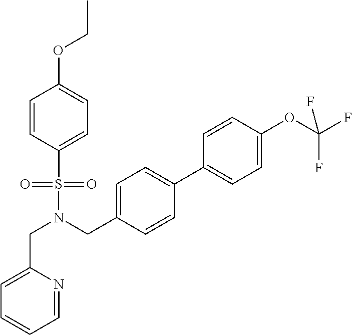 | 263 |
| 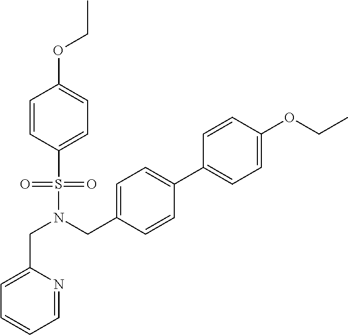 | 264 |
| 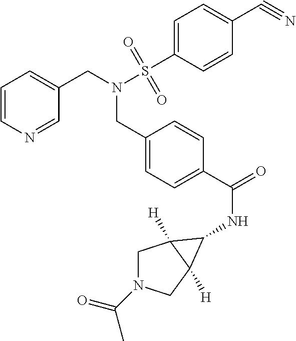 | 265 |
| 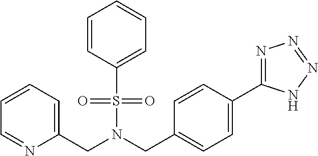 | 266 |
| 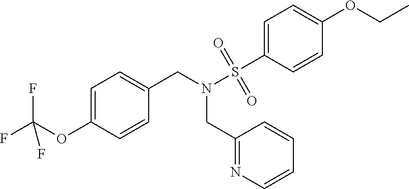 | 268 |
| 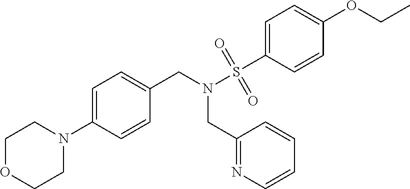 | 269 |
| 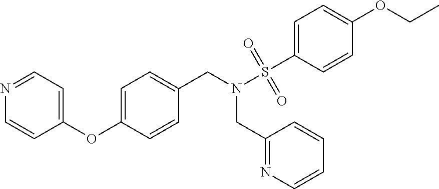 | 270 |
| 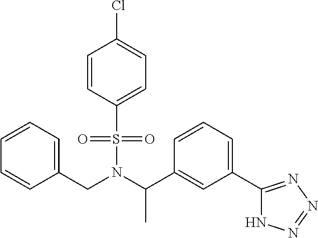 | 271 |
| 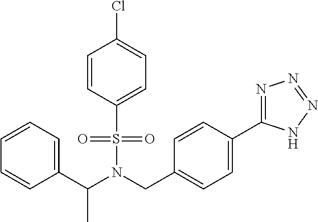 | 272 |
| 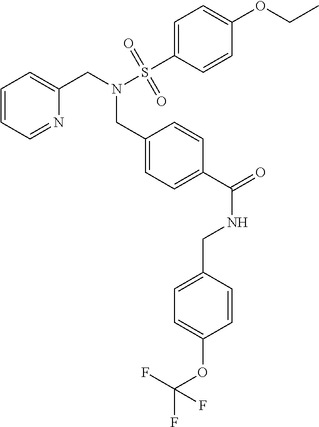 | 273 |
| 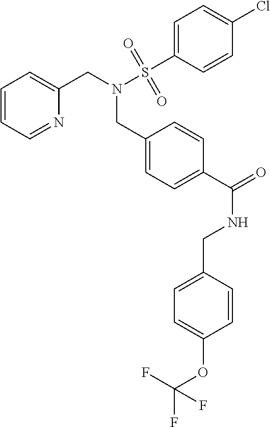 | 274 |
| 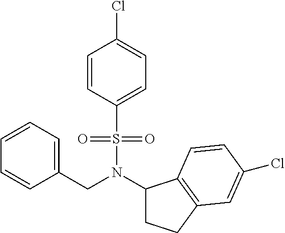 | 275 |
| 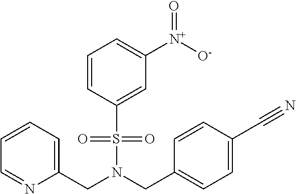 | 276 |
| 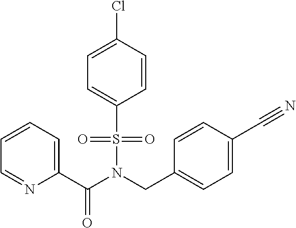 | 279 |
| 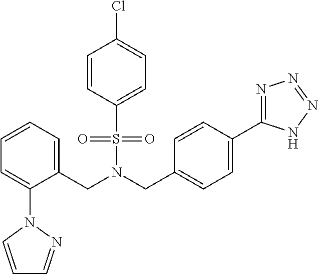 | 280 |
| 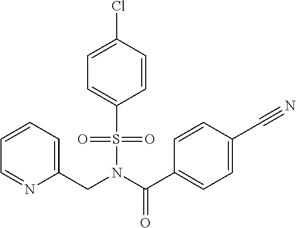 | 281 |
| 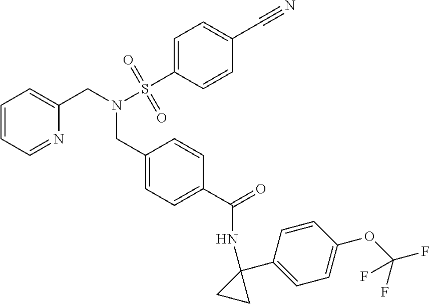 | 282 |
| 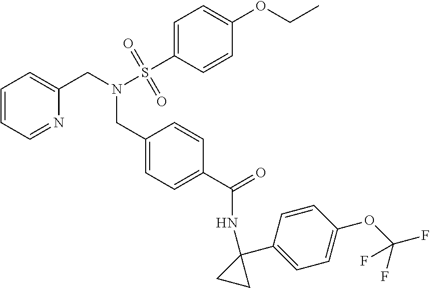 | 283 |
| 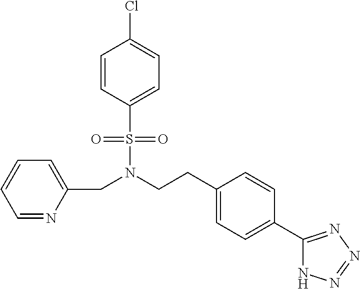 | 285 |
| 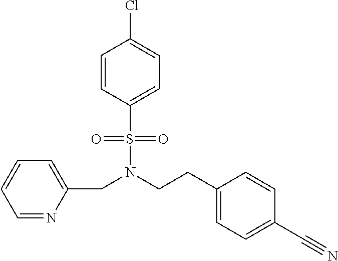 | 286 |
| 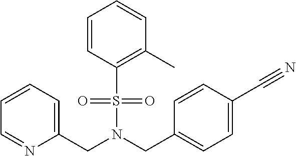 | 287 |
| 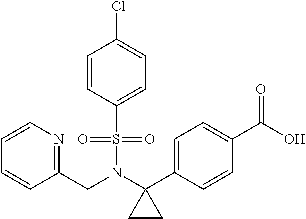 | 288 |
| 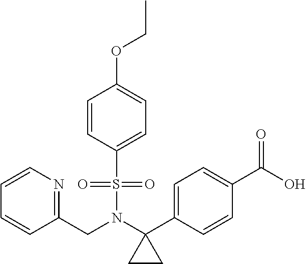 | 289 |
| 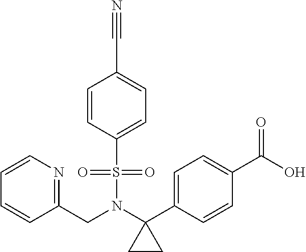 | 290 |
| 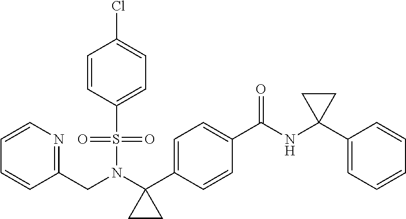 | 291 |
| 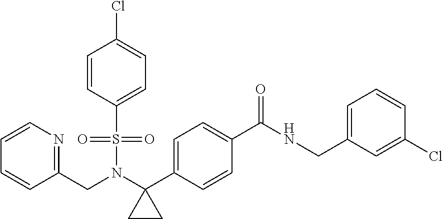 | 292 |
| 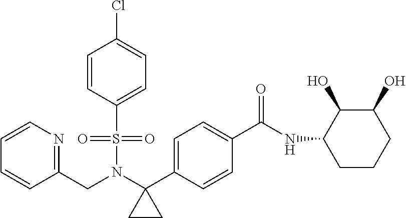 | 293 |
| 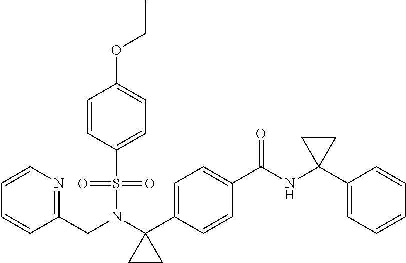 | 294 |
| 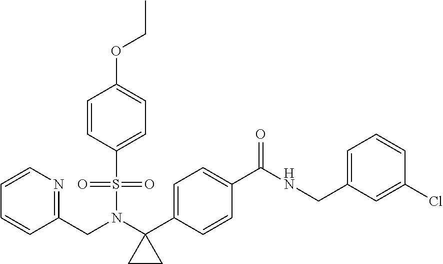 | 295 |
| 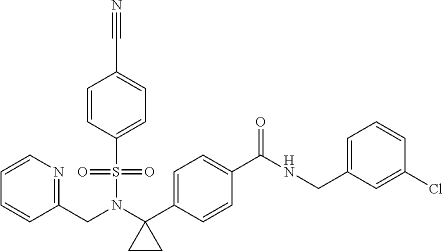 | 297 |
| 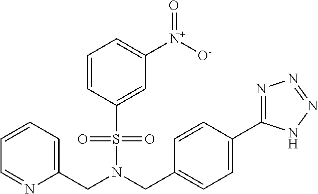 | 298 |
| 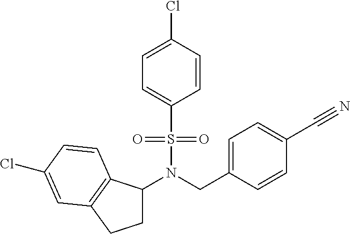 | 299 |
| 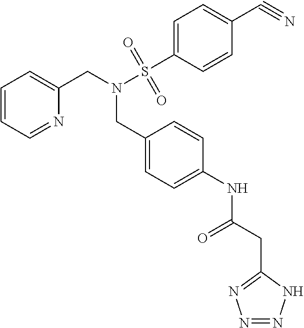 | 300 |
| 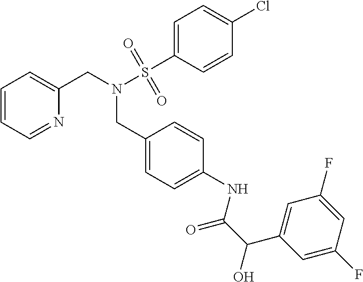 | 309 |
| 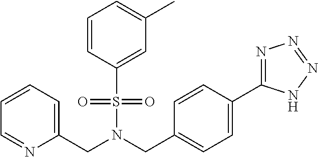 | 311 |
| 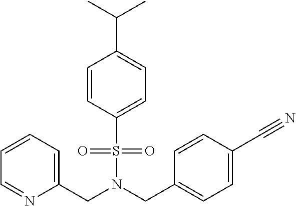 | 312 |
| 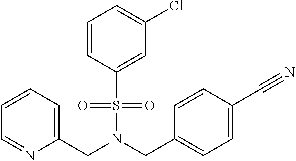 | 313 |
| 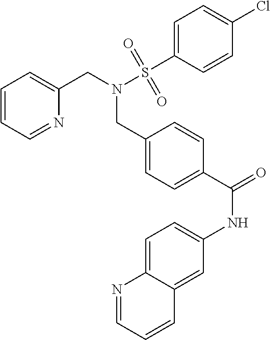 | 314 |
| 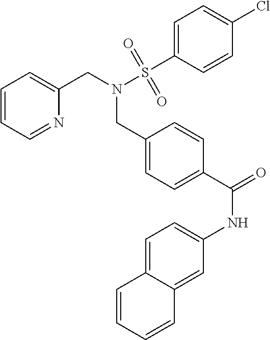 | 315 |
| 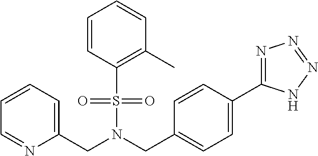 | 316 |
| 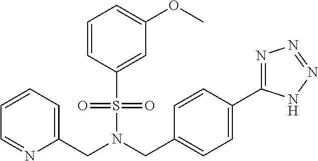 | 318 |
| 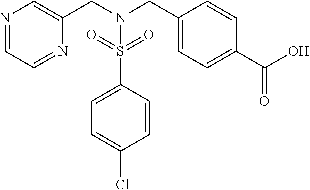 | 319 |
| 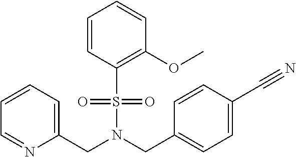 | 320 |
| 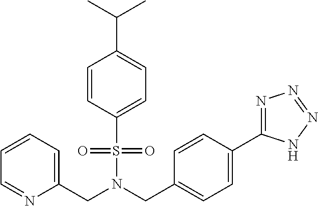 | 321 |
| 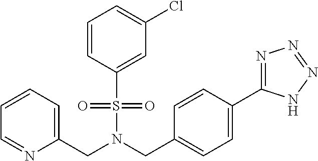 | 322 |
| 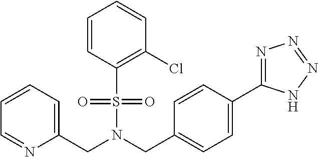 | 323 |
| 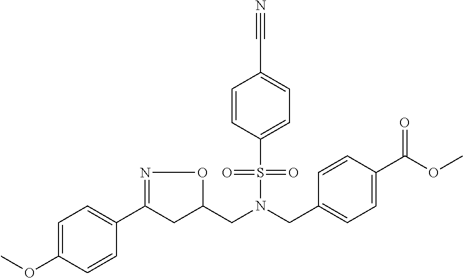 | 324 |
| 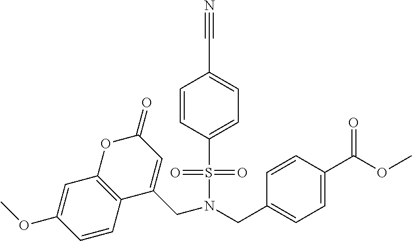 | 325 |
| 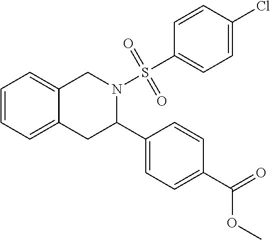 | 326 |
| 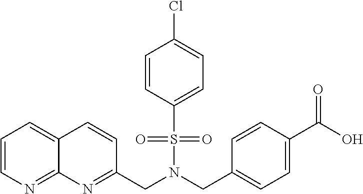 | 327 |
| 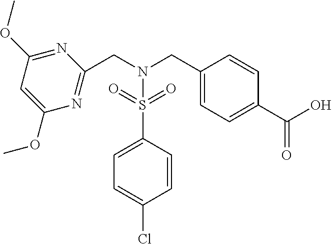 | 328 |
| 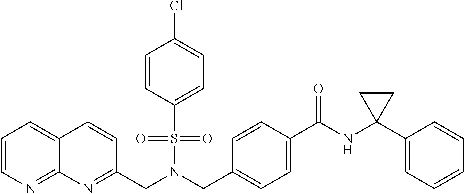 | 330 |
| 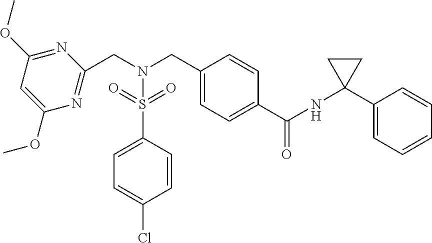 | 331 |
| 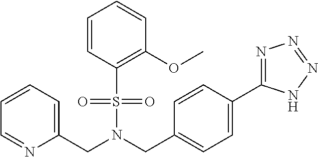 | 333 |
| 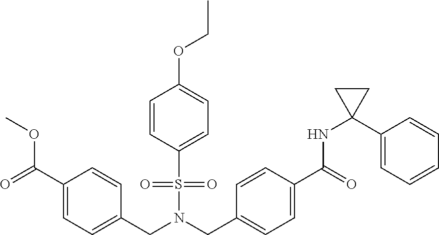 | 335 |
| 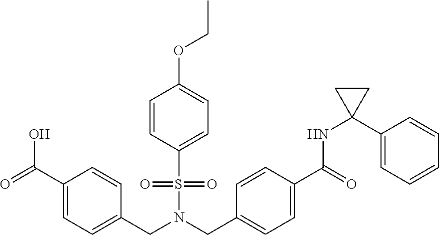 | 336 |
| 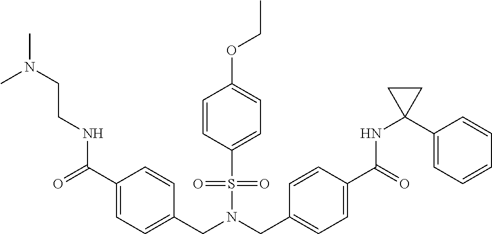 | 337 |
| 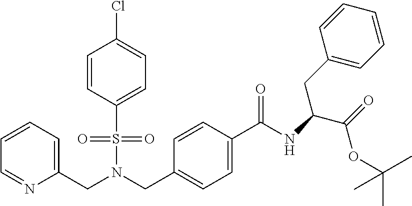 | 339 |
| 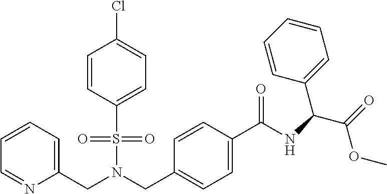 | 342 |
| 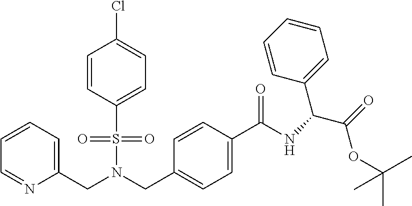 | 343 |
| 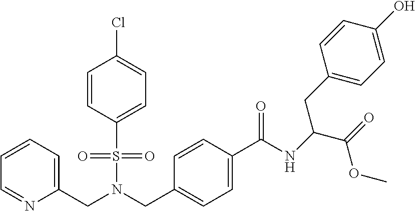 | 345 |
| 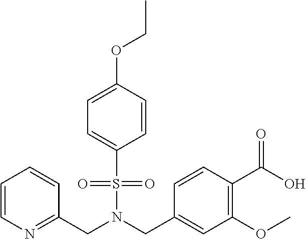 | 346 |
| 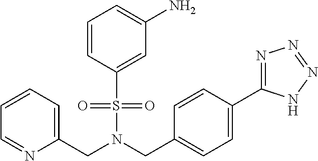 | 347 |
| 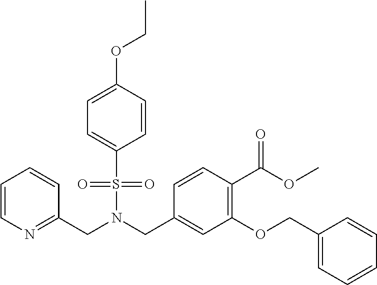 | 349 |
| 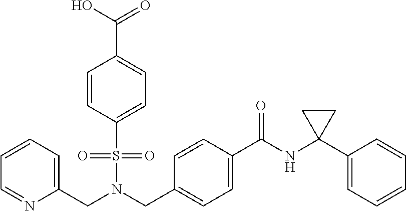 | 351 |
| 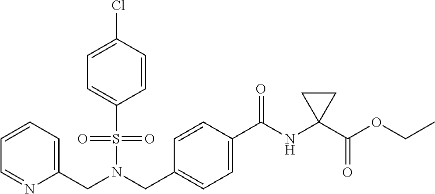 | 352 |
| 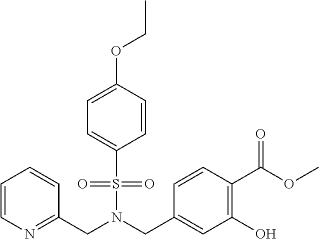 | 353 |
| 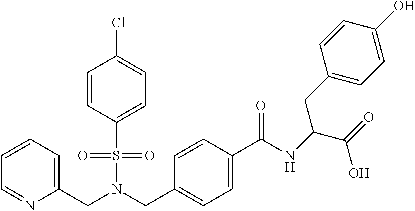 | 354 |
| 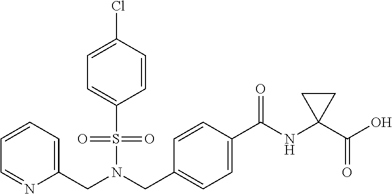 | 355 |
| 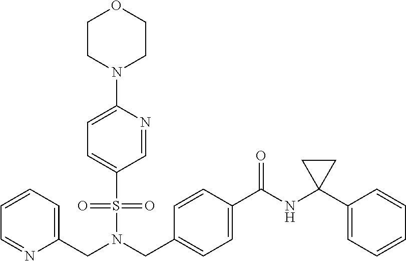 | 356 |
| 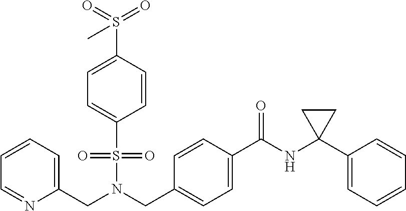 | 357 |
| 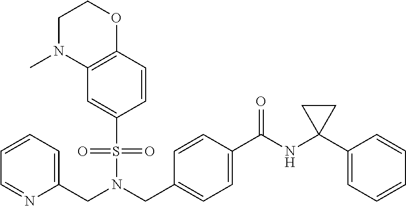 | 358 |
| 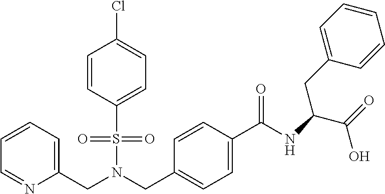 | 359 |
| 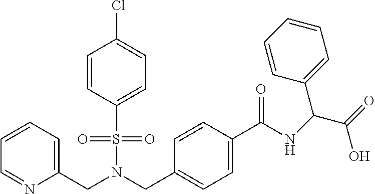 | 360 |
| 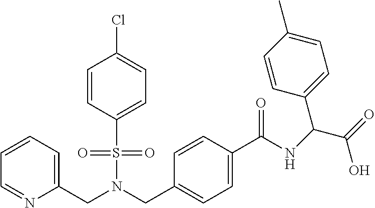 | 361 |
| 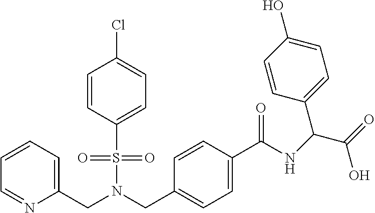 | 362 |
| 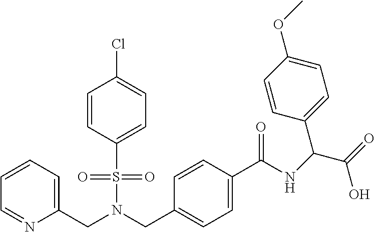 | 363 |
| 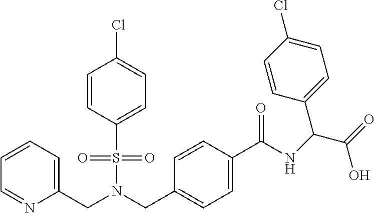 | 364 |
| 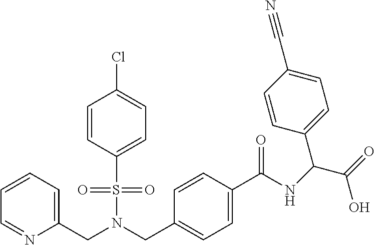 | 365 |
| 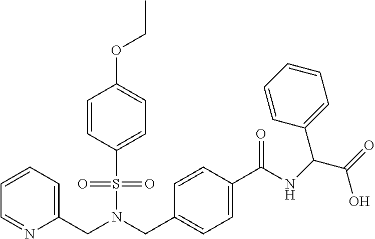 | 366 |
| 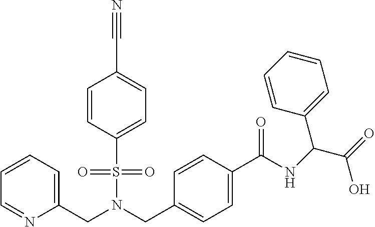 | 367 |
| 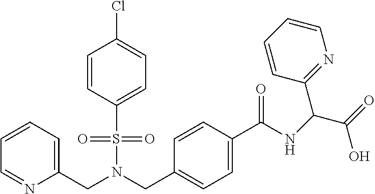 | 368 |
| 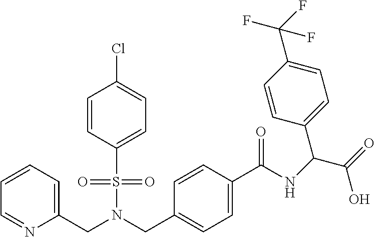 | 369 |
| 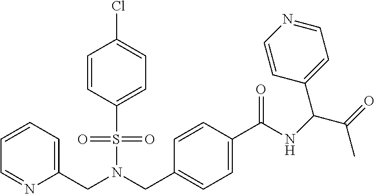 | 370 or |
| 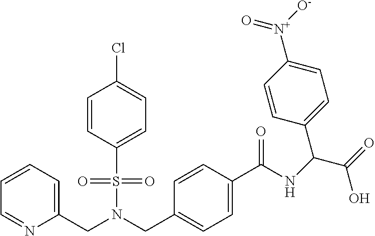 | 371 |

and pharmaceutically acceptable solvates, salts, stereoisomers and mixtures thereof.

22. A process for the preparation of the compounds of formula (I) according to claim 16, wherein R^{b} denotes CONHG′, and G′ denotes Het or a linear or branched (C1-C6)alkylene, wherein 1, 2 or 3H atoms may be replaced by OR³, CON(R³)₂, CO₂R³, an aryl group, and/or 2 geminal H atom may form a Cyc group, and wherein 1 or 2 CH₂ group may be replaced by SO₂, and salts thereof, characterized in that:
a) a compound of formula (V) 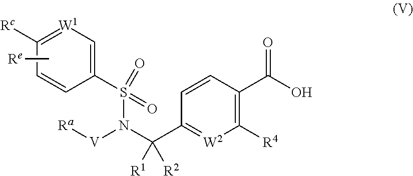
wherein R^{a}, R^{c}, R^{e}, V, R^{e}, R¹, R², R⁴, W¹ and W² are as defined in claim 16, is reacted with a compound of formula
H₂NG′;
or
b) a compound of formula (VIa) 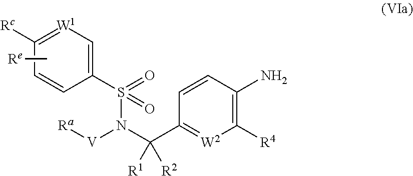
wherein R^{a}, R^{c}, R^{e}, V, R^{e}, R¹, R², R⁴, W¹ and W² are as defined in claim 16 is reacted with a compound of formula
HOOC-G′
wherein G′ denotes Het or a linear or branched (C1-C6)alkylene, wherein 1, 2 or 3 μl atoms may be replaced by OR³, CON(R³)₂, CO₂R³, an aryl group, and/or 2 geminal H atom may form a Cyc group, and wherein 1 or 2 CH₂ group may be replaced by SO₂; or
c) a compound of formula IX 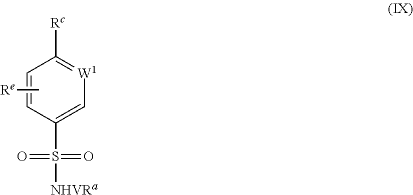
wherein R^{c}, R^{a}, R^{e}, W¹, and V are as defined in claim 16, is reacted with a compound of formula XIV 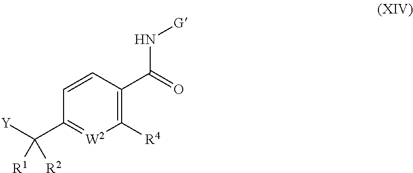
wherein R¹, R², R⁴, W² are as defined in claim 16 and Y is a leaving group.

23. A process for the preparation of the compounds of formula (I), wherein R^{b} denotes tetrazolyl, and salts thereof, characterized in that a compound of formula XIX 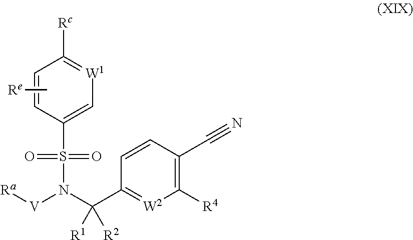
wherein R^{a}, R^{c}, R^{e}, W¹ and W² are as defined above, is reacted with TMS-N₃.

24. A kit or a set consisting of separate packs of:
(a) an effective amount of a compound according to claim 16; and
(b) an effective amount of a further active ingredient.

25. A pharmaceutical composition comprising at least one compound according to claim 16 and optionally excipients and/or adjuvants.

26. A pharmaceutical composition comprising at least one compound according to claim 16 and at least one further active ingredient.

27. A method of treating an autoimmune or chronic inflammatory disease selected from the group consisting of systemic lupus erythematosis, chronic rheumatoid arthritis, type I diabetes mellitus, inflammatory bowel disease, biliary cirrhosis, uveitis, multiple sclerosis, amyotrophic lateral sclerosis (ALS), Crohn's disease, ulcerative colitis, bullous pemphigoid, sarcoidosis, psoriasis, autoimmune myositis, Wegener's granulomatosis, ichthyosis, Graves ophthalmopathy and asthma comprising the administration of a compound according to claim 16 to a subject having said autoimmune or chronic inflammatory disease.

28. The method according to claim 27, wherein the immunoregulatory abnormality is multiple sclerosis.
